(19) United States
(12) Patent Application Publication
(10) Pub. No.: US 2025/0326769 A1
(43) Pub. Date: Oct. 23, 2025

(54) HETEROAROMATIC MACROCYCLIC ETHER CHEMOTHERAPEUTIC AGENTS

(71) Applicant: Nuvalent, Inc., Cambridge, MA (US)

(72) Inventors: Baudouin GERARD, Arlington, MA (US); Joshua Courtney HORAN, Somerville, MA (US); Henry Efrem PELISH, Newton, MA (US); Anupong TANGPEERACHAIKUL, Cambridge, MA (US)

(21) Appl. No.: 19/122,102

(22) PCT Filed: Oct. 18, 2023
(86) PCT No.: PCT/US2023/077180

§ 371 Date: Apr. 17, 2025

Related U.S. Application Data
(60) Provisional application No. 63/417,368, filed on Oct. 19, 2022.

(51) Int. Cl. C07D 513/22; A61K 31/439; A61K 45/06; C07D 491/22; C07D 498/22

(52) U.S. Cl. CPC C07D 513/22; A61K 31/439; A61K 45/06; C07D 491/22; C07D 498/22

(57) ABSTRACT

The application relates to heterocyclic heteroaromatic macrocyclic ether compounds of the general Formula (I), pharmaceutically acceptable salts of the compounds and pharmaceutical compositions thereof. The compounds act as selective inhibitors of ROS1 and/or ALK and are useful for treating or preventing cancer.

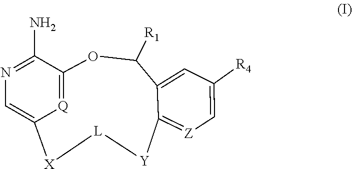

### 1. CROSS-REFERENCE TO RELATED APPLICATIONS

This application claims the benefit of priority of U.S. Provisional Application No. 63/417,368 filed Oct. 19, 2022, the entirety of which is incorporated herein by reference.

### 2. BACKGROUND

Receptor tyrosine kinases (RTKs) are cell surface enzymes that receive outside signals, such as whether to grow and divide, and transmit those signals in the cell through kinase activity. Many RTKs are proto-oncogenes; aberrant RTK activity can drive cell survival, growth and proliferation leading to cancer and related disorders. This aberrant kinase activity can be caused by mutations such as activating mutations in the kinase domain, gene rearrangements that result in fusion proteins containing the intact kinase domain, amplification and other means. RTK proto-oncogenes include ROS1, anaplastic lymphoma kinase (ALK), NTRK1 (encodes TRKA), NTRK2 (encodes TRKB), and NTRK3 (encodes TRKC).

ROS1 is an RTK proto-oncogene, with ROS1 rearrangements detected in non-small cell lung cancer (NSCLC), glioblastoma, inflammatory myofibroblastic tumor (IMT), cholangiocarcinoma, ovarian cancer, gastric cancer, colorectal cancer, angiosarcoma, and spitzoid melanoma. Oncogenic ROS1 gene fusions contain the kinase domain of ROS1 (3′ region) fused to the 5′ region of a variety of partner genes. Examples of ROS1 fusion partner genes observed in NSCLC include SLC34A2, CD74, TPM3, SDC4, EZR, LRIG3, KDELR2, CEP72, CLTL, CTNND2, GOPC, GPRC6A, LIMA1, LRIG3, MSN, MYO5C, OPRM1, SLC6A17 (putative), SLMAP, SRSF6, TFG, TMEM106B, TPD52L1, ZCCHC8 and CCDC6. Other fusion partners include CAPRIN1, CEP85L, CHCHD3, CLIP1 (putative), EEF1G, KIF21A (putative), KLC1, SART3, ST13 (putative), TRIM24 (putative), ERC1, FIP1L1, HLAA, KIAA1598, MYO5A, PPFIBP1, PWWP2A, FN1, YWHAE, CCDC30, NCOR2, NFKB2, APOB, PLG, RBP4, and GOLGB1.

ALK is an RTK proto-oncogene, with ALK rearrangements detected in many cancers, including NSCLC, anaplastic large cell lymphoma (ALCL), IMT, diffuse large B-cell lymphoma (DLBCL), esophageal squamous cell carcinoma (ESCC), renal medullary carcinoma, renal cell carcinoma, breast cancer, colon cancer, serous ovarian carcinoma, papillary thyroid cancer, and spitzoid tumors, and ALK activating mutations detected in neuroblastoma. Oncogenic ALK gene fusions contain the kinase domain of ALK (3′ region) fused to the 5′ region of more than 20 different partner genes, the most common being EML4 in NSCLC and NPM in ALCL. Other partner genes include TMP1, WDCP, GTF2IRD1, TPM3, TPM4, CLTC, LMNA, PRKAR1A, RANBP2, TFG, FN1, KLC1, VCL, STRN, HIP1, DCTN1, SQSTM1, TPR, CRIM1, PTPN3, FBXO36, ATIC and KIF5B.

NTRK1, NTRK2 and NTRK3 are RTK proto-oncogenes that encode TRK-family kinases, with NTRK1, NTRK2 and NTRK3 chromosomal rearrangements detected at low frequency in many cancers. For treatment of ROS1-positive or ALK-positive patients, however, TRK inhibition, particularly in the central nervous system (CNS), has been associated with adverse reactions, including dizziness/ataxia/gait disturbance, paraesthesia, weight gain and cognitive changes.

Existing agents used to treat oncogenic ROS1 and ALK have substantial deficiencies. These deficiencies may represent one or more of the following: associated TRK inhibition, limited CNS activity, and inadequate activity against resistance mutations. Treatment of ROS1-positive or ALK-positive patients accompanied by TRK inhibition is associated with adverse reactions, particularly in the CNS, including dizziness/ataxia/gait disturbance, paraesthesia, weight gain and cognitive changes. Additionally, there is a need for CNS-penetrant and TRK-sparing inhibitors of the wild type ROS1 kinase domain and ROS1 with acquired resistance mutations occurring either individually or in combination, including G2032R, D2033N, S1986F, S1986Y, L2026M, L1951R, E1935G, L1947R, G1971E, E1974K, L1982F, F2004C, F2004V, E2020K, C2060G, F2075V, V2089M, V2098I, G2101A, D2113N, D2113G, L2155S, L2032K, and L2086F. Likewise, there is a need for CNS-penetrant and TRK-sparing inhibitors of ALK with acquired resistance mutations. A variety of ALK drug resistance mutations, occurring either individually or in combination, have been reported, including G1202R, L1196M, G1269A, C1156Y, I1171T, I1171N, I1171S, F1174L, V1180L, S1206Y, E1210K, 1151Tins, F1174C, G1202del, D1203N, S1206Y, S1206C, L1152R, L1196Q, L1198P, L1198F, R1275Q, L1152P, C1156T, and F1245V.

### 3. SUMMARY

In one embodiment, provided herein are compounds of Formula (I):

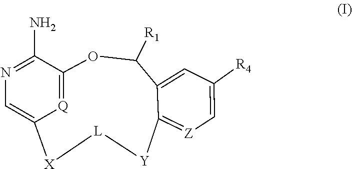

or a stereoisomer, a mixture of stereoisomers, or a pharmaceutically acceptable salt thereof, wherein Q, Z, L, X, Y, R₁, and R₄ are as defined herein or elsewhere.

In one embodiment, provided herein is a pharmaceutical composition suitable for use in a subject in the treatment or prevention of cancer comprising an effective amount of any of the compounds described herein (e.g., a compound provided herein, such as a compound of Formula (I), or a pharmaceutically acceptable salt thereof, and one or more pharmaceutically acceptable excipients. In certain embodiments, the pharmaceutical preparations may be for use in treating or preventing a condition or disease as described herein.

In one embodiment, provided herein are methods of treating cancer that is characterized by one or more mutations in the ROS1 or ALK genes, comprising administering to a subject in need thereof an effective amount of a compound provided herein (e.g., a compound of Formula (I) or any of the embodiments thereof provided herein). In certain embodiments the compound is an inhibitor of ROS1, other embodiments the compound is an inhibitor of ALK, in additional embodiments the compound is an inhibitor of ROS1 and ALK. In certain aspects, the human subject is in need of such treatment. In one embodiment, without being bound by a particular theory, one or more compound provided herein selectively inhibit an ALK mutation over TRK (e.g., TRKA, TRKB, and/or TRBC), wherein the ALK mutation is I1171X¹ (X¹ is N, S, or T) and/or D1203N.

These cancers include, but are not limited to, non-small cell lung cancer, inflammatory myofibroblastic tumor, ovarian cancer, spitzoid melanoma, glioblastoma, cholangiocarcinoma, gastric cancer, colorectal cancer, angiosarcoma, anaplastic large cell lymphoma, diffuse large B-cell lymphoma, esophageal squamous cell carcinoma, renal medullary carcinoma, renal cell carcinoma, breast cancer, papillary thyroid cancer, and neuroblastoma.

In some embodiments, the method of treating or preventing cancer may comprise administering a compound of Formula (I) conjointly with one or more other chemotherapeutic agent(s).

### 4. DETAILED DESCRIPTION

### 4.1 Definitions

Unless defined otherwise, all technical and scientific terms used herein have the meaning commonly understood by a person skilled in the art of the present disclosure. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

In some embodiments, chemical structures are disclosed with a corresponding chemical name. In case of conflict, the chemical structure controls the meaning, rather than the name.

As used herein and unless otherwise specified, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited are not substantially changed by the presence of more than that which is recited, but excludes prior art embodiments.

Unless specifically stated or obvious from context, as used herein, the term “or” is understood to be inclusive. Unless specifically stated or obvious from context otherwise, as used herein, the terms “a”, “an”, and “the” are understood to be singular or plural.

Stereoisomers as used herein refer to the various stereoisomeric forms of a compound that comprises one or more asymmetric centers or stereohindrance in the structure. In some embodiments, a stereoisomer is an enantiomer, a mixture of enantiomers, an atropisomer, a mixture of atropisomers, a tautomer, or a mixture of tautomers thereof. For example, the compounds described herein can be in the form of an individual enantiomer, diastereomer or geometric isomer (e.g., an atropisomer), or can be in the form of a mixture of stereoisomers, including racemic mixtures and mixtures enriched in one or more stereoisomer. In some embodiments, compounds provided herein may be atropisomers. In certain embodiments, atropisomers are stereoisomers arising because of hindered rotation about a single bond, where energy differences due to steric strain or other contributors create a barrier to rotation that is high enough to allow for isolation of individual conformers. Stereoisomers can be isolated from mixtures by methods known to those skilled in the art, including chiral high pressure liquid chromatography (HPLC) and the formation and crystallization of chiral salts; or in one embodiment isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H. Tables of Resolving Agents and Optical Resolutions p. 268 (E. L. Eliel, Ed., Univ. of Notre Dame Press, Notre Dame, IN 1972). Additionally provided herein are compounds as individual isomers substantially free of other isomers, and alternatively, as mixtures of various isomers.

The term “acyl” is art-recognized and refers to a group represented by the general formula hydrocarbylC(O)—, in one embodiment alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, in one embodiment alkylC(O)O—.

The term “alkoxy” refers to an alkyl group, in one embodiment a lower alkyl group, having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkenyl”, as used herein, refers to an aliphatic group containing at least one double bond and is intended to include both “unsubstituted alkenyls” and “substituted alkenyls”, the latter of which refers to alkenyl moieties having substituents replacing a hydrogen on one or more carbons of the alkenyl group. Such substituents may occur on one or more carbons that are included or not included in one or more double bonds. Moreover, such substituents include all those contemplated for alkyl groups, as discussed below, except where stability is prohibitive. For example, substitution of alkenyl groups by one or more alkyl, carbocyclyl, aryl, heterocyclyl, or heteroaryl groups is contemplated.

An “alkyl” group or “alkane” is a straight chained or branched non-aromatic hydrocarbon which is completely saturated. Typically, a straight chained or branched alkyl group has from 1 to about 20 carbon atoms, in one embodiment from 1 to about 10 unless otherwise defined. Examples of straight chained and branched alkyl groups include methyl, ethyl, n-propyl, iso-propyl, n-butyl, sec-butyl, tert-butyl, pentyl, hexyl, pentyl and octyl. A C₁-C₆ straight chained or branched alkyl group is also referred to as a “lower alkyl” group.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents, if not otherwise specified, can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

The term “C_{x-y}” when used in conjunction with a chemical moiety, such as, acyl, acyloxy, alkyl, alkenyl, alkynyl, or alkoxy is meant to include groups that contain from x to y carbons in the chain. For example, the term “C_{x-y} alkyl” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-tirfluoroethyl, etc. Co alkyl indicates a hydrogen where the group is in a terminal position, a bond if internal. The terms “C_{2-y} alkenyl” and “C_{2-y} alkynyl” refer to substituted or unsubstituted unsaturated aliphatic groups analogous in length and possible substitution to the alkyls described above, but that contain at least one double or triple bond respectively.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “alkynyl”, as used herein, refers to an aliphatic group containing at least one triple bond and is intended to include both “unsubstituted alkynyls” and “substituted alkynyls”, the latter of which refers to alkynyl moieties having substituents replacing a hydrogen on one or more carbons of the alkynyl group. Such substituents may occur on one or more carbons that are included or not included in one or more triple bonds. Moreover, such substituents include all those contemplated for alkyl groups, as discussed above, except where stability is prohibitive. For example, substitution of alkynyl groups by one or more alkyl, carbocyclyl, aryl, heterocyclyl, or heteroaryl groups is contemplated.

The term “amide”, as used herein, refers to a group

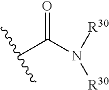

wherein each R³⁰ independently represents a hydrogen or hydrocarbyl group, or two R³⁰ are taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

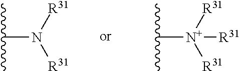

wherein each R³¹ independently represents a hydrogen or a hydrocarbyl group, or two R³¹ are taken together with the N atom to which they are attached complete a heterocycle having from 4 to 8 atoms in the ring structure. The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. In one embodiment, the ring is a 5- to 7-membered ring, in one embodiment a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

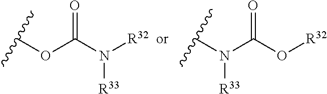

wherein R³² and R³³ independently represent hydrogen or a hydrocarbyl group, such as an alkyl group, or R³² and R³³ taken together with the intervening atom(s) complete a heterocycle having from 4 to 8 atoms in the ring structure.

The terms “carbocycle”, and “carbocyclic”, as used herein, refers to a saturated or unsaturated ring in which each atom of the ring is carbon. The term carbocycle includes both aromatic carbocycles and non-aromatic carbocycles. Non-aromatic carbocycles include both cycloalkane rings, in which all carbon atoms are saturated, and cycloalkene rings, which contain at least one double bond.

The term “carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

A “cycloalkyl” group is a cyclic hydrocarbon which is completely saturated. “Cycloalkyl” includes monocyclic and bicyclic rings. Typically, a monocyclic cycloalkyl group has from 3 to about 10 carbon atoms, more typically 3 to 8 carbon atoms unless otherwise defined. The second ring of a bicyclic cycloalkyl may be selected from saturated, unsaturated and aromatic rings. Cycloalkyl includes bicyclic molecules in which one, two or three or more atoms are shared between the two rings. The term “fused cycloalkyl” refers to a bicyclic cycloalkyl in which each of the rings shares two adjacent atoms with the other ring. The second ring of a fused bicyclic cycloalkyl may be selected from saturated, unsaturated and aromatic rings. A “cycloalkenyl” group is a cyclic hydrocarbon containing one or more double bonds.

The term “carbocyclylalkyl”, as used herein, refers to an alkyl group substituted with a carbocycle group.

The term “C₃₋₄ cycloalkylmethyl”, as used herein, refers to a methyl group substituted with a carbocycle group containing 3 to 4 carbon atoms.

The term “carbonate” is art-recognized and refers to a group —OCO₂—R³⁴, wherein R³⁴ represents a hydrocarbyl group.

The term “carboxy”, as used herein, refers to a group represented by the formula —CO₂H.

The term “ester”, as used herein, refers to a group —C(O)OR³⁵ wherein R³⁵ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The term “heteroalkyl”, as used herein, refers to a saturated or unsaturated chain of carbon atoms and at least one heteroatom, wherein no two heteroatoms are adjacent.

The terms “heteroaryl” and “hetaryl” include substituted or unsubstituted aromatic single ring structures, in one embodiment 5- to 7-membered rings, in one embodiment 5- to 6-membered rings, whose ring structures include at least one heteroatom, in one embodiment one to four heteroatoms, in one embodiment one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The asterisk (*) notation on a heteroarylene ring moiety corresponding to X or Y in the compound of Formula (I) identifies the ring atom of the moiety bonded to the L group between X and Y, as exemplified below:

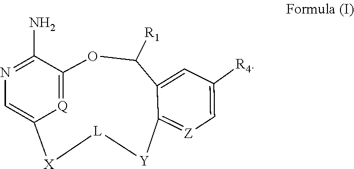

For example, “1*, 5-substituted-imidazolylene” for Y means substituted:

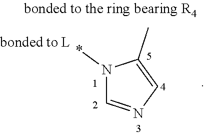 [SMILES: *n1cncc1C ; "."]

IUPAC numbering rules for heteroarylene rings are used throughout the specification to designate ring atom positions, as shown above. In this example, the 1-position of the imidazolylene is bonded to the L group, so it is indicated with the asterisk. The asterisk notation is used in both the names and structures of heteroarylenes for X and Y. Here, for Y the ring atom at the 5-position is not marked because it is bound to the ring bearing variable R₄.

For X, an exemplary ring would be “1,5*-substituted-imidazolylene” as shown below.

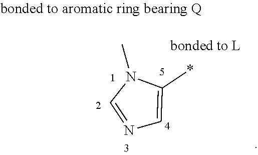 [SMILES: *c1cncn1C ; "."]

The ring atom bound to the L group (the 5-position in this example) is indicated with the asterisk in both the names and structures of ring X heteroarylenes. The ring atom bonded to the aromatic ring bearing Q is not marked.

The term “heteroatom” as used herein means an atom of any element other than carbon or hydrogen. in one embodiment, heteroatoms are nitrogen, oxygen, and sulfur.

The terms “heterocyclyl”, “heterocycle”, and “heterocyclic” refer to substituted or unsubstituted non-aromatic ring structures, in one embodiment 3- to 10-membered rings, more in one embodiment 3- to 7-membered rings, whose ring structures include at least one heteroatom, in one embodiment one to four heteroatoms, in one embodiment one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “heterocyclylalkyl”, as used herein, refers to an alkyl group substituted with a heterocycle group.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocyclyl, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer non-hydrogen atoms in the substituent, in one embodiment six or fewer. A “lower alkyl”, for example, refers to an alkyl group that contains ten or fewer carbon atoms, in one embodiment six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, in one embodiment from 5 to 7.

The term “silyl” refers to a silicon moiety with three hydrocarbyl moieties attached thereto.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. As used herein and unless otherwise specified, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that substituents can themselves be substituted, if appropriate. Unless specifically stated as “unsubstituted,” references to chemical moieties herein are understood to include substituted variants. For example, reference to an “aryl” group or moiety implicitly includes both substituted and unsubstituted variants.

The term “sulfate” is art-recognized and refers to the group —OSO₃H, or a pharmaceutically acceptable salt thereof.

The term “sulfonamide” is art-recognized and refers to the group represented by the general formulae

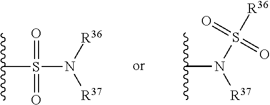

wherein R³⁶ and R³⁷ independently represent hydrogen or hydrocarbyl, such as alkyl, or R³⁶ and R³⁷ taken together with the intervening atom(s) complete a heterocycle having from 4 to 8 atoms in the ring structure.

The term “sulfoxide” is art-recognized and refers to the group —S(O)—R³⁸, wherein R³⁸ represents a hydrocarbyl.

The term “sulfonate” is art-recognized and refers to the group SO₃H, or a pharmaceutically acceptable salt thereof.

The term “sulfone” is art-recognized and refers to the group —S(O)₂—R³⁹, wherein R³⁹ represents a hydrocarbyl.

The term “thioalkyl”, as used herein, refers to an alkyl group substituted with a thiol group.

The term “thioester”, as used herein, refers to a group —C(O)SR⁴⁰ or —SC(O)R⁴⁰ wherein R¹⁰ represents a hydrocarbyl.

The term “thioether”, as used herein, is equivalent to an ether, wherein the oxygen is replaced with a sulfur.

The term “urea” is art-recognized and may be represented by the general formula

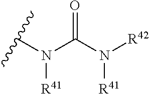

wherein R⁴¹ and R⁴² independently represent hydrogen or a hydrocarbyl, such as alkyl, or either occurrence of R⁴¹ taken together with R⁴² and the intervening atom(s) complete a heterocycle having from 4 to 8 atoms in the ring structure.

The term “protecting group” refers to a group of atoms that, when attached to a reactive functional group in a molecule, mask, reduce or prevent the reactivity of the functional group. Typically, a protecting group may be selectively removed as desired during the course of a synthesis. Examples of protecting groups can be found in Greene and Wuts, Protective Groups in Organic Chemistry, 3^{rd} Ed., 1999, John Wiley & Sons, NY and Harrison et al., Compendium of Synthetic Organic Methods, Vols. 1-8, 1971-1996, John Wiley & Sons, NY. Representative nitrogen protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“TES”), trityl and substituted trityl groups, allyloxycarbonyl, 9-fluorenylmethyloxycarbonyl (“FMOC”), nitro-veratryloxycarbonyl (“NVOC”) and the like. Representative hydroxyl protecting groups include, but are not limited to, those where the hydroxyl group is either acylated (esterified) or alkylated such as benzyl and trityl ethers, as well as alkyl ethers, tetrahydropyranyl ethers, trialkylsilyl ethers (e.g., TMS or TIPS groups), glycol ethers, such as ethylene glycol and propylene glycol derivatives and allyl ethers.

In certain embodiments, compounds provided herein may be racemic. In certain embodiments, compounds provided herein may be enriched in one enantiomer. For example, a compound provided herein may have greater than about 30% ee, about 40% ee, about 50% ee, about 60% ee, about 70% ee, about 80% ee, about 90% ee, or even about 95% or greater ee. In certain embodiments, compounds provided herein may have more than one stereocenter. In certain such embodiments, compounds provided herein may be enriched in one or more diastereomer. For example, a compound provided herein may have greater than about 30% de, about 40% de, about 50% de, about 60% de, about 70% de, about 80% de, about 90% de, or even about 95% or greater de.

In certain embodiments, the therapeutic preparation may be enriched to provide predominantly one enantiomer of a compound (e.g., of Formula (I)). An enantiomerically enriched mixture may comprise, for example, at least about 60 mol percent of one enantiomer, or in one embodiment at least about 75, about 90, about 95, or even about 99 mol percent. In certain embodiments, the compound enriched in one enantiomer is substantially free of the other enantiomer, wherein substantially free means that the substance in question makes up less than about 10%, or less than about 5%, or less than about 4%, or less than about 3%, or less than about 2%, or less than about 1% as compared to the amount of the other enantiomer, e.g., in the composition or compound mixture. For example, if a composition or compound mixture contains about 98 grams of a first enantiomer and about 2 grams of a second enantiomer, it would be said to contain about 98 mol percent of the first enantiomer and only about 2% of the second enantiomer.

In certain embodiments, the therapeutic preparation may be enriched to provide predominantly one diastereomer of a compound (e.g., of Formula (I)). A diastereomerically enriched mixture may comprise, for example, at least about 60 mol percent of one diastereomer, or in one embodiment at least about 75, about 90, about 95, or even about 99 mol percent.

In some embodiments, a moiety in a compound exists as a mixture of tautomers. A “tautomer” is a structural isomer of a moiety or a compound that readily interconverts with another structural isomer. For example, a pyrazole ring has two tautomers:

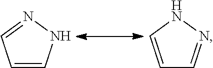 [SMILES: c1cn[nH]c1 ; c1cn[nH]c1]

which differ in the positions of the pi-bonds and a hydrogen atom. Unless explicitly stated otherwise, a drawing of one tautomer of a moiety or a compound encompasses all of the possible tautomers.

The term “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or other primates (e.g., cynomolgus monkeys, rhesus monkeys); mammals, including commercially relevant mammals such as cattle, pigs, horses, sheep, goats, cats, and/or dogs; and/or birds, including commercially relevant birds such as chickens, ducks, geese, quail, and/or turkeys. In one embodiment, subjects are humans.

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample. These effects are also called “prophylactic” effects. Thus, as used herein and unless otherwise specified, the terms “prevention” and “preventing” refer to an approach for obtaining beneficial or desired results including, but not limited, to prophylactic benefit. For prophylactic benefit, a therapeutic can be administered to a patient at risk of developing a particular disease, or to a patient reporting one or more of the physiological symptoms of a disease, even though a diagnosis of this disease may not have been made. In one embodiment, a therapeutic is administered prior to clinical manifestation of the unwanted condition (e.g., disease or other unwanted state of the subject) for prophylactic benefit (e.g., it protects the subject against developing the unwanted condition).

As used herein and unless otherwise specified, the terms “treatment” and “treating” refer to therapeutic or palliative measures. Beneficial or desired clinical results include, but are not limited to, alleviation, in whole or in part, of symptoms associated with a disease or disorder or condition, diminishment of the extent of disease, stabilized (i.e., not worsening) state of disease, delay or slowing of disease progression, amelioration or palliation of the disease state (e.g., one or more symptoms of the disease), and remission (whether partial or total), whether detectable or undetectable. “Treatment” can also mean prolonging survival as compared to expected survival if not receiving treatment. In one embodiment, “treatment” comprises administration of a therapeutic after manifestation of the unwanted condition (i.e., it is intended to diminish, ameliorate, or stabilize the existing unwanted condition or side effects thereof).

As used herein and unless otherwise specified, “cancer” refers to any malignant and/or invasive growth or tumor caused by abnormal cell growth, including solid tumors named for the type of cells that form them, cancer of blood, bone marrow, or the lymphatic system. Examples of solid tumors include but not limited to sarcomas and carcinomas. Examples of cancers of the blood include but not limited to leukemias, lymphomas and myeloma. Cancer includes, but not limited to a primary cancer that originates at a specific site in the body, a metastatic cancer that has spread from the place in which it started to other parts of the body, a recurrence from the original primary cancer after remission, and a second primary cancer that is a new primary cancer in a person with a history of previous cancer of different type from latter one.

As used herein and unless otherwise specified, “abnormal cell growth” refers to cell growth that is independent of normal regulatory mechanisms (e.g., loss of contact inhibition). Abnormal cell growth may be benign (not cancerous), or malignant (cancerous). In some embodiments of the methods provided herein, the abnormal cell growth is cancer.

In some embodiments, the abnormal cell growth is cancer mediated by an anaplastic lymphoma kinase (ALK). In some such embodiments, the ALK is a genetically altered ALK. In other embodiments, the abnormal cell growth is cancer mediated by ROS I kinase. In some such embodiments, the ROS1 kinase is a genetically altered ROS1 kinase. In some embodiments, the abnormal cell growth is cancer, in particular NSCLC. In some such embodiments, the NSCLC is mediated by ALK or ROS1. In specific embodiments, the cancer is NSCLC mediated by genetically altered ALK or genetically altered ROS1.

As used herein and unless otherwise indicated, the term “managing” encompasses preventing the recurrence of the particular disease or disorder in a patient who had suffered from it, lengthening the time a patient who had suffered from the disease or disorder remains in remission, reducing mortality rates of the patients, and/or maintaining a reduction in severity or avoidance of a symptom associated with the disease or condition being managed.

The term “prodrug” is intended to encompass compounds which, under physiologic conditions, are converted into the therapeutically active agents provided herein (e.g., a compound of Formula (I)). A common method for making a prodrug is to include one or more selected moieties which are hydrolyzed under physiologic conditions to reveal the desired molecule. In other embodiments, the prodrug is converted by an enzymatic activity of the subject. For example, esters or carbonates (e.g., esters or carbonates of alcohols or carboxylic acids) are prodrugs provided herein. In certain embodiments, some or all of the compounds of Formula (I) in a formulation represented above can be replaced with the corresponding suitable prodrug, e.g., wherein a hydroxyl in the parent compound is presented as an ester or a carbonate or carboxylic acid.

An “effective amount”, as used herein, refers to an amount that is sufficient to achieve a desired biological effect. A “therapeutically effective amount”, as used herein, refers to an amount that is sufficient to achieve a desired therapeutic effect. For example, a therapeutically effective amount can refer to an amount that is sufficient to improve at least one sign or symptom of cancer.

A “response” to a method of treatment can include a decrease in or amelioration of negative symptoms, a decrease in the progression of a disease or symptoms thereof, an increase in beneficial symptoms or clinical outcomes, a lessening of side effects, stabilization of disease, partial or complete remedy of disease, among others.

As used herein and unless otherwise indicated, the term “relapsed” refers to a disorder, disease, or condition that responded to prior treatment (e.g., achieved a complete response) then had progression. The prior treatment can include one or more lines of therapy.

As used herein and unless otherwise indicated, the term “refractory” refers to a disorder, disease, or condition that has not responded to prior treatment that can include one or more lines of therapy.

As used herein, and unless otherwise specified, the terms “about” and “approximately,” when used in connection with doses, amounts, or weight percents of ingredients of a composition or a dosage form, mean a dose, amount, or weight percent that is recognized by one of ordinary skill in the art to provide a pharmacological effect equivalent to that obtained from the specified dose, amount, or weight percent. In certain embodiments, the terms “about” and “approximately,” when used in this context, contemplate a dose, amount, or weight percent within 30%, within 20%, within 15%, within 10%, or within 5%, of the specified dose, amount, or weight percent.

The term “between” includes the endpoint numbers on both limits of the range. For example, the range described by “between 3 and 5” is inclusive of the numbers “3” and “5”.

As used herein and unless otherwise specified, the term “pharmaceutically acceptable salt” refers to those salts which are, within the scope of sound medical judgment, suitable for use in contact with the tissues of subjects without undue toxicity, irritation, allergic response and the like, and are commensurate with a reasonable benefit/risk ratio. Pharmaceutically acceptable salts are well known in the art. For example, Berge et al. describes pharmaceutically acceptable salts in detail in J. Pharmaceutical Sciences (1977) 66:1-19. In certain embodiments, pharmaceutically acceptable salts include, but are not limited to, alkyl, dialkyl, trialkyl or tetra-alkyl ammonium salts. In certain embodiments, pharmaceutically acceptable salts include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, pharmaceutically acceptable salts include, but are not limited to, Na, Ca, K, Mg, Zn or other metal salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Pharmaceutically acceptable anionic salts include, but are not limited to, acetate, aspartate, benzenesulfonate, benzoate, besylate, bicarbonate, bitartrate, bromide, camsylate, carbonate, chloride, citrate, decanoate, edetate, esylate, fumarate, gluceptate, gluconate, glutamate, glycolate, hexanoate, hydroxynaphthoate, iodide, isethionate, lactate, lactobionate, malate, maleate, mandelate, mesylate, methylsulfate, mucate, napsylate, nitrate, octanoate, oleate, pamoate, pantothenate, phosphate, polygalacturonate, propionate, salicylate, stearate, acetate, succinate, sulfate, tartrate, teoclate, and tosylate.

### 4.2 Compounds

In one embodiment, provided herein is a compound of Formula (I):

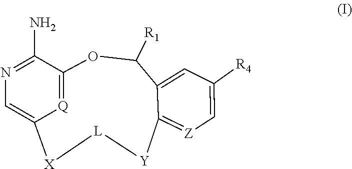

or a stereoisomer, a mixture of stereoisomers, or a pharmaceutically acceptable salt thereof, wherein:
Q is CH or N;
Z is CR₅ or N;
L is —CH₂—, C═O, or —O—;
X is a 5-membered heteroarylene, comprising 1 to 3 heteroatoms selected from the group consisting of nitrogen, oxygen and sulfur; wherein the 5-membered heteroarylene is substituted with 1, 2, or 3 occurrences of R₂;
Y is a 5- or 6-membered heteroarylene, comprising 1 to 3 heteroatoms selected from the group consisting of nitrogen, oxygen and sulfur; wherein the 5- or 6-membered heteroarylene is substituted with 0, 1, or 2 occurrences of R₃;
R₁ is selected from the group consisting of H, methyl, and hydroxymethyl;
each instance of R₂ is independently selected from the group consisting of H, CN, halo,

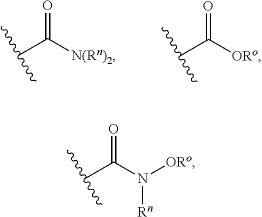

—CO—C₁₋₄ alkyl, 5-membered heteroaryl, C₁₋₄ alkyl-SO—, C₁₋₄ alkyl-SO₂—, C₁₋₄ alkoxy, C₁₋₄ alkyl, halo-C₁₋₄ alkyl, C₁₋₄ alkoxy-C₁₋₄ alkyl, C₃₋₄ cycloalkylmethyl, C₃₋₆ cycloalkyl, and C₃₋₆ heterocyclyl; provided that, when L is —CH₂—, at least one occurrence of R₂ is

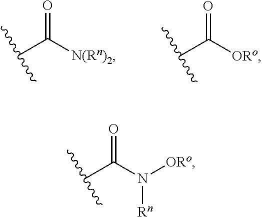

—CO—C₁₋₄ alkyl, 5-membered heteroaryl, C₁₋₄ alkyl-SO—, or C₁₋₄ alkyl-SO₂—; and wherein the heteroaryl, cycloalkyl, heterocyclyl, or alkyl is further substituted with 0, 1, 2, or 3 occurrences of C₁₋₄ alkyl or halogen as valency permits;
each instance of Rⁿ is independently H, C₁₋₄ alkyl, halo-C₁₋₄ alkyl, or C₃₋₆ cycloalkyl, or two Rⁿ groups are taken together with their intervening nitrogen to form a C₃₋₆ heterocycloalkyl optionally substituted with one or more occurrences of C₁₋₄ alkyl or halogen;
each instance of R^{o} is independently H or C₁₋₄ alkyl;
each instance of R₃ is independently selected from the group consisting of H, halo, CN, C₁₋₄ alkoxy, halo-C₁₋₄ alkyl, and C₁₋₄ alkyl; and
each of R₄ and R₅ is independently H or F.

In one embodiment, provided herein is a compound of Formula (I):

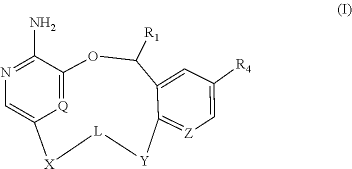

or a stereoisomer, a mixture of stereoisomers, or a pharmaceutically acceptable salt thereof, wherein:
Q is CH or N;
Z is CR₅ or N;
L is —CH₂—, C═O, or —O—;
X is a 5-membered heteroarylene, comprising 1 to 3 heteroatoms selected from the group consisting of nitrogen, oxygen and sulfur; wherein the 5-membered heteroarylene is substituted with 1, 2, or 3 occurrences of R²;
Y is a heteroarylene selected from the group consisting of 2*,3-substituted-furanylene, 2,3*-substituted-furanylene, 3*,4-substituted-furanylene, 1*,2-substituted-imidazolylene, 1*,5-substituted-imidazolylene, 1,5*-substituted-imidazolylene, 4,5*-substituted-1,2,3-oxadiazolylene, 3,4*-substituted-1,2-oxazolylene, 4*,5-substituted-1,2-oxazolylene, 4,5*-substituted-1,2-oxazolylene, 4,5*-substituted-1,3-oxazolylene, 1*,2-substituted-phenylene, 1,5*-substituted-pyrazolylene, 4*,5-substituted-pyrazolylene, 3,4*-substituted-pyridazinylene, 4*,5-substituted-pyridazinylene, 2,3*-substituted-pyridinylene, 3*,4-substituted-pyridinylene, 3,4*-substituted-pyridinylene, 4,5*-substituted-pyrimidinylene, 1*,2-substituted-pyrrolylene, 1,2*-substituted-pyrrolylene, 2,3*-substituted-pyrrolylene, 3*,4-substituted-pyrrolylene, 4,5*-substituted-1,2,3-thiadiazolylene, 3,4*-substituted-1,2-thiazolylene, 4*,5-substituted-1,2-thiazolylene, 4,5*-substituted-1,2-thiazolylene, 4*,5-substituted-1,3-thiazolylene, 4,5*-substituted-1,3-thiazolylene, 2*,3-substituted-thiophenylene, 2,3*-substituted-thiophenylene, 3*,4-substituted-thiophenylene, 4,5*-substituted-1,2,3-triazinylene, 4*,5-substituted-triazolylene, 1,5*-substituted-1,2,3-triazolylene, 4*,5-substituted-1,2,3-triazolylene, 1*,5-substituted-1,2,4-triazolylene, 1,5*-substituted-1,2,4-triazolylene, and 3,4*-substituted-1,2,4-triazolylene;
wherein the heteroarylene is substituted with 0, 1, or 2 occurrences of R₃;
* indicates the point of attachment of X or Y to the L group bonded to X and Y;
R₁ is selected from the group consisting of H, methyl, and hydroxymethyl;
each instance of R₂ is independently selected from the group consisting of H, CN, halo,

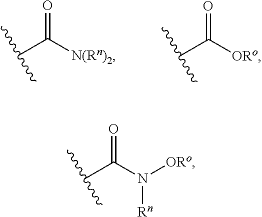

—CO—C₁₋₄ alkyl, 5-membered heteroaryl, C₁₋₄ alkyl-SO—, C₁₋₄ alkyl-SO₂—, C₁₋₄ alkoxy, C₁₋₄ alkyl, halo-C₁₋₄ alkyl, C₁₋₄ alkoxy-C₁₋₄ alkyl, C₃₋₄ cycloalkylmethyl, C₃₋₆ cycloalkyl, and C₃₋₆ heterocyclyl; provided that, when L is —CH₂—, at least one occurrence of R₂ is

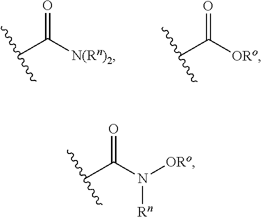

—CO—C₁₋₄ alkyl, 5-membered heteroaryl, C₁₋₄ alkyl-SO—, or C₁₋₄ alkyl-SO₂—; and wherein the heteroaryl, cycloalkyl, heterocyclyl, or alkyl is further substituted with 0, 1, 2, or 3 occurrences of C₁₋₄ alkyl or halogen as valency permits;
each instance of Rⁿ is independently H, C₁₋₄ alkyl, halo-C₁₋₄ alkyl, or C₃₋₆ cycloalkyl, or two Rⁿ groups are taken together with their intervening nitrogen to form a C₃₋₆ heterocycloalkyl optionally substituted with one or more occurrences of C₁₋₄ alkyl or halogen;
each instance of R^{o} is independently H or C₁₋₄ alkyl;
each instance of R₃ is independently selected from the group consisting of H, halo, CN, C₁₋₄ alkoxy, halo-C₁₋₄ alkyl, and C₁₋₄ alkyl; and
each of R₄ and R₅ is independently H or F.

In one embodiment, provided herein is a compound of Formula (I):

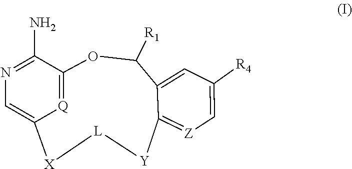

or a stereoisomer, a mixture of stereoisomers, or a pharmaceutically acceptable salt thereof, wherein:
Q is CH or N;
Z is CR₅ or N;
L is —CH₂—, C═O, —CH(OH)—, or —O—;
X is a 5-membered heteroarylene, comprising 1 to 3 heteroatoms selected from the group consisting of nitrogen, oxygen and sulfur; wherein the 5-membered heteroarylene is substituted with 1, 2, or 3 occurrences of R₂;
Y is a 5- or 6-membered heteroarylene, comprising 1 to 3 heteroatoms selected from the group consisting of nitrogen, oxygen and sulfur; wherein the 5- or 6-membered heteroarylene is substituted with 0, 1, or 2 occurrences of R₃;
R₁ is selected from the group consisting of H, methyl, and hydroxymethyl;
each instance of R₂ is independently selected from the group consisting of H, CN, halo,

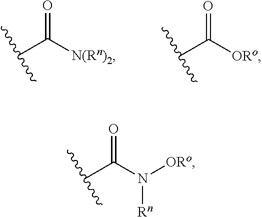

—S—C₁₋₄ alkyl, —CO—C₁₋₄ alkyl, 5-membered heteroaryl, C₁₋₄ alkyl-SO—, C₁₋₄ alkyl-SO₂—, C₁₋₄ alkoxy, C₁₋₄ alkyl, halo-C₁₋₄ alkyl, C₁₋₄ alkoxy-C₁₋₄ alkyl, C₃₋₄ cycloalkylmethyl, C₃₋₆ cycloalkyl, and C₃₋₆ heterocyclyl; provided that, when L is —CH₂—, at least one occurrence of R₂ is

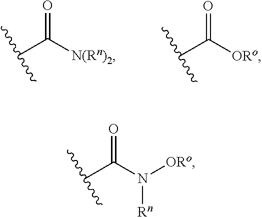

—CO—C₁₋₄ alkyl, 5-membered heteroaryl, —S—C₁₋₄ alkyl, C₁₋₄ alkyl-SO—, or C₁₋₄ alkyl-SO₂—; and wherein the heteroaryl, cycloalkyl, heterocyclyl, alkoxy, or alkyl is further substituted with 0, 1, 2, or 3 occurrences of C₁₋₄ alkyl, Si(C₁₋₄ alkyl)₃, or halogen as valency permits;
each instance of Rⁿ is independently H, C₁₋₄ alkyl, halo-C₁₋₄ alkyl, or C₃₋₆ cycloalkyl, or two Rⁿ groups are taken together with their intervening nitrogen to form a C₃₋₆ heterocycloalkyl optionally substituted with one or more occurrences of C₁₋₄ alkyl or halogen;
each instance of R^{o} is independently H or C₁₋₄ alkyl;
each instance of R₃ is independently selected from the group consisting of H, halo, CN, C₁₋₄ alkoxy, halo-C₁₋₄ alkyl, and C₁₋₄ alkyl; and
each of R₄ and R₅ is independently H or F.

In one embodiment, Si(C₁₋₄ alkyl)₃ is Si(Me)₃.

In some embodiments, L is —CH₂—. In some embodiments, L is C═O. In some embodiments, L is —O—. In some embodiments, L is —CH(OH)—. In some embodiments, L is —CH(OH)— and the carbon is of the S chirality. In some embodiments, L is —CH(OH)— and the carbon is of the R chirality.

In one embodiment, the compound is a compound of Formula (I-A):

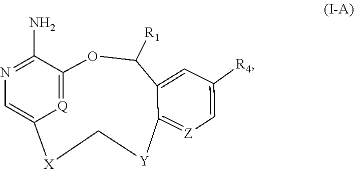

or a stereoisomer, a mixture of stereoisomers, or a pharmaceutically acceptable salt thereof.

In one embodiment, the compound is a compound of Formula (I-A-1):

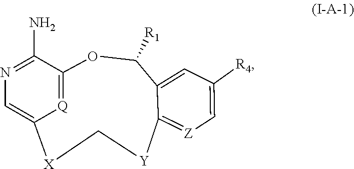

or a pharmaceutically acceptable salt thereof.

In one embodiment, the compound is a compound of Formula (I-A-2):

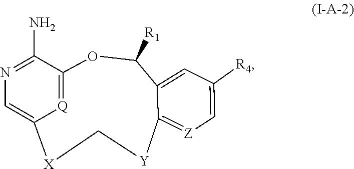

or a pharmaceutically acceptable salt thereof.

In one embodiment, the compound is a compound of Formula (I-B):

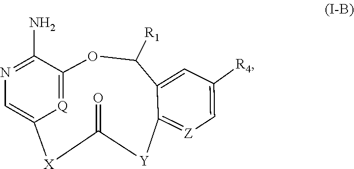

or a stereoisomer, a mixture of stereoisomers, or a pharmaceutically acceptable salt thereof.

In one embodiment, the compound is a compound of Formula (I-B-1):

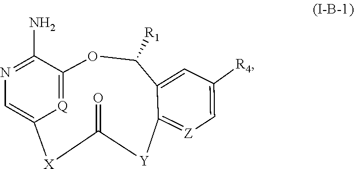

or a pharmaceutically acceptable salt thereof.

In one embodiment, the compound is a compound of Formula (I-B-2):

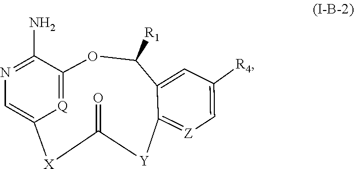

or a pharmaceutically acceptable salt thereof.

In one embodiment, the compound is a compound of Formula (I-C):

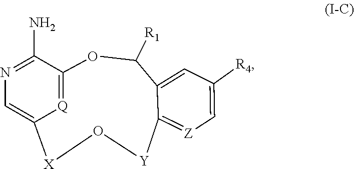

or a stereoisomer, a mixture of stereoisomers, or a pharmaceutically acceptable salt thereof.

In one embodiment, the compound is a compound of Formula (I-C-1):

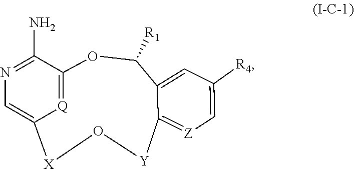

or a pharmaceutically acceptable salt thereof.

In one embodiment, the compound is a compound of Formula (I-C-2):

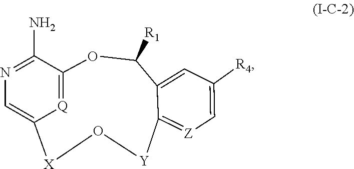

or a pharmaceutically acceptable salt thereof.

In one embodiment, the compound is a compound of Formula (I-D):

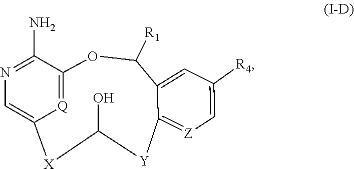

or a stereoisomer, a mixture of stereoisomers, or a pharmaceutically acceptable salt thereof.

In one embodiment, the compound is a compound of Formula (I-D-1):

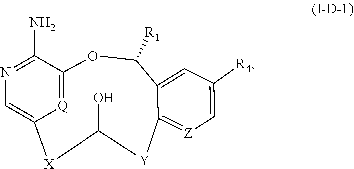

or a pharmaceutically acceptable salt thereof.

In one embodiment, the compound is a compound of Formula (I-D-1-1):

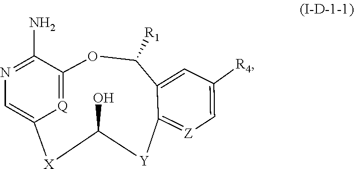

or a pharmaceutically acceptable salt thereof.

In one embodiment, the compound is a compound of Formula (I-D-1-2):

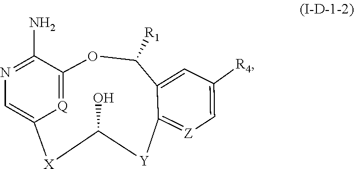

or a pharmaceutically acceptable salt thereof.

In one embodiment, in Y, the point of attachment to the L group bonded to X and Y and the point of attachment to the aromatic ring comprising Z are on adjacent atoms, and the 5- to 6-membered heteroarylene ring atom alpha to the point of attachment to the L group and beta to the point of attachment to the aromatic ring comprising Z is nitrogen.

In one embodiment, in Y, the point of attachment to the L group bonded to X and Y and the point of attachment to the aromatic ring comprising Z are on adjacent atoms, and the 5- to 6-membered heteroarylene ring atom alpha to the point of attachment to the L group and beta to the point of attachment to the aromatic ring comprising Z is carbon, oxygen, or sulfur.

In some embodiments, X is a 5-membered heteroaryl selected from the group consisting of pyrazolylene, isoxazolylene, isothiazolylene, imidazolylene, and triazolylene. In some embodiments, X is selected from the group consisting of pyrazolylene and triazolylene. In certain embodiments, X is selected from the group consisting of 4*,5-substituted-pyrazolylene, 4,5*-substituted-pyrazolylene, 1*,5-substituted-pyrazolylene, 4*,5-substituted-isoxazolylene, 3*,4-substituted-isoxazolylene, 3*,4-substituted-isothiazolylene, 4*,5-substituted-isothiazolylene, 4*,5-substituted-imidazolylene, 1*,5-substituted-imidazolylene, 1*,5-substituted-triazolylene, and 4*,5-substituted-triazolylene.

In some embodiments, X is a 5-membered heteroaryl selected from the group consisting of pyrazolylene, isoxazolylene, isothiazolylene, imidazolylene, and triazolylene. In some embodiments, X is selected from the group consisting of pyrazolylene and triazolylene. In certain embodiments, X is selected from the group consisting of 4*,5-substituted-pyrazolylene, 4,5*-substituted-pyrazolylene, 1*,5-substituted-pyrazolylene, 4*,5-substituted-isoxazolylene, 4,5*-substituted-isoxazolylene, 3*,4-substituted-isoxazolylene, 3*,4-substituted-isothiazolylene, 4*, 5-substituted-isothiazolylene, 4,5*-substituted-isothiazolylene, 4*,5-substituted-imidazolylene, 1*,5-substituted-imidazolylene, 1*,5-substituted-triazolylene, and 4*,5-substituted-triazolylene.

In one embodiment, X is a 5-membered heteroarylene selected from the group consisting of 3*,4-substituted-pyrazolylene, 4*,5-substituted-pyrazolylene, 4,5*-substituted-pyrazolylene, 1*, 5-substituted-pyrazolylene, 4*,5-substituted-imidazolylene, 1*,5-substituted-imidazolylene, or 4*,5-substituted-triazolylene, wherein * indicates the point of attachment of X or Y to the L group bonded to X and Y.

In certain embodiments, X is selected from the group consisting of

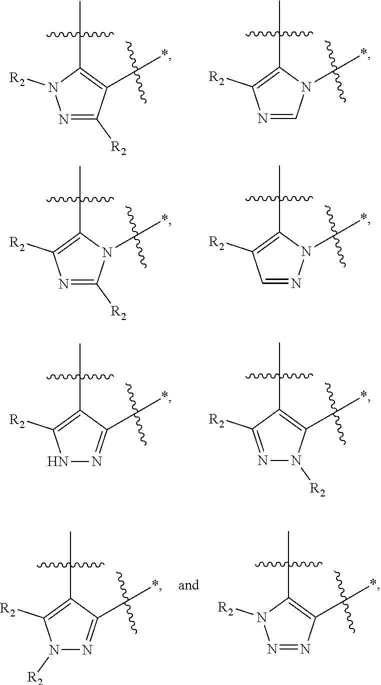

* indicates the point of attachment of X to the L group bonded to X and Y.

In certain embodiments, X is selected from the group consisting of:

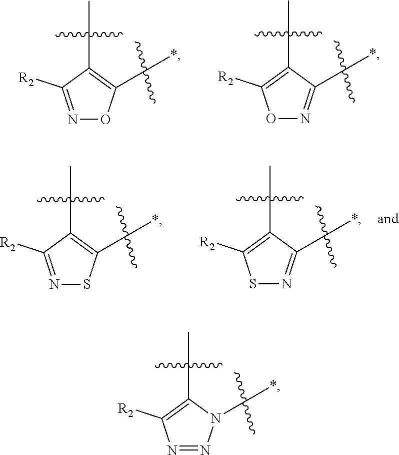

* indicates the point of attachment of X to the L group bonded to X and Y.

In one embodiment, X is a pyrazolylene. In one embodiment, X is not 3*,4-substituted-pyrazolylene. In one embodiment, X is not

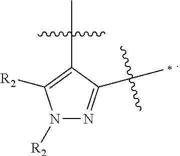

In one embodiment, X is not

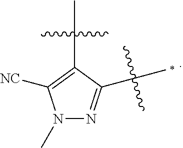 [SMILES: *c1nn(C)c(C#N)c1*]

In one embodiment, X is

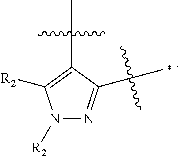

In one embodiment, X is

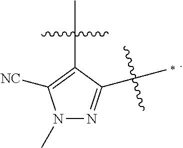 [SMILES: *c1nn(C)c(C#N)c1*]

In another embodiment, X is 3*,4-substituted-pyrazolylene. In another embodiment, X is 4*,5-substituted-pyrazolylene. In another embodiment, X is 4,5*-substituted-pyrazolylene. In another embodiment, X is 1*,5-substituted-pyrazolylene. In one embodiment, X is

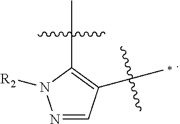

In one embodiment, X is

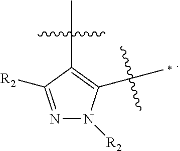

In one embodiment, X is

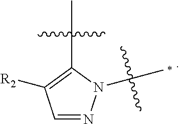

In one embodiment, X is

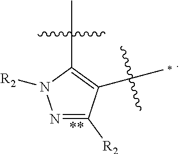

In one embodiment, X is

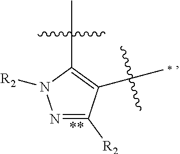

and the R₂ at ** position is

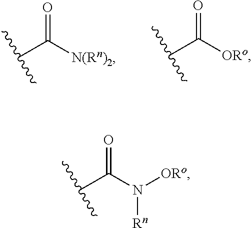

—S—C₁₋₄ alkyl, CN, —CO—C₁₋₄ alkyl, 5-membered heteroaryl, C₁₋₄ alkyl-SO—, or C₁₋₄ alkyl-SO₂—.

In one embodiment, X is isoxazolylene. In one embodiment, X is 4*,5-substituted-isoxazolylene. In one embodiment, X is 4,5*-substituted-isoxazolylene. In one embodiment, X is 3*,4-substituted-isoxazolylene. In one embodiment, X is

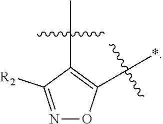

In one embodiment, X is

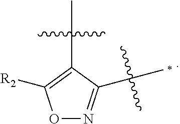

In one embodiment, X is isothiazolylene. In one embodiment, X is 3*,4-substituted-isothiazolylene. In one embodiment, X is 4*,5-substituted-isothiazolylene. In one embodiment, X is 4,5*-substituted-isothiazolylene. In one embodiment, X is

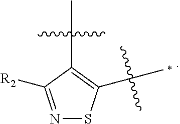

In one embodiment, X is

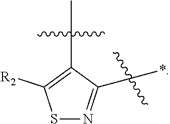

In one embodiment, X is imidazolylene. In one embodiment, X is 4*,5-substituted-imidazolylene. In one embodiment, X is 1*,5-substituted-imidazolylene. In one embodiment, X is

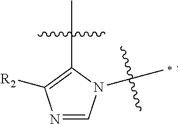

In one embodiment, X is triazolylene. In one embodiment, X is 1*,5-substituted-triazolylene. In one embodiment, X is 4*,5-substituted-triazolylene. In one embodiment, X is

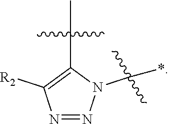

In one embodiment, X is

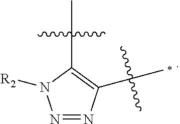

In some embodiments, X is substituted with 0 occurrence of R₂ (i.e., all open positions on X are H). In one embodiment, X is substituted with 1 occurrence of R₂ that is not H. In one embodiment, X is substituted with 2 occurrences of R₂ that are not H.

In some embodiments, at least one R₂ (e.g., one and only one R₂) is independently selected from the group consisting of H, CN, halo, C₁₋₄ alkoxy, C₁₋₄ alkyl, —S—C₁₋₄ alkyl, halo-C₁₋₄ alkyl, C₁₋₄ alkoxy-C₁₋₄ alkyl, C3.4 cycloalkylmethyl, C₃₋₆ cycloalkyl, and C₃₋₆ heterocyclyl. In some embodiments, at least one R₂ (e.g., one and only one R₂) is independently selected from the group consisting of H, fluoro, chloro, CN, methyl, and ethyl. In some embodiments, at least one R₂ (e.g., one and only one R₂) is independently selected from the group consisting of CN and methyl. In some embodiments, there are two R₂, and one R₂ is CN and the other R₂ is methyl.

In some embodiments, at least one R₂ (e.g., one and only one R₂) is

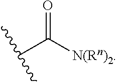

In some embodiments, at least one R₂ (e.g., one and only one R₂) is

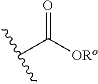

In some embodiments, at least one R₂ (e.g., one and only one R₂) is

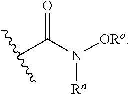

In some embodiments, at least one R₂ (e.g., one and only one R₂) is —CO—C₁₋₄ alkyl (e.g., —C(═O)—CH₃). In some embodiments, at least one R₂ (e.g., one and only one R₂) is 5-membered heteroaryl. In some embodiments, at least one R₂ (e.g., one and only one R₂) is C₁₋₄ alkyl-SO— (e.g., —SO—CH₃). In some embodiments, at least one R₂ (e.g., one and only one R₂) is C₁₋₄ alkyl-SO₂— (e.g., —SO₂—CH₃).

In some embodiments, one R₂ is 5-membered heteroaryl substituted with 1, 2, or 3 occurrences of C₁₋₄ alkyl (e.g., methyl). In some embodiments, one R₂ is 5-membered heteroaryl substituted with one C₁₋₄ alkyl (e.g., methyl).

In some embodiments, Rⁿ is H. In some embodiments, Rⁿ is methyl. In some embodiments, Rⁿ is ethyl. In some embodiments, Rⁿ is isopropyl. In some embodiments, Rⁿ is cyclopropyl. In some embodiments, Rⁿ is —CH₂CF₃. In some embodiments, two Rⁿ groups are taken together with their intervening nitrogen to form a C3.6 heterocycloalkyl optionally substituted with one or more occurrences of C₁₋₄ alkyl or halogen.

In some embodiments, R^{o} is H. In some embodiments, R^{o} is methyl.

In some embodiments, each R₂ is independently selected from CN, —CH₂-cyclopropyl, —CH₂CH₂OCH₃, —CO₂Et, methyl, ethyl, —C(═O)—N(CH₃)₂, —C(═O)—N(CH₃)ⁱPr, —C(═O)—N(CH₃)Et, —C(═O)—NH₂, —C(═O)—N(CH₃)(CH₂CF₃), —C(═O)—N(CH₃)(cyclopropyl), —C(═O)—CH₃, —C(═O)—N(CH₃)—OCH₃, CH₂—O(CH₂)₂—Si(CH₃)₃, —CH₂-cyclobutyl, CH₂-cyclopropyl, —(CH₂)₂—OCH₃,

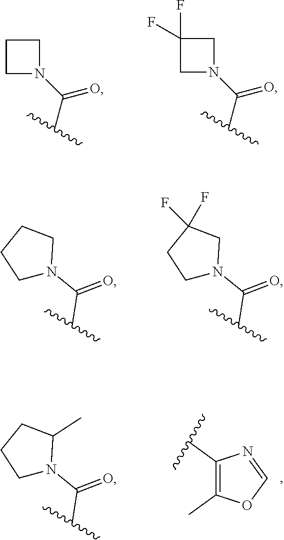

—SO—CH₃, and —SO₂—CH₃.

In some embodiments, Y is a 5-membered heteroarylene comprising 1 to 3 heteroatoms selected from the group consisting of nitrogen, oxygen and sulfur; wherein the 5-membered heteroarylene is substituted with 0, 1, or 2 occurrences of R₃. In some embodiments, Y is a 6-membered heteroarylene comprising 1 to 3 heteroatoms selected from the group consisting of nitrogen, oxygen and sulfur; wherein the 6-membered heteroarylene is substituted with 0, 1, or 2 occurrences of R₃. In some embodiments, Y is a heteroarylene selected from the group consisting of 2*,3-substituted-furanylene, 2,3*-substituted-furanylene, 3*,4-substituted-furanylene, 1*,2-substituted-imidazolylene, 1*,5-substituted-imidazolylene, 1,5*-substituted-imidazolylene, 4,5*-substituted-1,2,3-oxadiazolylene, 3,4*-substituted-1,2-oxazolylene, 4*,5-substituted-1,2-oxazolylene, 4,5*-substituted-1,2-oxazolylene, 4,5*-substituted-1,3-oxazolylene, 1*,2-substituted-phenylene, 1,5*-substituted-pyrazolylene, 4*,5-substituted-pyrazolylene, 3,4*-substituted-pyridazinylene, 4*,5-substituted-pyridazinylene, 2,3*-substituted-pyridinylene, 3*,4-substituted-pyridinylene, 3,4*-substituted-pyridinylene, 4,5*-substituted-pyrimidinylene, 1*,2-substituted-pyrrolylene, 1,2*-substituted-pyrrolylene, 2,3*-substituted-pyrrolylene, 3*,4-substituted-pyrrolylene, 4,5*-substituted-1,2,3-thiadiazolylene, 4*,5-substituted-1,2-thiazolylene, 4,5*-substituted-1,2-thiazolylene, 4*,5-substituted-1,3-thiazolylene, 4,5*-substituted-1,3-thiazolylene, 2*,3-substituted-thiophenylene, 2,3*-substituted-thiophenylene, 3*,4-substituted-thiophenylene, 4,5*-substituted-1,2,3-triazinylene, 1,5*-substituted-1,2,3-triazolylene, 4*,5-substituted-triazolylene, and 3,4*-substituted-1,2,4-triazolylene; wherein the heteroarylene is substituted with 0, 1, or 2 occurrences of R₃. In some embodiments, Y is selected from the group consisting of 1*, 5-substituted-pyrazolylene, 3*,4-substituted-pyrazolylene, 2,3*-substituted-pyridinylene, 3*,4-substituted-pyridinylene, 3,4*-substituted-pyridinylene, 4,5*-substituted 1,3-thiazolylene, 4*,5-substituted-1,2,3-triazolylene, 1*,5-substituted-1,2,4-triazolylene, 1,5*-substituted-1,2,4-triazolylene, and 4*,5-substituted 1,3-thiazolylene, wherein * indicates the point of attachment to the L bonded to X.

In certain embodiments, Y is selected from the group consisting of

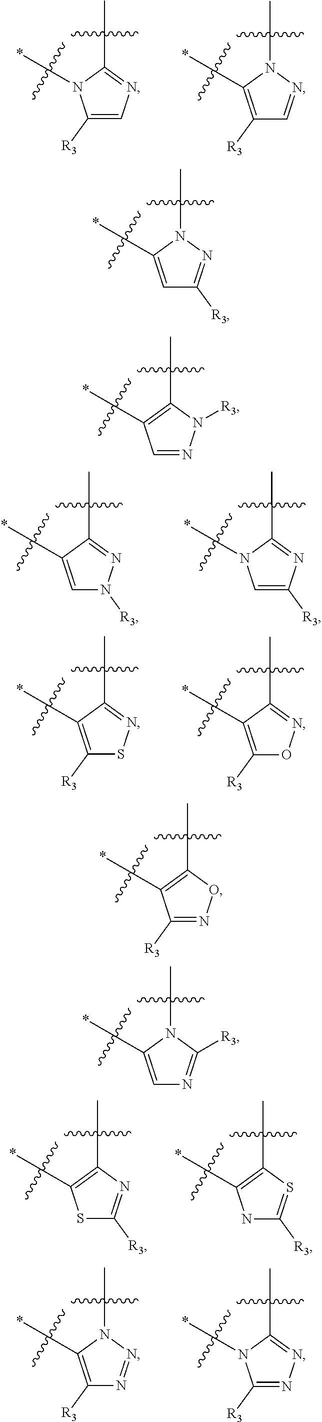

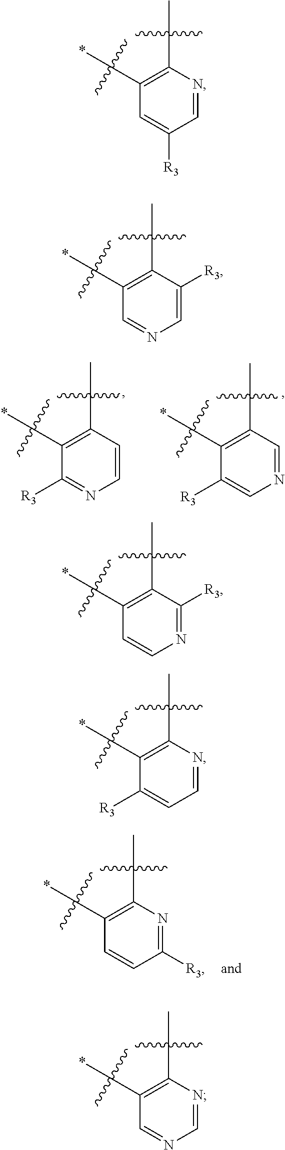

* indicates the point of attachment of Y to the L group bonded to X and Y.

In certain embodiments, Y is

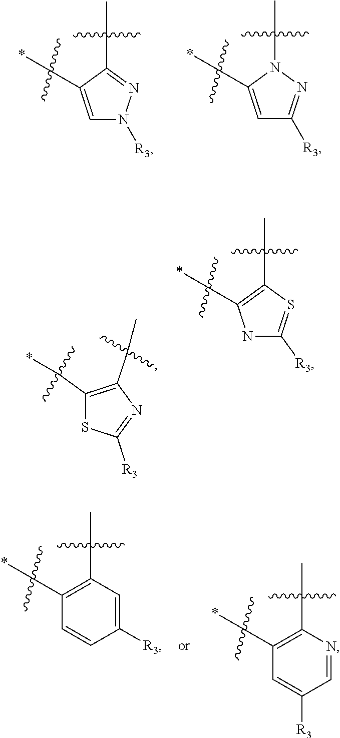

* indicates the point of attachment of Y to the L group bonded to X and Y.

In certain embodiments, Y is a 5-membered heteroarylene. In certain embodiments, Y is pyrazolylene. In certain embodiments, Y is 1,5*-substituted pyrazolylene. In certain embodiments, Y is 4*,5-substituted pyrazolylene. In certain embodiments, Y is 3,4*-substituted pyrazolylene. In certain embodiments, Y is

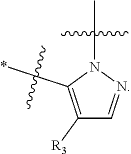

In certain embodiments, Y is

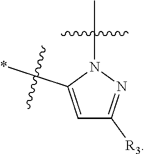

In certain embodiments, Y is

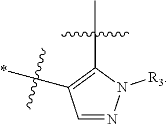

In certain embodiments, Y is

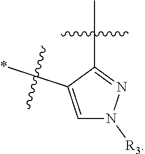

In certain embodiments, Y is imidazolylene. In certain embodiments, Y is 1*,2-substituted imidazolylene. In certain embodiments, Y is 5*,1-substituted imidazolylene. In certain embodiments, Y is

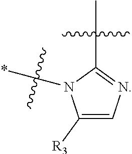

In certain embodiments, Y is

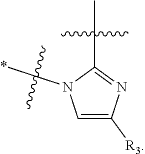

In certain embodiments, Y is

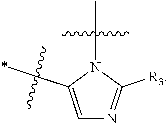

In certain embodiments, Y is 1,2-thiazolylene. In certain embodiments, Y is 3,4*-substituted 1,2-thiazolylene. In certain embodiments, Y is 4*,5-substituted 1,2-thiazolylene. In certain embodiments, Y is

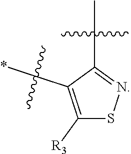

In certain embodiments, Y is

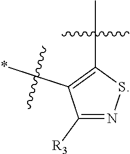

In certain embodiments, Y is 1,3-thiazolylene. In certain embodiments, Y is 4,5*-substituted 1,3-thiazolylene. In certain embodiments, Y is

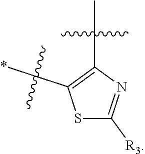

In certain embodiments, Y is 4*,5-substituted 1,3-thiazolylene. In certain embodiments, Y is

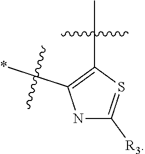

In certain embodiments, Y is 1,2-oxazolylene. In certain embodiments, Y is 3,4*-substituted 1,2-oxazolylene. In certain embodiments, Y is 4*,5-substituted 1,2-oxazolylene. In certain embodiments, Y is

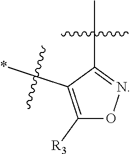

In certain embodiments, Y is

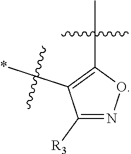

In certain embodiments, Y is triazolylene. In certain embodiments, Y is 1,5*-substituted 1,2,3-triazolylene. In certain embodiments, Y is 3,4*-substituted 1,2,4-triazolylene. In certain embodiments, Y is

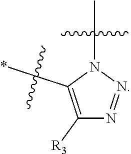

In certain embodiments, Y is

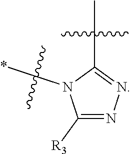

In certain embodiments, Y is a 6-membered heteroarylene. In certain embodiments, Y is pyridinylene. In certain embodiments, Y is 2,3*-substituted pyridinylene. In certain embodiments, Y is 3*,4-substituted pyridinylene. In certain embodiments, Y is 4*,3-substituted pyridinylene. In certain embodiments, Y is

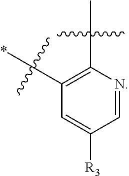

In certain embodiments, Y is

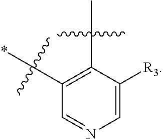

In certain embodiments, Y is

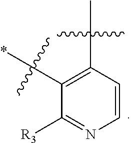

In certain embodiments, Y is

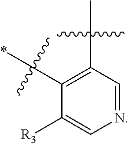

In certain embodiments, Y is

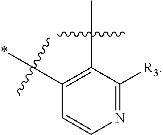

In certain embodiments, Y is

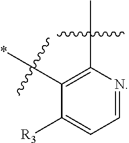

In certain embodiments, Y is

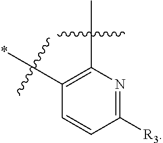

In certain embodiments, Y is

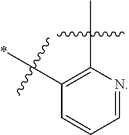

In certain embodiments, Y is pyrimidinylene. In certain embodiments, Y is 4,5*-substituted pyrimidinylene. In certain embodiments, Y is

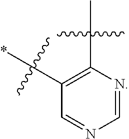

In certain embodiments, Y is substituted with 0 occurrence of R₃ (i.e., all open positions on Y are H). In certain embodiments, Y is substituted with 1 occurrence of R₃ that is not H. In certain embodiments, Y is substituted with 2 occurrences of R₃ that are not H.

In certain embodiments, R₃ is selected from the group consisting of H, halo, CN, C₁₋₄ alkoxy, halo-C₁₋₄ alkyl, and C₁₋₄ alkyl. In certain embodiments, R₃ is not H. In certain embodiments, R₃ is C₁₋₄ alkyl. In certain embodiments, R₃ is methyl. In certain embodiments, R₃ is ethyl. In certain embodiments, R₃ is halo. In certain embodiments, R₃ is fluoro. In certain embodiments, R₃ is chloro. In certain embodiments, R₃ is CN.

In some embodiments, Q is CH. In other embodiments, Q is N.

In some embodiments, Z is CR₅. In particular embodiments, R₅ is H. In particular embodiments, R₅ is F. In other embodiments, Z is N.

In some embodiments, R₁ is methyl. In some embodiments, R₁ is hydrogen. In some embodiments, R₁ is hydroxymethyl.

In some embodiments, R₄ is H. In other embodiments, R₄ is F.

In one embodiment of Formula (I-B), (I-B-1), or (I-B-2), X is

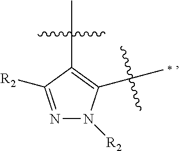

### and Y is

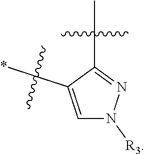

In another embodiment, X is

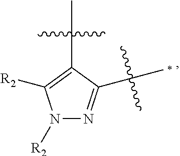

### and Y is

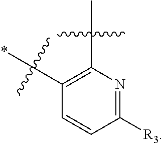

In another embodiment, X is

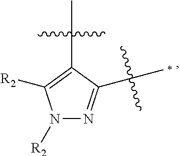

### and Y is

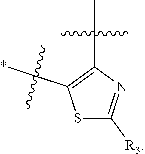

In another embodiment, X is

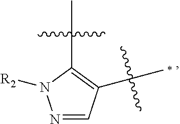

### and Y is

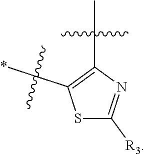

In another embodiment, X is

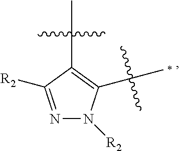

### and Y is

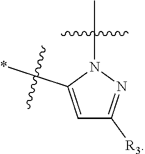

In another embodiment, X is

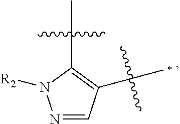

### and Y is

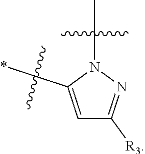

In another embodiment, X is

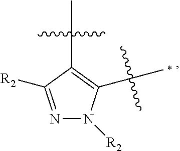

### and Y is

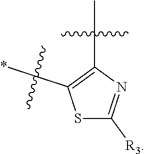

In another embodiment, X is

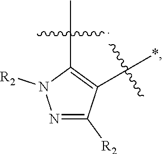

### and Y is

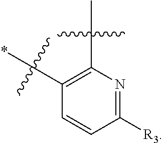

In another embodiment, X is

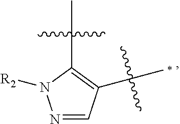

### and Y is

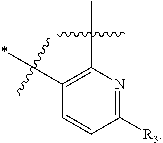

In another embodiment, X is

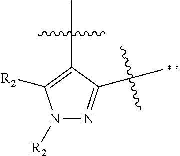

### and Y is

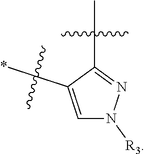

In one embodiment, R₂ is selected from the group consisting of H, methyl, ethyl, CN, —C(═O)—N(CH₃)₂, methoxyethyl, isopropyl, and cyclopropylmethyl. In one embodiment, R₃ is selected from the group consisting of H and methyl. In one embodiment, R₁ is selected from the group consisting of H and methyl.

In one embodiment, the compound is a compound of any one of the following formulas:

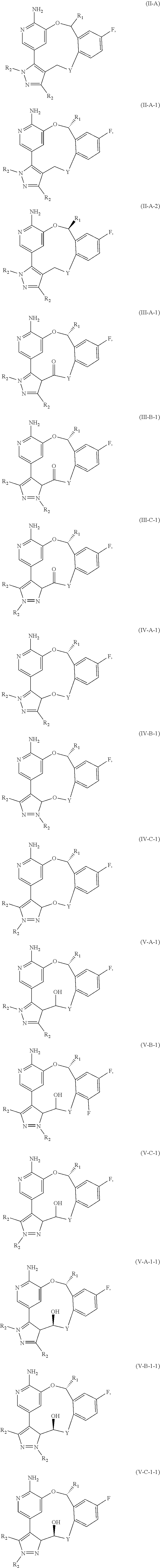

or a stereoisomer, a mixture of stereoisomers, or a pharmaceutically acceptable salt thereof.

In certain embodiments, provided herein is a compound in Table 1:

TABLE 1

| | |
|---|---|
| 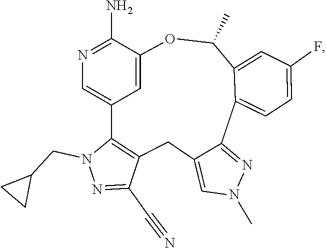 [SMILES: C[C@H]1Oc2cc(cnc2N)-c2c(c(C#N)nn2CC2CC2)Cc2cn(C)nc2-c2ccc(F)cc21] | 1 |
| 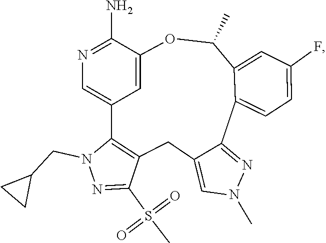 [SMILES: C[C@H]1Oc2cc(cnc2N)-c2c(c(S(C)(=O)=O)nn2CC2CC2)Cc2cn(C)nc2-c2ccc(F)cc21] | 2 |
| 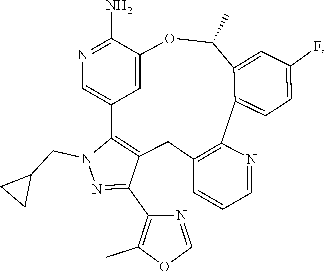 [SMILES: Cc1ocnc1-c1nn(CC2CC2)c2c1Cc1cccnc1-c1ccc(F)cc1[C@@H](C)Oc1cc-2cnc1N] | 3 |
| 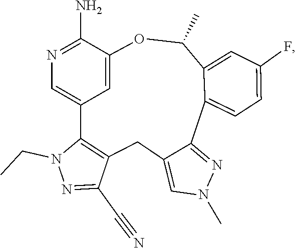 [SMILES: CCn1nc(C#N)c2c1-c1cnc(N)c(c1)O[C@H](C)c1cc(F)ccc1-c1nn(C)cc1C2] | 4 |
| 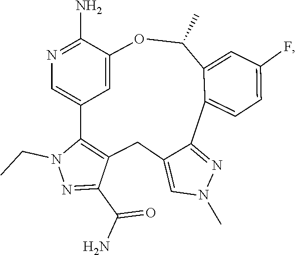 [SMILES: CCn1nc(C(N)=O)c2c1-c1cnc(N)c(c1)O[C@H](C)c1cc(F)ccc1-c1nn(C)cc1C2] | 5 |
| 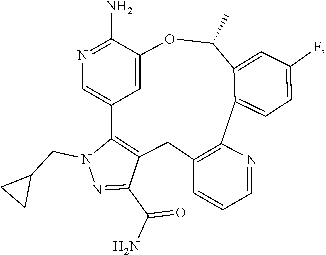 [SMILES: C[C@H]1Oc2cc(cnc2N)-c2c(c(C(N)=O)nn2CC2CC2)Cc2cccnc2-c2ccc(F)cc21] | 6 |
| 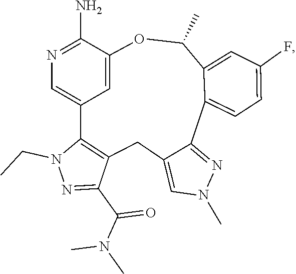 [SMILES: CCn1nc(C(=O)N(C)C)c2c1-c1cnc(N)c(c1)O[C@H](C)c1cc(F)ccc1-c1nn(C)cc1C2] | 7 |
| 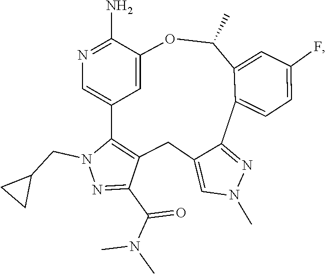 [SMILES: C[C@H]1Oc2cc(cnc2N)-c2c(c(C(=O)N(C)C)nn2CC2CC2)Cc2cn(C)nc2-c2ccc(F)cc21] | 8 |
| 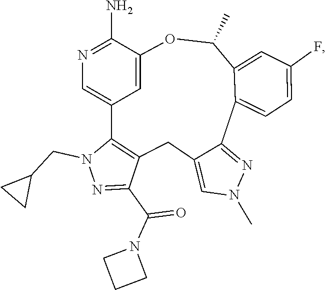 [SMILES: C[C@H]1Oc2cc(cnc2N)-c2c(c(C(=O)N3CCC3)nn2CC2CC2)Cc2cn(C)nc2-c2ccc(F)cc21] | 9 |
| 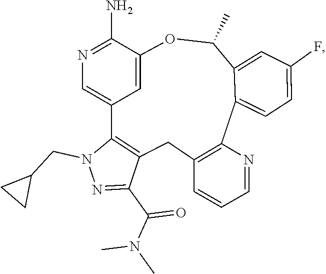 [SMILES: C[C@H]1Oc2cc(cnc2N)-c2c(c(C(=O)N(C)C)nn2CC2CC2)Cc2cccnc2-c2ccc(F)cc21] | 10 |
| 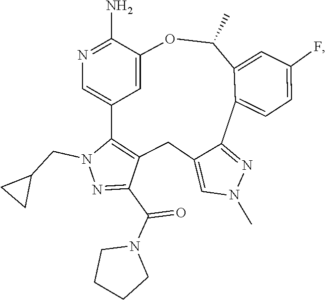 [SMILES: C[C@H]1Oc2cc(cnc2N)-c2c(c(C(=O)N3CCCC3)nn2CC2CC2)Cc2cn(C)nc2-c2ccc(F)cc21] | 11 |
| 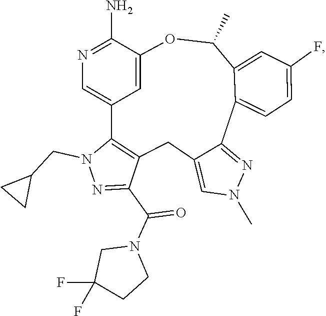 [SMILES: C[C@H]1Oc2cc(cnc2N)-c2c(c(C(=O)N3CCC(F)(F)C3)nn2CC2CC2)Cc2cn(C)nc2-c2ccc(F)cc21] | 12 |
| 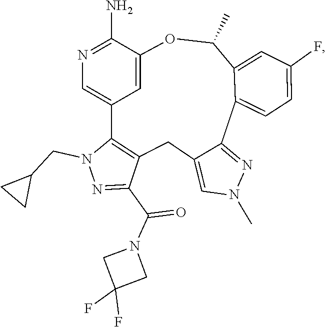 [SMILES: C[C@H]1Oc2cc(cnc2N)-c2c(c(C(=O)N3CC(F)(F)C3)nn2CC2CC2)Cc2cn(C)nc2-c2ccc(F)cc21] | 13 |
| 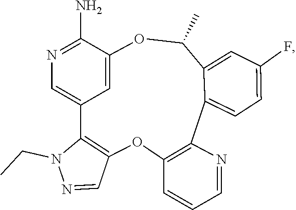 [SMILES: CCn1ncc2c1-c1cnc(N)c(c1)O[C@H](C)c1cc(F)ccc1-c1ncccc1O2] | 14 |
| 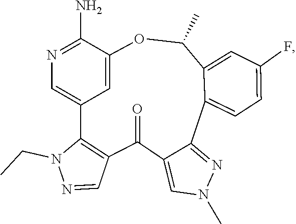 [SMILES: CCn1ncc2c1-c1cnc(N)c(c1)O[C@H](C)c1cc(F)ccc1-c1nn(C)cc1C2=O] | 15 |
| 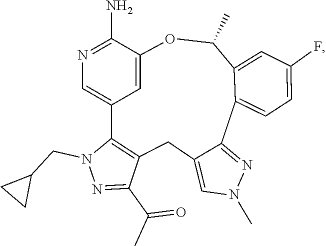 [SMILES: CC(=O)c1nn(CC2CC2)c2c1Cc1cn(C)nc1-c1ccc(F)cc1[C@@H](C)Oc1cc-2cnc1N] | 16 |
| 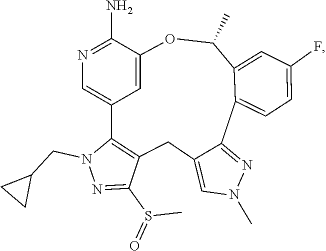 [SMILES: C[C@H]1Oc2cc(cnc2N)-c2c(c(S(C)=O)nn2CC2CC2)Cc2cn(C)nc2-c2ccc(F)cc21] | 17 |
| 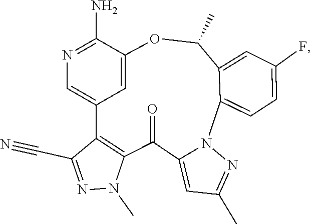 [SMILES: Cc1cc2n(n1)-c1ccc(F)cc1[C@@H](C)Oc1cc(cnc1N)-c1c(C#N)nn(C)c1C2=O] | 18 |
| 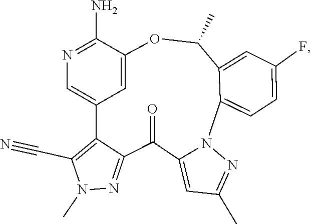 [SMILES: Cc1cc2n(n1)-c1ccc(F)cc1[C@@H](C)Oc1cc(cnc1N)-c1c(nn(C)c1C#N)C2=O] | 19 |
| 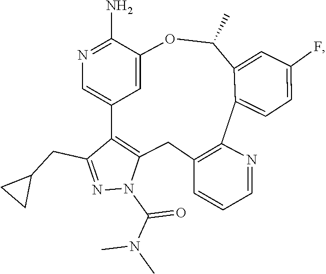 [SMILES: C[C@H]1Oc2cc(cnc2N)-c2c(CC3CC3)nn(C(=O)N(C)C)c2Cc2cccnc2-c2ccc(F)cc21] | 20 |
| 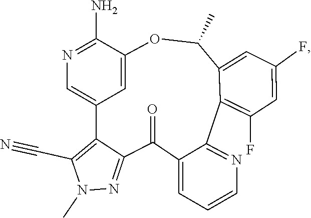 [SMILES: C[C@H]1Oc2cc(cnc2N)-c2c(nn(C)c2C#N)C(=O)c2cccnc2-c2c(F)cc(F)cc21] | 21 |
| 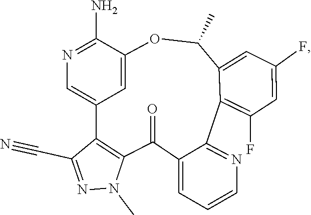 [SMILES: C[C@H]1Oc2cc(cnc2N)-c2c(C#N)nn(C)c2C(=O)c2cccnc2-c2c(F)cc(F)cc21] | 22 |
| 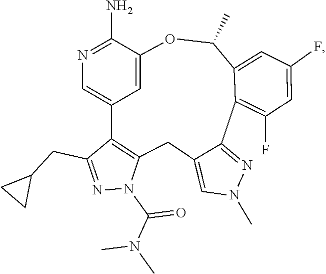 [SMILES: C[C@H]1Oc2cc(cnc2N)-c2c(CC3CC3)nn(C(=O)N(C)C)c2Cc2cn(C)nc2-c2c(F)cc(F)cc21] | 23 |
| 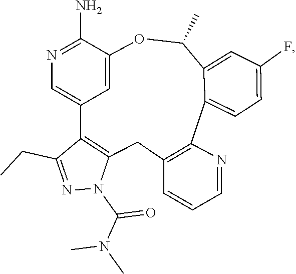 [SMILES: CCc1nn(C(=O)N(C)C)c2c1-c1cnc(N)c(c1)O[C@H](C)c1cc(F)ccc1-c1ncccc1C2] | 24 |
| 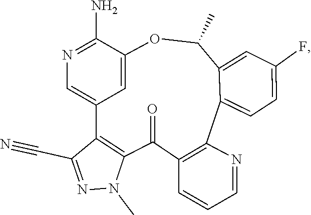 [SMILES: C[C@H]1Oc2cc(cnc2N)-c2c(C#N)nn(C)c2C(=O)c2cccnc2-c2ccc(F)cc21] | 25 |
| 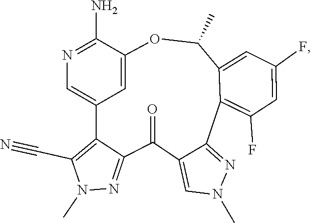 [SMILES: C[C@H]1Oc2cc(cnc2N)-c2c(nn(C)c2C#N)C(=O)c2cn(C)nc2-c2c(F)cc(F)cc21] | 26 |
| 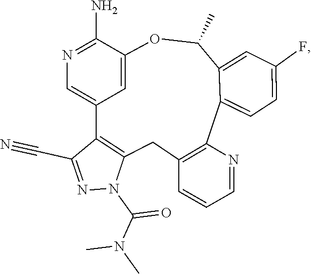 [SMILES: C[C@H]1Oc2cc(cnc2N)-c2c(C#N)nn(C(=O)N(C)C)c2Cc2cccnc2-c2ccc(F)cc21] | 27 |
| 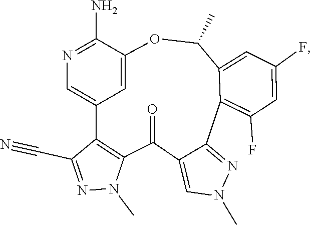 [SMILES: C[C@H]1Oc2cc(cnc2N)-c2c(C#N)nn(C)c2C(=O)c2cn(C)nc2-c2c(F)cc(F)cc21] | 28 |
| 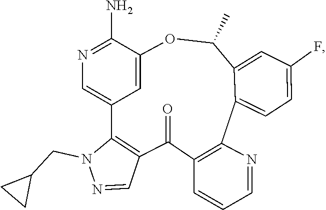 [SMILES: C[C@H]1Oc2cc(cnc2N)-c2c(cnn2CC2CC2)C(=O)c2cccnc2-c2ccc(F)cc21] | 29 |
| 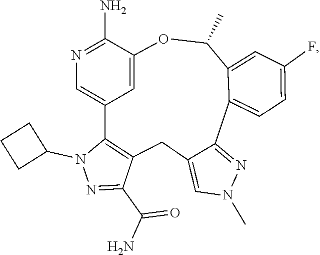 [SMILES: C[C@H]1Oc2cc(cnc2N)-c2c(c(C(N)=O)nn2C2CCC2)Cc2cn(C)nc2-c2ccc(F)cc21] | 30 |
| 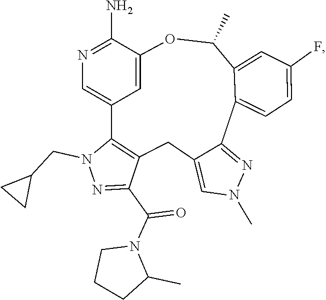 [SMILES: CC1CCCN1C(=O)c1nn(CC2CC2)c2c1Cc1cn(C)nc1-c1ccc(F)cc1[C@@H](C)Oc1cc-2cnc1N] | 31 |
| 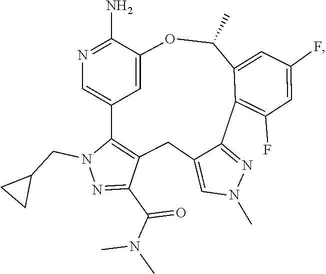 [SMILES: C[C@H]1Oc2cc(cnc2N)-c2c(c(C(=O)N(C)C)nn2CC2CC2)Cc2cn(C)nc2-c2c(F)cc(F)cc21] | 32 |
| 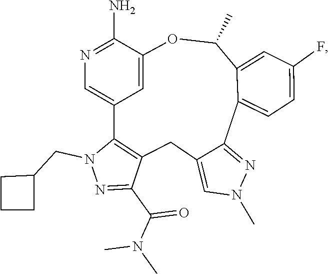 [SMILES: C[C@H]1Oc2cc(cnc2N)-c2c(c(C(=O)N(C)C)nn2CC2CCC2)Cc2cn(C)nc2-c2ccc(F)cc21] | 33 |
| 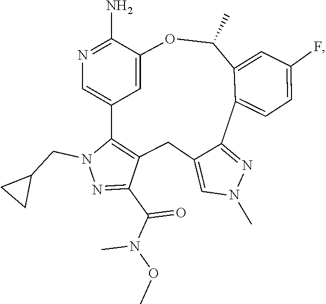 [SMILES: CON(C)C(=O)c1nn(CC2CC2)c2c1Cc1cn(C)nc1-c1ccc(F)cc1[C@@H](C)Oc1cc-2cnc1N] | 34 |
| 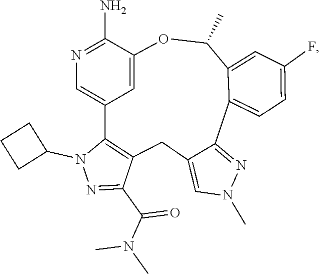 [SMILES: C[C@H]1Oc2cc(cnc2N)-c2c(c(C(=O)N(C)C)nn2C2CCC2)Cc2cn(C)nc2-c2ccc(F)cc21] | 35 |
| 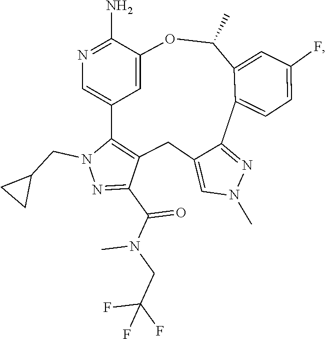 [SMILES: C[C@H]1Oc2cc(cnc2N)-c2c(c(C(=O)N(C)CC(F)(F)F)nn2CC2CC2)Cc2cn(C)nc2-c2ccc(F)cc21] | 36 |
| 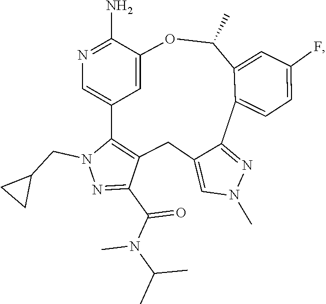 [SMILES: CC(C)N(C)C(=O)c1nn(CC2CC2)c2c1Cc1cn(C)nc1-c1ccc(F)cc1[C@@H](C)Oc1cc-2cnc1N] | 37 |
| 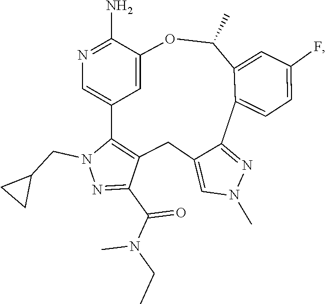 [SMILES: CCN(C)C(=O)c1nn(CC2CC2)c2c1Cc1cn(C)nc1-c1ccc(F)cc1[C@@H](C)Oc1cc-2cnc1N] | 38 |
| 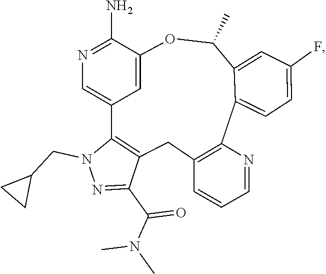 [SMILES: C[C@H]1Oc2cc(cnc2N)-c2c(c(C(=O)N(C)C)nn2CC2CC2)Cc2cccnc2-c2ccc(F)cc21] | 39 |
| 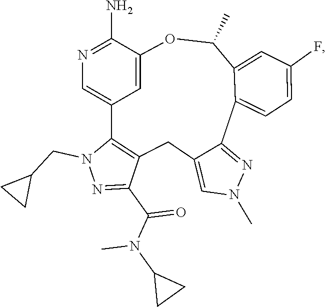 [SMILES: C[C@H]1Oc2cc(cnc2N)-c2c(c(C(=O)N(C)C3CC3)nn2CC2CC2)Cc2cn(C)nc2-c2ccc(F)cc21] | 40 |
| 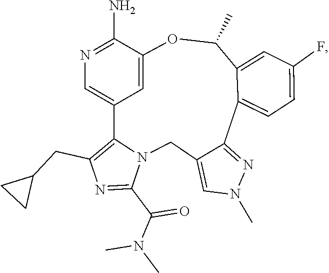 [SMILES: C[C@H]1Oc2cc(cnc2N)-c2c(CC3CC3)nc(C(=O)N(C)C)n2Cc2cn(C)nc2-c2ccc(F)cc21] | 41 |
| 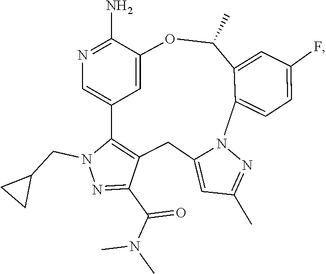 [SMILES: Cc1cc2n(n1)-c1ccc(F)cc1[C@@H](C)Oc1cc(cnc1N)-c1c(c(C(=O)N(C)C)nn1CC1CC1)C2] | 42 |
| 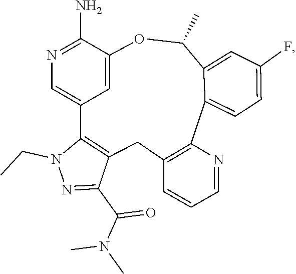 [SMILES: CCn1nc(C(=O)N(C)C)c2c1-c1cnc(N)c(c1)O[C@H](C)c1cc(F)ccc1-c1ncccc1C2] | 43 |
| 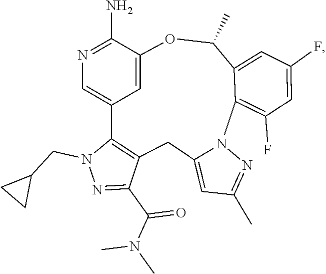 [SMILES: Cc1cc2n(n1)-c1c(F)cc(F)cc1[C@@H](C)Oc1cc(cnc1N)-c1c(c(C(=O)N(C)C)nn1CC1CC1)C2] | 44 |
| 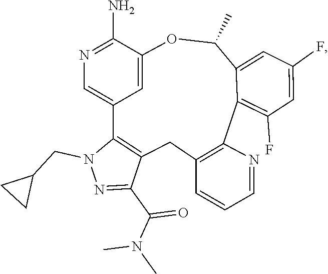 [SMILES: C[C@H]1Oc2cc(cnc2N)-c2c(c(C(=O)N(C)C)nn2CC2CC2)Cc2cccnc2-c2c(F)cc(F)cc21] | 45 |
| 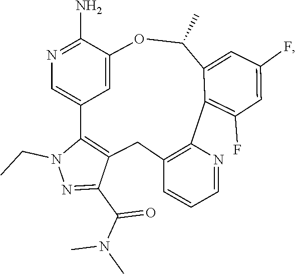 [SMILES: CCn1nc(C(=O)N(C)C)c2c1-c1cnc(N)c(c1)O[C@H](C)c1cc(F)cc(F)c1-c1ncccc1C2] | 46 |
| 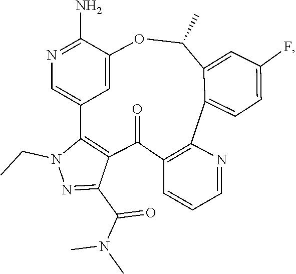 [SMILES: CCn1nc(C(=O)N(C)C)c2c1-c1cnc(N)c(c1)O[C@H](C)c1cc(F)ccc1-c1ncccc1C2=O] | 47 |
| 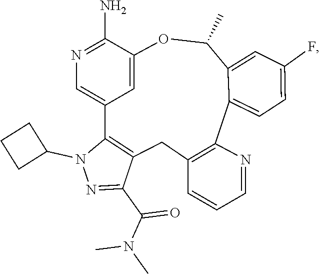 [SMILES: C[C@H]1Oc2cc(cnc2N)-c2c(c(C(=O)N(C)C)nn2C2CCC2)Cc2cccnc2-c2ccc(F)cc21] | 48 |
| 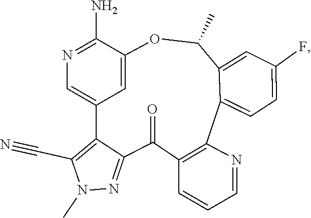 [SMILES: C[C@H]1Oc2cc(cnc2N)-c2c(nn(C)c2C#N)C(=O)c2cccnc2-c2ccc(F)cc21] | 49 |
| 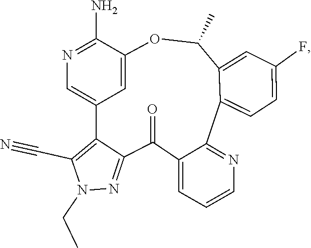 [SMILES: CCn1nc2c(c1C#N)-c1cnc(N)c(c1)O[C@H](C)c1cc(F)ccc1-c1ncccc1C2=O] | 50 |
| 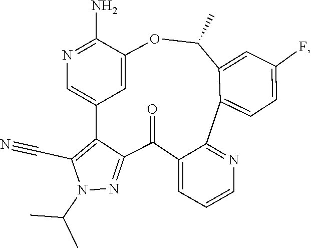 [SMILES: CC(C)n1nc2c(c1C#N)-c1cnc(N)c(c1)O[C@H](C)c1cc(F)ccc1-c1ncccc1C2=O] | 51 |
| 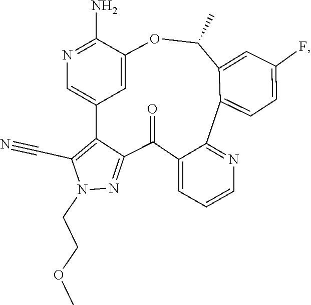 [SMILES: COCCn1nc2c(c1C#N)-c1cnc(N)c(c1)O[C@H](C)c1cc(F)ccc1-c1ncccc1C2=O] | 52 |
| 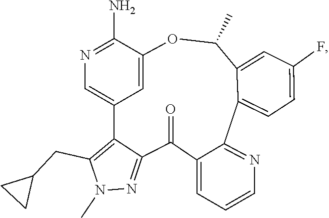 [SMILES: C[C@H]1Oc2cc(cnc2N)-c2c(nn(C)c2CC2CC2)C(=O)c2cccnc2-c2ccc(F)cc21] | 53 |
| 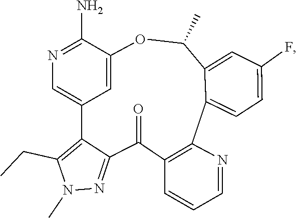 [SMILES: CCc1c2c(nn1C)C(=O)c1cccnc1-c1ccc(F)cc1[C@@H](C)Oc1cc-2cnc1N] | 54 |
| 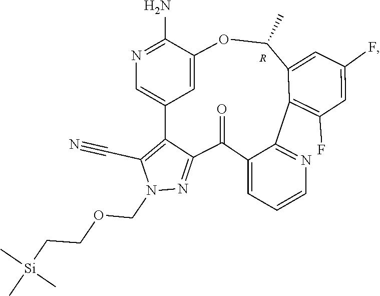 | 55 |
| 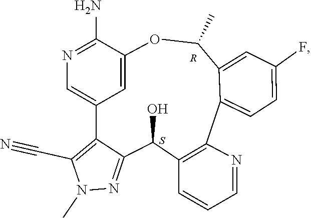 | 56 |
| 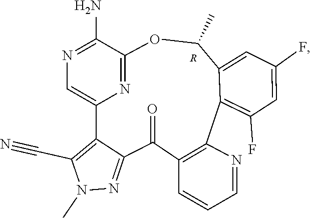 | 57 |
| 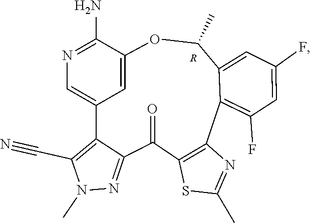 | 58 |
| 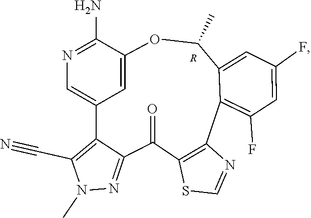 | 59 |
| 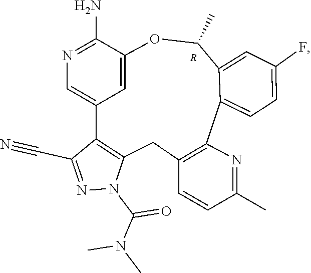 | 60 |
| 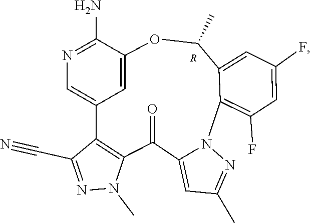 | 61 |
| 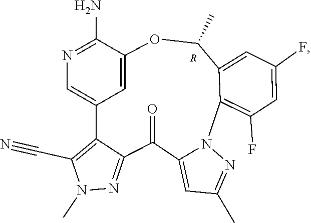 | 62 |
| 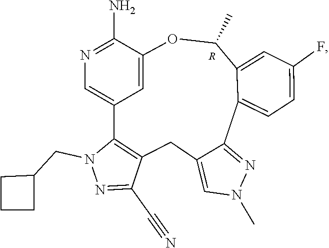 | 63 |
| 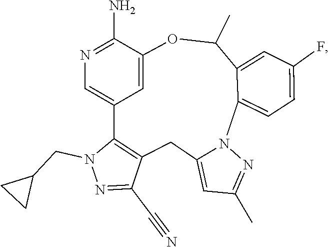 [SMILES: Cc1cc2n(n1)-c1ccc(F)cc1C(C)Oc1cc(cnc1N)-c1c(c(C#N)nn1CC1CC1)C2] | 64 |
| 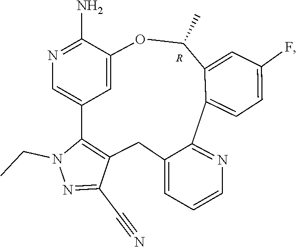 | 65 |
| 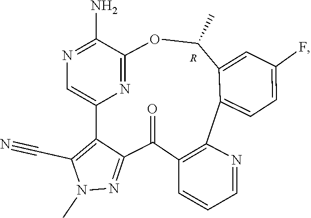 | 66 |
| 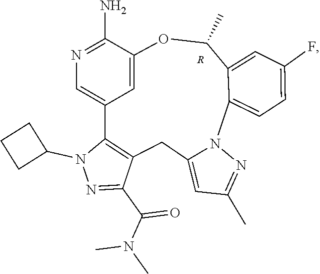 | 67 |
| 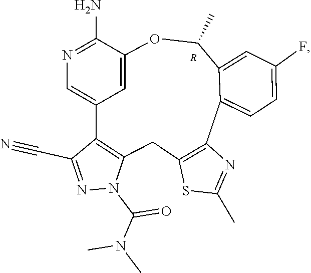 | 68 |
| 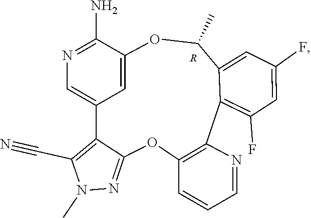 | 69 |
| 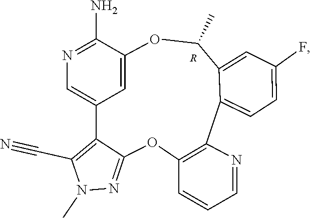 | 70 |
| 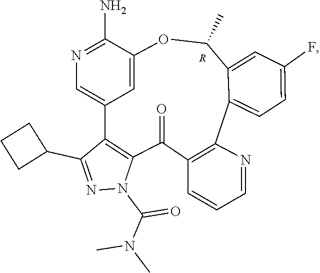 | 71 |
| 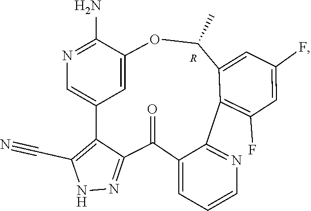 | 72 |
| 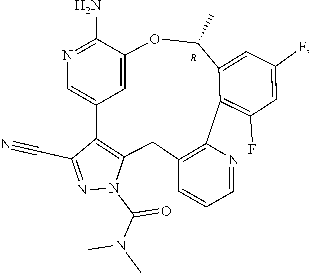 | 73 |
| 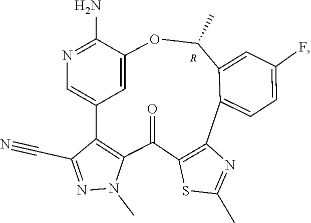 | 74 |
| 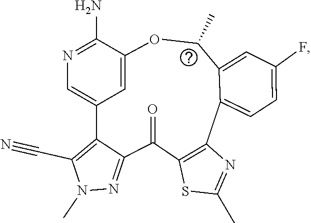 [SMILES: Cc1nc2c(s1)C(=O)c1nn(C)c(C#N)c1-c1cnc(N)c(c1)O[C@H](C)c1cc(F)ccc1-2] | 75 |
| 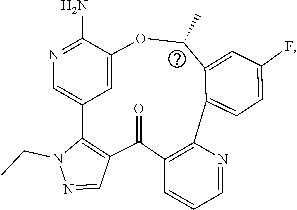 [SMILES: CCn1ncc2c1-c1cnc(N)c(c1)O[C@H](C)c1cc(F)ccc1-c1ncccc1C2=O] | 76 |
| 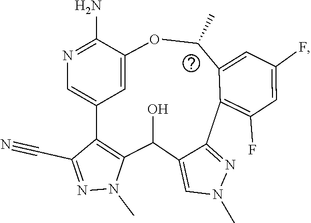 [SMILES: C[C@H]1Oc2cc(cnc2N)-c2c(C#N)nn(C)c2C(O)c2cn(C)nc2-c2c(F)cc(F)cc21] | 77 |
| 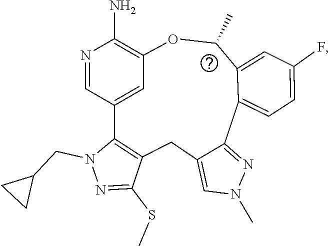 [SMILES: CSc1nn(CC2CC2)c2c1Cc1cn(C)nc1-c1ccc(F)cc1[C@@H](C)Oc1cc-2cnc1N] | 78 |
| 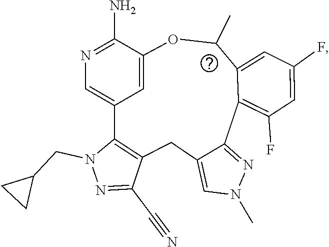 [SMILES: CC1Oc2cc(cnc2N)-c2c(c(C#N)nn2CC2CC2)Cc2cn(C)nc2-c2c(F)cc(F)cc21] | 79 |
| 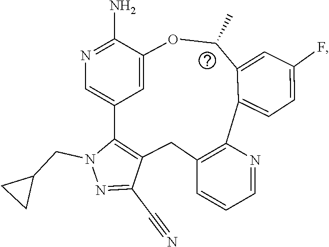 [SMILES: C[C@H]1Oc2cc(cnc2N)-c2c(c(C#N)nn2CC2CC2)Cc2cccnc2-c2ccc(F)cc21] | 80 |

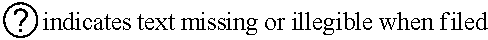

or a pharmaceutically acceptable salt thereof.

In certain embodiments, provided herein is a compound in Table 1A:

TABLE 1A

| | |
|---|---|
| 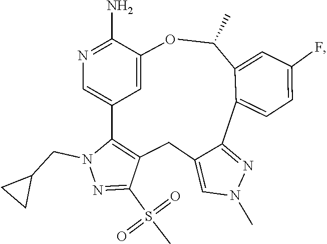 [SMILES: C[C@H]1Oc2cc(cnc2N)-c2c(c(S(C)(=O)=O)nn2CC2CC2)Cc2cn(C)nc2-c2ccc(F)cc21] | 2 |
| 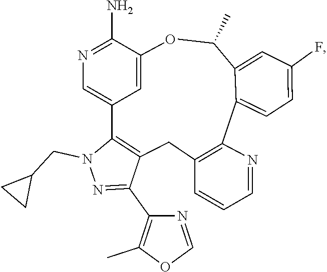 [SMILES: Cc1ocnc1-c1nn(CC2CC2)c2c1Cc1cccnc1-c1ccc(F)cc1[C@@H](C)Oc1cc-2cnc1N] | 3 |
| 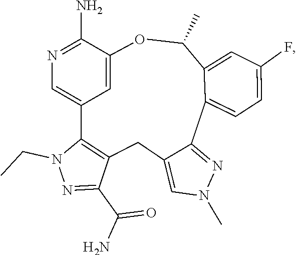 [SMILES: CCn1nc(C(N)=O)c2c1-c1cnc(N)c(c1)O[C@H](C)c1cc(F)ccc1-c1nn(C)cc1C2] | 5 |
| 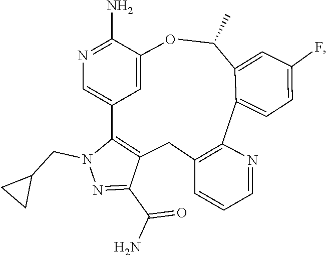 [SMILES: C[C@H]1Oc2cc(cnc2N)-c2c(c(C(N)=O)nn2CC2CC2)Cc2cccnc2-c2ccc(F)cc21] | 6 |
| 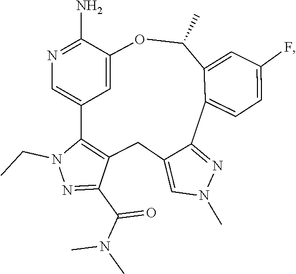 [SMILES: CCn1nc(C(=O)N(C)C)c2c1-c1cnc(N)c(c1)O[C@H](C)c1cc(F)ccc1-c1nn(C)cc1C2] | 7 |
| 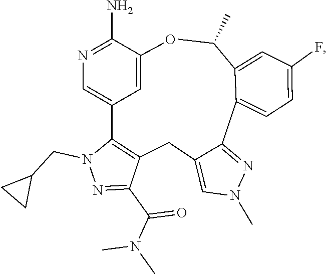 [SMILES: C[C@H]1Oc2cc(cnc2N)-c2c(c(C(=O)N(C)C)nn2CC2CC2)Cc2cn(C)nc2-c2ccc(F)cc21] | 8 |
| 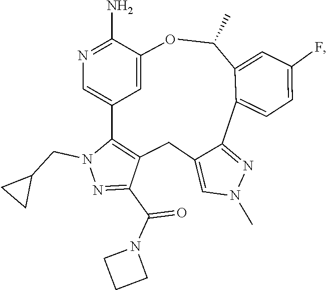 [SMILES: C[C@H]1Oc2cc(cnc2N)-c2c(c(C(=O)N3CCC3)nn2CC2CC2)Cc2cn(C)nc2-c2ccc(F)cc21] | 9 |
| 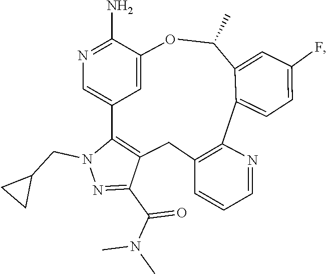 [SMILES: C[C@H]1Oc2cc(cnc2N)-c2c(c(C(=O)N(C)C)nn2CC2CC2)Cc2cccnc2-c2ccc(F)cc21] | 10 |
| 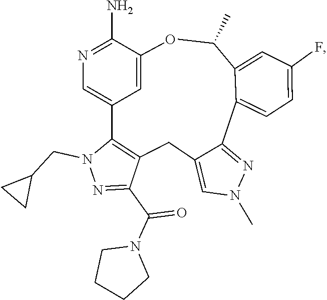 [SMILES: C[C@H]1Oc2cc(cnc2N)-c2c(c(C(=O)N3CCCC3)nn2CC2CC2)Cc2cn(C)nc2-c2ccc(F)cc21] | 11 |
| 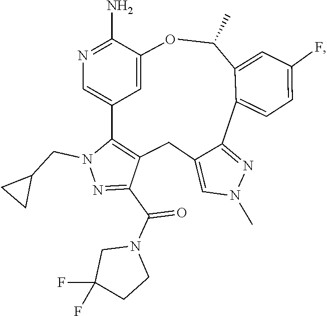 [SMILES: C[C@H]1Oc2cc(cnc2N)-c2c(c(C(=O)N3CCC(F)(F)C3)nn2CC2CC2)Cc2cn(C)nc2-c2ccc(F)cc21] | 12 |
| 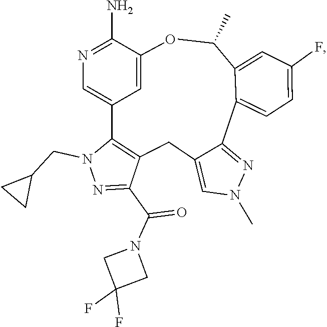 [SMILES: C[C@H]1Oc2cc(cnc2N)-c2c(c(C(=O)N3CC(F)(F)C3)nn2CC2CC2)Cc2cn(C)nc2-c2ccc(F)cc21] | 13 |
| 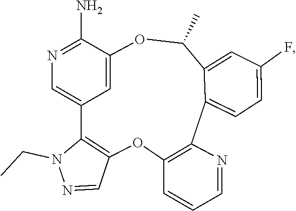 [SMILES: CCn1ncc2c1-c1cnc(N)c(c1)O[C@H](C)c1cc(F)ccc1-c1ncccc1O2] | 14 |
| 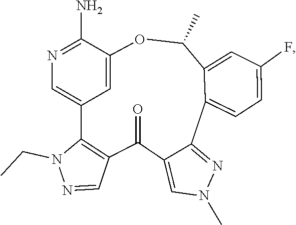 [SMILES: CCn1ncc2c1-c1cnc(N)c(c1)O[C@H](C)c1cc(F)ccc1-c1nn(C)cc1C2=O] | 15 |
| 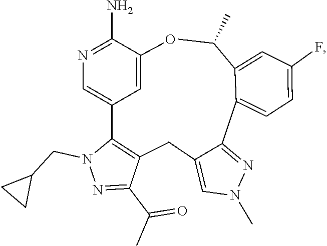 [SMILES: CC(=O)c1nn(CC2CC2)c2c1Cc1cn(C)nc1-c1ccc(F)cc1[C@@H](C)Oc1cc-2cnc1N] | 16 |
| 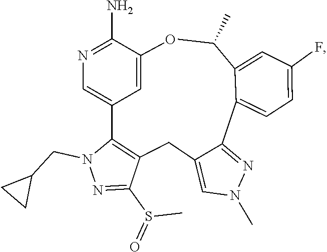 [SMILES: C[C@H]1Oc2cc(cnc2N)-c2c(c(S(C)=O)nn2CC2CC2)Cc2cn(C)nc2-c2ccc(F)cc21] | 17 |
| 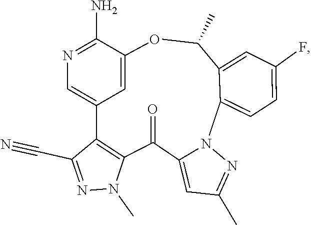 [SMILES: Cc1cc2n(n1)-c1ccc(F)cc1[C@@H](C)Oc1cc(cnc1N)-c1c(C#N)nn(C)c1C2=O] | 18 |
| 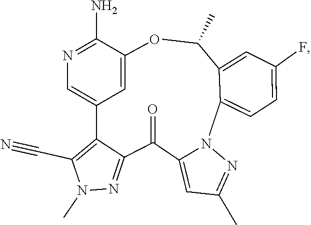 [SMILES: Cc1cc2n(n1)-c1ccc(F)cc1[C@@H](C)Oc1cc(cnc1N)-c1c(nn(C)c1C#N)C2=O] | 19 |
| 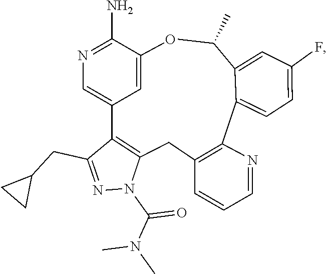 [SMILES: C[C@H]1Oc2cc(cnc2N)-c2c(CC3CC3)nn(C(=O)N(C)C)c2Cc2cccnc2-c2ccc(F)cc21] | 20 |
| 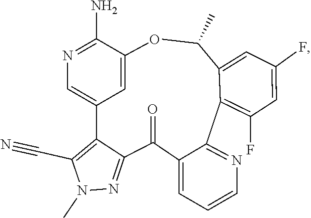 [SMILES: C[C@H]1Oc2cc(cnc2N)-c2c(nn(C)c2C#N)C(=O)c2cccnc2-c2c(F)cc(F)cc21] | 21 |
| 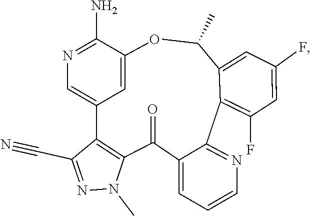 [SMILES: C[C@H]1Oc2cc(cnc2N)-c2c(C#N)nn(C)c2C(=O)c2cccnc2-c2c(F)cc(F)cc21] | 22 |
| 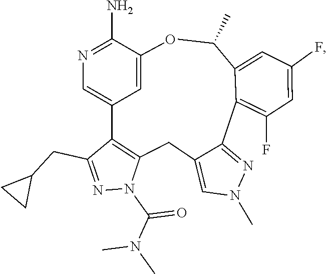 [SMILES: C[C@H]1Oc2cc(cnc2N)-c2c(CC3CC3)nn(C(=O)N(C)C)c2Cc2cn(C)nc2-c2c(F)cc(F)cc21] | 23 |
| 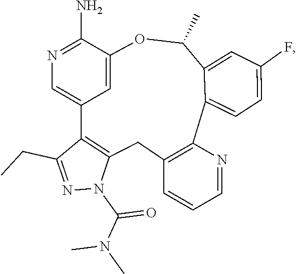 [SMILES: CCc1nn(C(=O)N(C)C)c2c1-c1cnc(N)c(c1)O[C@H](C)c1cc(F)ccc1-c1ncccc1C2] | 24 |
| 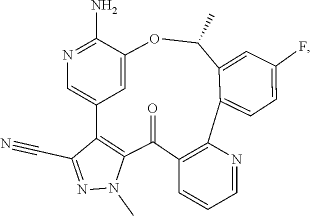 [SMILES: C[C@H]1Oc2cc(cnc2N)-c2c(C#N)nn(C)c2C(=O)c2cccnc2-c2ccc(F)cc21] | 25 |
| 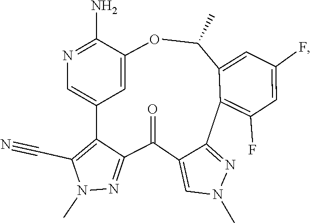 [SMILES: C[C@H]1Oc2cc(cnc2N)-c2c(nn(C)c2C#N)C(=O)c2cn(C)nc2-c2c(F)cc(F)cc21] | 26 |
| 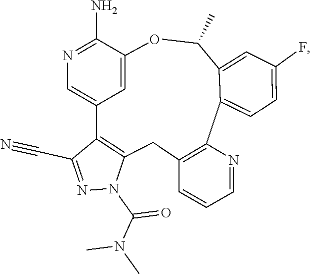 [SMILES: C[C@H]1Oc2cc(cnc2N)-c2c(C#N)nn(C(=O)N(C)C)c2Cc2cccnc2-c2ccc(F)cc21] | 27 |
| 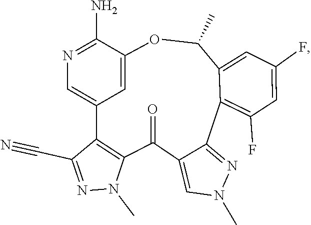 [SMILES: C[C@H]1Oc2cc(cnc2N)-c2c(C#N)nn(C)c2C(=O)c2cn(C)nc2-c2c(F)cc(F)cc21] | 28 |
| 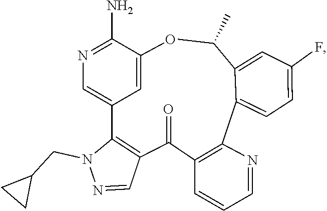 [SMILES: C[C@H]1Oc2cc(cnc2N)-c2c(cnn2CC2CC2)C(=O)c2cccnc2-c2ccc(F)cc21] | 29 |
| 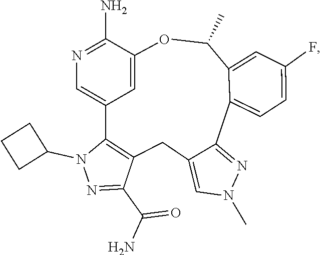 [SMILES: C[C@H]1Oc2cc(cnc2N)-c2c(c(C(N)=O)nn2C2CCC2)Cc2cn(C)nc2-c2ccc(F)cc21] | 30 |
| 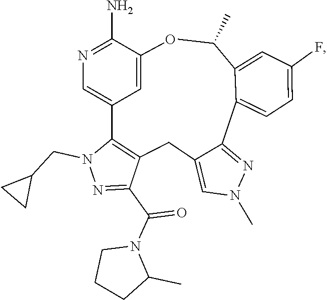 [SMILES: CC1CCCN1C(=O)c1nn(CC2CC2)c2c1Cc1cn(C)nc1-c1ccc(F)cc1[C@@H](C)Oc1cc-2cnc1N] | 31 |
| 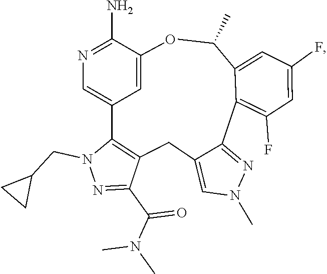 [SMILES: C[C@H]1Oc2cc(cnc2N)-c2c(c(C(=O)N(C)C)nn2CC2CC2)Cc2cn(C)nc2-c2c(F)cc(F)cc21] | 32 |
| 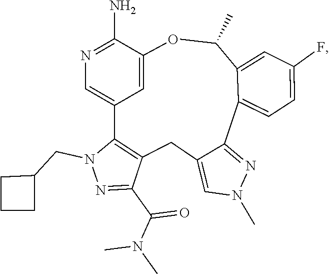 [SMILES: C[C@H]1Oc2cc(cnc2N)-c2c(c(C(=O)N(C)C)nn2CC2CCC2)Cc2cn(C)nc2-c2ccc(F)cc21] | 33 |
| 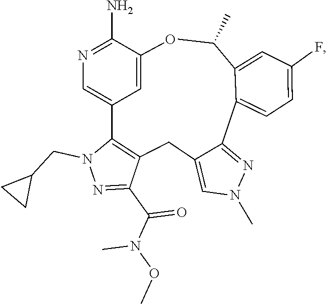 [SMILES: CON(C)C(=O)c1nn(CC2CC2)c2c1Cc1cn(C)nc1-c1ccc(F)cc1[C@@H](C)Oc1cc-2cnc1N] | 34 |
| 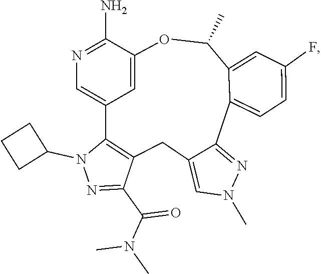 [SMILES: C[C@H]1Oc2cc(cnc2N)-c2c(c(C(=O)N(C)C)nn2C2CCC2)Cc2cn(C)nc2-c2ccc(F)cc21] | 35 |
| 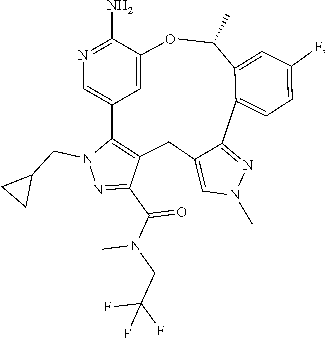 [SMILES: C[C@H]1Oc2cc(cnc2N)-c2c(c(C(=O)N(C)CC(F)(F)F)nn2CC2CC2)Cc2cn(C)nc2-c2ccc(F)cc21] | 36 |
| 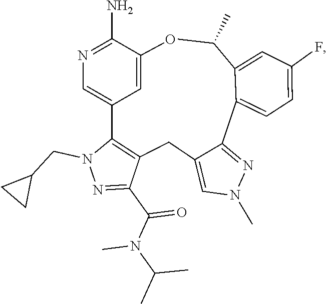 [SMILES: CC(C)N(C)C(=O)c1nn(CC2CC2)c2c1Cc1cn(C)nc1-c1ccc(F)cc1[C@@H](C)Oc1cc-2cnc1N] | 37 |
| 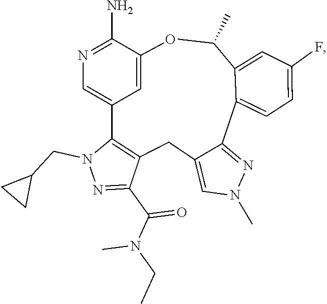 [SMILES: CCN(C)C(=O)c1nn(CC2CC2)c2c1Cc1cn(C)nc1-c1ccc(F)cc1[C@@H](C)Oc1cc-2cnc1N] | 38 |
| 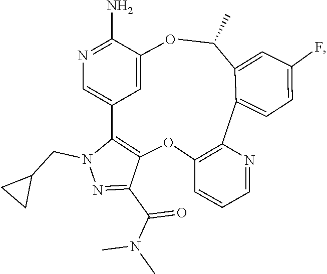 [SMILES: C[C@H]1Oc2cc(cnc2N)-c2c(c(C(=O)N(C)C)nn2CC2CC2)Oc2cccnc2-c2ccc(F)cc21] | 39 |
| 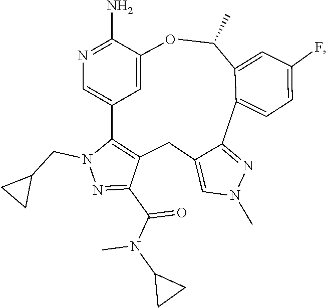 [SMILES: C[C@H]1Oc2cc(cnc2N)-c2c(c(C(=O)N(C)C3CC3)nn2CC2CC2)Cc2cn(C)nc2-c2ccc(F)cc21] | 40 |
| 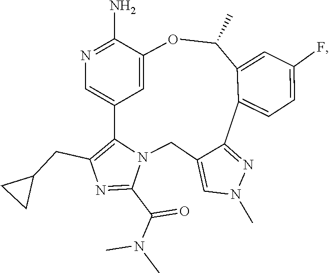 [SMILES: C[C@H]1Oc2cc(cnc2N)-c2c(CC3CC3)nc(C(=O)N(C)C)n2Cc2cn(C)nc2-c2ccc(F)cc21] | 41 |
| 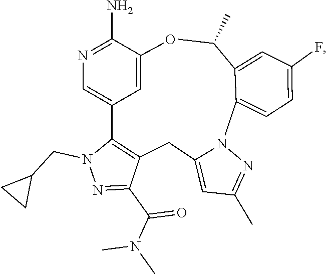 [SMILES: Cc1cc2n(n1)-c1ccc(F)cc1[C@@H](C)Oc1cc(cnc1N)-c1c(c(C(=O)N(C)C)nn1CC1CC1)C2] | 42 |
| 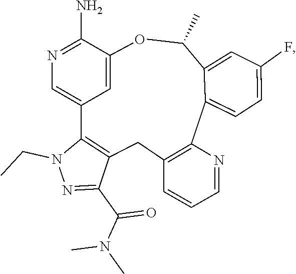 [SMILES: CCn1nc(C(=O)N(C)C)c2c1-c1cnc(N)c(c1)O[C@H](C)c1cc(F)ccc1-c1ncccc1C2] | 43 |
| 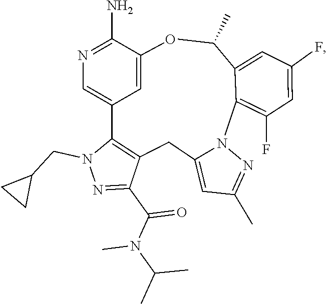 [SMILES: Cc1cc2n(n1)-c1c(F)cc(F)cc1[C@@H](C)Oc1cc(cnc1N)-c1c(c(C(=O)N(C)C(C)C)nn1CC1CC1)C2] | 44 |
| 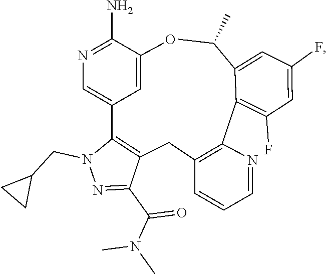 [SMILES: C[C@H]1Oc2cc(cnc2N)-c2c(c(C(=O)N(C)C)nn2CC2CC2)Cc2cccnc2-c2c(F)cc(F)cc21] | 45 |
| 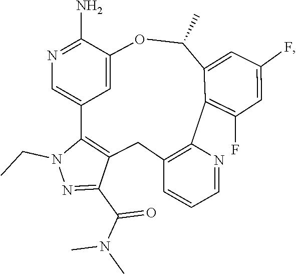 [SMILES: CCn1nc(C(=O)N(C)C)c2c1-c1cnc(N)c(c1)O[C@H](C)c1cc(F)cc(F)c1-c1ncccc1C2] | 46 |
| 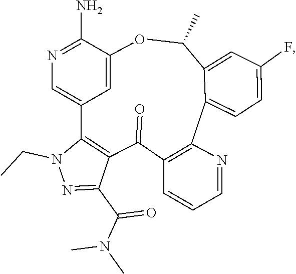 [SMILES: CCn1nc(C(=O)N(C)C)c2c1-c1cnc(N)c(c1)O[C@H](C)c1cc(F)ccc1-c1ncccc1C2=O] | 47 |
| 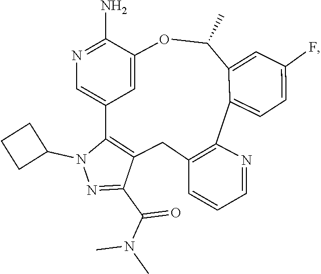 [SMILES: C[C@H]1Oc2cc(cnc2N)-c2c(c(C(=O)N(C)C)nn2C2CCC2)Cc2cccnc2-c2ccc(F)cc21] | 48 |
| 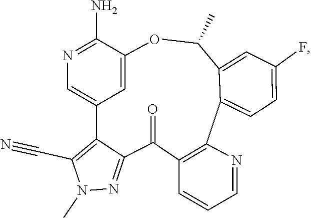 [SMILES: C[C@H]1Oc2cc(cnc2N)-c2c(nn(C)c2C#N)C(=O)c2cccnc2-c2ccc(F)cc21] | 49 |
| 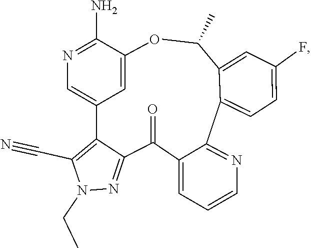 [SMILES: CCn1nc2c(c1C#N)-c1cnc(N)c(c1)O[C@H](C)c1cc(F)ccc1-c1ncccc1C2=O] | 50 |
| 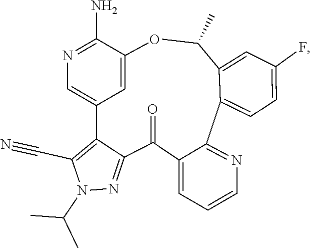 [SMILES: CC(C)n1nc2c(c1C#N)-c1cnc(N)c(c1)O[C@H](C)c1cc(F)ccc1-c1ncccc1C2=O] | 51 |
| 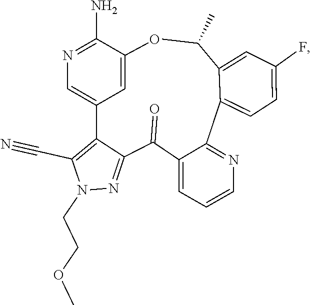 [SMILES: COCCn1nc2c(c1C#N)-c1cnc(N)c(c1)O[C@H](C)c1cc(F)ccc1-c1ncccc1C2=O] | 52 |
| 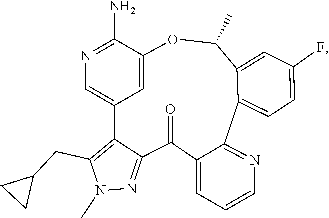 [SMILES: C[C@H]1Oc2cc(cnc2N)-c2c(nn(C)c2CC2CC2)C(=O)c2cccnc2-c2ccc(F)cc21] | 53 |
| 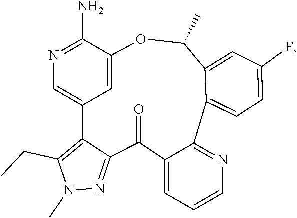 [SMILES: CCc1c2c(nn1C)C(=O)c1cccnc1-c1ccc(F)cc1[C@@H](C)Oc1cc-2cnc1N] | 54 |
| 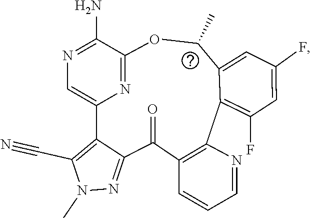 [SMILES: C[C@H]1Oc2nc(cnc2N)-c2c(nn(C)c2C#N)C(=O)c2cccnc2-c2c(F)cc(F)cc21] | 57 |
| 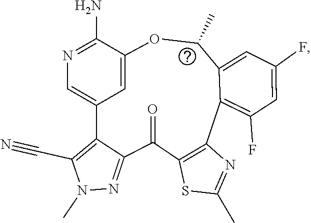 [SMILES: Cc1nc2c(s1)C(=O)c1nn(C)c(C#N)c1-c1cnc(N)c(c1)O[C@H](C)c1cc(F)cc(F)c1-2] | 58 |
| 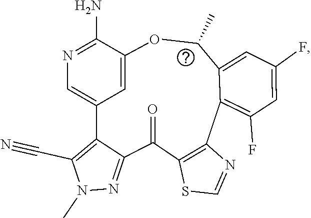 [SMILES: C[C@H]1Oc2cc(cnc2N)-c2c(nn(C)c2C#N)C(=O)c2scnc2-c2c(F)cc(F)cc21] | 59 |
| 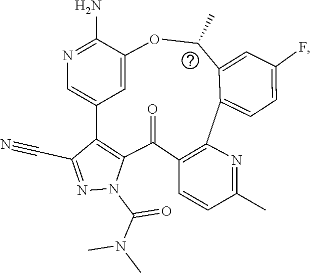 [SMILES: Cc1ccc2c(n1)-c1ccc(F)cc1[C@@H](C)Oc1cc(cnc1N)-c1c(C#N)nn(C(=O)N(C)C)c1C2=O] | 60 |
| 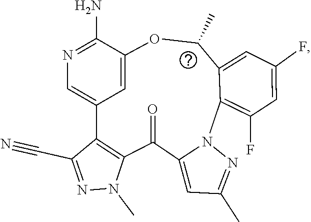 [SMILES: Cc1cc2n(n1)-c1c(F)cc(F)cc1[C@@H](C)Oc1cc(cnc1N)-c1c(C#N)nn(C)c1C2=O] | 61 |
| 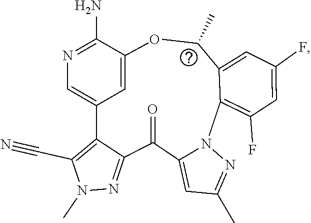 [SMILES: Cc1cc2n(n1)-c1c(F)cc(F)cc1[C@@H](C)Oc1cc(cnc1N)-c1c(nn(C)c1C#N)C2=O] | 62 |
| 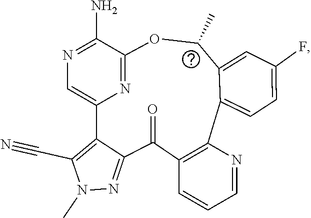 [SMILES: C[C@H]1Oc2nc(cnc2N)-c2c(nn(C)c2C#N)C(=O)c2cccnc2-c2ccc(F)cc21] | 66 |
| 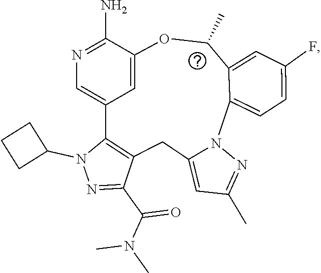 [SMILES: Cc1cc2n(n1)-c1ccc(F)cc1[C@@H](C)Oc1cc(cnc1N)-c1c(c(C(=O)N(C)C)nn1C1CCC1)C2] | 67 |
| 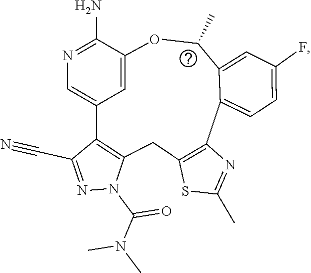 [SMILES: Cc1nc2c(s1)Cc1c(c(C#N)nn1C(=O)N(C)C)-c1cnc(N)c(c1)O[C@H](C)c1cc(F)ccc1-2] | 68 |
| 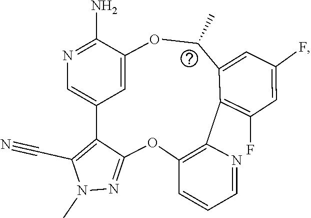 [SMILES: C[C@H]1Oc2cc(cnc2N)-c2c(nn(C)c2C#N)Oc2cccnc2-c2c(F)cc(F)cc21] | 69 |
| 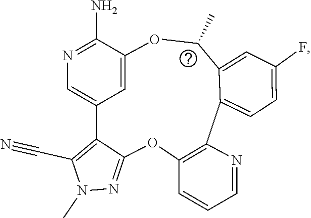 [SMILES: C[C@H]1Oc2cc(cnc2N)-c2c(nn(C)c2C#N)Oc2cccnc2-c2ccc(F)cc21] | 70 |
| 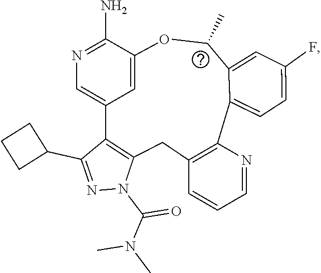 [SMILES: C[C@H]1Oc2cc(cnc2N)-c2c(C3CCC3)nn(C(=O)N(C)C)c2Cc2cccnc2-c2ccc(F)cc21] | 71 |
| 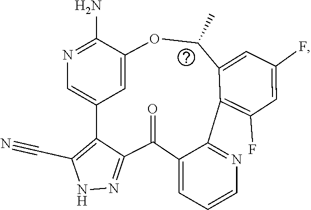 [SMILES: C[C@H]1Oc2cc(cnc2N)-c2c(n[nH]c2C#N)C(=O)c2cccnc2-c2c(F)cc(F)cc21] | 72 |
| 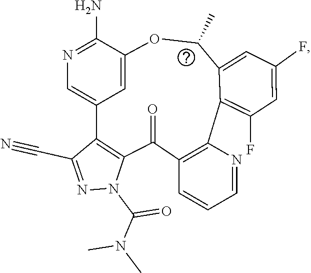 [SMILES: C[C@H]1Oc2cc(cnc2N)-c2c(C#N)nn(C(=O)N(C)C)c2C(=O)c2cccnc2-c2c(F)cc(F)cc21] | 73 |
| 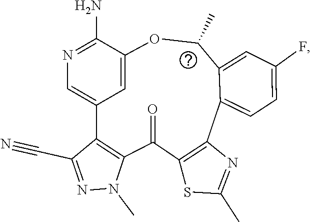 [SMILES: Cc1nc2c(s1)C(=O)c1c(c(C#N)nn1C)-c1cnc(N)c(c1)O[C@H](C)c1cc(F)ccc1-2] | 74 |
| 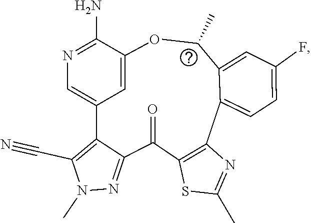 [SMILES: Cc1nc2c(s1)C(=O)c1nn(C)c(C#N)c1-c1cnc(N)c(c1)O[C@H](C)c1cc(F)ccc1-2] | 75 |
| 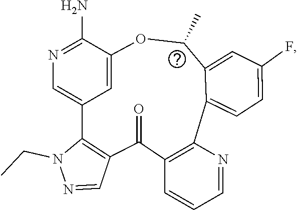 [SMILES: CCn1ncc2c1-c1cnc(N)c(c1)O[C@H](C)c1cc(F)ccc1-c1ncccc1C2=O] | 76 |

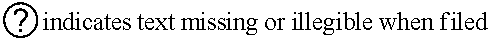

or a pharmaceutically acceptable salt thereof.

In certain embodiments, provided herein is a compound in Table 1B:

TABLE 1B

| | |
|---|---|
| 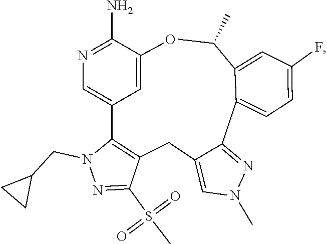 [SMILES: C[C@H]1Oc2cc(cnc2N)-c2c(c(S(C)(=O)=O)nn2CC2CC2)Cc2cn(C)nc2-c2ccc(F)cc21] | 2 |
| 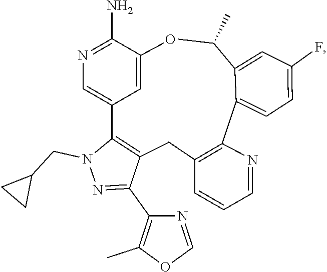 [SMILES: Cc1ocnc1-c1nn(CC2CC2)c2c1Cc1cccnc1-c1ccc(F)cc1[C@@H](C)Oc1cc-2cnc1N] | 3 |
| 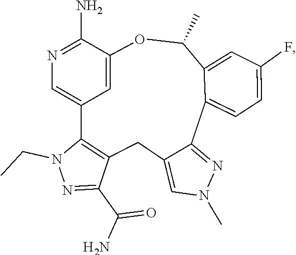 [SMILES: CCn1nc(C(N)=O)c2c1-c1cnc(N)c(c1)O[C@H](C)c1cc(F)ccc1-c1nn(C)cc1C2] | 5 |
| 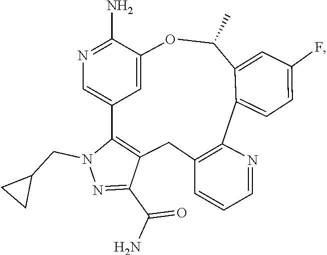 [SMILES: C[C@H]1Oc2cc(cnc2N)-c2c(c(C(N)=O)nn2CC2CC2)Cc2cccnc2-c2ccc(F)cc21] | 6 |
| 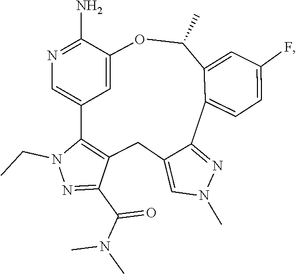 [SMILES: CCn1nc(C(=O)N(C)C)c2c1-c1cnc(N)c(c1)O[C@H](C)c1cc(F)ccc1-c1nn(C)cc1C2] | 7 |
| 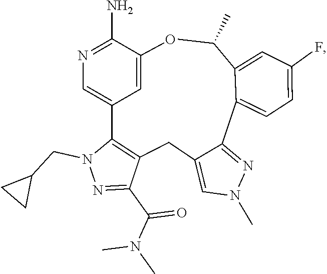 [SMILES: C[C@H]1Oc2cc(cnc2N)-c2c(c(C(=O)N(C)C)nn2CC2CC2)Cc2cn(C)nc2-c2ccc(F)cc21] | 8 |
| 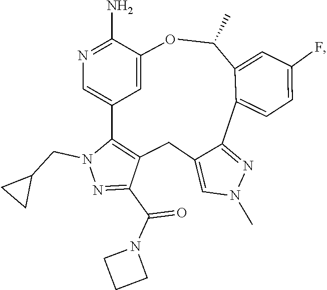 [SMILES: C[C@H]1Oc2cc(cnc2N)-c2c(c(C(=O)N3CCC3)nn2CC2CC2)Cc2cn(C)nc2-c2ccc(F)cc21] | 9 |
| 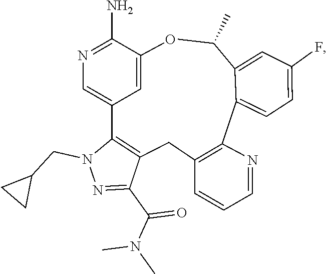 [SMILES: C[C@H]1Oc2cc(cnc2N)-c2c(c(C(=O)N(C)C)nn2CC2CC2)Cc2cccnc2-c2ccc(F)cc21] | 10 |
| 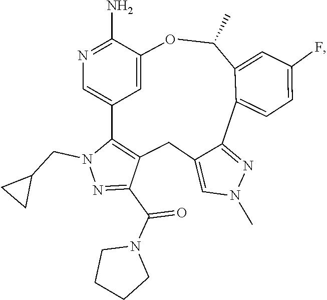 [SMILES: C[C@H]1Oc2cc(cnc2N)-c2c(c(C(=O)N3CCCC3)nn2CC2CC2)Cc2cn(C)nc2-c2ccc(F)cc21] | 11 |
| 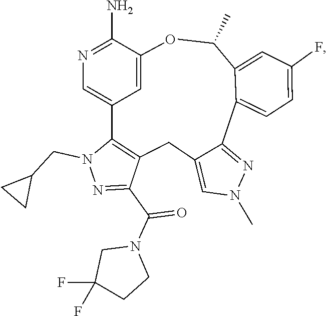 [SMILES: C[C@H]1Oc2cc(cnc2N)-c2c(c(C(=O)N3CCC(F)(F)C3)nn2CC2CC2)Cc2cn(C)nc2-c2ccc(F)cc21] | 12 |
| 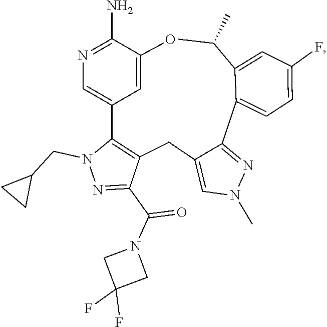 [SMILES: C[C@H]1Oc2cc(cnc2N)-c2c(c(C(=O)N3CC(F)(F)C3)nn2CC2CC2)Cc2cn(C)nc2-c2ccc(F)cc21] | 13 |
| 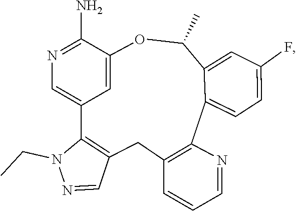 [SMILES: CCn1ncc2c1-c1cnc(N)c(c1)O[C@H](C)c1cc(F)ccc1-c1ncccc1C2] | 14 |
| 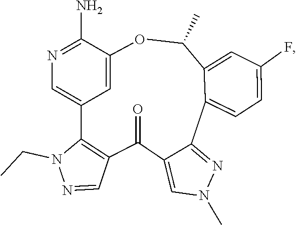 [SMILES: CCn1ncc2c1-c1cnc(N)c(c1)O[C@H](C)c1cc(F)ccc1-c1nn(C)cc1C2=O] | 15 |
| 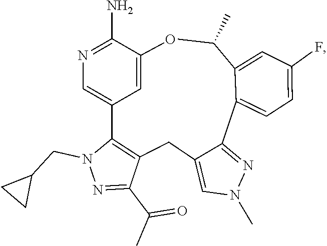 [SMILES: CC(=O)c1nn(CC2CC2)c2c1Cc1cn(C)nc1-c1ccc(F)cc1[C@@H](C)Oc1cc-2cnc1N] | 16 |
| 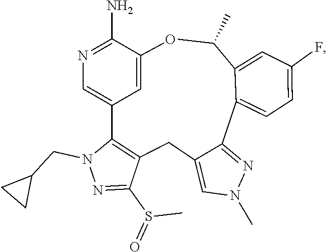 [SMILES: C[C@H]1Oc2cc(cnc2N)-c2c(c(S(C)=O)nn2CC2CC2)Cc2cn(C)nc2-c2ccc(F)cc21] | 17 |
| 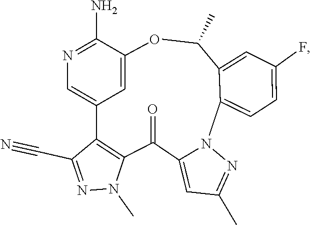 [SMILES: Cc1cc2n(n1)-c1ccc(F)cc1[C@@H](C)Oc1cc(cnc1N)-c1c(C#N)nn(C)c1C2=O] | 18 |
| 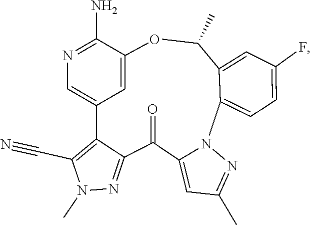 [SMILES: Cc1cc2n(n1)-c1ccc(F)cc1[C@@H](C)Oc1cc(cnc1N)-c1c(nn(C)c1C#N)C2=O] | 19 |
| 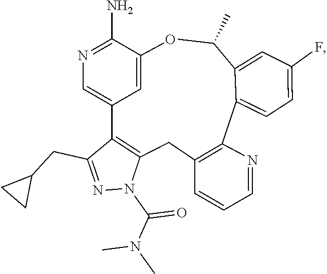 [SMILES: C[C@H]1Oc2cc(cnc2N)-c2c(CC3CC3)nn(C(=O)N(C)C)c2Cc2cccnc2-c2ccc(F)cc21] | 20 |
| 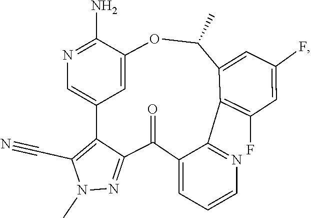 [SMILES: C[C@H]1Oc2cc(cnc2N)-c2c(nn(C)c2C#N)C(=O)c2cccnc2-c2c(F)cc(F)cc21] | 21 |
| 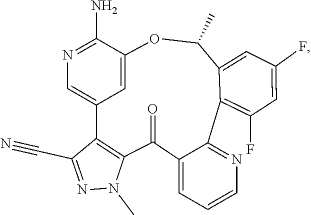 [SMILES: C[C@H]1Oc2cc(cnc2N)-c2c(C#N)nn(C)c2C(=O)c2cccnc2-c2c(F)cc(F)cc21] | 22 |
| 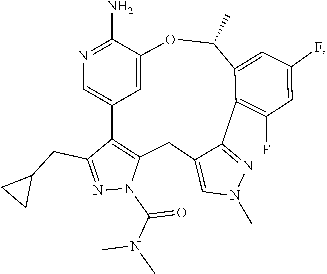 [SMILES: C[C@H]1Oc2cc(cnc2N)-c2c(CC3CC3)nn(C(=O)N(C)C)c2Cc2cn(C)nc2-c2c(F)cc(F)cc21] | 23 |
| 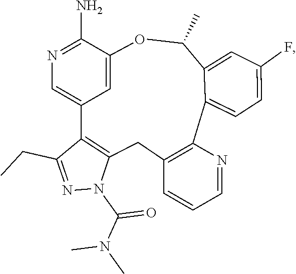 [SMILES: CCc1nn(C(=O)N(C)C)c2c1-c1cnc(N)c(c1)O[C@H](C)c1cc(F)ccc1-c1ncccc1C2] | 24 |
| 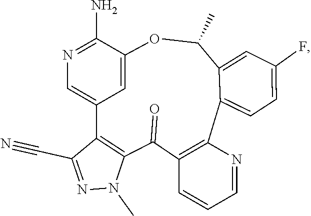 [SMILES: C[C@H]1Oc2cc(cnc2N)-c2c(C#N)nn(C)c2C(=O)c2cccnc2-c2ccc(F)cc21] | 25 |
| 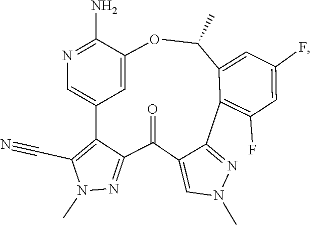 [SMILES: C[C@H]1Oc2cc(cnc2N)-c2c(nn(C)c2C#N)C(=O)c2cn(C)nc2-c2c(F)cc(F)cc21] | 26 |
| 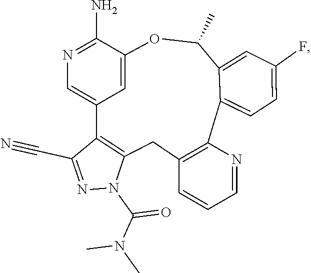 [SMILES: C[C@H]1Oc2cc(cnc2N)-c2c(C#N)nn(C(=O)N(C)C)c2Cc2cccnc2-c2ccc(F)cc21] | 27 |
| 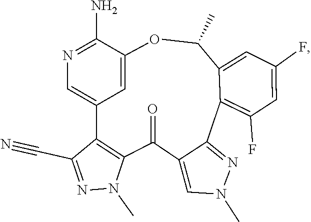 [SMILES: C[C@H]1Oc2cc(cnc2N)-c2c(C#N)nn(C)c2C(=O)c2cn(C)nc2-c2c(F)cc(F)cc21] | 28 |
| 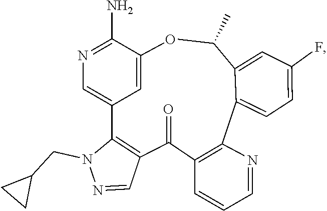 [SMILES: C[C@H]1Oc2cc(cnc2N)-c2c(cnn2CC2CC2)C(=O)c2cccnc2-c2ccc(F)cc21] | 29 |
| 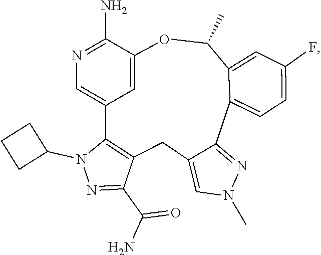 [SMILES: C[C@H]1Oc2cc(cnc2N)-c2c(c(C(N)=O)nn2C2CCC2)Cc2cn(C)nc2-c2ccc(F)cc21] | 30 |
| 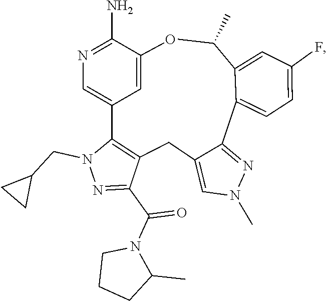 [SMILES: CC1CCCN1C(=O)c1nn(CC2CC2)c2c1Cc1cn(C)nc1-c1ccc(F)cc1[C@@H](C)Oc1cc-2cnc1N] | 31 |
| 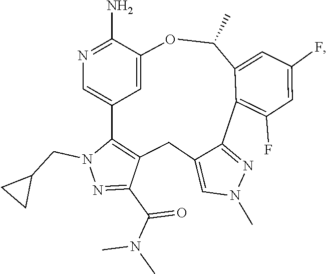 [SMILES: C[C@H]1Oc2cc(cnc2N)-c2c(c(C(=O)N(C)C)nn2CC2CC2)Cc2cn(C)nc2-c2c(F)cc(F)cc21] | 32 |
| 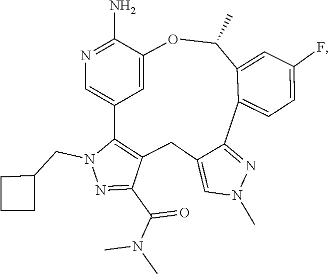 [SMILES: C[C@H]1Oc2cc(cnc2N)-c2c(c(C(=O)N(C)C)nn2CC2CCC2)Cc2cn(C)nc2-c2ccc(F)cc21] | 33 |
| 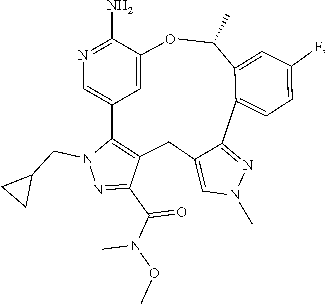 [SMILES: CON(C)C(=O)c1nn(CC2CC2)c2c1Cc1cn(C)nc1-c1ccc(F)cc1[C@@H](C)Oc1cc-2cnc1N] | 34 |
| 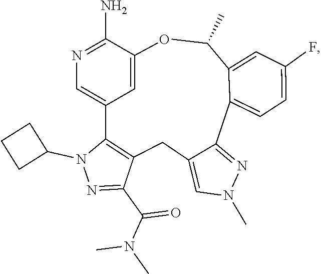 [SMILES: C[C@H]1Oc2cc(cnc2N)-c2c(c(C(=O)N(C)C)nn2C2CCC2)Cc2cn(C)nc2-c2ccc(F)cc21] | 35 |
| 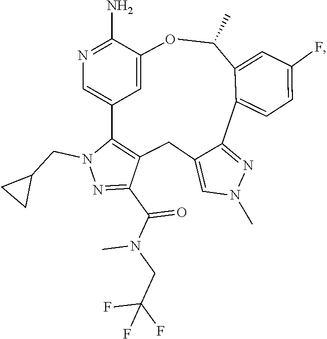 [SMILES: C[C@H]1Oc2cc(cnc2N)-c2c(c(C(=O)N(C)CC(F)(F)F)nn2CC2CC2)Cc2cn(C)nc2-c2ccc(F)cc21] | 36 |
| 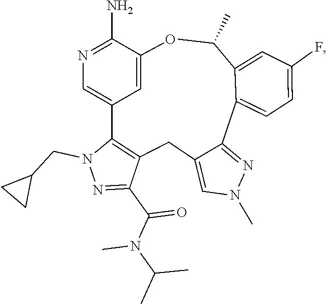 [SMILES: CC(C)N(C)C(=O)c1nn(CC2CC2)c2c1Cc1cn(C)nc1-c1ccc(F)cc1[C@@H](C)Oc1cc-2cnc1N] | 37 |
| 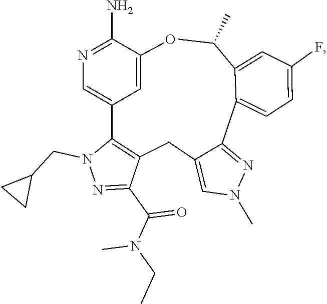 [SMILES: CCN(C)C(=O)c1nn(CC2CC2)c2c1Cc1cn(C)nc1-c1ccc(F)cc1[C@@H](C)Oc1cc-2cnc1N] | 38 |
| 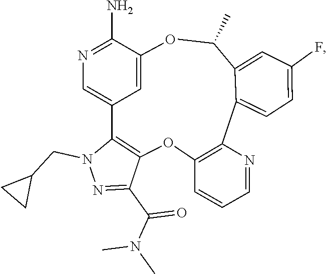 [SMILES: C[C@H]1Oc2cc(cnc2N)-c2c(c(C(=O)N(C)C)nn2CC2CC2)Oc2cccnc2-c2ccc(F)cc21] | 39 |
| 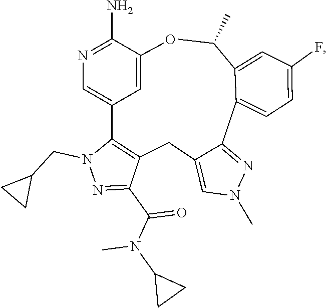 [SMILES: C[C@H]1Oc2cc(cnc2N)-c2c(c(C(=O)N(C)C3CC3)nn2CC2CC2)Cc2cn(C)nc2-c2ccc(F)cc21] | 40 |
| 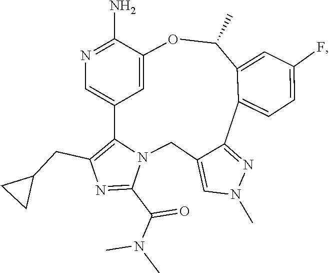 [SMILES: C[C@H]1Oc2cc(cnc2N)-c2c(CC3CC3)nc(C(=O)N(C)C)n2Cc2cn(C)nc2-c2ccc(F)cc21] | 41 |
| 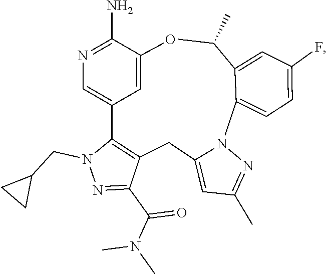 [SMILES: Cc1cc2n(n1)-c1ccc(F)cc1[C@@H](C)Oc1cc(cnc1N)-c1c(c(C(=O)N(C)C)nn1CC1CC1)C2] | 42 |
| 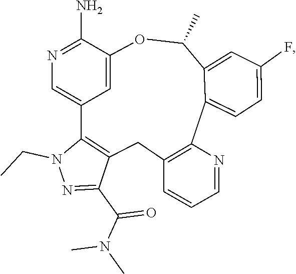 [SMILES: CCn1nc(C(=O)N(C)C)c2c1-c1cnc(N)c(c1)O[C@H](C)c1cc(F)ccc1-c1ncccc1C2] | 43 |
| 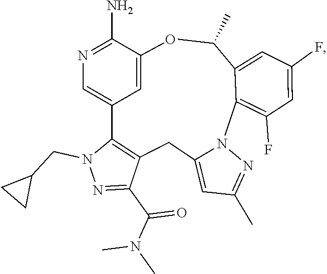 [SMILES: Cc1cc2n(n1)-c1c(F)cc(F)cc1[C@@H](C)Oc1cc(cnc1N)-c1c(c(C(=O)N(C)C)nn1CC1CC1)C2] | 44 |
| 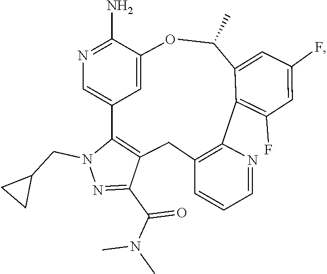 [SMILES: C[C@H]1Oc2cc(cnc2N)-c2c(c(C(=O)N(C)C)nn2CC2CC2)Cc2cccnc2-c2c(F)cc(F)cc21] | 45 |
| 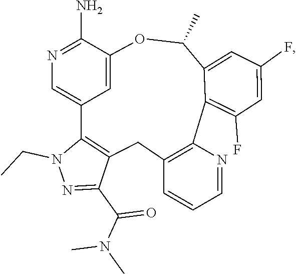 [SMILES: CCn1nc(C(=O)N(C)C)c2c1-c1cnc(N)c(c1)O[C@H](C)c1cc(F)cc(F)c1-c1ncccc1C2] | 46 |
| 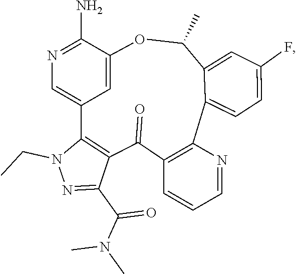 [SMILES: CCn1nc(C(=O)N(C)C)c2c1-c1cnc(N)c(c1)O[C@H](C)c1cc(F)ccc1-c1ncccc1C2=O] | 47 |
| 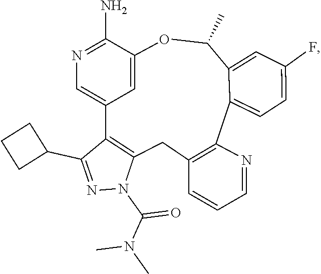 [SMILES: C[C@H]1Oc2cc(cnc2N)-c2c(C3CCC3)nn(C(=O)N(C)C)c2Cc2cccnc2-c2ccc(F)cc21] | 48 |
| 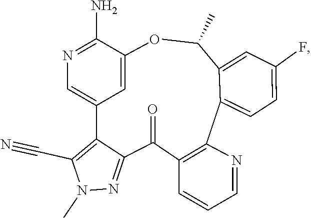 [SMILES: C[C@H]1Oc2cc(cnc2N)-c2c(nn(C)c2C#N)C(=O)c2cccnc2-c2ccc(F)cc21] | 49 |
| 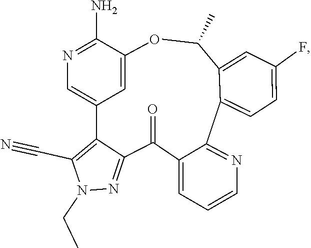 [SMILES: CCn1nc2c(c1C#N)-c1cnc(N)c(c1)O[C@H](C)c1cc(F)ccc1-c1ncccc1C2=O] | 50 |
| 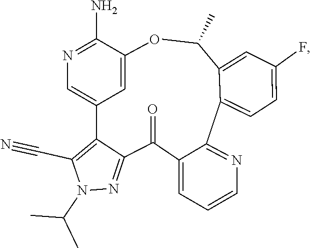 [SMILES: CC(C)n1nc2c(c1C#N)-c1cnc(N)c(c1)O[C@H](C)c1cc(F)ccc1-c1ncccc1C2=O] | 51 |
| 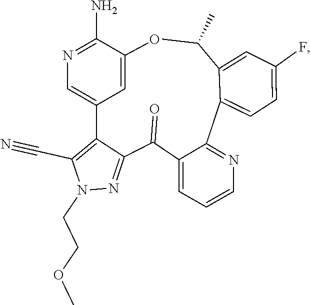 [SMILES: COCCn1nc2c(c1C#N)-c1cnc(N)c(c1)O[C@H](C)c1cc(F)ccc1-c1ncccc1C2=O] | 52 |
| 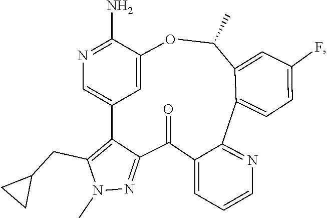 [SMILES: C[C@H]1Oc2cc(cnc2N)-c2c(nn(C)c2CC2CC2)C(=O)c2cccnc2-c2ccc(F)cc21] | 53 |
| 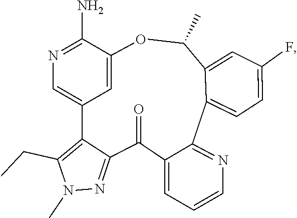 [SMILES: CCc1c2c(nn1C)C(=O)c1cccnc1-c1ccc(F)cc1[C@@H](C)Oc1cc-2cnc1N] | 54 |
| 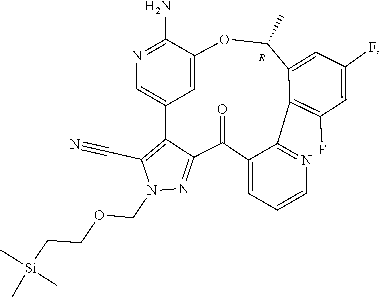 | 55 |
| 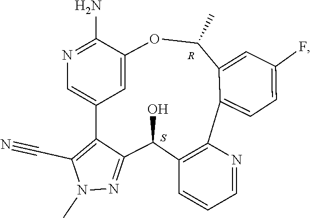 | 56 |
| 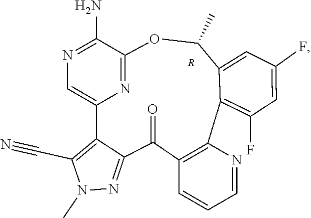 | 57 |
| 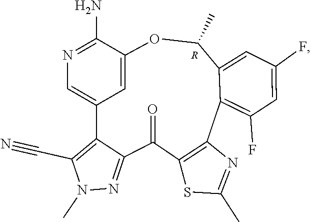 | 58 |
| 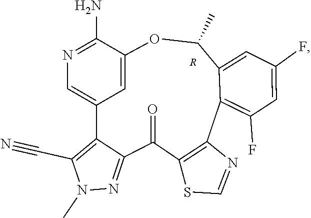 | 59 |
| 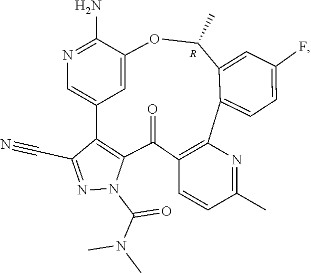 | 60 |
| 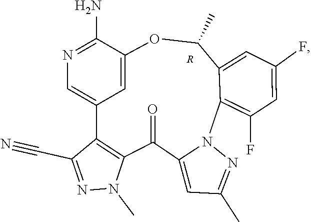 | 61 |
| 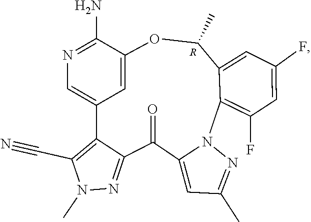 | 62 |
| 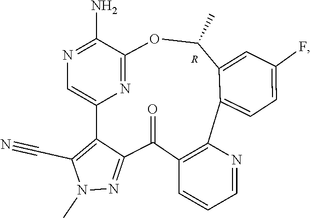 | 66 |
| 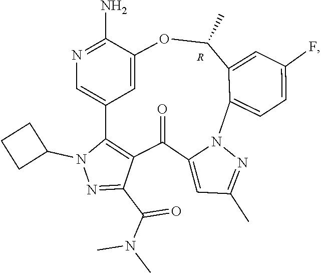 | 67 |
| 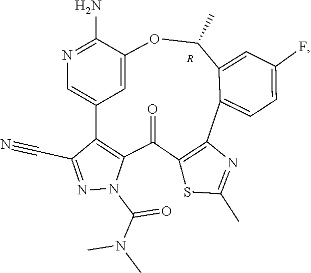 | 68 |
| 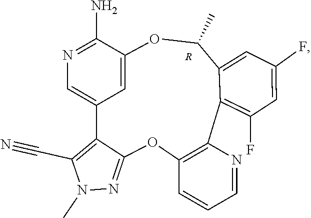 | 69 |
| 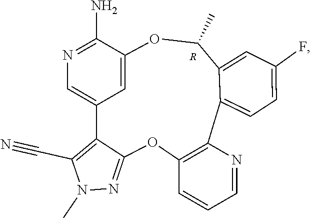 | 70 |
| 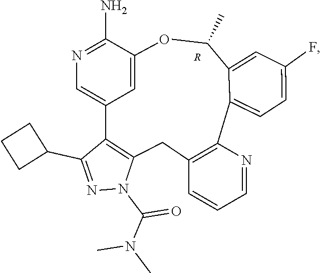 | 71 |
| 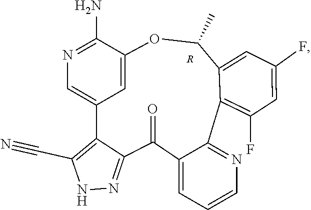 | 72 |
| 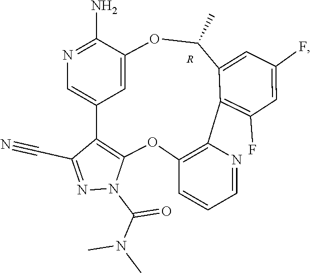 | 73 |
| 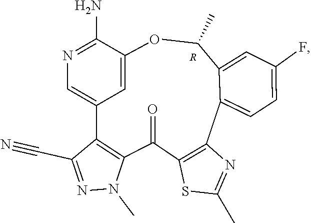 | 74 |
| 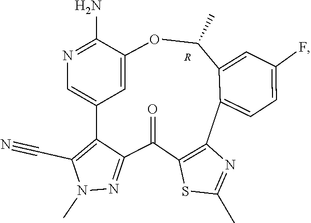 | 75 |
| 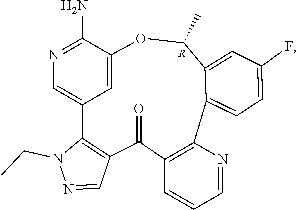 | 76 |
| 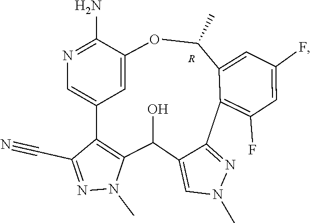 | 77 |
| 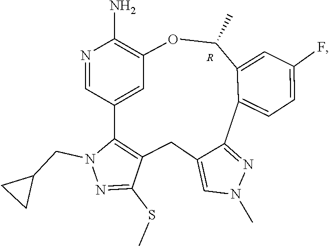 | 78 |

or a pharmaceutically acceptable salt thereof.

For any compound in Table 1, Table 1A, or Table 1B that has a chiral center due to the presence of non-hydrogen R₁, the R-enantiomer, the S-enantiomer, and the racemic compound of such compound are all specifically provided herein, even if not specifically shown in Table 1, Table 1A, or Table 1B.

In certain embodiments, provided herein is a pharmaceutically acceptable salt of a compound of Formula (I). In certain embodiments, provided herein is a pharmaceutically acceptable salt of any compound in Table 1, Table 1A, or Table 1B.

In certain embodiments, the pharmaceutically acceptable salt of the compound is selected from the group consisting of alkyl ammonium salts, dialkyl ammonium salts, trialkyl ammonium salts, tetra-alkyl ammonium salts, L-arginine salts, benenthamine salts, benzathine salts, betaine salts, calcium hydroxide salts, choline salts, deanol salts, diethanolamine salts, diethylamine salts, 2-(diethylamino)ethanol salts, ethanolamine salts, ethylenediamine salts, N-methylglucamine salts, hydrabamine salts, 1H-imidazole salts, lithium salts, L-lysine salts, magnesium salts, 4-(2-hydroxyethyl)morpholine salts, piperazine salts, potassium salts, 1-(2-hydroxyethyl)pyrrolidine salts, sodium salts, triethanolamine salts, tromethamine salts, Na salts, Ca salts, K salts, Mg salts, and Zn salts.

In specific embodiments, the pharmaceutically acceptable salt is a solvate selected from the group consisting of water, methanol, ethanol, and dimethylformamide.

In certain embodiments the compound is a pharmaceutical composition including a pharmaceutically acceptable carrier or excipient.

In specific embodiments, the composition is in a form selected from the group consisting of a tablet, a capsule, a granule, a lyophile for reconstitution, a powder, a solution, a syrup, a suppository, an injection, a transdermal delivery system, and a solution suitable for topical administration.

### 4.3 Methods of Use

Provided herein are methods of treating cancer comprising administering a compound provided herein, such as a compound of Formula (I), or a stereoisomer, a mixture of stereoisomers, or a pharmaceutically acceptable salt thereof.

Cancer is a disease of uncontrolled cell proliferation that results from alterations in certain genes. Some of these alterations occur in genes that encode receptor tyrosine kinases (RTKs), a family of membrane-bound proteins that transmit signals from outside the cell to promote cell survival, growth, and proliferation. Aberrant RTK activation can lead to excessive cell growth and hence cancer. Generally, RTKs contain an N-terminal domain that binds extracellular ligands, a transmembrane domain, and a C-terminal kinase domain that catalyzes intracellular signal transduction.

In some embodiments, the compound of Formula (I) is an inhibitor of human ROS1. ROS1 is an RTK encoded by the ROS1 gene. The ligands and biological functions of human ROS1 are unknown, but its homologs in some other species have been shown to bind extracellular ligands and stimulate cell differentiation. For example, mouse ROS1 is essential for male gamete maturation and reproduction. In humans, ROS1 chromosomal rearrangements are a well-documented cause of cancer, representing 1-2% of non-small cell lung cancer (NSCLC) and a subset of many other cancers. These rearrangements result in the fusion of the C-terminus of ROS1 with the N-terminus of various partner proteins, the most common of which is CD74. ROS1 fusions have constitutive kinase activity that drives tumor growth through MAPK, PI3K, and JAK/STAT signaling pathways. Small-molecule tyrosine kinase inhibitors (TKIs) have been used to target ROS1 fusions in cancer, including crizotinib and entrectinib. Crizotinib was the first FDA-approved TKI for the treatment of ROS1-positive NSCLC, with an overall response rate of 60-80% and median progression-free survival of 9-19 months. Despite an initial response, most patients acquire resistance to crizotinib and relapse. The predominant mechanism of resistance is the G2032R mutation in the solvent front, which dramatically reduces crizotinib affinity. No inhibitors with activity against ROS1-G2032R fusions have been FDA-approved, indicating a need in the art.

In some embodiments, the compound of Formula (I) is an inhibitor of human anaplastic lymphoma kinase (ALK). ALK, also known as cluster of differentiation 246 (CD246), is an RTK encoded by the ALK gene. ALK and ROS1 are evolutionarily related; both belong to the insulin receptor superfamily, and their kinase domains share around 80% sequence similarity. While the roles of ALK in humans remain inconclusive, much evidence from mouse studies suggests that it is important for the development of the nervous system. Like ROS1, ALK chromosomal rearrangements also lead to constitutively active fusion proteins that promote oncogenic transformation through MAPK, JAK/STAT, or other signaling pathways. ALK rearrangements represent 3-5% of NSCLC, roughly half of anaplastic large-cell lymphoma (ALCL), and a subset of many other cancers, with the predominant fusions being EML4-ALK for NSCLC and NPM1-ALK for ALCL. Oncogenic point mutations and amplification of ALK have also been observed, albeit at a much lower frequency than translocations. Crizotinib, ceritinib, alectinib, brigatinib, and lorlatinib are FDA-approved TKIs for the treatment of ALK-positive NSCLC and other cancers, either in front-line or after prior therapy. Crizotinib, for example, shows an overall response rate of 60-80% and median progression-free survival of 8-11 months, which is comparable to its activity in ROS1-positive NSCLC. Despite an initial response, many resistance mutations have emerged to the aforementioned FDA-approved TKIs. Some of these mutations, such as the combined L1196M gatekeeper and G1202R solvent front mutation, are resistant to all of the approved drugs. New treatments of ALK-positive cancer harboring resistance mutations are a need in the art.

In further embodiments, the compound of Formula (I) is an inhibitor of human tropomyosin receptor kinases (TRKs). The TRK family comprises receptor tyrosine kinases TRKA, TRKB, and TRKC, which are encoded by the NTRK1, NTRK2, and NTRK3 genes, respectively. Each TRK is activated by a different but overlapping set of neurotrophin ligands such as NGF, BDNF, and NT-3. All TRKs modulate similar downstream signaling pathways, consistent with sequence divergence in the ligand-binding domain but convergence in the kinase domain (90% similarity). TRKs play crucial roles in the nervous system of developing and adult mammals by regulating processes such as memory, movement, pain, and proprioception. Like ROS1 and ALK, NTRK rearrangements lead to constitutively active TRK fusions that drive oncogenic transformation through MAPK, PI3K, and other pathways. TRK fusions are found in many cancers and represent over 80% of the cases in secretory breast carcinoma, mammary analogue secretory carcinomas, infantile fibrosarcoma, and congenital mesoblastic nephroma. Thus, inhibition of TRKs is advantageous for treating cancers expressing TRK fusions.

Many existing ROS1 and ALK inhibitors also exhibit potent inhibition of native non-oncogenic TRKs. This is a substantial drawback because native TRKs play important functions in the nervous system, and inadvertent inhibition of native TRKs is associated with adverse reactions including dizziness, ataxia, gait disturbance, paraesthesia, weight gain, and cognitive changes. New therapies that spare TRKs while selectively targeting ROS1 and/or ALK, in their non-mutant and/or mutant forms, are a need in the art.

In certain embodiments, provided herein is a method of decreasing a level of ROS1 or ALK in a cell, comprising contacting the cell with a compound or a pharmaceutical composition or a pharmaceutical combination provided herein. In an embodiment, such contact occurs in a cell in a mammal such as a human. In an embodiment, such contact occurs in a cell in a human patient having a cancer provided herein.

In certain embodiments, the compound selectively inhibits ROS1 over TRK (e.g., TRKA, TRKB, and/or TRBC). By way of non-limiting example, the ratio of selectivity can be greater than a factor of about 5, greater than a factor of about 10, greater than a factor of about 50, greater than a factor of about 100, greater than a factor of about 200, greater than a factor of about 400, greater than a factor of about 600, greater than a factor of about 800, greater than a factor of about 1000, greater than a factor of about 1500, greater than a factor of about 2000, greater than a factor of about 5000, greater than a factor of about 10,000, or greater than a factor of about 20,000, where selectivity can be measured by ratio of IC₅₀ values, among other means. In certain embodiments, the selectivity of ROS1 over TRK is measured by the ratio of the IC₅₀ value against TRK to the IC₅₀ value against ROS1.

In certain embodiments, a compound provided herein selectively inhibits ALK. In certain embodiments, the compound selectively inhibits ALK over ROS1. By way of non-limiting example, the ratio of selectivity can be greater than a factor of about 1.5, greater than a factor of about 2, than a factor of about 3, greater than a factor of about 4, greater than a factor of about 5, or greater than a factor of about 10, where selectivity can be measured by ratio of IC₅₀ values, among other means. In certain embodiments, the selectivity of ALK over ROS1 is measured by the ratio of the IC₅₀ value against ROS1 to the IC₅₀ value against ALK.

In certain embodiments, the compound selectively inhibits ALK over TRK (e.g., TRKA, TRKB, and/or TRBC). By way of non-limiting example, the ratio of selectivity can be greater than a factor of about 5, greater than a factor of about 10, greater than a factor of about 50, greater than a factor of about 100, greater than a factor of about 200, greater than a factor of about 400, greater than a factor of about 600, greater than a factor of about 800, greater than a factor of about 1000, greater than a factor of about 1500, greater than a factor of about 2000, greater than a factor of about 5000, or greater than a factor of about 10,000, where selectivity can be measured by ratio of IC₅₀ values, among other means. In certain embodiments, the selectivity of ALK over TRK is measured by the ratio of the IC₅₀ value against TRK to the IC₅₀ value against ALK.

In one embodiment, without being bound by a particular theory, one or more compound provided herein selectively inhibit an ALK mutation over TRK (e.g., TRKA, TRKB, and/or TRBC), wherein the ALK mutation is I1171X¹ (X¹ is N, S, or T) and/or D1203N. In one embodiment, the compounds selectively inhibit I1171X¹ (X¹ is N, S, or T) over TRKA. In one embodiment, the compounds selectively inhibit I1171X¹ (X¹ is N, S, or T) over TRKB. In one embodiment, the compounds selectively inhibit I1171N over TRKB. In one embodiment, the compounds selectively inhibit D1203N over TRKB. In one embodiment, the compounds selectively inhibit I1171X¹ (X¹ is N, S, or T) and D1203N over TRKB. In one embodiment, the compounds selectively inhibit I1171N and D1203N over TRKB. In one embodiment, the ratio of selectivity is at least a factor of about 5. In one embodiment, the ratio of selectivity is at least a factor of about 10. In one embodiment, the ratio of selectivity is at least a factor of about 30. In one embodiment, the ratio of selectivity is at least a factor of about 50. In one embodiment, the ratio of selectivity is at least a factor of about 100. In one embodiment, the selectivity of ALK mutation over TRK is measured by the ratio of the IC₅₀ value against TRK to the IC₅₀ value against ALK mutation.

In certain embodiments, the compound selectively inhibits ROS1 and ALK over TRK (e.g., TRKA, TRKB, and/or TRBC). By way of non-limiting example, the ratio of selectivity can be greater than a factor of about 5, greater than a factor of about 10, greater than a factor of about 50, greater than a factor of about 100, greater than a factor of about 200, greater than a factor of about 400, greater than a factor of about 600, greater than a factor of about 800, greater than a factor of about 1000, greater than a factor of about 1500, greater than a factor of about 2000, greater than a factor of about 5000, greater than a factor of about 10,000, or greater than a factor of about 20,000, where selectivity can be measured by ratio of IC₅₀ values, among other means. In certain embodiments, the selectivity of ROS1 and ALK over TRK is measured by the ratio of the IC₅₀ value against TRK to the IC₅₀ value against ROS1 and ALK.

As used herein, in some embodiments, the selectivity or ratio of selectivity is measured in a biochemical assay or a cell proliferation assay. In some embodiments, the cell proliferation assay is a Ba/F3 proliferation assay.

In certain embodiments, provided herein is a method for selectively inhibiting ROS1 over TRK (e.g., TRKA, TRKB, and/or TRBC) wherein the inhibition takes place in a cell. In certain embodiments, the method comprises contacting ROS1 with an effective amount of a compound provided herein. In an embodiment, such contact occurs in a cell. In an embodiment, such contact occurs in a cell in a mammal such as a human. In an embodiment, such contact occurs in a cell in a human patient having a cancer provided herein.

In certain embodiments, provided herein is a method for selectively inhibiting ROS1 over TRK (e.g., TRKA, TRKB, and/or TRBC) wherein the inhibition takes place in a subject suffering from cancer, said method comprising administering an effective amount of a compound or a pharmaceutical composition provided herein to said subject. In certain embodiments, provided herein is a method of treating a subject suffering from a cancer associated with ROS1, said method comprising selectively inhibiting ROS1 over TRK (e.g., TRKA, TRKB, and/or TRBC) by administering an amount of a compound or a pharmaceutical composition provided herein to said subject, wherein said amount is sufficient for selective inhibiting ROS1 over TRK (e.g., TRKA, TRKB, and/or TRBC).

In certain embodiments, provided herein is a method for selectively inhibiting ALK over ROS1 wherein the inhibition takes place in a cell. In certain embodiments, provided herein is a method for selectively inhibiting ALK over TRK (e.g., TRKA, TRKB, and/or TRBC) wherein the inhibition takes place in a cell. In certain embodiments, the method comprises contacting ALK with an effective amount of a compound provided herein. In an embodiment, such contact occurs in a cell. In an embodiment, such contact occurs in a cell in a mammal such as a human. In an embodiment, such contact occurs in a cell in a human patient having a cancer provided herein.

In certain embodiments, provided herein is a method for selectively inhibiting ALK over ROS1 wherein the inhibition takes place in a subject suffering from cancer, said method comprising administering an effective amount of a compound or a pharmaceutical composition provided herein to said subject. In certain embodiments, provided herein is a method of treating a subject suffering from a cancer associated with ALK, said method comprising selectively inhibiting ALK over ROS1 by administering an amount of a compound or a pharmaceutical composition provided herein to said subject, wherein said amount is sufficient for selective inhibiting ALK over ROS1.

In certain embodiments, provided herein is a method for selectively inhibiting ALK over TRK (e.g., TRKA, TRKB, and/or TRBC) wherein the inhibition takes place in a subject suffering from cancer, said method comprising administering an effective amount of a compound or a pharmaceutical composition provided herein to said subject. In certain embodiments, provided herein is a method of treating a subject suffering from a cancer associated with ALK, said method comprising selectively inhibiting ALK over TRK (e.g., TRKA, TRKB, and/or TRBC) by administering an amount of a compound or a pharmaceutical composition provided herein to said subject, wherein said amount is sufficient for selective inhibiting ALK over TRK (e.g., TRKA, TRKB, and/or TRBC).

As used herein and unless otherwise specified, inhibition of ROS1 includes inhibition of wild type ROS1, or a mutation thereof, inhibition of ALK includes inhibition of wild type ALK, or a mutation thereof; and inhibition of TRK includes inhibition of wild type TRK, or a mutation thereof.

Cancers treated by methods provided herein include, but are not limited to, lung cancer, e.g., non-small cell lung cancer, inflammatory myofibroblastic tumor, ovarian cancer, e.g., serous ovarian carcinoma, melanoma, e.g., spitzoid melanoma, glioblastoma, bile duct cancer, e.g., cholangiocarcinoma, gastric cancer, colorectal cancer, angiosarcoma, anaplastic large cell lymphoma, diffuse large B-cell lymphoma, large B-cell lymphoma, esophageal cancer, e.g., esophageal squamous cell carcinoma, kidney cancer, e.g., renal medullary carcinoma or renal cell carcinoma, breast cancer, e.g., triple negative breast cancer, thyroid cancer, e.g., papillary thyroid cancer, neuroblastoma, epithelioid hemangioendothelioma, colon cancer, and spitzoid tumor.

Cancers treated by methods provided herein include cancers originating from one or more oncogenic proteins selected from ROS1, ALK, TRKA, TRKB, and TRKC. In certain embodiments, cancers treated by methods provided herein include cancers that are drug resistant to treatments directed at one or more oncogenic proteins selected from ROS1, ALK, TRKA, TRKB, and TRKC.

In one embodiment, the cancer in a method provided herein is anaplastic lymphoma kinase positive (ALK+). As used herein and unless otherwise specified, an “ALK positive” (ALK+) cancer, disease, or disorder refers to a cancer, disease, or disorder characterized by inappropriately high expression of an ALK gene and/or the presence of a mutation in an ALK gene and/or the presence of a partially deleted ALK protein. In one embodiment, “ALK positive” (ALK+) cancer, disease, or disorder refers to a cancer, disease, or disorder characterized by inappropriately high expression of an ALK gene and/or the presence of a mutation in an ALK gene. In one embodiment, “ALK positive” (ALK+) cancer, disease, or disorder refers to a cancer, disease, or disorder characterized by the presence of a partially deleted ALK protein (e.g., NB1, AskaSS). In one embodiment, the mutation alters the biological activity of an ALK nucleic acid molecule or polypeptide. As used herein and unless otherwise specified, a “mutation” or “mutant” of ALK comprises one or more deletions, substitutions, insertions, inversions, duplications, translocations, or amplifications in the amino acid or nucleotide sequences of ALK, or fragments thereof. As used herein and unless otherwise specified, an ALK “rearrangement” refers to genetic translocations involving the ALK gene that may result in ALK fusion genes and/or ALK fusion proteins. The ALK fusion can also include one or more deletions, substitutions, insertions, inversions, duplications, translocations, or amplifications or a fragment thereof, as long as the mutant retains kinase phosphorylation activity.

In certain embodiments, the ALK mutation comprises one or more ALK point mutations. In some embodiments, cancers treated by methods provided herein include one or more mutations in ALK kinase. In certain embodiments, the one or more ALK point mutations are selected from point mutations at T1151, L1152, C1156, 11171, F1174, V1180, L1196, L1198, G1202, D1203, S1206, E1210, F1245, G1269, and R1275. In certain embodiments, the one or more ALK point mutations are selected from G1202R, G1202K, L1196M, G1269A, G1269S, C1156Y, I1171T, I1171N, I1171S, F1174L, V1180L, S1206Y, E1210K, 1151Tins, F1174C, F1174L, G1202del, D1203N, S1206R, S1206C, L1152R, L1196Q, L1198P, L1198F, R1275Q, L1152P, C1156T, F1245V, and T1151 L1152insT. In certain embodiments, the ALK mutation is selected from the group consisting of G1202R, L1196M, G1269A, D1203N, I1171N, I1171S, I1171T, C1156Y, F1174L, S1206R, G1269S, and T1151 L1152insT. In certain embodiments, the ALK mutation is G1202R. In certain embodiments, the ALK mutation is L1196M. In certain embodiments, the ALK mutation is G1269A. In certain embodiments, the ALK mutation is L1198F. In certain embodiments, the ALK mutation is co-mutation of G1202R and one or more mutations selected from L1196M, G1269A, and L1198F. In certain embodiments, the ALK mutation is G1202R/L1196M dual mutation. In certain embodiments, the ALK mutation is G1202R/G1269A dual mutation. In certain embodiments, the ALK mutation is G1202R/L1198F dual mutation. In certain embodiments, the ALK mutation is I1171N. In certain embodiments, the ALK mutation is I1171S. In certain embodiments, the ALK mutation is I1171T. In certain embodiments, the ALK mutation is D1203N. In certain embodiments, the ALK mutation is F1174L. In certain embodiments, the ALK mutation is I1171N/D1203N dual mutation. In certain embodiments, the ALK mutation is I1171N/L1198F dual mutation. In certain embodiments, the ALK mutation is I1171N/L1198Y dual mutation. In certain embodiments, the ALK mutation is I1171N/L1198I dual mutation. In certain embodiments, the ALK mutation is I1171T/D1203N dual mutation. In certain embodiments, the ALK mutation is I1171S/D1203N dual mutation. In certain embodiments, the ALK mutation is I1171T/L1198Y dual mutation. In certain embodiments, the ALK mutation is I1171T/L1198F dual mutation. In certain embodiments, the ALK mutation is I1171T/L1198I dual mutation. In certain embodiments, the ALK mutation is I1171S/L1198Y dual mutation. In certain embodiments, the ALK mutation is I1171S/L1198F dual mutation. In certain embodiments, the ALK mutation is I1171S/L1198I dual mutation.

In one embodiment, the ALK mutation comprises one or more ALK point mutations. In some embodiments, cancers treated by methods of the present disclosure include one or more mutations in ALK kinase. In one embodiment, the one or more ALK point mutations are selected from point mutations at T1151, L1152, C1156, I1171, F1174, V1180, L1196, L1198, G1202, D1203, S1206, E1129, E1210, F1245, G1269, and R1275. In one embodiment, the one or more ALK point mutations is selected from R 10601H, F1174C/I/L/S/V, F1245C/J/L/V, R1275L/Q, T151M, M1166R, I1171N, I1171S, I1171N, I1183T, L1196M, A1200V, L1204F, L1240V, D1270G, Y1278S, R1192P, G1128A, G1286R, and T1343I. In one embodiment, the one or more ALK point mutations are selected from G1202R, G1202K, L1196M, G1269A, G1269V, C1156Y, I1171T, I1171N, I1171S, F1174I, F1174L, F1174S, V1180L, S1206Y, E1129K, E1210K, T1151M, T1151_L1152insT, F1174C, G1202del, D1203N, S1206Y, S1206C, S1206F, L1152R, L1196Q, L1198P, L1198F, L1198H, R1275Q, L1152P, C1156T, F1245C, T1151K, 11268V, F1174V, L1198Q, S1206A, and F1245V. In one embodiment, the ALK mutation is G1202R. In one embodiment, the ALK mutation is L1196M. In one embodiment, the ALK mutation is G1269A. In one embodiment, the ALK mutation is G1269V. In one embodiment, the ALK mutation is L1198F. In one embodiment, the ALK mutation is L1198H. In one embodiment, the ALK mutation is T1151M. In one embodiment, the ALK mutation is F1174L. In one embodiment, the ALK mutation is F1174I. In one embodiment, the ALK mutation is F1174S. In one embodiment, the ALK mutation is I1171N. In one embodiment, the ALK mutation is I1171S. In one embodiment, the ALK mutation is I1171T. In one embodiment, the ALK mutation is I1171N. In one embodiment, the ALK mutation is E1129K. In one embodiment, the ALK mutation is S1206F. In one embodiment, the ALK mutation is E1210K. In one embodiment, the ALK mutation is D1203N. In one embodiment, the ALK mutation is R1275G. In one embodiment, the ALK mutation is F1245C. In one embodiment, the ALK mutation is T1151K. In one embodiment, the ALK mutation is I1268V. In one embodiment, the ALK mutation is F1174V. In one embodiment, the ALK mutation is L1198Q. In one embodiment, the ALK mutation is S1206A.

As used herein and unless otherwise specified, a “co-mutation” refers to co-occurring mutations, i.e., when two or more mutations are present at the same time, for example in the same cell and on the same allele, in the same cell but on different alleles, or in different cells.

As used herein and unless otherwise specified, a “compound mutation” refers two or more mutations located on the same allele. A compound mutation is a subset of co-mutations. Compound mutations are also sometimes referred to as dual mutations if there are two mutations located on the same allele.

In certain embodiments, the ALK mutation is a compound mutation having 1171X¹ (X¹ is N, S, or T) in combination with one or more of the following mutations: D1203N, L1198X² (X² is Y, F, I), L1196X³ (X³ is M or Q), C1156X⁴ (X⁴ is Y or F), G1269A, F1174X⁵ (X⁵ is L, C, V, I, S), and G1202X⁶ (X⁶ is R, L, K). In certain embodiments, the ALK mutation is a compound mutation having 11171X¹ (X¹ is N, S, or T) in combination with one of the following mutations: D1203N, L1198X² (X² is Y, F, I), L1196X³ (X³ is M or Q), C1156X⁴ (X⁴ is Y or F), G1269A, F1174X⁵ (X⁵ is L, C, V, I, S), and G1202X⁶ (X⁶ is R, L, K). In certain embodiments, the ALK mutation is a compound mutation having I1171X¹ (X¹ is N, S, or T) in combination with one of the following mutations: D1203N, L1198X² (X² is Y, F, I).

In some embodiments, the ALK mutation is co-mutation of G1202R and one or more mutations selected from L1196M, G1269A, T1151M, F1174S, and L1198F. In one embodiment, the ALK mutation is G1202R/L1196M compound mutation. In one embodiment, the ALK mutation is G1202R/G1269A compound mutation. In one embodiment, the ALK mutation is G1202R/L1198F compound mutation. In one embodiment, the ALK mutation is G1202R/T1151M compound mutation. In one embodiment, the ALK mutation is G1202R/F1174S compound mutation. In one embodiment, the ALK mutation is G1202R/F1174L compound mutation. In one embodiment, the ALK mutation is co-mutation of C1156Y and one or more mutations selected from L1256F, S1206F, F1174V, and F1174I. In one embodiment, the ALK mutation is C1156Y/L1256F compound mutation. In one embodiment, the ALK mutation is C1156Y/S1206F compound mutation. In one embodiment, the ALK mutation is C1156Y/F1174V compound mutation. In one embodiment, the ALK mutation is C1156Y/F1174I compound mutation. In one embodiment, the ALK mutation is co-mutation of L1196M and one or more mutations selected from L1198H, I1179V, and L1256F. In one embodiment, the ALK mutation is L1196M/L1198H compound mutation. In one embodiment, the ALK mutation is L1196M/I1179V compound mutation. In one embodiment, the ALK mutation is L1196M/L1256F compound mutation.

In one embodiment, the ALK mutation is G1202R/L1196M dual mutation. In one embodiment, the ALK mutation is G1202R/G1269A dual mutation. In one embodiment, the ALK mutation is G1202R/L1198F dual mutation. In one embodiment, the ALK mutation is G1202R/T1151M dual mutation. In one embodiment, the ALK mutation is G1202R/F I174S dual mutation. In one embodiment, the ALK mutation is G1202R/F1174L dual mutation. In one embodiment, the ALK mutation is C1156Y/L1256F dual mutation. In one embodiment, the ALK mutation is C1156Y/S1206F dual mutation. In one embodiment, the ALK mutation is C1156Y/F1174V dual mutation. In one embodiment, the ALK mutation is C1156Y/F1174I dual mutation. In one embodiment, the ALK mutation is L1196M/L1198H dual mutation. In one embodiment, the ALK mutation is L1196M/I1179V dual mutation. In one embodiment, the ALK mutation is L1196M/L1256F dual mutation.

In certain embodiments, the ALK mutation comprises one or more ALK rearrangements (in certain embodiments, one rearrangement). In certain embodiments, the ALK mutation comprises one or more ALK fusions (in certain embodiments, one fusion). In some embodiments, cancers treated by methods provided herein include ALK fusions. In certain embodiments, the ALK fusion is with one of the fusion partners selected from EML4, TMP1, WDCP, GTF2IRD1, TPM3, TPM4, CLTC, LMNA, PRKAR1A, RANBP2, TFG, FN1, KLC1, VCL, STRN, HIP1, NPM1, DCTN1, SQSTM1, TPR, CRIM1, PTPN3, FBXO36, ATIC and KIF5B. In some embodiments, the ALK fusion is with NPM1, STRN, or EML4. In certain embodiments, the ALK mutation is EML4-ALK, a fusion between the echinoderm microtubule-associated protein-like 4 (EML4) gene and the ALK tyrosine kinase domain. There are many variants of EML4-ALK that differ by breakpoint junctions, with variant 1 (vi) and variant 3 (v3) being the most prevalent clinically. In one embodiment, the ALK mutation is NPM1-ALK. In one embodiment, the ALK mutation is STRN-ALK.

In one embodiment, the ALK mutation comprises one or more ALK rearrangements (in one embodiment, one rearrangement). In one embodiment, the ALK mutation comprises one or more ALK fusions (in one embodiment, one fusion). In some embodiments, cancers treated by methods of the present disclosure include ALK fusions. In one embodiment, the ALK fusion is with one of the fusion partners described in Ou et al., JTO Clinical and Research Reports, 1(1): 1-10, the entirety of which is incorporated herein by reference. In one embodiment, the ALK fusion is with one of the fusion partners selected from the group consisting of EML4, TFG, KIF5B, KLC1, STRN, HIP1, TPR, BIRC6, DCTN1, SQSTM1, SOCS5, SEC31A, CLTC, PRKAR1A, PPM1B, EIF2AK3, CRIM1, CEBPZ, PICALM, CLIP1, BCL11A, GCC2, LMO7, PHACTR1, CMTR1, VIT, DYSF, ITGAV, PLEKHA7, CUX1, VKORC1L1, FBXO36, SPTBN1, EML6, FBXO11, CLIP4, CAMKMT, NCOA1, MYT1L, SRBD1, SRD5A2, NYAP2, MPRIP, ADAM17, ALK, LPIN1, WDPCP, CEP55, ERC1, SLC16A7, TNIP2, ATAD2B, SLMAP, FBN1, SWAP70, TCF12, TRIM66, WNK3, AKAP8L, SPECC1L, PRKCB, CDK15, LCLAT1, YAP1, PLEKHM2, DCHS1, PPFIBP1, ATP13A4, C12orf75, EPAS1, FAM179A, FUT8, LIMD1, LINC00327, LOC349160, LYPD1, RBM20, TACR1, TANC1, TTC27, TUBBB, SMPD4, SORCS1, LINC00211, SOS1, C9orf3, CYBRD1, MTA3, THADA, TSPYL6, WDR37, and PLEKHH2. In one embodiment, the ALK fusion is with one of the fusion partners selected from the group consisting of EML4, TMP1, WDCP, GTF2IRD1, TPM3, TPM4, CLTC, LMNA, PRKAR1A, RANBP2, TFG, FN1, KLC1, VCL, STRN, HIP1, NPM1, DCTN1, SQSTM1, TPR, CRIM1, PTPN3, FBXO36, ATIC, MSN, ALO17, MYH9, LRRFIP1-ALK, TDRD15-ALK and KIF5B. In one embodiment, the ALK mutation is EML4-ALK, a fusion between the echinoderm microtubule-associated protein-like 4 (EML4) gene and the ALK tyrosine kinase domain. There are many variants of EML4-ALK that differ by breakpoint junctions, with variant 1 (v1) and variant 3 (v3) being the most prevalent clinically. In one embodiment, the ALK mutation is NPM1-ALK. In one embodiment, the ALK mutation is STRN-ALK.

In one embodiment, the ALK mutation comprises one ALK rearrangement and one or more ALK point mutations. In one embodiment, the ALK mutation is EML4-ALK wild type (“wt”) (variant 1). In one embodiment, the ALK mutation is EML4-ALK (variant 2). In one embodiment, the ALK mutation is EML4-ALK (variant 3). In one embodiment, the ALK mutation is EML4-ALK wt (variant 4, 5, 6, or 7). In one embodiment, the ALK mutation is EML4-ALK wt (variant 8, 9, 10, 11, 12, 13, 14 or 15). As used herein, each variant also includes the subvariants within the variant. In one embodiment, the ALK mutation is EML4-ALK G1202R. In one embodiment, the ALK mutation is EML4-ALK I1171N. In one embodiment, the ALK mutation is EML4-ALK I1171S. In one embodiment, the ALK mutation is EML4-ALK I1171T. In one embodiment, the ALK mutation is EML4-ALK L1196M. In one embodiment, the ALK mutation is EML4-ALK D1203N. In one embodiment, the ALK mutation is EML4-ALK L1196M/G1202R. In one embodiment, the ALK mutation is EML4-ALK G1202R/G1269A. In one embodiment, the ALK mutation is EML4-ALK G1202R/L1196M. In one embodiment, the ALK mutation is EML4-ALK G1202R/L1198F. In one embodiment, the ALK mutation is EML4-ALK G1202R/T1151M. In one embodiment, the ALK mutation is EML4-ALK G1202R/F1174S. In one embodiment, the ALK mutation is EML4-ALK G1202R/F11174L. In one embodiment, the ALK mutation is EML4-ALK I1171N/D1203N. In one embodiment, the ALK mutation is EML4-ALK I1171S/D1203N. In one embodiment, the ALK mutation is EML4-ALK I1171T/D1203N.

In some embodiments, the ALK mutation comprises one or more ALK point mutations. In some embodiments, cancers treated by methods provided herein include one or more mutations in ALK kinase. In certain embodiments, the one or more ALK point mutations are selected from point mutations at T1151, L1152, C1156, I1171, F1174, V1180, L1196, L1198, G1202, D1203, S1206, E1210, F1245, G1269, and R1275. In certain embodiments, the one or more ALK point mutations are selected from T1151_L1152insT, L1152R, T1151M, L1152P, C1156Y, C1156T, I1171T, I1171N, I1171S, F1174C, F1174S, F1174L, VI180L, L1196M, L1196Q, L1198P, L1198F, G1202R, G1202K, G1202del, D1203N, S1206Y, S1206C, E1210K, F1245V, G1269A, and R1275Q. In certain embodiments, the ALK mutation is selected from the group consisting of G1202R, L1196M, G1269A, D1203N, I1171N, I1171S, I1171T, C1156Y, F1174L, and T1151_L1152insT. In certain embodiments, the ALK mutation is G1202R. In certain embodiments, the ALK mutation is L1196M. In certain embodiments, the ALK mutation comprises F1174S or F1174L. In certain embodiments, the ALK mutation comprises R1275Q. In certain embodiments, the ALK mutation comprises T1151M. In certain embodiments, the ALK mutation comprises I1171T, I1171S, or I1171N. In one embodiment, the ALK mutation comprises one or more compound mutations. In one embodiment, the compound mutation is selected from G1202R/T1151M, G1202R/L1196M, G1202R/G1269A, G1202R/L1198F, G1202R/F1174S, I1171T/D1203N, I1171T/L1198Y, I1171T/1198F, I1171T/1198I, I1171S/D1203N, I1171S/L1198Y, I1171S/1198F, I1171S/1198I, I1171N/D1203N, I1171N/L1198Y, I1171N/1198F, and I1171N/1198I. In one embodiment, the compound mutation is G1202R/L1196M, G1202R/G1269A, G1202R/L1198F, or G1202R/F1174S. In one embodiment, the compound mutation is G1202R/L1196M. In one embodiment, the compound mutation is G1202R/G1269A. In one embodiment, the compound mutation is G1202R/L1198F. In one embodiment, the compound mutation is G1202R/F1174S. In one embodiment, the ALK positive solid tumor is characterized by the presence of a partially deleted ALK protein. In one embodiment, the ALK mutation is Ex2-3del. In one embodiment, the ALK mutation is Ex2-17del.

In one embodiment, the ALK positive solid tumor is characterized by the presence of a mutation in an ALK gene. In one embodiment, the ALK mutation comprises one or more ALK rearrangement, one or more ALK point mutation, or a combination thereof. In one embodiment, the ALK mutation comprises G1202R, F1174C, F1174L, I1171N, I1171S, I1171T, L1196M, V1180L, C1156Y, G1202del, G1202K, G1269A, F1174S, S1206Y, E1210K, T1151M, T1151_L1152insT, D1203N, S1206C, L1152R, L1196Q, L1198P, L1198F, R1275Q, L1152P, C1156T, or F1245V, or a combination thereof. In one embodiment, the ALK mutation comprises G1202R. In one embodiment, the ALK mutation comprises F1174S or F1174L. In one embodiment, the ALK mutation comprises I1171S. In one embodiment, the ALK mutation comprises I1171T. In one embodiment, the ALK mutation comprises I1171N. In one embodiment, the ALK mutation comprises F1171M. In one embodiment, the ALK mutation comprises D1203N and one selected from I1171S, I1171T, I1171N, and I1171M. In one embodiment, the ALK mutation comprises C1156Y and one selected from I1171S, I1171T, I1171N, and I1171M. In one embodiment, the ALK mutation comprises R1275Q. In one embodiment, the ALK mutation comprises T1151M. In one embodiment, the ALK mutation comprises one or more compound mutations. In one embodiment, the compound mutation is G1202R/L1196M, G1202R/G1269A, G1202R/L1198F, or G1202R/F11174S. In one embodiment, the compound mutation is G1202R/L1196M. In one embodiment, the compound mutation is G1202R/G1269A. In one embodiment, the compound mutation is G1202R/L1198F. In one embodiment, the compound mutation is G1202R/F1174S. In one embodiment, the compound mutation is I1171N/D1203N. In one embodiment, the compound mutation is I1171S/D1203N. In one embodiment, the compound mutation is I1171T/D1203N. In one embodiment, the compound mutation is I1171M/D1203N. In one embodiment, the ALK positive solid tumor is characterized by the presence of a partially deleted ALK protein. In one embodiment, the ALK mutation is Ex2-3del. In one embodiment, the ALK mutation is Ex2-17del.

In one embodiment, partially deleted ALK proteins influence proliferative and metastatic properties of cancer cells. ALK protein can become partially deleted through various mechanisms. The first mechanism is shedding, where the 80-kDa extracellular domain of the ALK protein is post-translationally cleaved near residue Asn654, leaving the 140-kDa C-terminal transmembrane and intracellular domains on the cell. Shedding has been observed in many ALK-expressing cell lines, most notably from a neuroblastoma disease background. Shedding increases cancer cell migration and proliferation in preclinical models of cancer, both in vitro and in vivo (Moog-Lutz, JBC (2005), Huang, Cell Reports (2021)). The second mechanism is alternative transcription initiation (ATI), where transcription of the ALK gene begins at an alternative initiation site downstream of the original site, resulting in the absence of exons 1-18 and part of exon 19. ALK ATIs have been identified in 11% of melanomas as well as a small portion of lung cancers and anaplastic thyroid cancers. Expression of ALK ATI transforms Ba/F3 and NIH3T3 cells, conferring them with oncogenic potential. One patient with ALK ATI has shown clinical response to an ALK inhibitor therapy, suggesting that ALK ATI may be a targetable driver mutation (Wiesner, Nature (2015)). The third mechanism is partial deletion of the ALK gene, for example through a chromosomal rearrangement event. Multiple deletion variants have been identified, including deletion of exons 2-3, exons 1-5, exons 4-11, and exons 2-17, and some of these variants have been shown to activate ALK signaling as well as transform Ba/F3 or NIH3T3 cells. ALK partial deletions have been detected in neuroblastomas, sarcomas, and lymphomas. (Okubo, Oncogene (2012); Cazes, Can Res (2013); Fransson, Genes Chromosomes & Cancer (2014); Fleuren, Can Res (2017); Fukuhara, Hematol Oncol (2017)).

In certain embodiments, the ALK mutation comprises one ALK rearrangement and one or more ALK point mutations. In certain embodiments, the ALK mutation is EML4-ALK wt (variant 1). In one embodiment, the ALK mutation is EML4-ALK (variant 2). In one embodiment, the ALK mutation is EML4-ALK (variant 3). In one embodiment, the ALK mutation is EML4-ALK wt (variant 4, 5, 6, or 7). In one embodiment, the ALK mutation is EML4-ALK G1202R. In one embodiment, the ALK mutation is EML4-ALK I1171N. In one embodiment, the ALK mutation is EML4-ALK I1171S. In one embodiment, the ALK mutation is EML4-ALK I1171T. In one embodiment, the ALK mutation is EML4-ALK L1196M. In one embodiment, the ALK mutation is EML4-ALK D1203N. In one embodiment, the ALK mutation is EML4-ALK L1196M/G1202R. In one embodiment, the ALK mutation is EML4-ALK G1202R/G1269A. In one embodiment, the ALK mutation is EML4-ALK G1202R/L1196M. In one embodiment, the ALK mutation is EML4-ALK G1202R/L1198F. In one embodiment, the ALK mutation is EML4-ALK G1202R/T1151M. In one embodiment, the ALK mutation is EML4-ALK G1202R/F1174S. In one embodiment, the ALK mutation is EML4-ALK G1202R/F1174L. In certain embodiments, the ALK mutation is EML4-ALK I1171N/D1203N. In certain embodiments, the ALK mutation is EML4-ALK I1171N/L1198F. In certain embodiments, the ALK mutation is EMLL4-ALK (variant 1) G1202R. In certain embodiments, the ALK mutation is EML4-ALK (variant 2) G1202R. In certain embodiments, the ALK mutation is EML4-ALK (variant 3) G1202R. In certain embodiments, the ALK mutation is EML4-ALK (variant 1) L1196M/G1202R. In certain embodiments, the ALK mutation is EML4-ALK (variant 2) L1196M/G1202R. In certain embodiments, the ALK mutation is EML4-ALK (variant 3) L1196M/G1202R. In certain embodiments, the ALK mutation is EML4-ALK (variant 1) G1202R/G1269A. In certain embodiments, the ALK mutation is EML4-ALK (variant 2) G1202R/G1269A. In certain embodiments, the ALK mutation is EML4-ALK (variant 3) G1202R/G1269A. In certain embodiments, the ALK mutation is EML4-ALK (variant 1) G1202R/L1198F. In certain embodiments, the ALK mutation is EML4-ALK (variant 2) G1202R/L1198F. In certain embodiments, the ALK mutation is EML4-ALK (variant 3) G1202R/L1198F. In certain embodiments, the ALK mutation is EML4-ALK (variant 1) I1171N. In certain embodiments, the ALK mutation is EML4-ALK (variant 1) I1171S. In certain embodiments, the ALK mutation is EML4-ALK (variant 1) I1171T. In certain embodiments, the ALK mutation is EML4-ALK (variant 1) L1196M. In certain embodiments, the ALK mutation is EML4-ALK (variant 1) D1203N. In certain embodiments, the ALK mutation is EML4-ALK (variant 1) I1171N/D1203N. In certain embodiments, the ALK mutation is EML4-ALK (variant 1)I1171N/L1198F. In certain embodiments, the ALK mutation is EML4-ALK (variant 2) I1171N. In certain embodiments, the ALK mutation is EML4-ALK (variant 2) I1171S. In certain embodiments, the ALK mutation is EMLL4-ALK (variant 2) I1171T. In certain embodiments, the ALK mutation is EML4-ALK (variant 2) L1196M. In certain embodiments, the ALK mutation is EML4-ALK (variant 2) D1203N. In certain embodiments, the ALK mutation is EML4-ALK (variant 2) I1171N/D1203N. In certain embodiments, the ALK mutation is EML4-ALK (variant 2) I1171N/L1198F. In certain embodiments, the ALK mutation is EML4-ALK (variant 3) I1171N. In certain embodiments, the ALK mutation is EML4-ALK (variant 3) I1171S. In certain embodiments, the ALK mutation is EML4-ALK (variant 3) I1171T. In certain embodiments, the ALK mutation is EML4-ALK (variant 3) L1196M. In certain embodiments, the ALK mutation is EML4-ALK (variant 3) D1203N. In certain embodiments, the ALK mutation is EML4-ALK (variant 3) I1171N/D1203N. In certain embodiments, the ALK mutation is EML4-ALK (variant 3) I171N/L1198F.

In certain embodiments, one or more of the mutations disclosed herein results in a partially deleted ALK protein. In certain embodiments, the ALK+ cancer is characterized by a partially deleted ALK protein (e.g., the partially deleted ALK protein identified in NB-1 (e.g., ex2-3del) and Aska-SS (e.g., ex2-17del) cell lines). In some embodiments, the ALK+ cancer is characterized by ALK F1174L mutation (e.g., the mutation identified in Kelly and SH-SY5Y cell lines.

In certain embodiments, the ALK+ cancer is determined by an FDA-approved test or other tests known in the art. The tests that can be used include, e.g., FoundationOne CDx™ (F1CDx) (a sequencing based in vitro diagnostic device for detection of substitutions, insertion and deletion alterations (indels), and copy number alterations (CNAs) in 324 genes and select gene rearrangements, as well as genomic signatures including microsatellite instability (MSI) and tumor mutational burden (TMB) using DNA isolated from formalin-fixed paraffin embedded (FFPE) tumor tissue specimens); VENTANA ALK (D5F3) CDx Assay (qualitative detection of the anaplastic lymphoma kinase (ALK) protein in formalin-fixed, paraffin-embedded (FFPE) non-small cell lung carcinoma (NSCLC) tissue stained with the BenchMark XT or BenchMark ULTRA automated staining instrument); and Vysis ALK Break Apart FISH Probe Kit test (a qualitative test to detect rearrangements involving the ALK gene via fluorescence in situ hybridization (FISH) in formalin-fixed, paraffin-embedded (FFPE) non-small cell lung cancer (NSCLC) tissue specimens). In certain embodiments, the test is a fluorescence in situ hybridization (FISH) test, e.g., Vysis ALK Break Apart FISH Probe Kit test. Additional information for FDA-approved tests can be found at, e.g., https://www.fda.gov/MedicalDevices/ProductsandMedicalProcedures/InVitroDiagnostics; and additional information for Vysis ALK Break Apart FISH Probe Kit can be found at, e.g., https://www.molecular.abbott/us/en/products/oncology/vysis-alk-break-apart-fish-probe-kit; the entirety of which are incorporated herein by reference.

Also provided are methods of treating a subject having a cancer (e.g., a ALK positive cancer) that include: determining whether a cancer cell in a sample obtained from a subject having a cancer and previously administered a first ALK inhibitor, has one or more ALK inhibitor resistance mutations; and administering a compound of Formula (I) or a pharmaceutically acceptable salt or solvate thereof as a monotherapy or in combination with another anticancer agent to the subject if the subject has a cancer cell that has one or more ALK inhibitor resistance mutations. In some embodiments, the one or more ALK inhibitor resistance mutations confer increased resistance to a cancer cell or tumor to treatment with the first ALK inhibitor. In some embodiments, the one or more ALK inhibitor resistance mutations include one or more ALK inhibitor resistance mutations. For example, the one or more ALK inhibitor resistance mutations can include a substitution at one or more of amino acid positions 1202, 1196, 1269, 1156, 1171, 1174, 1180, 1206, 1210, 1151, 1174, 1203, 1206, 1152, 1196, 1198, 1275, 1152, 1156, and 1245, e.g., G1202R, L1196M, G1269A, C1156Y, I1171T, I1171N, I1171S, F1174L, V1180L, S1206Y, E1210K, 1151Tins, F1174C, G1202del, D1203N, S1206Y, S1206C, L1152R, L1196Q, L1198P, L1198F, R1275Q, L1152P, C1156T, andF1245V. In some embodiments, another anticancer agent is any anticancer agent known in the art. For example, another anticancer agent can be another ALK inhibitor (e.g., a second ALK inhibitor).

In certain embodiments, the cancer in a method provided herein is ROS1 positive (ROS1+). As used herein and unless otherwise specified, a “ROS1 positive” (ROS1+) cancer, disease, or disorder refers to a cancer, disease, or disorder characterized by inappropriately high expression of a ROS1 gene and/or the presence of a mutation in a ROS1 gene. In certain embodiments, the mutation alters the biological activity of a ROS1 nucleic acid molecule or polypeptide. As used herein and unless otherwise specified, a “mutation” or “mutant” of ROS1 comprises one or more deletions, substitutions, insertions, inversions, duplications, translocations, or amplifications in the amino acid or nucleotide sequences of ROS I, or fragments thereof. As used herein and unless otherwise specified, a ROS1 “rearrangement” refers to genetic translocations involving the ROS1 gene that may result in ROS1 fusion genes and/or ROS1 fusion proteins. The ROS1 fusion can also include one or more deletions, substitutions, insertions, inversions, duplications, translocations, or amplifications or a fragment thereof, as long as the mutant retains kinase phosphorylation activity.

In certain embodiments, the ROS1 mutation comprises one or more ROS1 point mutations. In some embodiments, cancers treated by methods provided herein include one or more mutations in ROS1 kinase. In certain embodiments, the one or more ROS1 point mutations are selected from point mutations at E1935, L1947, L1951, G1971, E1974, L1982, S1986, F2004, E2020, L2026, G2032, D2033, C2060, F2075, L2086, V2089, V2098, G2101, D2113, and L2155. In certain embodiments, the one or more ROS1 point mutations are selected from G2032R, G2032K, D2033N, S1986F, S1986Y, L2026M, L1951R, E1935G, L1947R, G1971E, E1974K, L1982F, F2004C, F2004V, E2020K, C2060G, F2075V, V2089M, V2098I, G2101A, D2113N, D2113G, L2155S, and L2086F. In certain embodiments, the ROS1 mutation is G2032R. In certain embodiments, the ROS1 mutation is S1986F. In certain embodiments, the ROS1 mutation is S1986Y. In certain embodiments, the ROS1 mutation is L2026M. In certain embodiments, the ROS1 mutation is D2033N. In certain embodiments, the ROS1 mutation is L2086F. In certain embodiments, the ROS1 mutation is F2004C. In certain embodiments, the ROS1 mutation is F2004V. In certain embodiments, the ROS1 mutation is G2101A. In certain embodiments, the ROS1 mutation is L1982F. In certain embodiments, the ROS1 mutation is co-mutation of G2032R and one or more of S1986F, S1986Y, F2004C, F2004V, L2026M, or D2033N.

In certain embodiments, the ROS1 mutation comprises one or more ROS1 rearrangements (in certain embodiments, one rearrangement). In certain embodiments, the ROS1 mutation comprises one or more ROS1 fusions (in certain embodiments, one fusion). In some embodiments, cancers treated by methods provided herein include ROS1 fusions. In certain embodiments, the ROS1 fusion is with one of the fusion partners selected from SLC34A2, CD74, TPM3, SDC4, EZR, LRIG3, KDELR2, CEP72, CLTL, CTNND2, GOPC (e.g., GOPC-S, GOPC-L), GPRC6A, LIMA1, LRIG3, MSN, MYO5C, OPRM1, SLC6A17 SLMAP, SRSF6, TFG, TMEM106B, TPD52L1, ZCCHC8, CCDC6, CAPRIN1, CEP85L, CHCHD3, CLIP1, EEF1G, KIF21A, KLC1, SART3, ST13, TRIM24, ERC1, FIP1L1, HLAA, KIAA1598, MYO5A, PPFIBP1, PWWP2A, FN1, YWHAE, CCDC30, NCOR2, NFKB2, APOB, PLG, RBP4, and GOLGB1. In certain embodiments, the ROS1 fusion is CD74-ROS1 fusion. In certain embodiments, the ROS1 fusion is SDC4-ROS1 fusion. In certain embodiments, the ROS1 fusion is EZR-ROS1 fusion. In certain embodiments, the ROS1 fusion is SLC34A2-ROS1 fusion. In certain embodiments, the ROS1 fusion is GOPC-ROS1 fusion (e.g., GOPC-ROS1-S, GOPC-ROS1-L). In certain embodiments, the ROS1 fusion is CEP85L-ROS1 fusion.

In certain embodiments, the ROS1 mutation comprises one ROS1 rearrangements and one or more ROS1 point mutations. In certain embodiments, the ROS1 mutation comprises one or more ROS1 rearrangements from CD74-ROS1, EZR-ROS1, SLC34A2-ROS1, GOPC-ROS1 (e.g., GOPC-ROS1-S, GOPC-ROS1-L), and CEP85L-ROS1, and one or more ROS1 point mutations selected from F2004C, F2004V, and G2032R. In certain embodiments, the ROS1 mutation comprises one or more ROS1 rearrangements from CD74-ROS1, EZR-ROS1, and SLC34A2-ROS1, and ROS1 point mutation of G2101A.

In certain embodiments, the ROS1 mutation is CD74-ROS1 F2004C. In certain embodiments, the ROS1 mutation is CD74-ROS1 F2004V. In certain embodiments, the ROS1 mutation is CD74-ROS1 G2101A. In certain embodiments, the ROS1 mutation is CD74-ROS1 G2032R. In certain embodiments, the ROS1 mutation is CD74-ROS1 S1986F. In certain embodiments, the ROS1 mutation is CD74-ROS1 L2026M. In certain embodiments, the ROS1 mutation is CD74-ROS1 D2033N. In certain embodiments, the ROS1 mutation is EZR-ROS1 F2004C. In certain embodiments, the ROS1 mutation is EZR-ROS1 F2004V. In certain embodiments, the ROS1 mutation is EZR-ROS1 G2101A. In certain embodiments, the ROS1 mutation is EZR-ROS1 G2032R. In certain embodiments, the ROS1 mutation is SLC34A2-ROS1 F2004C. In certain embodiments, the ROS1 mutation is SLC34A2-ROS1 F2004V. In certain embodiments, the ROS1 mutation is SLC34A2-ROS1 G2101A. In certain embodiments, the ROS1 mutation is SLC34A2-ROS1 G2032R. In certain embodiments, the ROS1 mutation is GOPC-ROS1 F2004C (e.g., GOPC-ROS1-S F2004C, GOPC-ROS1-L F2004C). In certain embodiments, the ROS1 mutation is GOPC-ROS1 F2004V (e.g., GOPC-ROS1-S F2004V, GOPC-ROS1-L F2004V). In certain embodiments, the ROS1 mutation is GOPC-ROS1 G2032R (e.g., GOPC-ROS1-S G2032R, GOPC-ROS1-L G2032R). In certain embodiments, the ROS1 mutation is CEP85L-ROS1 F2004C. In certain embodiments, the ROS1 mutation is CEP85L-ROS1 F2004V. In certain embodiments, the ROS1 mutation is CEP85L-ROS1 G2032R. In certain embodiments, the ROS1 mutation is GOPC-ROS1 L1982F (e.g., GOPC-ROS1-S L1982F, GOPC-ROS1-L L1982F). In certain embodiments, the ROS1 mutation is CD74-ROS1 L1982F.

In certain embodiments, the ROS1+ cancer is determined by an FDA-approved test or other tests known in the art. The tests that can be used include, e.g., Oncomine™ Dx Target Test by Thermo Fisher Scientific. (a qualitative in vitro diagnostic test that uses targeted high-throughput, parallel-sequencing technology to detect sequence variations in 23 genes in DNA and RNA isolated from formalin-fixed, paraffin-embedded tumor (FFPE) tissue samples from patients with non-small cell lung cancer (NSCLC) using the Ion PGM Dx System); Vysis ROS1 Break Apart FISH Probe Kit (a qualitative test to detect rearrangements involving ROS1 gene rearrangements at 6q22 via fluorescence in situ hybridization (FISH) in formalin-fixed, paraffin-embedded (FFPE) non-small cell lung cancer (NSCLC) tissue specimens) or RTReal Time-Polymerase Chain Reaction (RT-PCR) or NGSNext Generation Sequencing via a local diagnostic test.

Also provided are methods of treating a subject having a cancer (e.g., a ROS1 positive cancer) that include: determining whether a cancer cell in a sample obtained from a subject having a cancer and previously administered a first ROS1 inhibitor, has one or more ROS1 inhibitor resistance mutations; and administering a compound of Formula (I) or a pharmaceutically acceptable salt or solvate thereof as a monotherapy or in conjunction with another anticancer agent to the subject if the subject has a cancer cell that has one or more ROS1 inhibitor resistance mutations. In some embodiments, the one or more ROS1 inhibitor resistance mutations confer increased resistance to a cancer cell or tumor to treatment with the first ROS1 inhibitor. In some embodiments, the one or more ROS1 inhibitor resistance mutations include one or more ROS1 inhibitor resistance mutations. For example, the one or more ROS1 inhibitor resistance mutations can include a substitution at one or more of amino acid positions 2032, 2033, 1986, 2026, 1951, 1935, 1947, 1971, 1974, 1982, 2004, 2020, 2060, 2075, 2089, 2098, 2101, 2113, 2155, 2032, and 2086, e.g., G2032R, D2033N, S1986F, S1986Y, L2026M, L1951R, E1935G, L1947R, G1971E, E1974K, L1982F, F2004C, F2004V, E2020K, C2060G, F2075V, V2089M, V2098I, G2101A, D2113N, D2113G, L2155S, L2032K, and L2086F. In some embodiments, another anticancer agent is any anticancer agent known in the art. For example, another anticancer agent can be another ROS1 inhibitor (e.g., a second ROS1 inhibitor).

In certain embodiments, a compound provided herein is a CNS-penetrating compound. In certain embodiments, after the administration of an effective amount of a compound provided herein (e.g., orally or intravenously), the compound is able to penetrate CNS (e.g., blood-brain barrier) and achieve a concentration in CNS (e.g., brain) that is still sufficient to inhibit (e.g., selectively inhibit) ROS1 or ALK or both.

In certain embodiments, provided herein is a method for treating CNS metastases of a cancer, comprising administering to a subject in need thereof an effective amount of a compound provided herein, e.g., a compound of Formula (I), or a stereoisomer, a mixture of stereoisomers, or a pharmaceutically acceptable salt thereof. In certain embodiments, the CNS metastases is brain metastases. In certain embodiments, the cancer is a ROS1+ cancer. In certain embodiments, the cancer is an ALK+ cancer.

In one embodiment, the solid tumor (or cancer) is leukocyte receptor tyrosine kinase (LTK) positive. In one embodiment, the solid tumor is LTK positive NSCLC. In one embodiment, the solid tumor is LTK positive breast invasive ductal carcinoma, prostate adenocarcinoma, pancreatic adenocarcinoma, adenocarcinoma of unknown primary, or bladder urothelial carcinoma. In one embodiment, the solid tumor is LTK positive lung cancer. In one embodiment, the solid tumor is LTK positive NSCLC. In one embodiment, the solid tumor (or cancer) has an LTK mutation. In one embodiment, the LTK mutation is G269A, F218I, N257T, A13fs, or A214fs. In one embodiment, the solid tumor (or cancer) has an LTK fusion. In one embodiment, the LTK fusion is CLIP1-LTK. See Cooper A J, Sequist L V, Johnson T W, Lin JJ. LTK fusions: A new target emerges in non-small cell lung cancer. Cancer Cell. 2022 Jan. 10; 40(1):23-25; and Izumi, H., Matsumoto, S., Liu, J. et al. The CLIP1-LTK fusion is an oncogenic driver in non-small-cell lung cancer. Nature 600, 319-323 (2021), each of which are incorporated herein by reference in their entirety.

In some embodiments, the compound is an inhibitor of human tropomyosin receptor kinase A, B, or C. In certain embodiments, the IC50 of the compound for inhibition of mutant or non-mutant ROS1 or ALK is no more than one-fifth of the IC50 of the compound for inhibition of wild-type tropomyosin receptor kinase A, B, or C. TRK inhibition, particularly in the central nervous system (CNS), has been associated with adverse reactions, including dizziness/ataxia/gait disturbance, paraesthesia, weight gain and cognitive changes.

In some embodiments, provided is a method of minimizing adverse events in a subject in need of treatment for cancer (e.g., a ROS1 positive cancer or an ALK positive cancer), the method comprising administering to the subject a therapeutically effective amount of a compound provided herein, e.g., a compound of Formula (I), a stereoisomer, a mixture of stereoisomers, or a pharmaceutically acceptable salt thereof, and wherein the method minimizes adverse events associated with TRK inhibitors. In some embodiments, the cancer is a ROS1-associated cancer or an ALK-associated (or ALK+) cancer. In some embodiments, the adverse events are TRK-related CNS adverse events.

As used herein “minimizing” adverse events refers to a reduction in the incidence of adverse events in a subject or patient population compared to the paradigmatic incidence of adverse events in a subject or patient population treated with TRK inhibitors (e.g., entrectinib, repotrectinib, or lorlatinib). In some embodiments, the incidence of an adverse event refers to the frequency or percentage of a specific adverse event over a subject or patient population. In some embodiments, the incidence of an adverse event refers to the total number of adverse events experienced by an individual subject. In some embodiments, minimizing adverse events refers to minimizing TRK-related CNS adverse events. In some embodiments, minimizing TRK-related CNS adverse events means less than 40% of the patient population has a TRK-related CNS adverse event. In some embodiments, minimizing TRK-related CNS adverse events means less than 35%, less than 30%, less than 25%, less than 20%, less than 15%, less than 10% or less than 5% of the patient population has a TRK-related CNS adverse event. In some embodiments, minimizing TRK-related CNS adverse events means less than 12% of the patient population have more than one TRK-related CNS adverse event. In some embodiments, minimizing TRK-related CNS adverse events means less than 11%, less than 10%, less than 9%, less than 8%, less than 7%, less than 6%, less than 5%, less than 4%, or less than 3% of the patient population have more than one TRK-related CNS adverse event.

In some embodiments, TRK-related CNS adverse events refers to one or more of the following: dizziness, ataxia, gait disturbance, paraesthesia, weight gain, hyperphagia, paresthesias, abnormal movement, cognitive changes, speech effects (e.g., dysarthria, slow speech, or speech disorder), mood disorder (e.g., irritability, anxiety, depression, affect lability, personality change, mood swings, affective disorder, aggression, agitation, mood altered, depressed mood, euphoric mood, or mania), and cognitive disorder (e.g., memory impairment, cognitive disorder, amnesia, confusion, disturbance in attention, delirium, mental impairment, attention deficit/hyperactivity disorder, dementia, or reading disorder).

In certain embodiments, provided herein is a method for preventing or limiting TRK-related CNS side effect or adverse event in a cancer treatment, comprising administering to a subject in need thereof an effective amount of a compound provided herein, e.g., a compound of Formula (I), or a stereoisomer, a mixture of stereoisomers, or a pharmaceutically acceptable salt thereof. In certain embodiments, the method prevents the occurrence of the TRK-related CNS adverse event. In certain embodiments, the method limits the frequency of occurrence of the TRK-related CNS adverse event. In certain embodiments, the method limits the severity of the TRK-related side effect. In certain embodiments, provided herein is a method for treating CNS metastases of a cancer with reduced TRK-related side effect, comprising administering to a subject in need thereof an effective amount of a compound provided herein, e.g., a compound of Formula (I), or a stereoisomer, a mixture of stereoisomers, or a pharmaceutically acceptable salt thereof. In certain embodiments, the reduction/limiting/prevention in CNS side effect or adverse event is determined in a statistical sample, as compared to a standard of care treatment, e.g., an approved ROS1 and/or ALK inhibitor (e.g., crizotinib, entrectinib, lorlatinib, or repotrectinib) for ROS1+ and/or ALK+ cancer. In certain embodiments, the TRK-related side effect is a TRKB-related CNS side effect. In certain embodiments, the TRK-related CNS side effect or adverse event is dizziness, ataxia, gait disturbance, paraesthesia, weight gain, cognitive impairment, a mood disorder, or sleep disturbance.

In certain embodiments, provided herein is a method for treating cancer, comprising administering to a subject in need thereof a therapeutically effective amount of a compound provided herein, e.g., a compound of Formula (I), or a stereoisomer, a mixture of stereoisomers, or a pharmaceutically acceptable salt thereof. In certain embodiments, the cancer is a ROS1-associated cancer. In certain embodiments, the cancer is a ROS1+ cancer. In certain embodiments, the cancer is an ALK-associated cancer. In certain embodiments, the cancer is an ALK+ cancer. In certain embodiments, the cancer is identified to be ROS1+. In certain embodiments, the cancer is identified to be ALK+.

In certain embodiments, provided herein is a method for treating a ROS1+ cancer, comprising administering to a subject in need thereof a therapeutically effective amount of a compound provided herein, e.g., a compound of Formula (I), or a stereoisomer, a mixture of stereoisomers, or a pharmaceutically acceptable salt thereof.

In certain embodiments, provided herein is a method for treating an ALK+ cancer, comprising administering to a subject in need thereof a therapeutically effective amount of a compound provided herein, e.g., a compound of Formula (I), or a stereoisomer, a mixture of stereoisomers, or a pharmaceutically acceptable salt thereof.

In certain embodiments, provided herein is a method for treating cancer in a subject, comprising: (i) identifying the cancer in the subject to be ROS1+, and (ii) administering to the subject a therapeutically effective amount of a compound provided herein, e.g., a compound of Formula (I), or a stereoisomer, a mixture of stereoisomers, or a pharmaceutically acceptable salt thereof.

In certain embodiments, provided herein is a method for treating cancer in a subject, comprising: (i) identifying the cancer in the subject to be ALK+, and (ii) administering to the subject a therapeutically effective amount of a compound provided herein, e.g., a compound of Formula (I), or a stereoisomer, a mixture of stereoisomers, or a pharmaceutically acceptable salt thereof.

In certain embodiments, the cancer (or ROS1+ cancer, or ALK+ cancer) is a solid tumor. In certain embodiments, the cancer (or ROS1+ cancer, or ALK+ cancer) is lung cancer, e.g., non-small cell lung cancer (NSCLC), glioblastoma, inflammatory myofibroblastic tumor (IMT), bile duct cancer, e.g., cholangiocarcinoma, ovarian cancer, e.g., serous ovarian carcinoma, gastric cancer, colorectal cancer, angiosarcoma, melanoma, e.g., spitzoid melanoma, epithelioid hemangioendothelioma, esophageal cancer, e.g., esophageal squamous cell carcinoma (ESCC), kidney cancer, e.g., renal medullary carcinoma or renal cell carcinoma, breast cancer, e.g., triple negative breast cancer, colon cancer, thyroid cancer, e.g., papillary thyroid cancer, spitzoid tumor, or neuroblastoma.

In certain embodiments, the cancer is lung cancer. In certain embodiments, the cancer is non-small cell lung cancer. In certain embodiments, the cancer is ROS1+ non-small cell lung cancer. In certain embodiments, the cancer is ALK+ non-small cell lung cancer. In certain embodiments, the cancer is relapsed or refractory non-small cell lung cancer. In certain embodiments, the cancer is relapsed or refractory ROS1+ non-small cell lung cancer. In certain embodiments, the cancer is relapsed or refractory ALK+ non-small cell lung cancer. In certain embodiments, the cancer is newly diagnosed non-small cell lung cancer. In certain embodiments, the cancer is newly diagnosed ROS1+ non-small cell lung cancer. In certain embodiments, the cancer is newly diagnosed ALK+ non-small cell lung cancer.

In certain embodiments, the cancer is glioblastoma. In certain embodiments, the cancer is ROS1+ glioblastoma. In certain embodiments, the cancer is ALK+ glioblastoma. In certain embodiments, the cancer is relapsed or refractory glioblastoma. In certain embodiments, the cancer is relapsed or refractory ROS1+ glioblastoma. In certain embodiments, the cancer is relapsed or refractory ALK+ glioblastoma. In certain embodiments, the cancer is newly diagnosed glioblastoma. In certain embodiments, the cancer is newly diagnosed ROS1+ glioblastoma. In certain embodiments, the cancer is newly diagnosed ALK+ glioblastoma.

In certain embodiments, the cancer is IMT. In certain embodiments, the cancer is ROS1+ IMT. In certain embodiments, the cancer is ALK+ IMT. In certain embodiments, the cancer is relapsed or refractory TMT. In certain embodiments, the cancer is relapsed or refractory ROS1+ IMT. In certain embodiments, the cancer is relapsed or refractory ALK+ IMT. In certain embodiments, the cancer is newly diagnosed IMT. In certain embodiments, the cancer is newly diagnosed ROS1+ IMT. In certain embodiments, the cancer is newly diagnosed ALK+ IMT.

In certain embodiments, the cancer is bile duct cancer. In certain embodiments, the cancer is cholangiocarcinoma. In certain embodiments, the cancer is ROS1+ cholangiocarcinoma. In certain embodiments, the cancer is ALK+ cholangiocarcinoma. In certain embodiments, the cancer is relapsed or refractory cholangiocarcinoma. In certain embodiments, the cancer is relapsed or refractory ROS1+ cholangiocarcinoma. In certain embodiments, the cancer is relapsed or refractory ALK+ cholangiocarcinoma. In certain embodiments, the cancer is newly diagnosed cholangiocarcinoma. In certain embodiments, the cancer is newly diagnosed ROS1+ cholangiocarcinoma. In certain embodiments, the cancer is newly diagnosed ALK+ cholangiocarcinoma.

In certain embodiments, the cancer is ovarian cancer. In certain embodiments, the cancer is ROS1+ ovarian cancer. In certain embodiments, the cancer is ALK+ ovarian cancer. In certain embodiments, the cancer is relapsed or refractory ovarian cancer. In certain embodiments, the cancer is relapsed or refractory ROS1+ ovarian cancer. In certain embodiments, the cancer is relapsed or refractory ALK+ ovarian cancer. In certain embodiments, the cancer is newly diagnosed ovarian cancer. In certain embodiments, the cancer is newly diagnosed ROS1+ ovarian cancer. In certain embodiments, the cancer is newly diagnosed ALK+ ovarian cancer. In certain embodiments, the ovarian cancer is serous ovarian carcinoma. In certain embodiments, the ovarian cancer is high grade serous ovarian carcinoma.

In certain embodiments, the cancer is gastric cancer. In certain embodiments, the cancer is ROS1+ gastric cancer. In certain embodiments, the cancer is ALK+ gastric cancer. In certain embodiments, the cancer is relapsed or refractory gastric cancer. In certain embodiments, the cancer is relapsed or refractory ROS1+ gastric cancer. In certain embodiments, the cancer is relapsed or refractory ALK+ gastric cancer. In certain embodiments, the cancer is newly diagnosed gastric cancer. In certain embodiments, the cancer is newly diagnosed ROS1+ gastric cancer. In certain embodiments, the cancer is newly diagnosed ALK+ gastric cancer.

In certain embodiments, the cancer is colorectal cancer. In certain embodiments, the cancer is ROS1+ colorectal cancer. In certain embodiments, the cancer is ALK+ colorectal cancer. In certain embodiments, the cancer is relapsed or refractory colorectal cancer. In certain embodiments, the cancer is relapsed or refractory ROS1+ colorectal cancer. In certain embodiments, the cancer is relapsed or refractory ALK+ colorectal cancer. In certain embodiments, the cancer is newly diagnosed colorectal cancer. In certain embodiments, the cancer is newly diagnosed ROS1+ colorectal cancer. In certain embodiments, the cancer is newly diagnosed ALK+ colorectal cancer.

In certain embodiments, the cancer is angiosarcoma. In certain embodiments, the cancer is ROS1+ angiosarcoma. In certain embodiments, the cancer is ALK+ angiosarcoma. In certain embodiments, the cancer is relapsed or refractory angiosarcoma. In certain embodiments, the cancer is relapsed or refractory ROS1+ angiosarcoma. In certain embodiments, the cancer is relapsed or refractory ALK+ angiosarcoma. In certain embodiments, the cancer is newly diagnosed angiosarcoma. In certain embodiments, the cancer is newly diagnosed ROS1+ angiosarcoma. In certain embodiments, the cancer is newly diagnosed ALK+ angiosarcoma.

In certain embodiments, the cancer is melanoma. In certain embodiments, the cancer is spitzoid tumor. In certain embodiments, the cancer is spitzoid melanoma. In certain embodiments, the cancer is ROS1+ spitzoid melanoma. In certain embodiments, the cancer is ALK+ spitzoid melanoma. In certain embodiments, the cancer is relapsed or refractory spitzoid melanoma. In certain embodiments, the cancer is relapsed or refractory ROS1+ spitzoid melanoma. In certain embodiments, the cancer is relapsed or refractory ALK+ spitzoid melanoma. In certain embodiments, the cancer is newly diagnosed spitzoid melanoma. In certain embodiments, the cancer is newly diagnosed ROS1+ spitzoid melanoma. In certain embodiments, the cancer is newly diagnosed ALK+ spitzoid melanoma.

In certain embodiments, the cancer is epithelioid hemangioendothelioma. In certain embodiments, the cancer is ROS1+ epithelioid hemangioendothelioma. In certain embodiments, the cancer is ALK+ epithelioid hemangioendothelioma. In certain embodiments, the cancer is relapsed or refractory epithelioid hemangioendothelioma. In certain embodiments, the cancer is relapsed or refractory ROS1+ epithelioid hemangioendothelioma. In certain embodiments, the cancer is relapsed or refractory ALK+ epithelioid hemangioendothelioma. In certain embodiments, the cancer is newly diagnosed epithelioid hemangioendothelioma. In certain embodiments, the cancer is newly diagnosed ROS1+ epithelioid hemangioendothelioma. In certain embodiments, the cancer is newly diagnosed ALK+ epithelioid hemangioendothelioma.

In certain embodiments, the cancer is esophageal cancer. In certain embodiments, the cancer is ESCC. In certain embodiments, the cancer is ROS1+ESCC. In certain embodiments, the cancer is ALK+ESCC. In certain embodiments, the cancer is relapsed or refractory ESCC. In certain embodiments, the cancer is relapsed or refractory ROS1+ESCC. In certain embodiments, the cancer is relapsed or refractory ALK+ESCC. In certain embodiments, the cancer is newly diagnosed ESCC. In certain embodiments, the cancer is newly diagnosed ROS1+ESCC. In certain embodiments, the cancer is newly diagnosed ALK+ESCC.

In certain embodiments, the cancer is kidney cancer. In certain embodiments, the cancer is renal medullary carcinoma. In certain embodiments, the cancer is ROS1+ renal medullary carcinoma. In certain embodiments, the cancer is ALK+ renal medullary carcinoma. In certain embodiments, the cancer is relapsed or refractory renal medullary carcinoma. In certain embodiments, the cancer is relapsed or refractory ROS1+ renal medullary carcinoma. In certain embodiments, the cancer is relapsed or refractory ALK+ renal medullary carcinoma. In certain embodiments, the cancer is newly diagnosed renal medullary carcinoma. In certain embodiments, the cancer is newly diagnosed ROS1+ renal medullary carcinoma. In certain embodiments, the cancer is newly diagnosed ALK+ renal medullary carcinoma. In certain embodiments, the cancer is renal cell carcinoma. In certain embodiments, the cancer is ROS1+ renal cell carcinoma. In certain embodiments, the cancer is ALK+ renal cell carcinoma. In certain embodiments, the cancer is relapsed or refractory renal cell carcinoma. In certain embodiments, the cancer is relapsed or refractory ROS1+ renal cell carcinoma. In certain embodiments, the cancer is relapsed or refractory ALK+ renal cell carcinoma. In certain embodiments, the cancer is newly diagnosed renal cell carcinoma. In certain embodiments, the cancer is newly diagnosed ROS1+ renal cell carcinoma. In certain embodiments, the cancer is newly diagnosed ALK+ renal cell carcinoma.

In certain embodiments, the cancer is breast cancer. In certain embodiments, the cancer is ROS1+ breast cancer. In certain embodiments, the cancer is ALK+ breast cancer. In certain embodiments, the cancer is relapsed or refractory breast cancer. In certain embodiments, the cancer is relapsed or refractory ROS1+ breast cancer. In certain embodiments, the cancer is relapsed or refractory ALK+ breast cancer. In certain embodiments, the cancer is newly diagnosed breast cancer. In certain embodiments, the cancer is newly diagnosed ROS1+ breast cancer. In certain embodiments, the cancer is newly diagnosed ALK+ breast cancer. In certain embodiments, the breast cancer is triple negative breast cancer.

In certain embodiments, the cancer is colon cancer. In certain embodiments, the cancer is ROS1+ colon cancer. In certain embodiments, the cancer is ALK+ colon cancer. In certain embodiments, the cancer is relapsed or refractory colon cancer. In certain embodiments, the cancer is relapsed or refractory ROS1+ colon cancer. In certain embodiments, the cancer is relapsed or refractory ALK+ colon cancer. In certain embodiments, the cancer is newly diagnosed colon cancer. In certain embodiments, the cancer is newly diagnosed ROS1+ colon cancer. In certain embodiments, the cancer is newly diagnosed ALK+ colon cancer.

In certain embodiments, the cancer is thyroid cancer. In certain embodiments, the cancer is papillary thyroid cancer. In certain embodiments, the cancer is ROS1+ papillary thyroid cancer. In certain embodiments, the cancer is ALK+ papillary thyroid cancer. In certain embodiments, the cancer is relapsed or refractory papillary thyroid cancer. In certain embodiments, the cancer is relapsed or refractory ROS1+ papillary thyroid cancer. In certain embodiments, the cancer is relapsed or refractory ALK+ papillary thyroid cancer. In certain embodiments, the cancer is newly diagnosed papillary thyroid cancer. In certain embodiments, the cancer is newly diagnosed ROS1+ papillary thyroid cancer. In certain embodiments, the cancer is newly diagnosed ALK+ papillary thyroid cancer.

In certain embodiments, the cancer is neuroblastoma. In certain embodiments, the cancer is ROS1+ neuroblastoma. In certain embodiments, the cancer is ALK+ neuroblastoma. In certain embodiments, the cancer is relapsed or refractory neuroblastoma. In certain embodiments, the cancer is relapsed or refractory ROS1+ neuroblastoma. In certain embodiments, the cancer is relapsed or refractory ALK+ neuroblastoma. In certain embodiments, the cancer is newly diagnosed neuroblastoma. In certain embodiments, the cancer is newly diagnosed ROS1+ neuroblastoma. In certain embodiments, the cancer is newly diagnosed ALK+ neuroblastoma.

In certain embodiments, the cancer (or ROS1+ cancer, or ALK+ cancer) is a hematological cancer. In certain embodiments, the cancer (or ROS1+ cancer, or ALK+ cancer) is lymphoma. In certain embodiments, the lymphoma is non-Hodgkin lymphoma. In certain embodiments, the lymphoma is anaplastic large cell lymphoma (ALCL), diffuse large B-cell lymphoma (DLBCL), or large B-cell lymphoma. In addition to hematological cancer, methods for treating other blood disorder or hematologic malignancy that is ROS1+ or ALK+ are also provided herein.

In certain embodiments, the cancer is ALCL. In certain embodiments, the cancer is ROS1+ ALCL. In certain embodiments, the cancer is ALK+ ALCL. In certain embodiments, the cancer is relapsed or refractory ALCL. In certain embodiments, the cancer is relapsed or refractory ROS1+ ALCL. In certain embodiments, the cancer is relapsed or refractory ALK+ ALCL. In certain embodiments, the cancer is newly diagnosed ALCL. In certain embodiments, the cancer is newly diagnosed ROS1+ ALCL. In certain embodiments, the cancer is newly diagnosed ALK+ ALCL.

In certain embodiments, the cancer is DLBCL. In certain embodiments, the cancer is ROS1+ DLBCL. In certain embodiments, the cancer is ALK+ DLBCL. In certain embodiments, the cancer is relapsed or refractory DLBCL. In certain embodiments, the cancer is relapsed or refractory ROS1+ DLBCL. In certain embodiments, the cancer is relapsed or refractory ALK+ DLBCL. In certain embodiments, the cancer is newly diagnosed DLBCL. In certain embodiments, the cancer is newly diagnosed ROS1+ DLBCL. In certain embodiments, the cancer is newly diagnosed ALK+ DLBCL.

In certain embodiments, the cancer is large B-cell lymphoma. In certain embodiments, the cancer is ROS1+ large B-cell lymphoma. In certain embodiments, the cancer is ALK+ large B-cell lymphoma. In certain embodiments, the cancer is relapsed or refractory large B-cell lymphoma. In certain embodiments, the cancer is relapsed or refractory ROS1+ large B-cell lymphoma. In certain embodiments, the cancer is relapsed or refractory ALK+ large B-cell lymphoma. In certain embodiments, the cancer is newly diagnosed large B-cell lymphoma. In certain embodiments, the cancer is newly diagnosed ROS1+ large B-cell lymphoma. In certain embodiments, the cancer is newly diagnosed ALK+ large B-cell lymphoma.

In certain embodiments, the cancer (or ROS1+ cancer, or ALK+ cancer) is new diagnosed. In certain embodiments, the cancer (or ROS1+ cancer, or ALK+ cancer) is previously untreated.

In certain embodiments, the cancer (or ROS1+ cancer, or ALK+ cancer) is relapsed or refractory. In certain embodiments, the cancer is relapsed. In certain embodiments, the cancer (or ROS1+ cancer, or ALK+ cancer) is refractory.

In certain embodiments, the subject is previously untreated. In certain embodiments, the subject is treatment naïve to tyrosine kinase inhibitor (TKI) therapy. In certain embodiments, the subject has received one or more prior lines of therapy. In certain embodiments, the subject has received two or more prior lines of therapy. In certain embodiments, the subject has developed resistance to one or more of the prior lines of therapy. In certain embodiments, the prior therapy comprises a tyrosine kinase inhibitor (TKI). In certain embodiments, the prior therapy comprises one or more of crizotinib, ceritinib, alectinib, brigatinib, lorlatinib, entrectinib, repotrectinib, cabozantinib, foretinib, taletrectinib, merestinib, masitinib, and ensartinib. In certain embodiments, the prior therapy comprises one or more chemotherapies. In certain embodiments, the one or more chemotherapies are in addition to the TKI therapy.

In certain embodiments, the cancer (or ROS1+ cancer, or ALK+ cancer) is resistant to a tyrosine kinase inhibitor (TKI).

In certain embodiments, the cancer is resistant lung cancer. In certain embodiments, the cancer is resistant non-small cell lung cancer. In certain embodiments, the cancer is non-small cell lung cancer resistant to a TKI. In certain embodiments, the cancer is ROS1+ non-small cell lung cancer resistant to a TKI. In certain embodiments, the cancer is ALK+ non-small cell lung cancer resistant to a TKI.

In certain embodiments, the cancer is lung cancer (e.g., NSCLC), and the cancer is relapsed after, or refractory to, prior treatment by a TKI.

In certain embodiments, a compound provided herein is administered as first-line treatment. In certain embodiments, a compound provided herein is administered as second-line treatment. In certain embodiments, a compound provided herein is administered as third or fourth-line treatment.

In certain embodiments, the cancer (or ROS1+ cancer, or ALK+ cancer) is metastatic. In certain embodiments, the cancer has CNS metastases. In certain embodiments, the cancer has brain metastases. In certain embodiments, the cancer is metastatic non-small cell lung cancer (NSCLC). In certain embodiments, the cancer is metastatic ROS1+ NSCLC. In certain embodiments, the cancer is metastatic ALK+ NSCLC.

In certain embodiments, provided herein is a method for treating a patient with metastatic ALK+ non-small cell lung cancer (NSCLC), comprising administering to the patient a therapeutically effective amount of a compound provided herein, e.g., a compound of Formula (I), or a stereoisomer, a mixture of stereoisomers, or a pharmaceutically acceptable salt thereof.

In certain embodiments, provided herein is a method for treating a patient with metastatic ROS1+ non-small cell lung cancer (NSCLC), comprising administering to the patient a therapeutically effective amount of a compound provided herein, e.g., a compound of Formula (I), or a stereoisomer, a mixture of stereoisomers, or a pharmaceutically acceptable salt thereof.

In certain embodiments, the patient is an adult patient. In certain embodiments, the patient is a pediatric patient.

In certain embodiments, provided herein is a method for treating an adult patient with metastatic ROS1+ NSCLC, comprising administering to the patient a therapeutically effective amount of a compound provided herein, e.g., a compound of Formula (I), or a stereoisomer, a mixture of stereoisomers, or a pharmaceutically acceptable salt thereof.

In certain embodiments, provided herein is a method for treating an adult patient with metastatic ROS1+ NSCLC, comprising administering to the patient a therapeutically effective amount of a compound provided herein, e.g., a compound of or Formula (I), or a stereoisomer, a mixture of stereoisomers, or a pharmaceutically acceptable salt thereof, wherein the patient has progressed on or is intolerant of at least 1 prior TKI therapy.

In certain embodiments, provided herein is a method for treating an adult patient with metastatic NSCLC that is ROS1+ with solvent front mutation G2032R, comprising administering to the patient a therapeutically effective amount of a compound provided herein, e.g., a compound of Formula (I), or a stereoisomer, a mixture of stereoisomers, or a pharmaceutically acceptable salt thereof, wherein the patient has progressed on or is intolerant of at least 1 prior TKI therapy.

In certain embodiments, provided herein is a method for treating a ROS1-associated (or ROS1+) cancer in a subject in need thereof, wherein the cancer has developed resistance to a tyrosine kinase inhibitor (TKI), the method comprising administering to the subject a therapeutically effective amount of a compound provided herein, e.g., a compound of Formula (I), or a stereoisomer, a mixture of stereoisomers, or a pharmaceutically acceptable salt thereof.

In certain embodiments, provided herein is a method for treating a ROS I-associated (or ROS1+) cancer in a subject in need thereof, wherein the cancer has developed resistance to a tyrosine kinase inhibitor (TKI), and wherein the cancer has been identified as having one or more ROS1 inhibitor resistance mutations, the method comprising administering to the subject a therapeutically effective amount of a compound provided herein, e.g., a compound of Formula (I), or a stereoisomer, a mixture of stereoisomers, or a pharmaceutically acceptable salt thereof. In certain embodiments, the one or more ROS1 inhibitor resistance mutations comprise one or more amino acid substitutions at an amino acid position selected from 1986, 2004, 2026, 2032, and 2033. In certain embodiments, the one or more ROS1 inhibitor resistance mutations comprise one or more amino acid substitutions selected from S1986F, S1986Y, F2004C, F2004V, L2026M, G2032R, D2033N, L2086F, and G2101A. In certain embodiments, the one or more ROS1 inhibitor resistance mutations is G2032R. In certain embodiments, the one or more ROS1 inhibitor resistance mutations comprise G2032R and one or more of S1986F, S1986Y, F2004C, F2004V, L2026M, D2033N, or G2101A. In certain embodiments, the ROS1 inhibitor resistance mutation is L2086F.

In certain embodiments, provided herein is a method for treating an ALK-associated (or ALK+) cancer in a subject in need thereof, wherein the cancer has developed resistance to a tyrosine kinase inhibitor (TKI), the method comprising administering to the subject a therapeutically effective amount of a compound provided herein, e.g., a compound of Formula (I), or a stereoisomer, a mixture of stereoisomers, or a pharmaceutically acceptable salt thereof.

In certain embodiments, provided herein is a method for treating a ALK-associated (or ALK+) cancer in a subject in need thereof, wherein the cancer has developed resistance to a tyrosine kinase inhibitor (TKI), and wherein the cancer has been identified as having one or more ALK inhibitor resistance mutations, the method comprising administering to the subject a therapeutically effective amount of a compound provided herein, e.g., a compound of Formula (I), or a stereoisomer, a mixture of stereoisomers, or a pharmaceutically acceptable salt thereof. In certain embodiments, the one or more ALK inhibitor resistance mutations comprise one or more amino acid substitutions at an amino acid position selected from 1196, 1198, 1202, and 1269. In certain embodiments, the one or more ALK inhibitor resistance mutations comprise one or more amino acid substitutions selected from L1196M, L1198F, G1202R, and G1269A. In certain embodiments, the one or more ALK inhibitor resistance mutations is G1202R. In certain embodiments, the one or more ALK inhibitor resistance mutations comprise G1202R and one or more of L1196M, L1198F, and G1269A.

In certain embodiments, provided herein is a method for treating an adult patient with metastatic NSCLC that is ALK+ with mutation G1202R, comprising administering to the patient a therapeutically effective amount of a compound provided herein, e.g., a compound of Formula (I), or a stereoisomer, a mixture of stereoisomers, or a pharmaceutically acceptable salt thereof, wherein the patient has progressed on or is intolerant of at least 1 prior TKI therapy.

In certain embodiments, provided herein is a method for treating an ALK-associated (or ALK+) cancer in a subject in need thereof, wherein the cancer has developed resistance to a tyrosine kinase inhibitor (TKI), the method comprising administering to the subject a therapeutically effective amount of a compound provided herein, e.g., a compound of Formula (1), or a stereoisomer, a mixture of stereoisomers, or a pharmaceutically acceptable salt thereof.

In certain embodiments, the TKI is a ROS1 inhibitor. In certain embodiments, the TKI is an ALK inhibitor. In certain embodiments, the TKI is crizotinib, ceritinib, alectinib, brigatinib, lorlatinib, entrectinib, repotrectinib, cabozantinib, foretinib, merestinib, taletrectinib, masitinib, or ensartinib. In certain embodiments, the TKI is crizotinib. In certain embodiments, the TKI is entrectinib. In certain embodiments, the TKI is alectinib. In certain embodiments, the TKI is lorlatinib.

In certain embodiments, the cancer or disease is in a pediatric patient (including an infantile patient). In certain embodiments, the cancer is systemic anaplastic large cell lymphoma (ALCL) that is ALK+ in pediatric patients 1 year of age or older, and young adults. In another embodiment, the cancer is relapsed or refractory systemic anaplastic large cell lymphoma (ALCL) that is ALK+ in pediatric patients 1 year of age or older, and young adults. In certain embodiments, the cancer is systemic anaplastic large cell lymphoma (ALCL) that is ROS1+ in pediatric patients 1 year of age or older, and young adults. In another embodiment, the cancer is relapsed or refractory systemic anaplastic large cell lymphoma (ALCL) that is ROS1+ in pediatric patients 1 year of age or older, and young adults.

In certain embodiments, the methods for treating or preventing cancer can be demonstrated by one or more responses such as increased apoptosis, inhibition of tumor growth, reduction of tumor metastasis, inhibition of tumor metastasis, reduction of microvessel density, decreased neovascularization, inhibition of tumor migration, tumor regression, and increased survival of the subject.

### 4.4 Combination Treatments

In some embodiments, the method of treating or preventing cancer may comprise administering a compound of Formula (I) conjointly with one or more other chemotherapeutic agent(s).

As used herein and unless otherwise specified, by “conjointly” or “in combination with”, it is not intended to imply that the other agent and the compound of Formula (I) must be administered at the same time and/or formulated for delivery together, although these methods of delivery are also provided herein. The compound provided herein can be administered concurrently with, prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks before), or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks after), one or more other agents (e.g., one or more other additional agents). In general, each therapeutic agent is administered at a dose and/or on a time schedule determined for that particular agent. The other therapeutic agent can be administered with the compound provided herein in a single composition or separately in a different composition. Triple therapy is also contemplated herein.

Chemotherapeutic agents that may be conjointly administered with compounds provided herein include: 1-amino-4-phenylamino-9,10-dioxo-9,10-dihydroanthracene-2-sulfonate (acid blue 25), 1-amino-4-[4-hydroxyphenyl-amino]-9,10-dioxo-9,10-dihydroanthracene-2-sulfonate, 1-amino-4-[4-aminophenylamino]-9,10-dioxo-9,10-dihydroanthracene-2-sulfonate, 1-amino-4-[1-naphthylamino]-9,10-dioxo-9,10-dihydroanthracene-2-sulfonate, 1-amino-4-[4-fluoro-2-carboxyphenylamino]-9,10-dioxo-9,10-dihydroanthracene-2-sulfonate, 1-amino-4-[2-anthracenylamino]-9,10-dioxo-9,10-dihydroanthracene-2-sulfonate, ABT-263, afatinib dimaleate, axitinib, aminoglutethimide, amsacrine, anastrozole, APCP, asparaginase, AZD5363, Bacillus Calmette-Guérin vaccine (bcg), bicalutamide, bleomycin, bortezomib, β-methylene-ADP (AOPCP), buserelin, busulfan, cabazitaxel, cabozantinib, campothecin, capecitabine, carboplatin, carfilzomib, carmustine, ceritinib, chlorambucil, chloroquine, cisplatin, cladribine, clodronate, cobimetinib, colchicine, crizotinib, cyclophosphamide, cyproterone, cytarabine, dacarbazine, dactinomycin, daunorubicin, demethoxyviridin, dexamethasone, dichloroacetate, dienestrol, diethylstilbestrol, docetaxel, doxorubicin, epirubicin, eribulin, erlotinib, estradiol, estramustine, etoposide, everolimus, exemestane, filgrastim, fludarabine, fludrocortisone, fluorouracil, fluoxymesterone, flutamide, gefitinib, gemcitabine, genistein, goserelin, GSK1120212, hydroxyurea, idarubicin, ifosfamide, imatinib, interferon, irinotecan, ixabepilone, lenalidomide, letrozole, leucovorin, leuprolide, levamisole, lomustine, lonidamine, mechlorethamine, medroxyprogesterone, megestrol, melphalan, mercaptopurine, mesna, metformin, methotrexate, miltefosine, mitomycin, mitotane, mitoxantrone, MK-2206, mutamycin, N-(4-sulfamoylphenylcarbamothioyl) pivalamide, NF279, NF449, nilutamide, nocodazole, octreotide, olaparib, oxaliplatin, paclitaxel, pamidronate, pazopanib, pemexetred, pentostatin, perifosine, PF-04691502, plicamycin, pomalidomide, porfimer, PPADS, procarbazine, quercetin, raltitrexed, ramucirumab, reactive blue 2, rituximab, rolofylline, romidepsin, rucaparib, selumetinib, sirolimus, sodium 2,4-dinitrobenzenesulfonate, sorafenib, streptozocin, sunitinib, suramin, talazoparib, tamoxifen, temozolomide, temsirolimus, teniposide, testosterone, thalidomide, thioguanine, thiotepa, titanocene dichloride, tonapofylline, topotecan, trametinib, trastuzumab, tretinoin, veliparib, vinblastine, vincristine, vindesine, vinorelbine, and vorinostat (SAHA). In other embodiments, chemotherapeutic agents that may be conjointly administered with compounds provided herein include: ABT-263, dexamethasone, 5-fluorouracil, PF-04691502, romidepsin, and vorinostat (SAHA). In other embodiments, chemotherapeutic agents that may be conjointly administered with compounds provided herein include: 1-amino-4-phenylamino-9,10-dioxo-9,10-dihydroanthracene-2-sulfonate (acid blue 25), 1-amino-4-[4-hydroxyphenyl-amino]-9,10-dioxo-9,10-dihydroanthracene-2-sulfonate, 1-amino-4-[4-aminophenylamino]-9,10-dioxo-9,10-dihydroanthracene-2-sulfonate, 1-amino-4-[1-naphthylamino]-9,10-dioxo-9,10-dihydroanthracene-2-sulfonate, 1-amino-4-[4-fluoro-2-carboxyphenylamino]-9,10-dioxo-9,10-dihydroanthracene-2-sulfonate, 1-amino-4-[2-anthracenyl amino]-9,10-dioxo-9,10-dihydroanthracene-2-sulfonate, APCP, β-methylene-ADP (AOPCP), capecitabine, cladribine, cytarabine, fludarabine, doxorubicin, gemcitabine, N-(4-sulfamoylphenylcarbamothioyl) pivalamide, NF279, NF449, PPADS, quercetin, reactive blue 2, rolofylline sodium 2,4-dinitrobenzenesulfonate, sumarin, and tonapofylline.

Many combination therapies have been developed for the treatment of cancer. In certain embodiments, compounds provided herein (e.g., compounds of Formula (I)) may be conjointly administered with one or more combination therapies. Examples of combination therapies with which compounds provided herein may be conjointly administered are included in Table 2.

TABLE 2
Exemplary combinatorial therapies for the treatment of cancer

| Name | Therapeutic agents |
|---|---|
| ABV | Doxorubicin, Bleomycin, Vinblastine |
| ABVD | Doxorubicin, Bleomycin, Vinblastine, Dacarbazine |
| AC (Breast) | Doxorubicin, Cyclophosphamide |
| AC (Sarcoma) | Doxorubicin, Cisplatin |
| AC (Neuroblastoma) | Cyclophosphamide, Doxorubicin |
| ACE | Cyclophosphamide, Doxorubicin, Etoposide |
| ACe | Cyclophosphamide, Doxorubicin |
| AD | Doxorubicin, Dacarbazine |
| AP | Doxorubicin, Cisplatin |
| ARAC-DNR | Cytarabine, Daunorubicin |
| B-CAVe | Bleomycin, Lomustine, Doxorubicin, Vinblastine |
| BCVPP | Carmustine, Cyclophosphamide, Vinblastine, Procarbazine, Prednisone |
| BEACOPP | Bleomycin, Etoposide, Doxorubicin, Cyclophosphamide, Vincristine, |
| | Procarbazine, Prednisone, Filgrastim |
| BEP | Bleomycin, Etoposide, Cisplatin |
| BIP | Bleomycin, Cisplatin, Ifosfamide, Mesna |
| BOMP | Bleomycin, Vincristine, Cisplatin, Mitomycin |
| CA | Cytarabine, Asparaginase |
| CABO | Cisplatin, Methotrexate, Bleomycin, Vincristine |
| CAF | Cyclophosphamide, Doxorubicin, Fluorouracil |
| CAL-G | Cyclophosphamide, Daunorubicin, Vincristine, Prednisone, |
| | Asparaginase |
| CAMP | Cyclophosphamide, Doxorubicin, Methotrexate, Procarbazine |
| CAP | Cyclophosphamide, Doxorubicin, Cisplatin |
| CAV | Cyclophosphamide, Doxorubicin, Vincristine |
| CAVE ADD | CAV and Etoposide |
| CA-VP16 | Cyclophosphamide, Doxorubicin, Etoposide |
| CC | Cyclophosphamide, Carboplatin |
| CDDP/VP-16 | Cisplatin, Etoposide |
| CEF | Cyclophosphamide, Epirubicin, Fluorouracil |
| CEPP(B) | Cyclophosphamide, Etoposide, Prednisone, with or without/ Bleomycin |
| CEV | Cyclophosphamide, Etoposide, Vincristine |
| CF | Cisplatin, Fluorouracil or Carboplatin Fluorouracil |
| CHAP | Cyclophosphamide or Cyclophosphamide, Altretamine, Doxorubicin, |
| | Cisplatin |
| ChlVPP | Chlorambucil, Vinblastine, Procarbazine, Prednisone |
| CHOP | Cyclophosphamide, Doxorubicin, Vincristine, Prednisone |
| CHOP-BLEO | Add Bleomycin to CHOP |
| CISCA | Cyclophosphamide, Doxorubicin, Cisplatin |
| CLD-BOMP | Bleomycin, Cisplatin, Vincristine, Mitomycin |
| CMF | Methotrexate, Fluorouracil, Cyclophosphamide |
| CMFP | Cyclophosphamide, Methotrexate, Fluorouracil, Prednisone |
| CMFVP | Cyclophosphamide, Methotrexate, Fluorouracil, Vincristine, |
| | Prednisone |
| CMV | Cisplatin, Methotrexate, Vinblastine |
| CNF | Cyclophosphamide, Mitoxantrone, Fluorouracil |
| CNOP | Cyclophosphamide, Mitoxantrone, Vincristine, Prednisone |
| COB | Cisplatin, Vincristine, Bleomycin |
| CODE | Cisplatin, Vincristine, Doxorubicin, Etoposide |
| COMLA | Cyclophosphamide, Vincristine, Methotrexate, Leucovorin, Cytarabine |
| COMP | Cyclophosphamide, Vincristine, Methotrexate, Prednisone |
| Cooper Regimen | Cyclophosphamide, Methotrexate, Fluorouracil, Vincristine, |
| | Prednisone |
| COP | Cyclophosphamide, Vincristine, Prednisone |
| COPE | Cyclophosphamide, Vincristine, Cisplatin, Etoposide |
| COPP | Cyclophosphamide, Vincristine, Procarbazine, Prednisone |
| CP(Chronic | Chlorambucil, Prednisone |
| lymphocytic leukemia) | |
| CP (Ovarian Cancer) | Cyclophosphamide, Cisplatin |
| CVD | Cisplatin, Vinblastine, Dacarbazine |
| CVI | Carboplatin, Etoposide, Ifosfamide, Mesna |
| CVP | Cyclophosphamide, Vincristine, Prednisome |
| CVPP | Lomustine, Procarbazine, Prednisone |
| CYVADIC | Cyclophosphamide, Vincristine, Doxorubicin, Dacarbazine |
| DA | Daunorubicin, Cytarabine |
| DAT | Daunorubicin, Cytarabine, Thioguanine |
| DAV | Daunorubicin, Cytarabine, Etoposide |
| DCT | Daunorubicin, Cytarabine, Thioguanine |
| DHAP | Cisplatin, Cytarabine, Dexamethasone |
| DI | Doxorubicin, Ifosfamide |
| DTIC/Tamoxifen | Dacarbazine, Tamoxifen |
| DVP | Daunorubicin, Vincristine, Prednisone |
| EAP | Etoposide, Doxorubicin, Cisplatin |
| EC | Etoposide, Carboplatin |
| EFP | Etoposide, Fluorouracil, Cisplatin |
| ELF | Etoposide, Leucovorin, Fluorouracil |
| EMA 86 | Mitoxantrone, Etoposide, Cytarabine |
| EP | Etoposide, Cisplatin |
| EVA | Etoposide, Vinblastine |
| FAC | Fluorouracil, Doxorubicin, Cyclophosphamide |
| FAM | Fluorouracil, Doxorubicin, Mitomycin |
| FAMTX | Methotrexate, Leucovorin, Doxorubicin |
| FAP | Fluorouracil, Doxorubicin, Cisplatin |
| F-CL | Fluorouracil, Leucovorin |
| FEC | Fluorouracil, Cyclophosphamide, Epirubicin |
| FED | Fluorouracil, Etoposide, Cisplatin |
| FL | Flutamide, Leuprolide |
| FZ | Flutamide, Goserelin acetate implant |
| HDMTX | Methotrexate, Leucovorin |
| Hexa-CAF | Altretamine, Cyclophosphamide, Methotrexate, Fluorouracil |
| IDMTX/6-MP | Methotrexate, Mercaptopurine, Leucovorin |
| IE | Ifosfamide, Etoposide, Mesna |
| IfoVP | Ifosfamide, Etoposide, Mesna |
| IPA | Ifosfamide, Cisplatin, Doxorubicin |
| M-2 | Vincristine, Carmustine, Cyclophosphamide, Prednisone, Melphalan |
| MAC-III | Methotrexate, Leucovorin, Dactinomycin, Cyclophosphamide |
| MACC | Methotrexate, Doxorubicin, Cyclophosphamide, Lomustine |
| MACOP-B | Methotrexate, Leucovorin, Doxorubicin, Cyclophosphamide, |
| | Vincristine, Bleomycin, Prednisone |
| MAID | Mesna, Doxorubicin, Ifosfamide, Dacarbazine |
| m-BACOD | Bleomycin, Doxorubicin, Cyclophosphamide, Vincristine, |
| | Dexamethasone, Methotrexate, Leucovorin |
| MBC | Methotrexate, Bleomycin, Cisplatin |
| MC | Mitoxantrone, Cytarabine |
| MF | Methotrexate, Fluorouracil, Leucovorin |
| MICE | Ifosfamide, Carboplatin, Etoposide, Mesna |
| MINE | Mesna, Ifosfamide, Mitoxantrone, Etoposide |
| mini-BEAM | Carmustine, Etoposide, Cytarabine, Melphalan |
| MOBP | Bleomycin, Vincristine, Cisplatin, Mitomycin |
| MOP | Mechlorethamine, Vincristine, Procarbazine |
| MOPP | Mechlorethamine, Vincristine, Procarbazine, Prednisone |
| MOPP/ABV | Mechlorethamine, Vincristine, Procarbazine, Prednisone, Doxorubicin, |
| | Bleomycin, Vinblastine |
| MP (multiple | Melphalan, Prednisone |
| myeloma) | |
| MP (prostate cancer) | Mitoxantrone, Prednisone |
| MTX/6-MO | Methotrexate, Mercaptopurine |
| MTX/6-MP/VP | Methotrexate, Mercaptopurine, Vincristine, Prednisone |
| MTX-CDDPAdr | Methotrexate, Leucovorin, Cisplatin, Doxorubicin |
| MV (breast cancer) | Mitomycin, Vinblastine |
| MV (acute myelocytic | Mitoxantrone, Etoposide |
| leukemia) | |
| M-VAC Methotrexate | Vinblastine, Doxorubicin, Cisplatin |
| MVP Mitomycin | Vinblastine, Cisplatin |
| MVPP | Mechlorethamine, Vinblastine, Procarbazine, Prednisone |
| NFL | Mitoxantrone, Fluorouracil, Leucovorin |
| NOVP | Mitoxantrone, Vinblastine, Vincristine |
| OPA | Vincristine, Prednisone, Doxorubicin |
| OPPA | Add Procarbazine to OPA. |
| PAC | Cisplatin, Doxorubicin |
| PAC-I | Cisplatin, Doxorubicin, Cyclophosphamide |
| PA-CI | Cisplatin, Doxorubicin |
| PCV | Lomustine, Procarbazine, Vincristine |
| PFL | Cisplatin, Fluorouracil, Leucovorin |
| POC | Prednisone, Vincristine, Lomustine |
| ProMACE | Prednisone, Methotrexate, Leucovorin, Doxorubicin, |
| | Cyclophosphamide, Etoposide |
| ProMACE/cytaBOM | Prednisone, Doxorubicin, Cyclophosphamide, Etoposide, Cytarabine, |
| | Bleomycin, Vincristine, Methotrexate, Leucovorin, Cotrimoxazole |
| PRoMACE/MOPP | Prednisone, Doxorubicin, Cyclophosphamide, Etoposide, |
| | Mechlorethamine, Vincristine, Procarbazine, Methotrexate, Leucovorin |
| Pt/VM | Cisplatin, Teniposide |
| PVA | Prednisone, Vincristine, Asparaginase |
| PVB | Cisplatin, Vinblastine, Bleomycin |
| PVDA | Prednisone, Vincristine, Daunorubicin, Asparaginase |
| SMF | Streptozocin, Mitomycin, Fluorouracil |
| TAD | Mechlorethamine, Doxorubicin, Vinblastine, Vincristine, Bleomycin, |
| | Etoposide, Prednisone |
| TTT | Methotrexate, Cytarabine, Hydrocortisone |
| Topo/CTX | Cyclophosphamide, Topotecan, Mesna |
| VAB-6 | Cyclophosphamide, Dactinomycin, Vinblastine, Cisplatin, Bleomycin |
| VAC | Vincristine, Dactinomycin, Cyclophosphamide |
| VACAdr | Vincristine, Cyclophosphamide, Doxorubicin, Dactinomycin, |
| | Vincristine |
| VAD | Vincristine, Doxorubicin, Dexamethasone |
| VATH | Vinblastine, Doxorubicin, Thiotepa, Flouxymesterone |
| VBAP | Vincristine, Carmustine, Doxorubicin, Prednisone |
| VBCMP | Vincristine, Carmustine, Melphalan, Cyclophosphamide, Prednisone |
| VC | Vinorelbine, Cisplatin |
| VCAP | Vincristine, Cyclophosphamide, Doxorubicin, Prednisone |
| VD | Vinorelbine, Doxorubicin |
| VelP | Vinblastine, Cisplatin, Ifosfamide, Mesna |
| VIP | Etoposide, Cisplatin, Ifosfamide, Mesna |
| VM | Mitomycin, Vinblastine |
| VMCP | Vincristine, Melphalan, Cyclophosphamide, Prednisone |
| VP | Etoposide, Cisplatin |
| V-TAD | Etoposide, Thioguanine, Daunorubicin, Cytarabine |
| 5 + 2 | Cytarabine, Daunorubicin, Mitoxantrone |
| 7 + 3 | Cytarabine with/, Daunorubicin or Idarubicin or Mitoxantrone |
| “8 in 1” | Methylprednisolone, Vincristine, Lomustine, Procarbazine, |
| | Hydroxyurea, Cisplatin, Cytarabine, Dacarbazine |

In certain embodiments, the conjoint therapies provided herein comprise conjoint administration with other types of chemotherapeutic agents, such as immuno-oncology agents. Cancer cells often have specific cell surface antigens that can be recognized by the immune system. Thus, immuno-oncology agents, such as monoclonal antibodies, can selectively bind to cancer cell antigens and effect cell death. Other immuno-oncology agents can suppress tumor-mediated inhibition of the native immune response or otherwise activate the immune response and thus facilitate recognition of the tumor by the immune system. Exemplary antibody immuno-oncology agents, include, but are not limited to, abagovomab, adecatumumab, afutuzumab, alemtuzumab, anatumomab mafenatox, apolizumab, blinatumomab, BMS-936559, catumaxomab, durvalumab, epacadostat, epratuzumab, indoximod, inotuzumab ozogamicin, intelumumab, ipilimumab, isatuximab, lambrolizumab, MED14736, MPDL3280A, nivolumab, obinutuzumab, ocaratuzumab, ofatumumab, olatatumab, pembrolizumab, pidilizumab, rituximab, ticilimumab, samalizumab, and tremelimumab. In some embodiments, the antibody immuno-oncology agents are selected from anti-CD73 monoclonal antibody (mAb), anti-CD39 mAb, anti-PD-1 mAb, and anti-CTLA4 mAb. Thus, in some embodiments, the methods provided herein comprise conjoint administration of one or more immuno-oncology agents, such as the agents mentioned above.

In some embodiments, the combination therapy comprises conjoint administration of a compound provided herein, such as a compound of Formula (I), with SH2 inhibitors, such as CGP78850, CPG85793, C90, C126, G7-18NATE, G7-B1, and NSC642056.

In some embodiments, the combination therapy comprises conjoint administration of a compound provided herein, such as a compound of Formula (I), with MEK inhibitors, such as trametinib, cobimetinib, binimetinib, selumetinib, PD-325901, CI-1040, and TAK-733.

In some embodiments, the combination therapy comprises conjoint administration of a compound provided herein, such as a compound of Formula (I), with a MET inhibitor selected from JNJ-38877605, PF-04217903, foretinib, AMG 458, tivantinib, cabozantinib, crizotinib, capmatinib hydrochloride, tepotinib hydrochloride, and savolitinib.

In some embodiments, the combination therapy comprises conjoint administration of a compound provided herein, such as Formula (I), with a SHP2 inhibitor selected from TNO-155, RMC-4630, JAB-3068, or RLY-1971.

In some embodiments, the combination therapy comprises conjoint administration of a compound provided herein, such as a compound of Formula (I), with a RAS inhibitor selected from aliskiren, captopril, losartan, irbesartan, olmesartan, candesartan, valsartan, fimasartan, azilsartan, telmisartan, eprosartan, benazepril, enalapril, lisinopril, perindopril, quinapril, ramipril, and trandolapril.

In some embodiment, the combination therapy comprises administration of a compound provided herein, e.g., a compound of Formula (I), in combination with a TKI. In certain embodiments, the TKI is a ROS1 inhibitor. In certain embodiments, the TKI is an ALK inhibitor. In certain embodiments, the TKI is crizotinib, ceritinib, alectinib, brigatinib, lorlatinib, entrectinib, repotrectinib, cabozantinib, foretinib, taletrectinib, merestinib, masitinib, or ensartinib. In certain embodiments, the TKI is crizotinib. In certain embodiments, the TKI is entrectinib. In certain embodiments, the TKI is alectinib. In certain embodiments, the TKI is brigatinib.

In some embodiments, the combination therapy comprises conjoint administration of a compound provided herein, such as a compound of Formula (I), with anti-PD-1 therapy. In certain embodiments, the combination therapy comprises conjoint administration of a compound provided herein, such as a compound of Formula (I), with oxaliplatin. In other embodiments, the combination therapy comprises conjoint administration of a compound provided herein, such as a compound of Formula (I), with doxorubicin.

In certain embodiments, a compound provided herein may be conjointly administered with non-chemical methods of cancer treatment. In certain embodiments, a compound provided herein may be conjointly administered with radiation therapy. In certain embodiments, a compound provided herein may be conjointly administered with surgery, with thermoablation, with focused ultrasound therapy, with cryotherapy, or with any combination of these.

In certain embodiments, a compound provided herein may be conjointly administered with an agent that inhibits CD47/SIRPα interaction. In certain embodiments, the agent that inhibits CD47/SIRPα interaction is a CD47 inhibitor. In certain embodiments, the CD47 inhibitor is an anti-CD47 antibody. In certain embodiments, the anti-CD47 antibody is AO-176, CC-90002, GenSci-059, IMC-002, lemzoparlimab, letaplimab, ligufalimab, magrolimab, MIL-95, SHR-1603, ZL-1201, STI-6643, SRF231, TQB2928, or SGN-CD47M. In certain embodiments, the anti-CD47 antibody is magrolimab. In certain embodiments, the CD47 inhibitor is a small molecule. In certain embodiments, the CD47 inhibitor is RRx-001. In certain embodiments, the agent that inhibits CD47/SIRPα interaction is an anti-CD47 bispecific antibody. In certain embodiments, the anti-CD47 bispecific antibody is BAT-7104, HX-009, IBI-322, IMM-0306, JMT-601, SG-12473, SIRPα-Fc-CD40L, TG-1801, HX009, PF-07257876, DVD-Ig SL/LL, SIRPa-gamma-CD20 HC, CD20-2GL-SIRPα HC, CD20-4GL-SIRPa HC, bi-scFv RTX-CD47, LQ007, HMBD004A, HMBD004B, NI-1801, NI-2401, NI-2601, PT-886, PT-796, PT-217, IMM-26011, IMM-2902, SG3847, BH-29XX, PMC-122, ABP-160, IMM-2505, TJ-L1C4, IAB, SL-172154, DSP107, TJ C4GM, or IMM-0207. In certain embodiments, the agent that inhibits CD47/SIRPα interaction is a SIRPα inhibitor. In certain embodiments, the SIRPα inhibitor is an anti-SIRPα antibody. In certain embodiments, the anti-SIRPα antibody is BI-765063, CC-95251, GS-0189, HSIRPB, H21, ES004, ALO08, ADU-1805, or Abx701. In certain embodiments, the SIRPα inhibitor is a small molecule. In certain embodiments, the agent that inhibits CD47/SIRPα interaction is a SIRPα/Fc fusion protein antibody. In certain embodiments, the SIRPα/Fc fusion protein antibody is DSP-107, evorpacept, IMM-01, TTI-621, or TTI-622.

In certain embodiments, compounds provided herein may be conjointly administered with one or more other compounds provided herein. Moreover, such combinations may be conjointly administered with other therapeutic agents, such as other agents suitable for the treatment of cancer, immunological or neurological diseases, such as the agents identified above. In certain embodiments, conjointly administering one or more additional chemotherapeutic agents with a compound provided herein provides a synergistic effect. In certain embodiments, conjointly administering one or more additional chemotherapeutic agents provides an additive effect.

### 4.5 Pharmaceutical Compositions

In certain embodiments, provided herein is a pharmaceutical preparation suitable for use in a human patient, comprising any of the compounds shown above (e.g., a compound provided herein, such as a compound of Formula (I), and one or more pharmaceutically acceptable excipients. In certain embodiments, the pharmaceutical preparations may be for use in treating or preventing a condition or disease as described herein. Any of the compounds provided herein may be used in the manufacture of medicaments for the treatment of any diseases or conditions provided herein.

The compositions and methods provided herein may be utilized to treat a subject in need thereof. In certain embodiments, the subject is a mammal such as a human, or a non-human mammal. In one embodiment, when administered to subject, such as a human, the composition or the compound is administered as a pharmaceutical composition comprising, for example, a compound provided herein and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In one embodiment, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as an eye drop.

A pharmaceutically acceptable carrier can contain physiologically acceptable agents that act, for example, to stabilize, increase solubility or to increase the absorption of a compound such as a compound provided herein. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-microemulsifying drug delivery system. The pharmaceutical composition (preparation) also can be a liposome or other polymer matrix, which can have incorporated therein, for example, a compound provided herein. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of a subject without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the subject. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); anally, rectally or vaginally (for example, as a pessary, cream or foam); parenterally (including intramuscularly, intravenously, subcutaneously or intrathecally as, for example, a sterile solution or suspension); nasally; intraperitoneally; subcutaneously; transdermally (for example as a patch applied to the skin); and topically (for example, as a cream, ointment or spray applied to the skin, or as an eye drop). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the subject being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, from about 5 percent to about 70 percent, or from about 10 percent to about 30 percent.

Methods of preparing these formulations or compositions include the step of bringing into association an active compound, such as a compound provided herein, with the carrier and, optionally, one or more accessory ingredients. In general, the formulations are prepared by uniformly and intimately bringing into association a compound provided herein with liquid carriers, or finely divided solid carriers, or both, and then, if necessary, shaping the product.

Formulations provided herein suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound provided herein as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof, (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions for rectal, vaginal, or urethral administration may be presented as a suppository, which may be prepared by mixing one or more active compounds with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the pharmaceutical compositions for administration to the mouth may be presented as a mouthwash, or an oral spray, or an oral ointment.

Alternatively or additionally, compositions can be formulated for delivery via a catheter, stent, wire, or other intraluminal device. Delivery via such devices may be especially useful for delivery to the bladder, urethra, ureter, rectum, or intestine.

Formulations which are suitable for vaginal administration also include pessaries, tampons, creams, gels, pastes, foams or spray formulations containing such carriers as are known in the art to be appropriate.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound provided herein to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also provided herein. Exemplary ophthalmic formulations are described in U.S. Publication Nos. 2005/0080056, 2005/0059744, 2005/0031697 and 2005/004074 and U.S. Pat. No. 6,583,124, the contents of which are incorporated herein by reference. If desired, liquid ophthalmic formulations have properties similar to that of lacrimal fluids, aqueous humor or vitreous humor or are compatible with such fluids. A route of administration is local administration (e.g., topical administration, such as eye drops, or administration via an implant).

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions provided herein include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods provided herein, active compounds can be given per se or as a pharmaceutical composition containing, for example, 0.1 to 99.5% (e.g., 0.5 to 90%) of active ingredient in combination with a pharmaceutically acceptable carrier.

Methods of introduction may also be provided by rechargeable or biodegradable devices. Various slow-release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the subject being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the subject's condition, the disorder being treated, the stability of the compound, and, if desired, another type of therapeutic agent being administered with the compound provided herein. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods provided herein will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments, the active compound may be administered two or three times daily. In certain embodiments, the active compound will be administered once daily.

In certain embodiments, compounds provided herein may be used alone or conjointly administered with another type of therapeutic agent. As used herein, the phrase “conjoint administration” refers to any form of administration of two or more different therapeutic compounds such that the second compound is administered while the previously administered therapeutic compound is still effective in the body (e.g., the two compounds are simultaneously effective in the subject, which may include synergistic effects of the two compounds). For example, the different therapeutic compounds can be administered either in the same formulation or in a separate formulation, either concomitantly or sequentially. In certain embodiments, the different therapeutic compounds can be administered within one hour, 12 hours, 24 hours, 36 hours, 48 hours, 72 hours, or a week of one another. Thus, a subject who receives such treatment can benefit from a combined effect of different therapeutic compounds.

In certain embodiments, conjoint administration of compounds provided herein with one or more additional therapeutic agent(s) (e.g., one or more additional chemotherapeutic agent(s)) provides improved efficacy relative to each individual administration of the compound provided herein (e.g., compound of Formula I or Ia) or the one or more additional therapeutic agent(s). In certain such embodiments, the conjoint administration provides an additive effect, wherein an additive effect refers to the sum of each of the effects of individual administration of the compound provided herein and the one or more additional therapeutic agent(s).

Also provided herein is use of pharmaceutically acceptable salts of compounds provided herein in the compositions and methods provided herein. In certain embodiments, contemplated salts provided herein include, but are not limited to, alkyl, dialkyl, trialkyl or tetra-alkyl ammonium salts. In certain embodiments, contemplated salts provided herein include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts provided herein include, but are not limited to, Na, Ca, K, Mg, Zn or other metal salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Pharmaceutically acceptable anionic salts include acetate, aspartate, benzenesulfonate, benzoate, besylate, bicarbonate, bitartrate, bromide, camsylate, carbonate, chloride, citrate, decanoate, edetate, esylate, fumarate, gluceptate, gluconate, glutamate, glycolate, hexanoate, hydroxynaphthoate, iodide, isethionate, lactate, lactobionate, malate, maleate, mandelate, mesylate, methylsulfate, mucate, napsylate, nitrate, octanoate, oleate, pamoate, pantothenate, phosphate, polygalacturonate, propionate, salicylate, stearate, acetate, succinate, sulfate, tartrate, teoclate, and tosylate.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

The disclosure now being generally described, it will be more readily understood by reference to the following examples which are included merely for purposes of illustration of certain aspects and embodiments provided herein, and are not intended to limit the disclosure.

### 5. EXAMPLES

### General Synthetic Methods

The compounds provided herein can be prepared by a variety of synthetic methods, as further described and illustrated herein. It will be understood by those with skill in the art that the following general synthetic methods are representative and not intended to be limiting. Racemic compounds can be enantiomerically enriched via chiral, preparative, SFC or HPLC separation. Stereogenic sp³ centers drawn without up- or down-wedge annotation represent an unquantified mixture of configurations at that position. Stereogenic sp³ centers drawn with up- or down-wedge annotations represent a stereo-enrichment for the configuration drawn. Stereogenic sp³ centers drawn with up- or down-wedge further annotated with “or1” represent stereo-enrichment for a single, unknown configuration.

| | |
|---|---|
| Abbreviations | Definition |

Solvents

| | |
|---|---|
| EA, EtOAc | ethyl acetate |
| PE, pet. ether | petroleum ether |
| THF | tetrahydrofuran |
| DCM | dichloromethane |
| DMF | N,N-dimethylformamide |
| DMA | N,N-dimethylacetamide |
| NMP | N-methyl-2-pyrrolidone |
| DMSO | dimethyl sulfoxide |
| IPA | isopropyl alcohol |
| DME | dimethoxyethane |
| MeCN, ACN | acetonitrile |
| DCE | dichloroethane |

Reagents

| | |
|---|---|
| DAST | diethylaminosulfur trifluoride |
| DIAD | diisopropyl azodicarboxylate |
| DEAD | diethyl azodicarboxylate |
| DBAD | di-tert-butyl azodicarboxylate |
| DIPEA, DIEA | N,N-diisopropylethylamine |
| TEA | triethylamine |
| ATP | adenosine triphosphate |
| TFA | trifluoroacetic acid |
| FA | formic acid |
| DIBAL, DIBAL-H, | diisobutylaluminium hydride |
| DIBALH | |
| AcOH, HOAc | acetic acid |
| TES | triethylsilane |
| n-BuLi, BuLi | n-butyllithium |
| LDA | lithium diisopropylamide |
| NBS | N-bromosuccinimide |
| NIS | N-iodosuccinimide |
| NCS | N-chlorosuccinimide |
| DMP | Dess-Martin periodinane |
| DEA | diethylamine |
| DMF-DMA | 1,1-dimethoxy-N,N-dimethylmethanamine |
| TMP | 2,2,6,6-tetramethylpiperidine |
| NMO | N-methylmorpholine N-oxide |
| TBSCI | tert-butyldimethylsilyl chloride |
| KOAc, AcOK | potassium acetate |
| NaOAc, AcONa | sodium acetate |
| SEMCl | 2-(trimethylsilyl)ethoxymethyl chloride |
| tBuLi, t-BuLi | tert-butyllithium |
| NFSI | N-fluorobenzenesulfonimide |
| AIBN | azobisisobutyronitrile |
| EDCI | 1-ethyl-3-(3-dimethylaminopropyl)carbodiimide |
| HOBT | hydroxybenzotriazole |
| TBAF | tetra-n-butylammonium fluoride |
| HATU | 1-[bis(dimethylamino)methylene]-1H-1,2,3- |
| | triazolo[4,5-b]pyridinium 3-oxide |
| | hexafluorophosphate |
| XPhos | 2-dicyclohexylphosphino-2′,4′,6′- |
| | triisopropylbiphenyl |
| cataCXium A | di(1-adamantyl)-n-butylphosphine |
| DPPP | 1,3-bis(diphenylphosphino)propane |
| DPPF | 1,1′-bis(diphenylphosphino)ferrocene |
| TfOH | triflic acid |
| HMTA | 1,3,5,7-tetraazaadamantane |
| PMBCl | p-methoxybenzyl chloride |
| HEPES | 4-(2-hydroxyethyl)-1-piperazineethanesulfonic acid |
| EGTA | ethylene glycol-bis(2-aminoethylether)- |
| | N,N,N′,N′-tetraacetic acid |
| MCPBA | meta-chloroperbenzoic acid |
| BI-DIME, BIDIME | 3-(t-butyl)-4-(2,6-dimethoxyphenyl)-2,3- |
| | dihydrobenzo[d][1,3]oxaphosphole |
| DMAP | N,N-dimethylpyridin-4-amine |
| DMEDA | N,N′-dimethylethane-1,2-diamine |
| LiHMDS | lithium bis(trimethylsilyl)amide |
| NaHMDS | sodium bis(trimethylsilyl)amide |
| MOMCl | chloro(methoxy)methane |
| TFAA | trifluoroacetic anhydride |
| DBA | dibenzylideneacetone |

Other

| | |
|---|---|
| HPLC | high-pressure liquid chromatography |
| Prep | preparative |
| wt | wild-type |
| rt, r.t., RT | room-temperature |
| SFC | supercritical fluid chromatography |
| V/V | volume/volume |
| LC/MS, LC-MS, | liquid chromatography-mass spectrometry |
| LCMS | |
| MS | mass spectrometry |
| ESI, ES+, ES− | electrospray ionization |
| NMR | nuclear magnetic resonance |
| ppm | parts per million |
| sat | saturated |
| aq | aqueous |
| TLC | thin layer chromatography |
| t_{R} | retention time |

### Method A

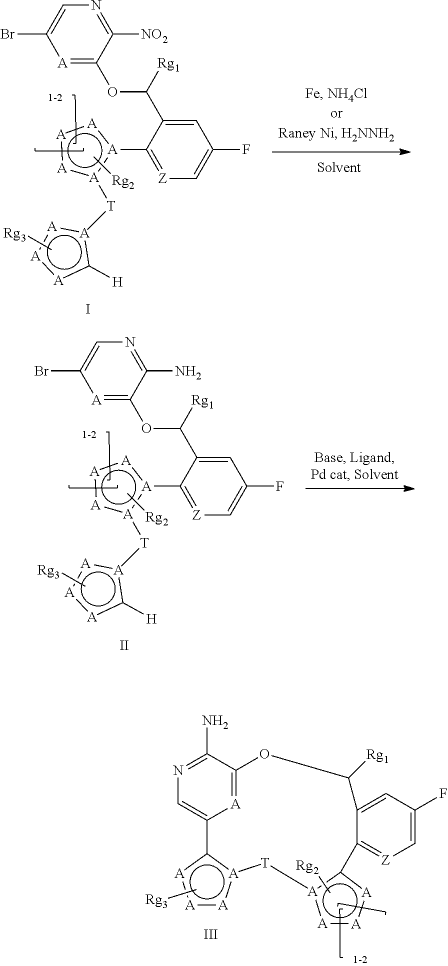

Nitropyridine I may be reduced using Fe metal conditions to provide aminopyridines of type II. This transformation can also be effected using Raney nickel and hydrazine; or in cases where the substrate contains an isoxazole moiety, yields can be improved by using SnCl2 conditions instead. Intramolecular ring closure of II may be effected using C—H insertion cross-coupling conditions to afford compounds of type III. Potassium acetate or potassium pivalate are effective bases for the macrocyclization step.

### Method B

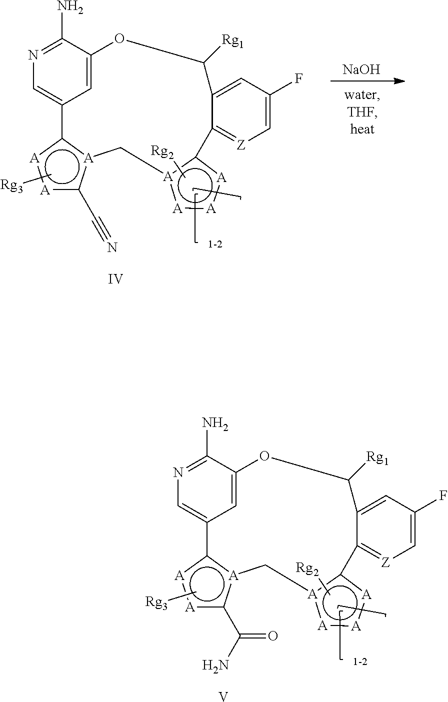

Nitriles of type IV may be converted to primary amides of type V using sodium hydroxide in tetrahydrofuran. Alternate hydroxide sources include, but are not limited to, lithium hydroxide, potassium hydroxide, cesium hydroxide, or tetraalkylammonium hydroxides such as Triton B.

### Method C

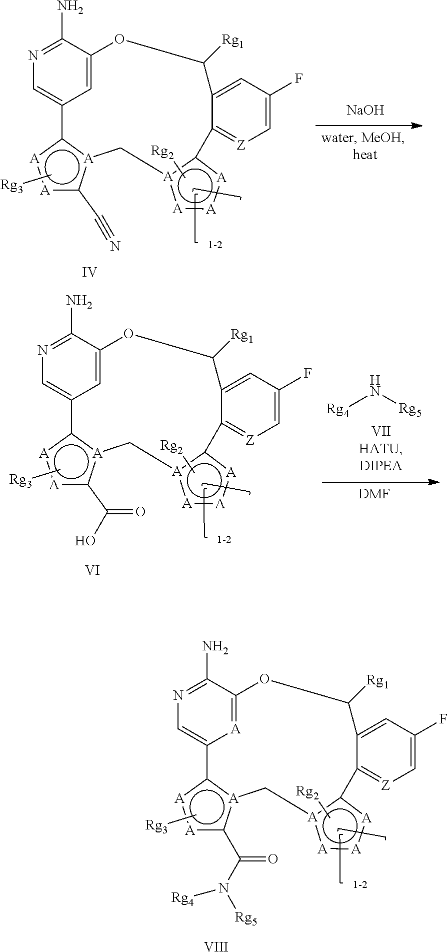

Reaction of nitriles IV with sodium hydroxide in methanol affords carboxylic acids of type VI. Lithium hydroxide, potassium hydroxide, cesium hydroxide, or tetraalkylammonium hydroxides such as Triton B, can be used as alternatives to sodium hydroxide for this transformation. Carboxylic acids of type VI can be reacted with amines of type VII in the presence of an amide coupling reagent to give carboxamides of type VIII. Suitable amide coupling reagents include, but are not limited to, HATU, EDCI, TBTU, CDI, and T3P.

### Method D

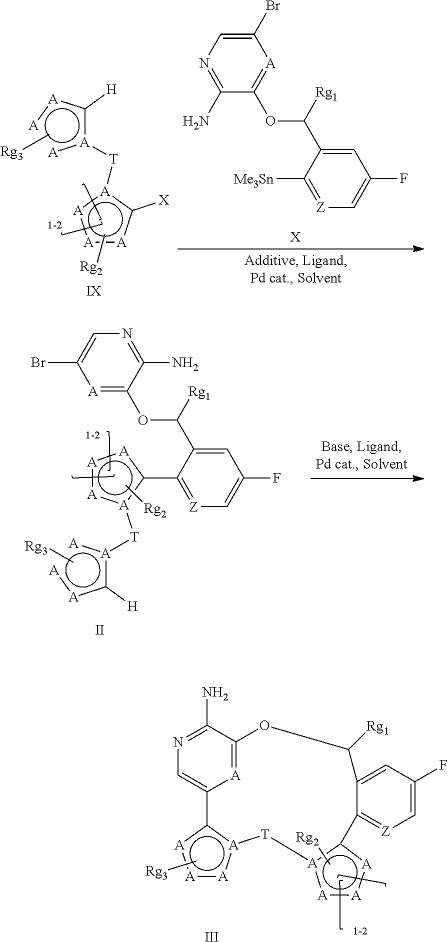

Halide IX may be coupled with stannane X using Stille coupling conditions to provide compounds of type II. Various additives including (but not limited to) LiCl or CuI may be optionally employed to facilitate this reaction. Intramolecular ring closure of halide II may be effected using C—H insertion cross-coupling conditions to afford compounds III. Potassium acetate or potassium pivalate are effective bases for the macrocyclization step.

### Method E

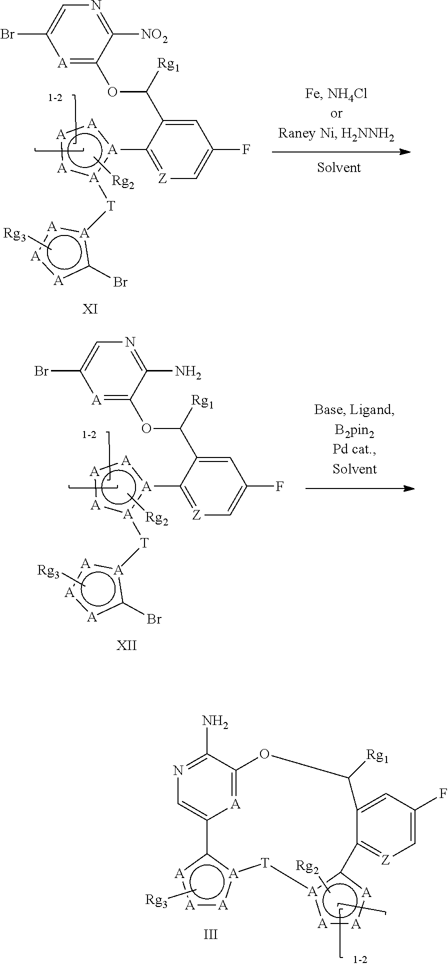

Nitropyridine XI may be reduced using Fe metal conditions to provide aminopyridines of type XII. This transformation can also be effected using Raney nickel and hydrazine; or in cases where the substrate contains an isoxazole moiety, yields can be improved by using SnCl₂ conditions instead. Intramolecular ring closure of compound XII may be effected using two-step, one-pot borylation/Suzuki cross-coupling conditions to afford compounds III.

### Method F

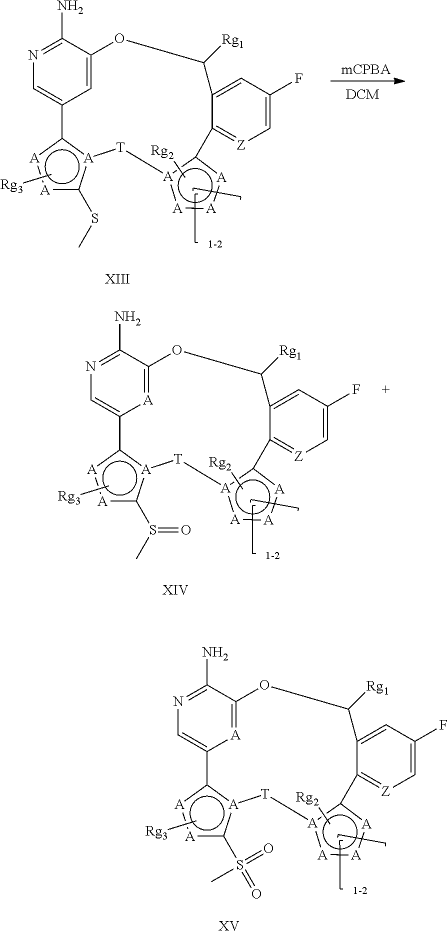

Thioether XIII may be converted to a mixture of sulfoxide XIV and sulfone XV using meta-chloroperoxybenzoic acid. The products may be separated and isolated using chromatographic methods.

### Method G

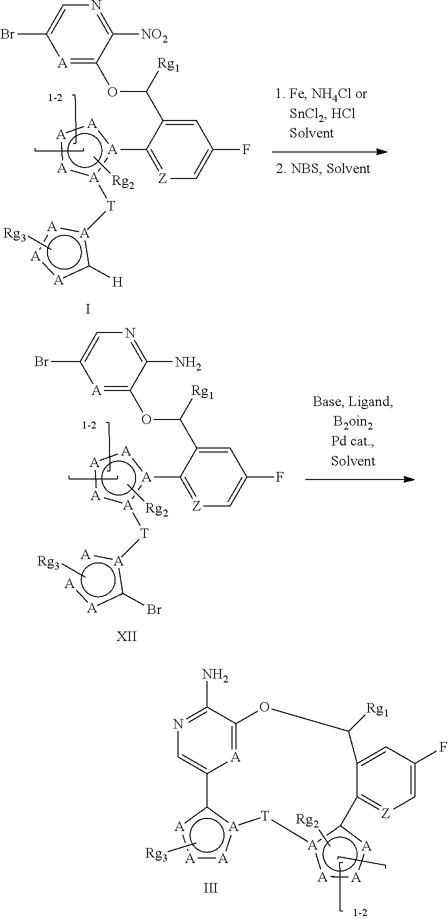

Nitropyridine I may be reduced using iron metal conditions to provide an aminopyridine intermediate that can be converted to bromide XII with NBS. The initial iron reduction step can also be effected using Raney nickel and hydrazine; or in cases where the substrate contains an isoxazole moiety, yields can be improved by using SnCl₂ conditions instead. Intramolecular ring closure of compound XII may be effected using two-step, one-pot borylation/Suzuki cross-coupling conditions to afford compounds III.

### Method H

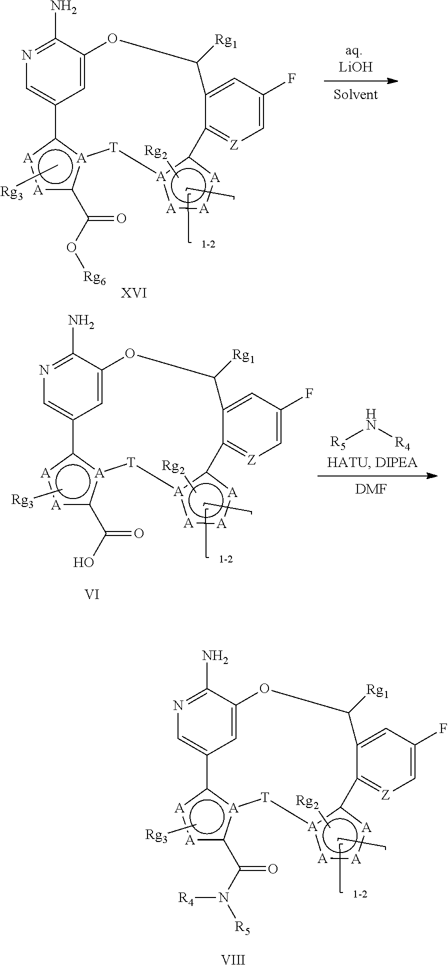

Reaction of esters XVI with aqueous lithium hydroxide in an organic solvent affords carboxylic acids of type VI. Sodium hydroxide, potassium hydroxide, cesium hydroxide, or tetraalkylammonium hydroxides such as Triton B, can be used as alternatives to lithium hydroxide for this transformation. Carboxylic acids of type VI can react with amines of type VII in the presence of an amide coupling reagent to give carboxamides of type VIII. Suitable amide coupling reagents include, but are not limited to, HATU, EDCI, TBTU, CDI, and T3P.

### Method I

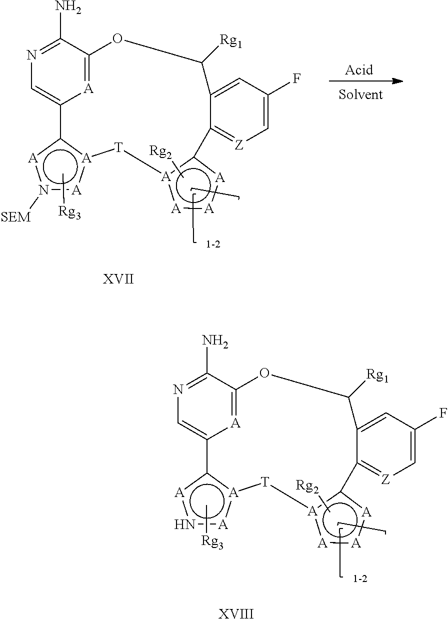

Deprotection of SEM ethers XVII with an acid can afford pyrazoles XVIII. Suitable acids include, but are not limited to, TFA, and HCl.

### Method J

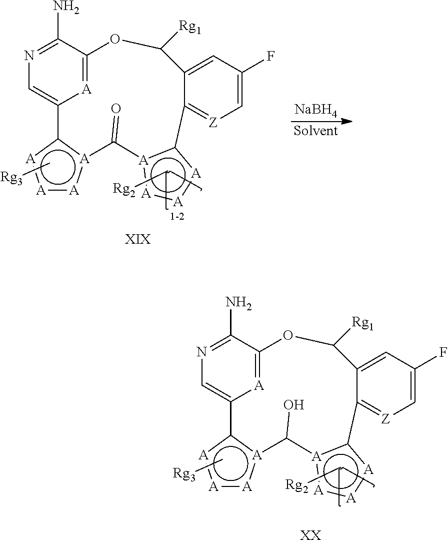

Ketone XIX may be reduced to alcohol XX using a reducing agent such as sodium borohydride in an alcohol solvent such as methanol.

### Method K

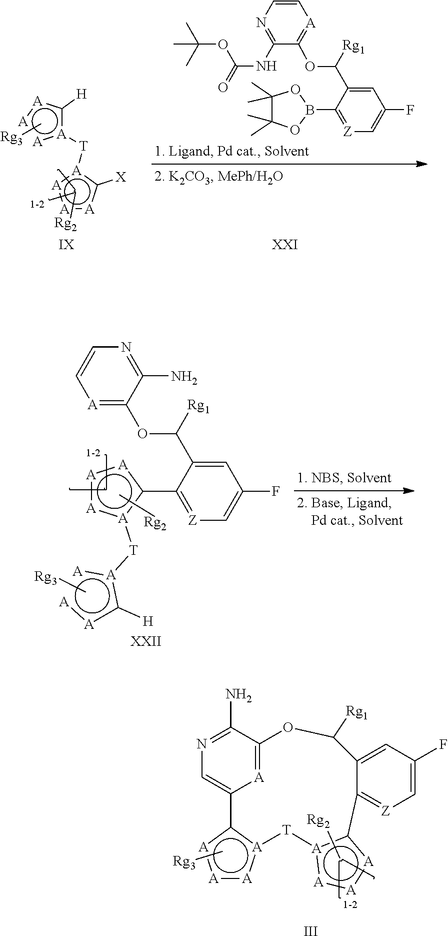

Halide IX may be coupled with boronate XXI using Suzuki coupling conditions to provide, after base-promoted Boc deprotection, aminopyridine XXII. Bromination of the aminopyridine ring with NBS followed by intramolecular ring closure using C—H insertion cross-coupling conditions can afford macrocycle III. Potassium acetate or potassium pivalate are effective bases for the macrocyclization step.

### Method L

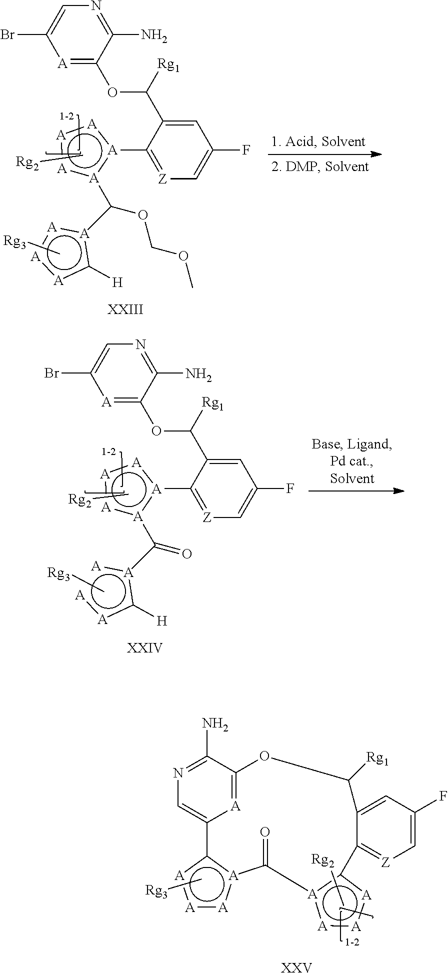

MOM ether XXIII may be deprotected under acidic conditions and the exposed alcohol can be oxidized using Dess-Martin periodinane to provide ketone XXIV. Appropriate acids include, but are not limited to, TFA and HCl. Intramolecular ring closure of XXIV may be effected using C—H insertion cross-coupling conditions to afford macrocycle XXV Potassium acetate or potassium pivalate are effective bases for the macrocyclization step.

### Method M

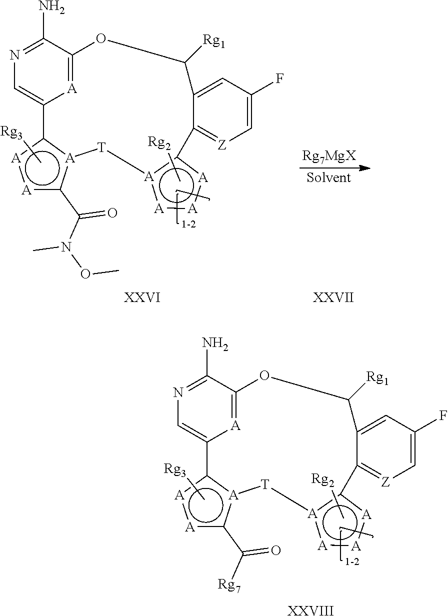

Weinreb amide XXVII may be converted to ketone XXVIII through reaction with Grignard reagent XXVII.

As used in general methods A, B, C, D, E, F, G, H, I, J, K, L and M, each instance of A is independently O, N, S, NR_{g8}, CR_{g9}, or C(R_{g9})₂, as valence permits, wherein each instance of R_{g8} and R_{g9} is independently H or C₁₋₆ alkyl. Each instance of R_{g1} is independently H, methyl, or hydroxymethyl. Each instance of R_{g2} is independently H, halo, CN, C₁₋₄ alkoxy, halo-C₁₋₄ alkyl, and C₁₋₄ alkyl. Each instance of R_{g3} is independently H, CN, halo,

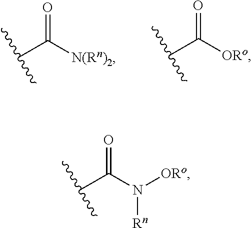

—CO—C₁₋₄ alkyl, 5-membered heteroaryl, C₁₋₄ alkyl-SO—, C₁₋₄ alkyl-SO₂—, C₁₋₄ alkoxy, C₁₋₄ alkyl, halo-C₁₋₄ alkyl, C₁₋₄ alkoxy-C₁₋₄ alkyl, C₃₋₄ cycloalkylmethyl, C₃₋₆ cycloalkyl, and C₃₋₆ heterocyclyl; wherein the heteroaryl, cycloalkyl, heterocyclyl, or alkyl is further substituted with 0, 1, or 3 occurrences of C₁₋₄alkyl or halogen as valency permits. Each instance of Rₙ is independently H, C₁₋₄ alkyl, halo-C₁₋₄ alkyl, or C₃₋₆ cycloalkyl, or two Rₙ groups are taken together with their intervening nitrogen to form a C₃₋₆ heterocycloalkyl optionally substituted with one or more occurrences of C₁₋₄ alkyl or halogen. Each instance of R_{g4} and R_{g5} is independently substituted or unsubstituted C₁₋₄ alkyl; or R_{g4} and R_{g5} are taken together with their intervening nitrogen to form a C₃₋₆ heterocycloalkyl optionally substituted with one or more occurrences of C₁₋₄ alkyl or halogen. Each instance of R_{g6} and R_{g7} is independently substituted or unsubstituted C₁₋₄ alkyl. Z is CR₅ or N and R₅ is H or F. T is —CH₂—, —O—, —CH(OH)—, or —C(═O)—.

### Analytical Methods

The LCMS data were collected using one of the following methods:

| LCMS | |
|---|---|
| Method | Method Details |
| C1 | Instrument: Agilent 1260II-6120B |
| | Column: YMC Triart C18, 50 × 4.6 mm, 5 μm |
| | Mobile phase: A is H₂O (+0.1% FA) and B is CH₃CN |
| | Run Time: 20% B (0.01 min); 20-95% B (2.09 min); 95% B (0.5 min); 20% B (0.4 min) |
| | Flow rate: 2.5 mL/min |
| | Column temperature: 35° C. |
| | Wavelength: 220 nm / 254 nm |
| F | Instrument: SHIMADZU LCMS-2020 |
| | Column: Shim-pack GIST C18, 50 × 4.6 mm, 5 μm |
| | Mobile phase: A is H₂O (+0.1% FA) and B is CH₃CN |
| | Run Time: 20% B (0.1 min); 20-95% B (1.7 min); 95% B (0.7 min); 20% B (0.4 min) |
| | Flow rate: 2.5 mL /min |
| | Column temperature: 35° C. |
| | Wavelength: 220 nm / 254 nm |
| N | Instrument: SHIMADZU LCMS-2020 |
| | Column: YMC Triart C18, 50 × 4.6 mm, 5 μm |
| | Mobile phase: A is H₂O (+0.05% FA) and B is CH₃CN |
| | Run Time: 20% B (0.01 min), gradient 20-95% B (1.79 min), then 95% B (0.7 min), then |
| | 20% B (0.4 min). |
| | Flow rate: 2.5 mL /min |
| | Column temperature: 35° C. |
| | Wavelength: 220 nm / 254 nm |
| Q | Instrument: SHIMADZU LCMS-2020 |
| | Column: YMC Triart C18, 50 × 4.6 mm, 5 μm |
| | Mobile phase: A is H₂O\CH₃CN\FA = 90\10\0.05 and B is CH₃CN |
| | Run Time: 20% B (0.1 min), gradient 20-95% B (1.7 min), then 95% B (0.7 min), then |
| | 20% B (0.4 min). |
| | Flow rate: 2.3 mL /min |
| | Column temperature: 35° C. |
| | Wavelength: 220 nm / 254 nm |
| R | Instrument: SHIMADZU LCMS-2020 |
| | Column: YMC Triart C18, 50 × 4.6 mm, 5 μm |
| | Mobile phase: A is H₂O\CH₃CN\FA = 90\10\0.05 and B is CH₃CN |
| | Run Time: 20% B (0.01 min), gradient 20-95% B (1.79 min), then 95% B (0.7 min) |
| | Flow rate: 2.3 mL /min |
| | Column temperature: 35° C. |
| | Wavelength: 220 nm / 254 nm |
| U | Instrument: SHIMADZU 2020 |
| | Column: YMC Triart C18, 50 × 4.6 mm, 5 μm |
| | Mobile phase: A is H₂O/MeCN/FA = 90/10/0.05 and B is CH₃CN |
| | Run Time: 20% B (0.01 min); 20-95% B (1.79 min); 95% B (0.7 min) |
| | Flow rate: 2.5 mL/min |
| | Column temperature: 35° C. |
| | Wavelength: 220 nm / 254 nm |
| W | Instrument: SHIMADZU 2020 |
| | Column: Shim-Pack Scepter C18, 33 × 3.0 mm, 3 μm |
| | Mobile phase: A is H₂O/MeCN/FA = 90/10/0.05 and B is CH₃CN |
| | Run Time: 40% B (0.01 min); 40-95% B (1.09 min); 95% B (0.4 min) |
| | Flow rate: 1.2 mL/min |
| | Column temperature: 35° C. |
| | Wavelength: 220 nm / 254 nm |
| W1 | Instrument: SHIMADZU 2020 |
| | Column: YMC-Triart C18, 50 × 4.6 mm, 5 μm |
| | Mobile phase: A is H₂O/MeCN/FA = 90/10/0.05 and B is CH₃CN |
| | Run Time: 40% B (0.01 min); 40-95% B (1.09 min); 95% B (0.4 min) |
| | Flow rate: 1.2 mL/min |
| | Column temperature: 35° C. |
| | Wavelength: 220 nm / 254 nm |
| Y1 | Instrument: SHIMADZU 2020 |
| | Column: YMC Triart C18, 50 × 4.6 mm, 5 μm |
| | Mobile phase: A is H₂O/MeCN/FA = 90/10/0.05 and B is CH₃CN |
| | Run Time: 0% B (0.01 min); 0-50% B (1.79 min); 50% B (0.7 min) |
| | Flow rate: 2.5 mL/min |
| | Column temperature: 35° C. |
| | Wavelength: 220 nm / 254 nm |
| Y2 | Instrument: SHIMADZU 2020 |
| | Column: YMC Triart C18, 50 × 4.6 mm, 5 μm |
| | Mobile phase: A is H₂O/MeCN/FA = 90/10/0.05 and B is CH₃CN |
| | Run Time: 0% B (0.01 min); 0-70% B (1.79 min); 70% B (0.7 min) |
| | Flow rate: 2.3 mL/min |
| | Column temperature: 35° C. |
| | Wavelength: 220 nm / 254 nm |

### Synthetic Examples

### Intermediates

### Synthesis of 5-ethyl-1-methyl-1H-pyrazole-3-carbaldehyde

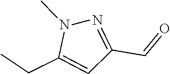

To a mixture of methyl 5-bromo-1-methyl-1H-pyrazole-3-carboxylate (4.50 g, 20.5 mmol) in i-PrOH (50 mL) was added potassium ethenyltrifluoroboranuide (6.05 g, 45.19 mmol), Pd(dppf)Cl₂ (1.5 g, 2.05 mmol) and TEA (4.16 g, 41.1 mmol). The mixture was degassed and purged with N₂ three times. The mixture was stirred at 80° C. for 12 h. The mixture was concentrated. The residue was purified by flash column chromatography on silica gel (0-3% MeOH in DCM) to give methyl 1-methyl-5-vinyl-1H-pyrazole-3-carboxylate (3.40 g, yield: 99.6%) as a light-yellow solid. LC/MS ESI (m/z): 167.1 [M+H]⁺.

To a solution of methyl 1-methyl-5-vinyl-1H-pyrazole-3-carboxylate (3.40 g, 7.58 mmol) in MeOH (30 mL) was added Pd/C (220 mg, 0.1 mmol, 10 wt %). The mixture was degassed and purged with H₂ three times. The mixture was stirred at 20° C. for 2 h. The mixture was filtered, and the filtrate was concentrated to afford methyl 5-ethyl-1-methyl-1H-pyrazole-3-carboxylate (3.2 g, 95% yield) as a yellow solid. LC/MS (ESI) (m/z): 169.1 [M+H]⁺.

To a stirred solution of methyl 5-ethyl-1-methyl-1H-pyrazole-3-carboxylate (3.20 g, 19.0 mmol) in THF (40 mL) was added DIBAL-H (1.5 M in THF, 19 mL, 29 mmol) at −60° C. The reaction was stirred at −60° C. for 2 h. The mixture was quenched with sat. aq. Rochelle salt (100 mL) and extracted with EtOAc (90 mL×3). The combined organic layers were washed with brine, dried by Na₂SO₄ and concentrated. The residue was purified by column chromatography on silica gel (10→100% EtOAc in PE) to give 5-ethyl-1-methyl-1H-pyrazole-3-carbaldehyde (2.5 g, 95.1% yield) as a light-yellow oil. LC/MS (ESI) (m/z): 139.1 [M+H]⁺.

### Synthesis of 1-ethyl-4-iodo-1H-pyrazole-3-carbonitrile

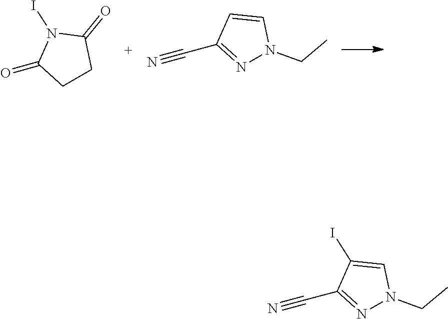

To a stirred solution of 1-ethyl-1H-pyrazole-3-carbonitrile (3.30 g, 27.3 mmol) in MeCN (20 mL) was added TFA (3.11 g, 27.3 mmol) at 0° C. A solution of NIS (7.98 g, 35.3 mmol) in MeCN (20 mL) was then added dropwise at 0° C. The reaction was stirred at 25° C. for 16 h. The mixture was quenched with sat. aq. NaHCO₃ (30 mL) and partially concentrated to remove the MeCN. The mixture was extracted with EtOAc (100×3 mL). The combined organic phase was washed with brine (50 mL×2), dried with anhydrous Na₂SO₄ and concentrated. The residue was purified by flash column chromatography on silica gel (15→20% EtOAc in PE) to give 1-ethyl-4-iodo-1H-pyrazole-3-carbonitrile (3.70 g, yield: 55.0%) as transparent oil. LC/MS (ESI) m/z: 248 [M+H]⁺.

The following intermediates were synthesized using a similar protocol (with m/z (ESI) values):

1-cyclobutyl-4-iodo-1H-pyrazole-3-carbonitrile

| | |
|---|---|
| 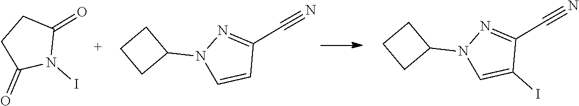 | 274 [M + H]⁺ |

1-(cyclobutylmethyl)-4-iodo-1H-pyrazole-3-carbonitrile

| | |
|---|---|
| 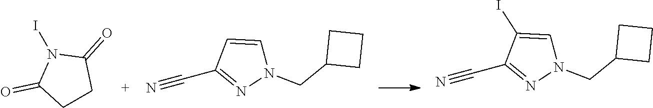 | 288 [M + H]⁺ |

1-(cyclopropylmethyl)-4-iodo-1H-pyrazole-3-carbonitrile

| | |
|---|---|
| 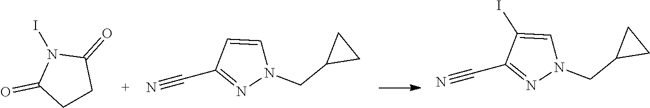 | 274 [M + H]⁺ |

### Synthesis of 1-cyclobutyl-1H-pyrazole-3-carbonitrile

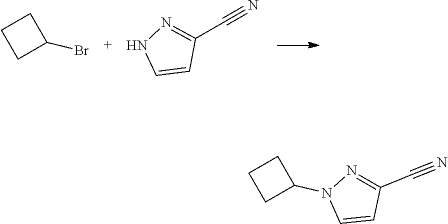 [SMILES: BrC1CCC1 ; N#Cc1cc[nH]n1 ; N#Cc1ccn(C2CCC2)n1]

A mixture of 1H-pyrazole-3-carbonitrile 5.00 g, 53.7 mmol) and NaH 1.93 g, 80.6 mmol, 60% in mineral oil) in DMF (50 mL) was stirred at 0° C. for 0.5 h under N2. A solution of bromocyclobutane (10.9 g, 80.6 mmol) in DMF (100 mL) was added and the reaction was stirred at r.t. for 16 h. The mixture was quenched with sat. aq. NH4Cl (50 mL) and extracted with EtOAc (100 mL×3). The combined organic phase was washed with H₂O (40 mL×3), brine (40 mL×2), dried over anhydrous Na₂SO₄ and concentrated. The residue was purified by flash column chromatography on silica gel (50→100% EtOAc in PE) to give 1-cyclobutyl-1H-pyrazole-3-carbonitrile (3.50 g, 44.3% yield) as a clean oil. LC/MS (ESI) (m/z): 148 [M+H]⁺.

The following intermediates were synthesized using a similar protocol (with m/z (ESI) values):

1-(cyclobutylmethyl)-1H-pyrazole-3-carbonitrile

| | |
|---|---|
| 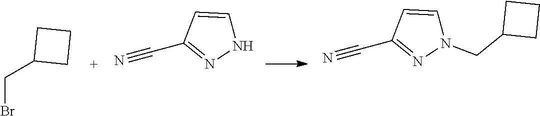 | 162 [M + H]⁺ |

1-(cyclopropylmethyl)-1H-pyrazole-3-carbonitrile

| | |
|---|---|
| 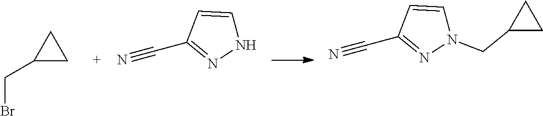 [SMILES: BrCC1CC1 ; N#Cc1cc[nH]n1 ; N#Cc1ccn(CC2CC2)n1] | 148 [M + H]⁺ |

ethyl 5-cyclobutyl-1-((2-(trimethylsilyl)ethoxy)methyl)-1H-pyrazole-3-carboxylate

| | |
|---|---|
| 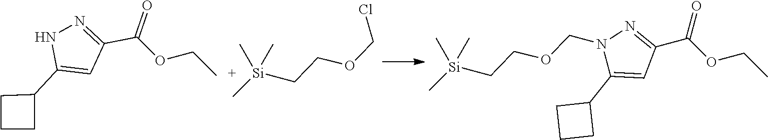 | 325 [M + H]⁺ |

ethyl 3-ethyl-1-((2-(trimethylsilyl)ethoxy)methyl)-1H-pyrazole-5-carboxylate

| | |
|---|---|
| 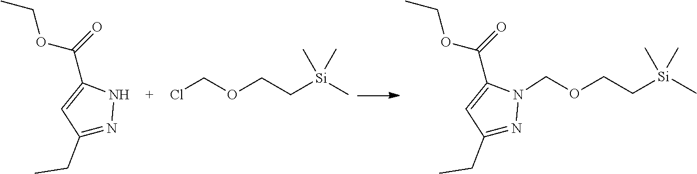 | 299 [M + H]⁺ |

### Synthesis of (2-chloro-6-methylpyridin-3-yl)boronic acid

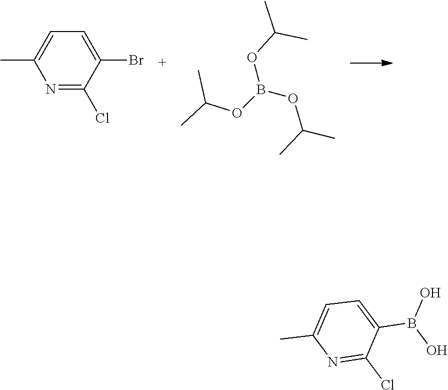

To a solution of 3-bromo-2-chloro-6-methylpyridine (5 g, 24.22 mmol) in THF (50 mL) was added n-BuLi (1.6 M in hexane, 18 mL, 29.06 mmol) at −70° C. The mixture was stirred at −70° C. for 1 h. Then triisopropyl borate (5.47 g, 29.06 mmol) was added dropwise and the mixture was stirred at 20° C. for 3 h. The reaction was quenched with aq. NaOH (5%, 30 mL). The aq. layer was separated and acidified to pH 3 with aq. HCl (1 M), then extracted with EtOAc (50 mL×3). The combined organic layer was dried over anhydrous Na₂SO₄, filtered and concentrated to give (2-chloro-6-methylpyridin-3-yl)boronic acid (1.6 g, 38% yield). LC/MS (ESI) (m/z): 172 [M+H]⁺.

### Synthesis of (3-bromo-1-methyl-1H-pyrazol-5-yl)(cyclopropyl)methanol

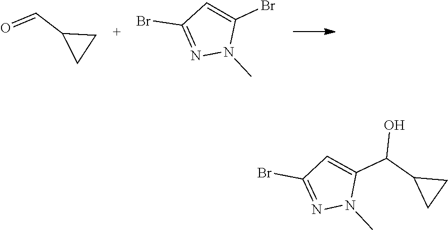

To a solution of 3,5-dibromo-1-methyl-1H-pyrazole (1.00 g, 4.17 mmol) in THF (10 mL) was added n-BuLi (1.6 M in hexane, 3.13 mL, 5.00 mmol) at −78° C. The mixture was stirred at −78° C. for 15 min. A solution of cyclopropanecarbaldehyde (0.311 mL, 4.168 mmol) in THE (10 mL) was added and the reaction was stirred at −78° C. for 2 h. The solution was quenched with sat. aq. NH₄Cl at 0° C. Then extracted with EtOAc (30 mL×3). The combined organic phases were washed with brine (30 mL), dried over anhydrous Na₂SO₄ and concentrated. The residue was purified by flash column chromatography on silica gel (0→30% EtOAc in PE) to give (3-bromo-1-methyl-1H-pyrazol-5-yl)(cyclopropyl)methanol (330 mg, yield: 34%) as a yellow oil. LC/MS (ESI) (m/z): 231 [M+H]⁺.

### Synthesis of ethyl 5-cyano-1-methylpyrazole-3-carboxylate

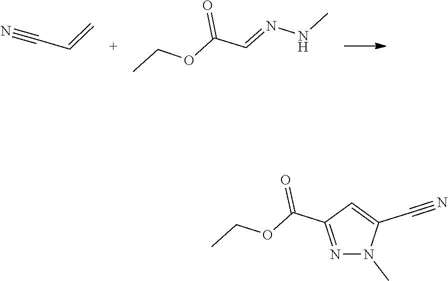

A solution of ethyl (E)-2-(2-methylhydrazineylidene)acetate (50.0 g, 384 mmol), prop-2-enenitrile (38.2 mL, 576 mmol), benzoyl peroxide (232 g, 960 mmol) and iodine (19.4 g, 76.8 mmol) in MeCN (750 mL) was stirred at 80° C. for 16 h. The residue was diluted with EtOAc (300 mL) and washed with sat. aq. NaHCO₃ (200 mL) and sat. aq. Na₂S₂O₃ (200 mL). The organic phase was washed with brine, dried with anhydrous Na₂SO₄ and concentrated. The residue was purified by flash column chromatography on silica gel (30→60% EtOAc in PE) to afford ethyl 5-cyano-1-methylpyrazole-3-carboxylate (20.0 g, yield: 29.1%) as yellow solid. LC/MS (ESI) (m/z): 180 [M+H]⁺.

### Synthesis of 3-bromo-5-(cyclopropylmethyl)-1-methyl-1H-pyrazole

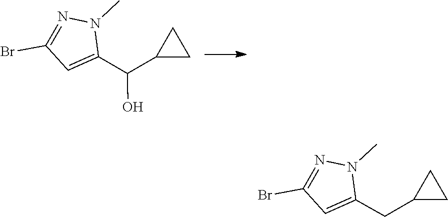

To a mixture of (3-bromo-1-methyl-1H-pyrazol-5-yl)(cyclopropyl)methanol (330 mg, 1.428 mmol) and TFA (4 mL) was added TES (2.306 mL, 14.280 mmol) at 25° C. The mixture was thrice degassed and purged with N₂, and then stirred at 50° C. for 16 h. The mixture was concentrated, diluted with sat. aq. NaHCO₃, and then extracted with EtOAc (30 mL×3). The combined organic phases were washed with brine (30 mL), dried over anhydrous Na₂SO₄ and concentrated. The residue was purified by flash column chromatography on silica gel (0→50% EtOAc in PE) to give 3-bromo-5-(cyclopropylmethyl)-1-methyl-1H-pyrazole (205 mg, yield: 66.7%) as a yellow oil. LC/MS (ESI) (m/z): 215 [M+H]⁺.

The following intermediates were synthesized using a similar protocol (with m/z (ESI) values):

4-((2-chloropyridin-3-yl)methyl)-1-ethyl-1H-pyrazole-3-carbonitrile

| | |
|---|---|
| 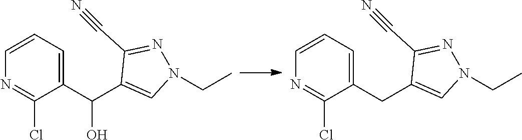 [SMILES: CCn1cc(C(O)c2cccnc2Cl)c(C#N)n1 ; CCn1cc(Cc2cccnc2Cl)c(C#N)n1] | 247 [M + H]⁺ |

1-(cyclopropylmethyl)-4-((1-(4-fluoro-2-iodophenyl)-3-methyl-1H-pyrazol-5-yl)methyl)-1H-
pyrazole-3-carbonitrile

| | |
|---|---|
| 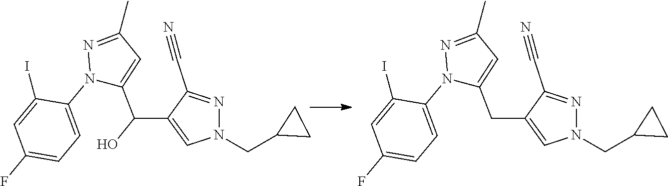 [SMILES: Cc1cc(C(O)c2cn(CC3CC3)nc2C#N)n(-c2ccc(F)cc2I)n1 ; Cc1cc(Cc2cn(CC3CC3)nc2C#N)n(-c2ccc(F)cc2I)n1] | 462 [M + H]⁺ |

4-((2-chloropyridin-3-yl)methyl)-1-(cyclopropylmethyl)-1H-pyrazole-3-carbonitrile

| | |
|---|---|
| 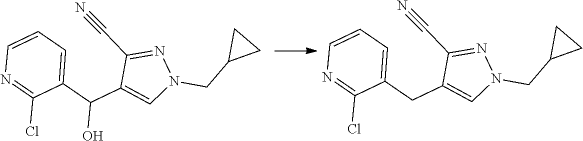 [SMILES: N#Cc1nn(CC2CC2)cc1C(O)c1cccnc1Cl ; N#Cc1nn(CC2CC2)cc1Cc1cccnc1Cl] | 273 [M + H]⁺ |

4-((3-bromo-1-methyl-1H-pyrazol-4-yl)methyl)-1-ethyl-1H-pyrazole-3-carbonitrile

| | |
|---|---|
| 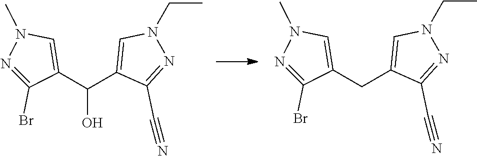 [SMILES: CCn1cc(C(O)c2cn(C)nc2Br)c(C#N)n1 ; CCn1cc(Cc2cn(C)nc2Br)c(C#N)n1] | 294 [M + H]⁺ |

3-bromo-4-((1-(cyclopropylmethyl)-3-(methylthio)-1H-pyrazol-4-yl)methyl)-1-methyl-1H-
pyrazole

| | |
|---|---|
| 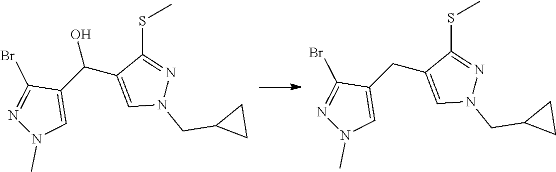 [SMILES: CSc1nn(CC2CC2)cc1C(O)c1cn(C)nc1Br ; CSc1nn(CC2CC2)cc1Cc1cn(C)nc1Br] | 341 [M + H]⁺ |

4-((2-bromopyridin-3-yl)methyl)-1-(cyclopropylmethyl)-1H-pyrazole-3-carbonitrile

| | |
|---|---|
| 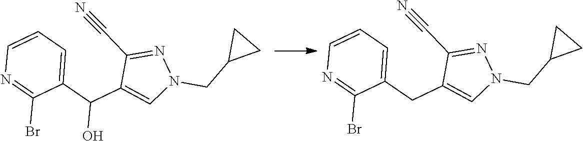 [SMILES: N#Cc1nn(CC2CC2)cc1C(O)c1cccnc1Br ; N#Cc1nn(CC2CC2)cc1Cc1cccnc1Br] | 317 [M + H]⁺ |

4-{[1-(2-bromo-4,6-difluorophenyl)-3-methyl-1H-pyrazol-5-yl]methyl}-1-(cyclopropylmethyl)-
1H-pyrazole-3-carbonitrile

| | |
|---|---|
| 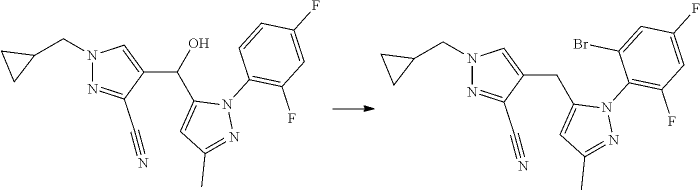 [SMILES: Cc1cc(C(O)c2cn(CC3CC3)nc2C#N)n(-c2ccc(F)cc2F)n1 ; Cc1cc(Cc2cn(CC3CC3)nc2C#N)n(-c2c(F)cc(F)cc2Br)n1] | 432 [M + H]⁺ |

4-((2-bromopyridin-3-yl)methyl)-1-ethyl-1H-pyrazole-3-carbonitrile

| | |
|---|---|
| 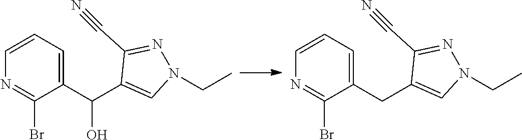 [SMILES: CCn1cc(C(O)c2cccnc2Br)c(C#N)n1 ; CCn1cc(Cc2cccnc2Br)c(C#N)n1] | 291 [M + H]⁺ |

4-((2-chloropyridin-3-yl)methyl)-1-cyclobutyl-1H-pyrazole-3-carbonitrile

| | |
|---|---|
| 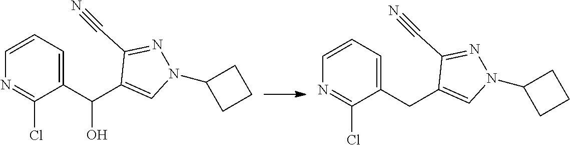 [SMILES: N#Cc1nn(C2CCC2)cc1C(O)c1cccnc1Cl ; N#Cc1nn(C2CCC2)cc1Cc1cccnc1Cl] | 273 [M + H]⁺ |

5-((2,4-dibromothiazol-5-yl)methyl)-1H-pyrazole-3-carbonitrile

| | |
|---|---|
| 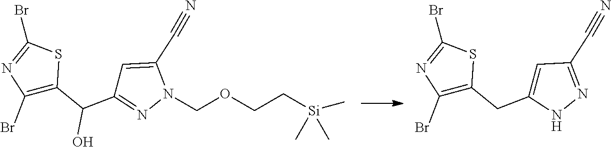 [SMILES: C[Si](C)(C)CCOCn1nc(C(O)c2sc(Br)nc2Br)cc1C#N ; N#Cc1cc(Cc2sc(Br)nc2Br)[nH]n1] | 347 [M + H]⁺ |

2-bromo-3-((5-cyclobutyl-1H-pyrazol-3-yl)methyl)pyridine

| | |
|---|---|
| 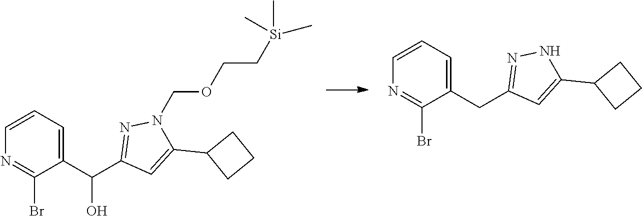 [SMILES: C[Si](C)(C)CCOCn1nc(C(O)c2cccnc2Br)cc1C1CCC1 ; Brc1ncccc1Cc1cc(C2CCC2)[nH]n1] | 292 [M + H]⁺ |

2-chloro-3-((3-(cyclopropylmethyl)-1H-pyrazol-5-yl)methyl)pyridine

| | |
|---|---|
| 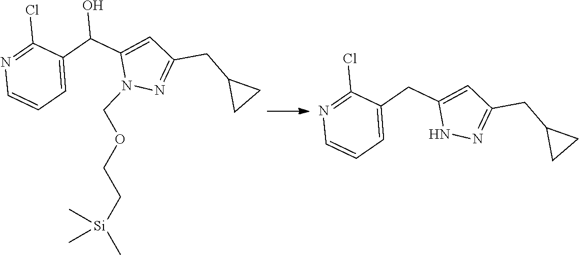 [SMILES: C[Si](C)(C)CCOCn1nc(CC2CC2)cc1C(O)c1cccnc1Cl ; Clc1ncccc1Cc1cc(CC2CC2)n[nH]1] | 248 [M + H]⁺ |

2-chloro-3-((3-ethyl-1H-pyrazol-5-yl)methyl)pyridine

| | |
|---|---|
| 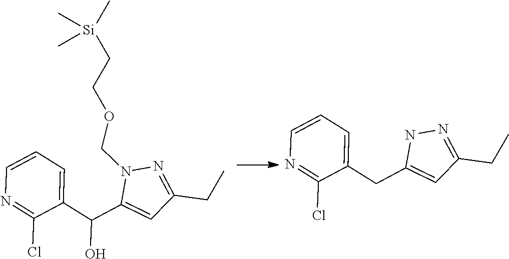 [SMILES: CCc1cc(C(O)c2cccnc2Cl)n(COCC[Si](C)(C)C)n1 ; CCc1cc(Cc2cccnc2Cl)[nH]n1] | 222 [M + H]⁺ |

### Synthesis of 5-(hydroxymethyl)-2-methylpyrazole-3-carbonitrile

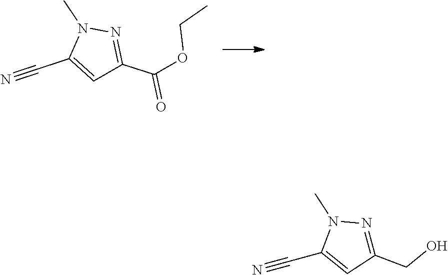

To a stirred solution of ethyl 5-cyano-1-methylpyrazole-3-carboxylate (10.0 g, 55.8 mmol) and MeOH (3.5 g, 112 mmol) in THE (100 mL) was added LiBH₄ (55.8 mL, 112 mmol, 2 M in THF) at 0° C. The reaction was stirred at 25° C. for 16 h. The mixture was quenched with sat. aq. NH₄Cl (100 mL) at 0° C. and then extracted with EtOAc (100 mL). The organic phase was dried over anhydrous Na₂SO₄ and concentrated. The residue was purified by flash column chromatography on silica gel (0-50% EtOAc in PE) to give 5-(hydroxymethyl)-2-methylpyrazole-3-carbonitrile (3.60 g, yield: 47.0%) as a white solid. LC/MS (ESI) (m/z): 138 [M+H]⁺.

The following intermediates were synthesized using a similar protocol (with m/z (ESI) values):

ethyl 1-(4-fluoro-2-(1-hydroxyethyl)phenyl)-3-methyl-1H-pyrazole-5-carboxylate

| | |
|---|---|
| 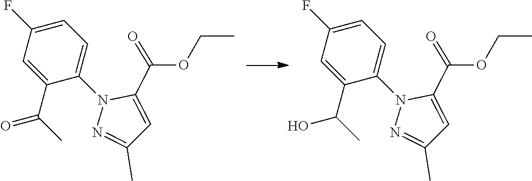 [SMILES: CCOC(=O)c1cc(C)nn1-c1ccc(F)cc1C(C)=O ; CCOC(=O)c1cc(C)nn1-c1ccc(F)cc1C(C)O] | 293 [M + H]⁺ |

1-(cyclopropylmethyl)-4-({1-[2,4-difluoro-6-(1-hydroxyethyl)phenyl]-3-methyl-1H-pyrazol-5-
yl}methyl)-N,N-dimethyl-1H-pyrazole-3-carboxamide

| | |
|---|---|
| 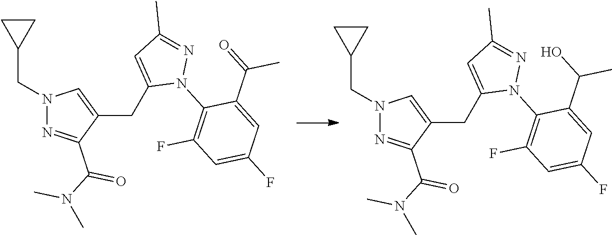 [SMILES: CC(=O)c1cc(F)cc(F)c1-n1nc(C)cc1Cc1cn(CC2CC2)nc1C(=O)N(C)C ; Cc1cc(Cc2cn(CC3CC3)nc2C(=O)N(C)C)n(-c2c(F)cc(F)cc2C(C)O)n1] | 444 [M + H]⁺ |

### Synthesis of ethyl 5-cyclobutyl-1H-pyrazole-3-carboxylate

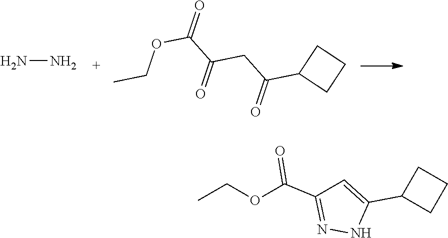

To a solution of ethyl 4-cyclobutyl-2,4-dioxobutanoate (8.3 g, 41.69 mmol) in EtOH (40 mL) was added hydrazine (2.4 g, 62.54 mmol, 85% in water) and the reaction was stirred at 80° C. for 2 h. The mixture was concentrated, diluted with AcOH (7 mL), and the solution was stirred at 25° C. for 8 h. The mixture was concentrated, diluted with sat. aq. NaHCO₃ and extracted with EtOAc (20 mL×3). The combined organic phases were washed with brine, dried over anhydrous Na₂SO₄, and concentrated. The residue was purified by flash column chromatography on silica gel (0→30% EtOAc in PE) to give ethyl 5-cyclobutyl-1H-pyrazole-3-carboxylate (6.9 g, yield: 85.1%) as a yellow oil. LC/MS ESI (m/z): 195 [M+H]⁺

The following intermediate was synthesized using a similar protocol (with m/z (ESI) values):

ethyl 5-(cyclopropylmethyl)-1H-pyrazole-3-carboxylate

| | |
|---|---|
| 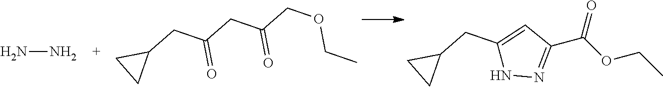 | 195 [M + H]⁺ |

### Synthesis of ethyl 5-cyclopropyl-2,4-dioxopentanoate

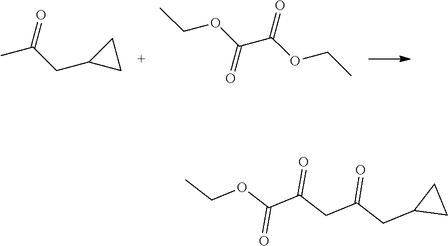

To a solution of 1-cyclopropylpropan-2-one (1.50 g, 15.3 mmol) and diethyl oxalate (1.70 g, 15.3 mmol) in THF (20 mL) was added t-BuOK (15.3 mL, 15.3 mmol, 1 M in THF) at 0° C. and the mixture was stirred at 25° C. for 3 h. The reaction was quenched with sat. aq. NH₄Cl solution (30 mL) and extracted with EtOAc (50 mL×3). The combined organic phase was washed with brine (50 mL×2), dried with anhydrous Na₂SO₄ and concentrated to give ethyl 5-cyclopropyl-2,4-dioxopentanoate (1.80 g, crude) as a brown oil. LC/MS (ESI) (m/z): 199 [M+H]⁺.

### Synthesis of 5-(cyclopropylmethyl)-3-iodo-1-methyl-1H-pyrazole

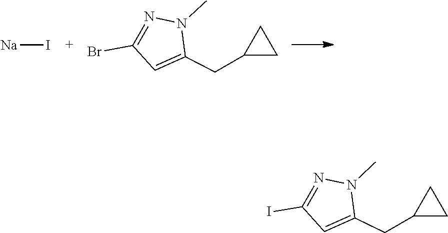

To a solution of 3-bromo-5-(cyclopropylmethyl)-1-methyl-1H-pyrazole (185 mg, 0.860 mmol) in dioxane (8 mL) was added DMEDA (0.009 mL, 0.086 mmol), NaI (1.29 g, 8.601 mmol) and CuI (8.19 mg, 0.043 mmol). The mixture was degassed, purged with N₂ three times, and then stirred at 110° C. for 16 h in a sealed tube. The mixture was filtered, and the filtrate was concentrated. The residue was purified by flash column chromatography on silica gel (0→50% EtOAc in PE) to give 5-(cyclopropylmethyl)-3-iodo-1-methyl-1H-pyrazole (187 mg, yield: 83%) as a yellow oil. LC/MS (ESI) (m/z): 263 [M+H]⁺.

### Synthesis of 5-formyl-2-methylpyrazole-3-carbonitrile

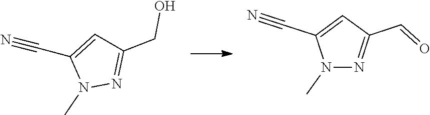

To a stirred solution of 5-(hydroxymethyl)-2-methylpyrazole-3-carbonitrile (1.00 g, 7.29 mmol) in DCM (10 mL) was added DMP (4.64 g, 10.9 mmol) at 0° C. The reaction was stirred at 25° C. for 16 h. The mixture was quenched with sat. aq. NaHCO₃ (50 mL) and extracted with DCM (50 mL×3). The organic phase was dried over anhydrous Na₂SO₄ and concentrated. The residue was purified by flash column chromatography on silica gel (0→25% EtOAc in PE) to give 5-formyl-2-methylpyrazole-3-carbonitrile (860 mg, yield: 87.3%) as a yellow oil. LC/MS (ESI) (m/z): 136 [M+H]⁺.

The following intermediate was synthesized using a similar protocol (with m/z (ESI) values):

(2-bromopyridin-3-yl)(5-(cyclopropylmethyl)-1-methyl-1H-pyrazol-3-yl)methanone

| | |
|---|---|
| 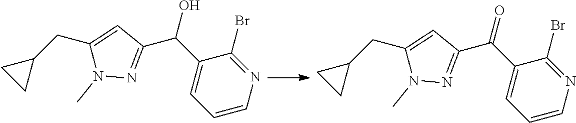 [SMILES: Cn1nc(C(O)c2cccnc2Br)cc1CC1CC1 ; Cn1nc(C(=O)c2cccnc2Br)cc1CC1CC1] | 320 [M + H]⁺ |

### Synthesis of ethyl 1-(cyclopropylmethyl)-4-hydroxy-1H-pyrazole-3-carboxylate

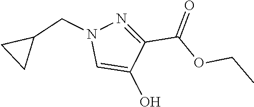

A solution of ethyl 4-formyl-1H-pyrazole-3-carboxylate (1.00 g, 5.95 mmol) and Cs₂CO₃ (2.91 g, 8.93 mmol) in DF (5 mL) was cooled to −10° C. (Bromomethyl)cyclopropane (1.19 g, 8.93 mmol) was added and the reaction was stirred for 2 h at −10° C. The mixture was quenched with sat. aq. NH₄Cl (20 mL) and extracted with EtOAc (30 mL×3). The combined organic phases were washed with H₂O (30 mL×3), brine (50 mL×2), dried with anhydrous Na₂SO₄, and concentrated. The residue was purified by flash column chromatography on silica gel (0→30% EtOAc in PE) to afford ethyl 1-(cyclopropylmethyl)-4-formyl-1H-pyrazole-3-carboxylate (800 mg, 61.0% yield) as a yellow solid. LC/MS (ESI): m/z 223 [M+H]⁺.

To a solution of ethyl 1-(cyclopropylmethyl)-4-formyl-1H-pyrazole-3-carboxylate (1.40 g, 6.30 mmol) in CHCl₃ (28 mL) was added m-CPBA (1.63 g, 9.45 mmol). The reaction mixture was stirred at 60° C. for 2 h. The mixture was quenched with sat. aq. Na₂S203 (30 mL) and extracted with DCM (30 mL×3). The combined organic phase was washed with sat. aq. NaHCO₃ (30 mL), dried over anhydrous Na₂SO₄, and concentrated. The residue was dissolved in ethanol (30 mL) and aq. HCl (5 M, 30 mL). The mixture was stirred at r.t. for 10 min then extracted with EtOAc (30 mL×3). The combined organic phase was washed with brine, dried over anhydrous Na₂SO₄, and concentrated. The residue was purified by flash column chromatography on silica gel (0→100% EtOAc in PE) to afford ethyl 1-(cyclopropylmethyl)-4-hydroxy-1H-pyrazole-3-carboxylate (1.10 g, 83.0% yield) as a white solid. LC/MS ESI (m/z): 211 [M+H]⁺.

### Synthesis of 2-chloro-3-((1-ethyl-1H-pyrazol-4-yl)oxy)pyridine

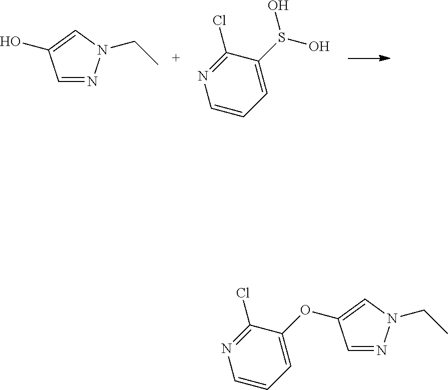

To a mixture of 1-ethyl-1H-pyrazol-4-ol (500 mg, 4.46 mmol), (2-chloropyridin-3-yl)boronic acid (1.40 g, 8.92 mmol) and 4 Å molecular sieves in DCM (20 mL) was added Cu(OAc)₂ (810 mg, 4.46 mmol) and TEA (1.2 mL, 8.91 mmol). The reaction mixture was stirred for 18 h at 25° C. The mixture was filtered, and the filtrate was concentrated. The residue was purified by flash column chromatography on silica gel (0→50% EtOAc in PE) to give 2-chloro-3-((1-ethyl-1H-pyrazol-4-yl)oxy)pyridine (45.0 mg, 4.50% yield) as a yellow oil. LCMS (ESI): m/z: 224 [M+H]⁺.

### Synthesis of ethyl 1-(cyclopropylmethyl)-3-(methylthio)-1H-pyrazole-4-carboxylate

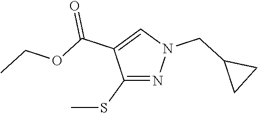 [SMILES: CCOC(=O)c1cn(CC2CC2)nc1SC]

A solution of ethyl 2-cyano-3,3-bis(methylthio)acrylate (1.35 g, 6.21 mmol) and (cyclopropylmethyl)hydrazine (535 mg, 6.21 mmol) in i-PrOH (40 mL) was added DIEA (3.08 mL, 18.6 mmol). The reaction mixture was stirred at 85° C. under sealed tube overnight. The mixture was concentrated. The residue was purified by flash column chromatography on silica gel (0→100% EtOAc in PE) to afford ethyl 5-amino-1-(cyclopropylmethyl)-3-(methylsulfanyl)-1H-pyrazole-4-carboxylate (850 mg, 54.0% yield) as yellow solid. LC/MS (ESI) (m/z): 256 [M+H]⁺.

A solution of ethyl 5-amino-1-(cyclopropylmethyl)-3-(methylsulfanyl)-1H-pyrazole-4-carboxylate (600 mg, 2.35 mmol) in THF (24 mL) was added 3-methylbutyl nitrite (1.27 mL, 9.40 mmol). The reaction mixture was refluxed for 2 h. The residue was purified by flash column chromatography on silica gel (0→100% EtOAc in PE) to afford ethyl 1-(cyclopropylmethyl)-3-(methylthio)-1H-pyrazole-4-carboxylate (500 mg, 89.0% yield) as yellow solid. LC/MS (ESI) (m/z): 241 [M+H]⁺.

### Synthesis of 4-(cyclopropyl(hydroxy)methyl)-N,N-dimethyl-1H-imidazole-1-sulfonamide

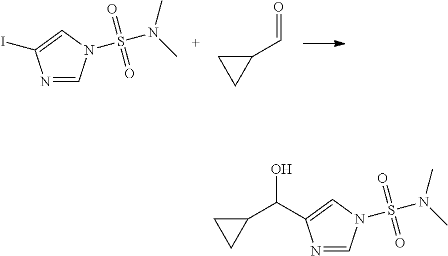

To a solution of 4-iodo-N,N-dimethyl-1H-imidazole-1-sulfonamide (5.50 g, 0.0200 mol) in THF (50 mL) was added i-PrMgCl·LiCl (1.3 M in THF, 15.5 mL, 0.0200 mol) at 0° C. The mixture was stirred at 0° C. for 1 h. A solution of cyclopropanecarbaldehyde (1.65 mL, 0.0200 mol) in THF (10 mL) was added and stirring was continued at 0° C. for 1 h. The reaction mixture was quenched with ice-water (50 mL) and extracted with EtOAc (50 mL×3). The combined organic phases were washed with brine (30 mL×2), dried over anhydrous Na₂SO₄, filtered and concentrated. The residue was purified by flash column chromatography on silica gel (0→50% EtOAc in PE) to give 4-(cyclopropyl(hydroxy)methyl)-N,N-dimethyl-1H-imidazole-1-sulfonamide (2.35 g, 52.4%) as a yellow oil. LC/MS ESI (m/z): 246 [M+H]⁺.

The following intermediates were synthesized using a similar protocol (with m/z (ESI) values):

4-((3-bromo-1-methyl-1H-pyrazol-4-yl)(hydroxy)methyl)-1-(cyclobutylmethyl)-1H-pyrazole-3-
carbonitrile

| | |
|---|---|
| 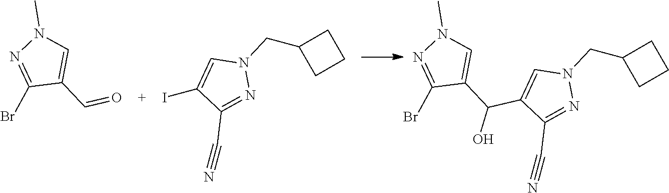 | 350 [M + H]⁺ |

4-((3-bromo-1-methyl-1H-pyrazol-4-yl)(hydroxy)methyl)-1-ethyl-1H-pyrazole-3-carbonitrile

| | |
|---|---|
| 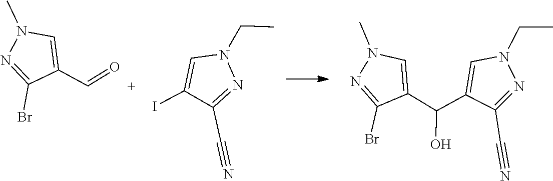 | 310 [M + H]⁺ |

(3-bromo-1-methyl-1H-pyrazol-4-yl)(1-(cyclopropylmethyl)-3-(methylthio)-1H-pyrazol-4-
yl)methanol

| | |
|---|---|
| 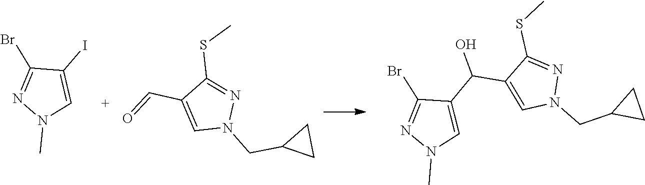 | 357 [M + H]⁺ |

(2-bromopyridin-3-yl)(5-(cyclopropylmethyl)-1-methyl-1H-pyrazol-3-yl)methanol

| | |
|---|---|
| 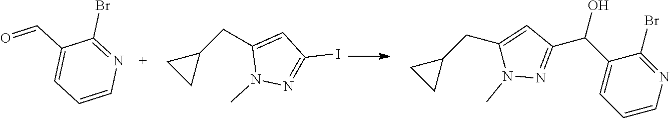 [SMILES: O=Cc1cccnc1Br ; Cn1nc(I)cc1CC1CC1 ; Cn1nc(C(O)c2cccnc2Br)cc1CC1CC1] | 322 [M + H]⁺ |

### Synthesis of (2-bromopyridin-3-yl)(1-ethyl-1H-pyrazol-4-yl)methanol

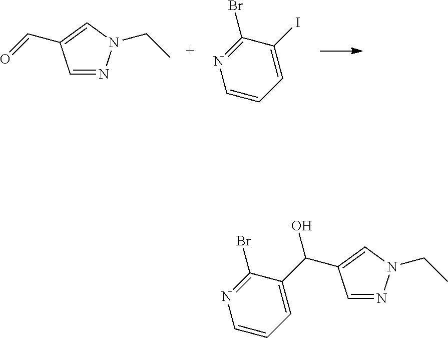

To a solution of 2-bromo-3-iodopyridine (3.57 g, 12.6 mmol) in THF (10 mL) was added i-PrMgCl (9.67 mL, 12.6 mmol, 1.3 M in THF) at 0° C. and the mixture was stirred at 0° C. for 0.5 h. A solution of 1-ethylpyrazole-4-carbaldehyde (1.3 g, 10.472 mmol) in THE (10 mL) was then added and the reaction was stirred at 0° C. for 1 h, then at 50° C. for 12 h. The reaction was quenched with sat. aq. NH₄Cl (30 mL) and extracted with EtOAc (20 mL×3). The combined organic layers were dried over anhydrous Na₂SO₄, filtered, and concentrated. The residue was purified by column chromatography on silica gel (10→100% EtOAc in PE) to give (2-bromopyridin-3-yl)(1-ethyl-1H-pyrazol-4-yl)methanol (1.97 g, yield: 66.7%) as a yellow solid. LC/MS (ESI) (m/z): 282 [M+H]⁺.

The following intermediates were synthesized using a similar protocol (with m/z (ESI) values):

4-((3-iodo-1-methyl-1H-pyrazol-4-yl)(hydroxy)methyl)-1-(cyclopropylmethyl)-1H-pyrazole-3-
carbonitrile

| | |
|---|---|
| 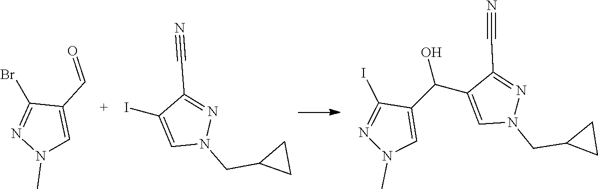 | 384 [M + H]⁺ |

(2-bromopyridin-3-yl)(5-ethyl-1-methyl-1H-pyrazol-3-yl)methanol

| | |
|---|---|
| 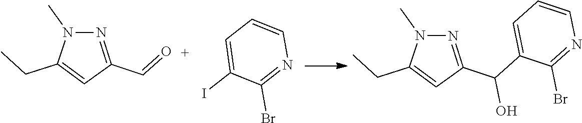 | 296 [M + H]⁺ |

3-((2-bromopyridin-3-yl)(hydroxy)methyl)-1-methyl-1H-pyrazole-5-carbonitrile

| | |
|---|---|
| 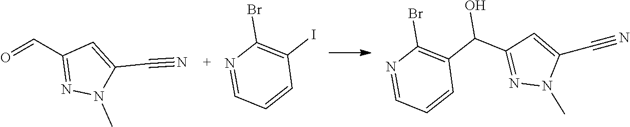 | 293 [M + H]⁺ |

### Synthesis of (1-ethyl-1H-pyrazol-4-yl)(3-iodo-1-methyl-1H-pyrazol-4-yl)methanone

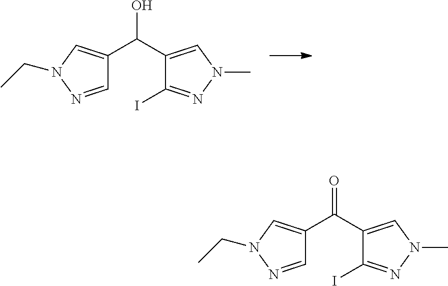 [SMILES: CCn1cc(C(O)c2cn(C)nc2I)cn1 ; CCn1cc(C(=O)c2cn(C)nc2I)cn1]

To a solution of (1-ethyl-1H-pyrazol-4-yl)(3-iodo-1-methyl-1H-pyrazol-4-yl)methanol (200 mg, 0.602 mmol) in DCM (30 mL) was added MnO₂ (523 mg, 6.02 mmol). The reaction was stirred at 20° C. for 16 h. The mixture was filtered, and the filtrate was concentrated. The residue was purified by flash column chromatography on silica gel (30→50% EtOAc in PE) to afford (1-ethyl-1H-pyrazol-4-yl)(3-iodo-1-methyl-1H-pyrazol-4-yl)methanone (142 mg, yield: 71.4%) as a white solid. LC/MS (ESI) (m/z): 331 [M+H]⁺.

The following intermediates were synthesized using a similar protocol (with m/z (ESI) values):

1-(2-(1-((tert-butyldimethylsilyl)oxy)ethyl)-4-fluorophenyl)-3-methyl-1H-pyrazole-5-
carbaldehyde

| | |
|---|---|
| 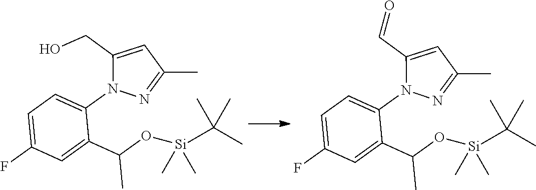 [SMILES: Cc1cc(CO)n(-c2ccc(F)cc2C(C)O[Si](C)(C)C(C)(C)C)n1 ; Cc1cc(C=O)n(-c2ccc(F)cc2C(C)O[Si](C)(C)C(C)(C)C)n1] | 363 [M + H]⁺ |

3-(1-(2-(1-((tert-butyldimethylsilyl)oxy)ethyl)-4-fluorophenyl)-3-methyl-1H-pyrazole-5-
carbonyl)-1-((2-(trimethylsilyl)ethoxy)methyl)-1H-pyrazole-5-carbonitrile

| | |
|---|---|
| 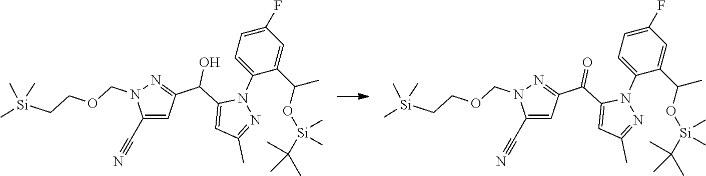 | 584 [M + H]⁺ |

1-(cyclopropylmethyl)-3-(methylthio)-1H-pyrazole-4-carbaldehyde

| | |
|---|---|
| 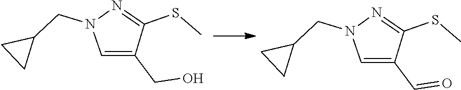 | 197 [M + H]⁺ |

3-(2-chloronicotinoyl)-1-((2-(trimethylsilyl)ethoxy)methyl)-1H-pyrazole-5-carbonitrile

| | |
|---|---|
| 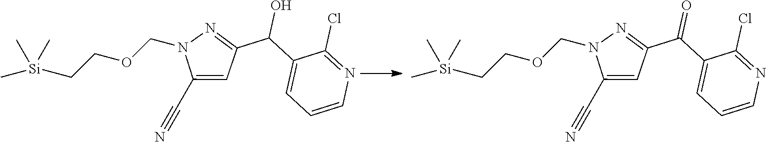 [SMILES: C[Si](C)(C)CCOCn1nc(C(O)c2cccnc2Cl)cc1C#N ; C[Si](C)(C)CCOCn1nc(C(=O)c2cccnc2Cl)cc1C#N] | 363 [M + H]⁺ |

5-cyclobutyl-1-((2-(trimethylsilyl)ethoxy)methyl)-1H-pyrazole-3-carbaldehyde

| | |
|---|---|
| 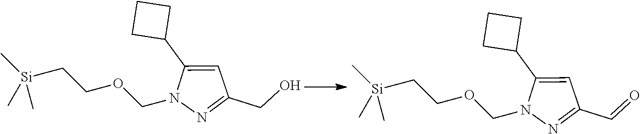 [SMILES: C[Si](C)(C)CCOCn1nc(CO)cc1C1CCC1 ; C[Si](C)(C)CCOCn1nc(C=O)cc1C1CCC1] | 281 [M + H]⁺ |

3-(2-bromonicotinoyl)-1-((2-(trimethylsilyl)ethoxy)methyl)-1H-pyrazole-5-carbonitrile

| | |
|---|---|
| 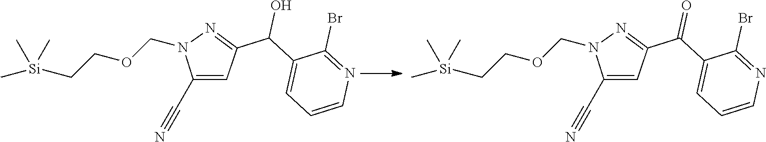 [SMILES: C[Si](C)(C)CCOCn1nc(C(O)c2cccnc2Br)cc1C#N ; C[Si](C)(C)CCOCn1nc(C(=O)c2cccnc2Br)cc1C#N] | 407 [M + H]⁺ |

4-(2-bromonicotinoyl)-1-ethyl-N,N-dimethyl-1H-pyrazole-3-carboxamide

| | |
|---|---|
| 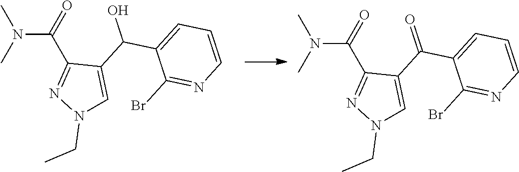 [SMILES: CCn1cc(C(O)c2cccnc2Br)c(C(=O)N(C)C)n1 ; CCn1cc(C(=O)c2cccnc2Br)c(C(=O)N(C)C)n1] | 351 [M + H]⁺ |

(2-bromopyridin-3-yl)(1-ethyl-1H-pyrazol-4-yl)methanone

| | |
|---|---|
| 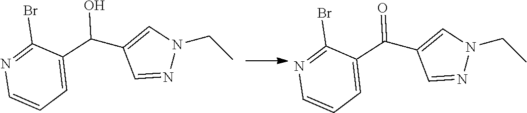 [SMILES: CCn1cc(C(O)c2cccnc2Br)cn1 ; CCn1cc(C(=O)c2cccnc2Br)cn1] | 280 [M + H]⁺ |

3-(cyclopropylmethyl)-1-((2-(trimethylsilyl)ethoxy)methyl)-1H-pyrazole-5-carbaldehyde

| | |
|---|---|
| 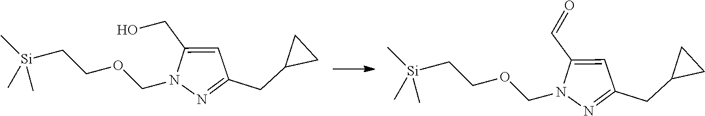 [SMILES: C[Si](C)(C)CCOCn1nc(CC2CC2)cc1CO ; C[Si](C)(C)CCOCn1nc(CC2CC2)cc1C=O] | 281 [M + H]⁺ |

3-ethyl-1-((2-(trimethylsilyl)ethoxy)methyl)-1H-pyrazole-5-carbaldehyde

| | |
|---|---|
| 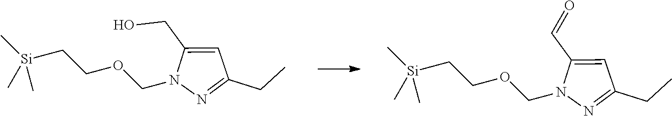 [SMILES: CCc1cc(CO)n(COCC[Si](C)(C)C)n1 ; CCc1cc(C=O)n(COCC[Si](C)(C)C)n1] | 255 [M + H]⁺ |

### Synthesis of 4-(cyclopropylmethyl)-N,N-dimethyl-1H-imidazole-1-sulfonamide

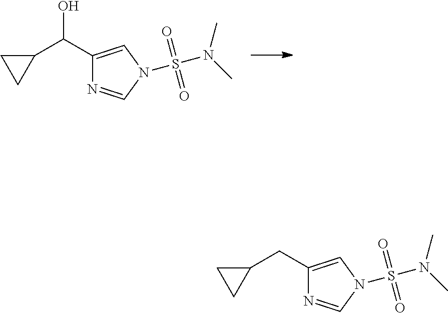 [SMILES: CN(C)S(=O)(=O)n1cnc(C(O)C2CC2)c1 ; CN(C)S(=O)(=O)n1cnc(CC2CC2)c1]

To a mixture of 4-(cyclopropyl(hydroxy)methyl)-N,N-dimethyl-1H-imidazole-1-sulfonamide (2.35 g, 9.58 mmol) and TFA (7 mL) was added TES (11.2 g, 95.9 mmol). The reaction was stirred at r.t. for 1 h. The mixture was concentrated, the residue was diluted with sat. aq. NaHCO₃ (25 mL), and then extracted with EtOAc (35 mL×3). The combined organic phases were dried over anhydrous Na₂S04 and concentrated. The residue was purified by flash column chromatography on silica gel (0→50% a EtOAc in PE) to give 4-(cyclopropylmethyl)-N,N-dimethyl-1H-imidazole-1-sulfonamide (2.00 g, yield: 91.3%) as a yellow solid. LC/MS (ESI) (m/z): 230 [M+H]⁺.

The following intermediates were synthesized using a similar protocol (with m/z (ESI) values):

4-((3-bromo-1-methyl-1H-pyrazol-4-yl)methyl)-1-(cyclobutylmethyl)-1H-pyrazole-3-
carbonitrile

| | |
|---|---|
| 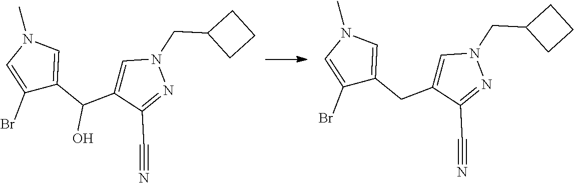 [SMILES: Cn1cc(Br)c(C(O)c2cn(CC3CCC3)nc2C#N)c1 ; Cn1cc(Br)c(Cc2cn(CC3CCC3)nc2C#N)c1] | 334 [M + H]⁺ |

4-((1-(2-bromo-4-fluorophenyl)-3-methyl-1H-pyrazol-5-yl)methyl)-1-cyclobutyl-1H-pyrazole-
3-carbonitrile

| | |
|---|---|
| 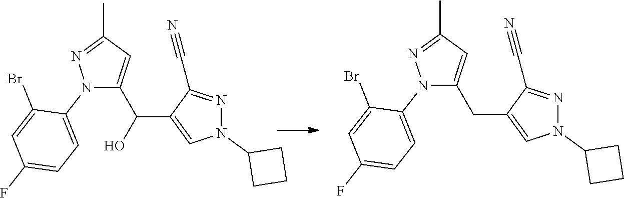 [SMILES: Cc1cc(C(O)c2cn(C3CCC3)nc2C#N)n(-c2ccc(F)cc2Br)n1 ; Cc1cc(Cc2cn(C3CCC3)nc2C#N)n(-c2ccc(F)cc2Br)n1] | 414 [M + H]⁺ |

3-bromo-4-((3-(cyclopropylmethyl)-1H-pyrazol-5-yl)methyl)-1-methyl-1H-pyrazole

| | |
|---|---|
| 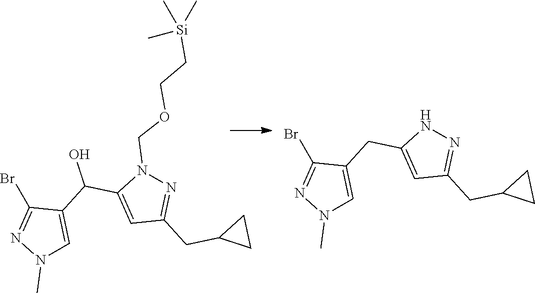 [SMILES: Cn1cc(C(O)c2cc(CC3CC3)nn2COCC[Si](C)(C)C)c(Br)n1 ; Cn1cc(Cc2cc(CC3CC3)n[nH]2)c(Br)n1] | 295 [M + H]⁺ |

### Synthesis of 3-formyl-1-((2-(trimethylsilyl)ethoxy)methyl)-1H-pyrazole-5-carbonitrile

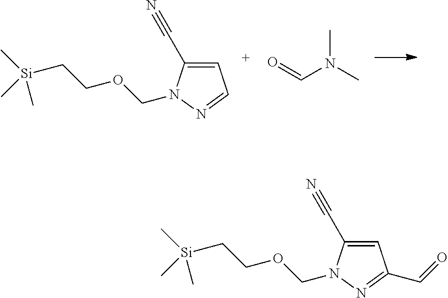

To a solution of 1-((2-(trimethylsilyl)ethoxy)methyl)-1H-pyrazole-5-carbonitrile (5.30 g, 23.7 mmol)) in THE (25 mL) was added 2,2,6,6-Tetramethylpiperidinylmagnesium chloride lithium chloride complex solution (35.6 mL, 35.6 mmol, 1M in hexane) dropwise under the atmosphere of N₂ at −15° C. After stirring for 1 h under the same conditions, DMF (3.671 mL, 47.5 mmol) was added dropwise at −15° C. and the mixture was stirred at the same temperature for 1 h. The mixture was quenched with sat. aq. NH₄Cl (50 mL) and extracted with EtOAc (80 mL×3). The combined organic phases were washed with brine (50 mL×2), dried over anhydrous Na₂SO₄ and concentrated. The residue was purified by flash column chromatography on silica gel (0→10% EtOAc in PE) to give 3-formyl-1-((2-(trimethylsilyl)ethoxy)methyl)-1H-pyrazole-5-carbonitrile (3.00 g, yield: 50.3%) as yellow oil. LC/MS (ESI) (m/z): 252 [M+H]⁺

The following intermediates were synthesized using a similar protocol (with m/z (ESI) values):

3-((1-(2-(1-((tert-butyldimethylsilyl)oxy)ethyl)-4-fluorophenyl)-3-methyl-1H-pyrazol-5-
yl)(hydroxy)methyl)-1-((2-(trimethylsilyl)ethoxy)methyl)-1H-pyrazole-5-carbonitrile

| | |
|---|---|
| 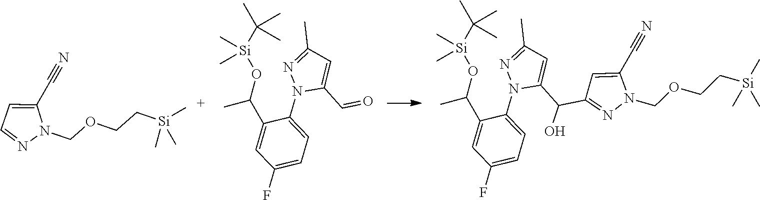 | 586 [M + H]⁺ |

3-((2-chloropyridin-3-yl)(hydroxy)methyl)-1-((2-(trimethylsilyl)ethoxy)methyl)-1H-pyrazole-5-
carbonitrile

| | |
|---|---|
| 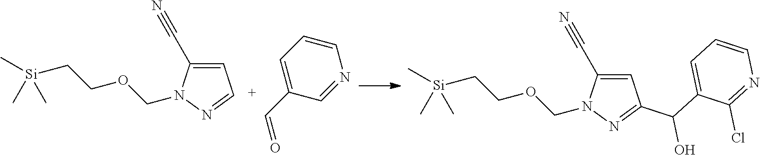 | 365 [M + H]⁺ |

3-(hydroxy(3-iodo-1-methyl-1H-pyrazol-4-yl)methyl)-1-((2-(trimethylsilyl)ethoxy)methyl)-1H-
pyrazole-5-carbonitrile

| | |
|---|---|
| 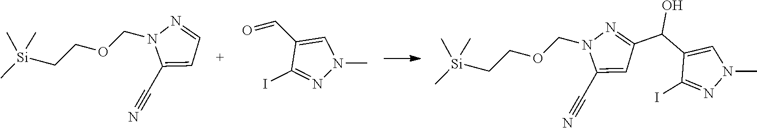 | 460 [M + H]⁺ |

### Synthesis of 3-((2-bromopyridin-3-yl)oxy)-1-methyl-1H-pyrazole-3-carboxylic acid

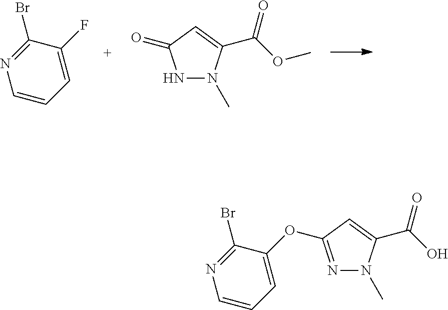 [SMILES: Fc1cccnc1Br ; COC(=O)c1cc(=O)[nH]n1C ; Cn1nc(Oc2cccnc2Br)cc1C(=O)O]

To a solution of methyl 2-methyl-5-oxo-2,5-dihydro-1H-pyrazole-3-carboxylate (500 mg, 3.20 mmol) and 2-bromo-3-fluoropyridine (1.13 g, 6.41 mmol) in DMF (10 mL) was added Cs₂CO₃ (5.22 g, 16.0 mmol). The mixture was stirred at 60° C. for 48 h. The mixture was concentrated, diluted with H₂O (10 mL), then acidified with aq. HCl (4 M) at 0° C. The mixture was filtered, and the filter cake was washed with H₂O (10 mL) to afford the white solid. The white solid was dried in vacuum to give 3-((2-bromopyridin-3-yl)oxy)-1-methyl-1H-pyrazole-5-carboxylic acid (680 mg, yield: 71.3%) as a white solid. LC/MS (ESI) (m/z): 298 [M+H]⁺.

The following intermediate was synthesized using a similar protocol (with m/z (ESI) values):

ethyl 4-((2-bromopyridin-3-yl)oxy)-1-(cyclopropylmethyl)-1H-pyrazole-3-carboxylate

| | |
|---|---|
| 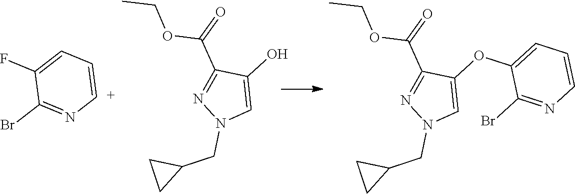 [SMILES: FC1=CC=[C+]N=C1Br ; CCOC(=O)c1nn(CC2CC2)cc1O ; CCOC(=O)c1nn(CC2CC2)cc1Oc1cccnc1Br] | 366 [M + H]⁺ |

### Synthesis of 3-(4-bromo-2-methylthiazole-5-carbonyl)-1-methyl-1H-pyrazole-5-carbonitrile and 5-(4-bromo-2-methylthiazole-5-carbonyl)-1-methyl-1H-pyrazole-3-carbonitrile

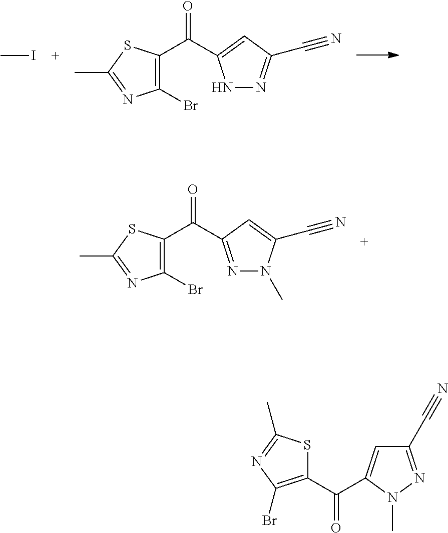

To a solution of 5-(4-bromo-2-methylthiazole-5-carbonyl)-1H-pyrazole-3-carbonitrile (90 mg, 0.25 mmol) and K₂CO₃ (69 mg, 0.49 mmol) in DMF (1 mL) was added iodomethane (39 mg, 0.27 mmol) dropwise at 0° C. The reaction was stirred at 25° C. for 16 h. The mixture was concentrated and diluted with EtOAc (15 mL), washed with brine (10 mL), dried over Na₂SO₄ and concentrated. The residue was purified by column chromatography on silica gel (0→25% EtOAc in PE) to give 3-(4-bromo-2-methylthiazole-5-carbonyl)-1-methyl-1H-pyrazole-5-carbonitrile and 5-(4-bromo-2-methylthiazole-5-carbonyl)-1-methyl-1H-pyrazole-3-carbonitrile (60 mg, yield: 64.5%) as a yellow oil mixture. LC/MS (ESI) (m/z): 311 [M+H]⁺.

The following intermediates were synthesized using a similar protocol (with m/z (ESI) values):

| 3-(1-(4-fluoro-2-(1-hydroxyethyl)phenyl)-3-methyl-1H-pyrazole-5-carbonyl)-1-methyl-1H- | |
|---|---|
| pyrazole-5-carbonitrile and 5-(1-(4-fluoro-2-(1-hydroxyethyl)phenyl)-3-methyl-1H-pyrazole-5- | |
| carbonyl)-1-methyl-1H-pyrazole-3-carbonitrile | |
| 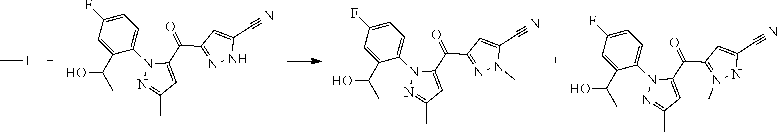 | 354 [M + H]⁺ |
| 3-(2-chloronicotinoyl)-1-methyl-1H-pyrazole-5-carbonitrile and 5-(2-chloronicotinoyl)-1- | |
| methyl-1H-pyrazole-3-carbonitrile | |
| 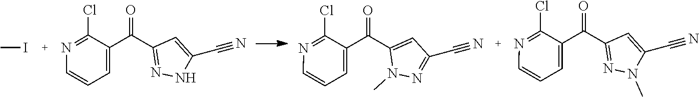 | 247 [M + H]⁺ |
| 3-(3-iodo-1-methyl-1H-pyrazole-4-carbonyl)-1-methyl-1H-pyrazole-5-carbonitrile | |
| 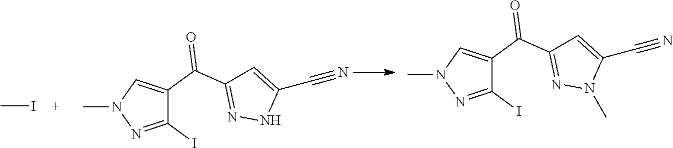 | 342 [M + H]⁺ |
| 3-(1-(2,4-difluoro-6-(1-hydroxyethyl)phenyl)-3-methyl-1H-pyrazole-5-carbonyl)-1-methyl-1H- | |
| pyrazole-5-carbonitrile | |
| 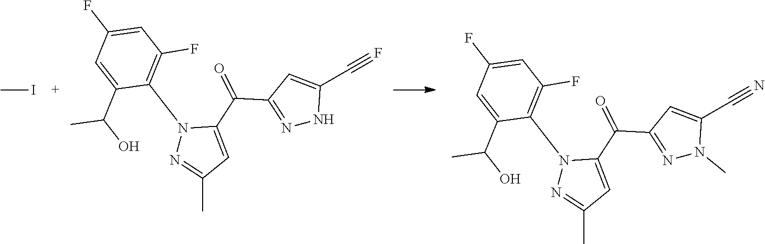 | 372 [M + H]⁺ |
| 3-(2-bromonicotinoyl)-1-methyl-1H-pyrazole-5-carbonitrile and 5-(2-bromonicotinoyl)-1- | |
| methyl-1H-pyrazole-3-carbonitrile | |
| 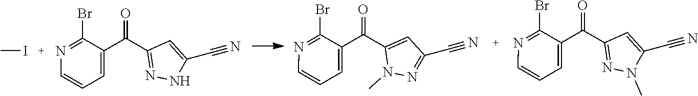 | 291 [M + H]⁺ |
| 3-(2-bromonicotinoyl)-1-isopropyl-1H-pyrazole-5-carbonitrile and 5-(2-bromonicotinoyl)-1- | |
| isopropyl-1H-pyrazole-3-carbonitrile | |
| 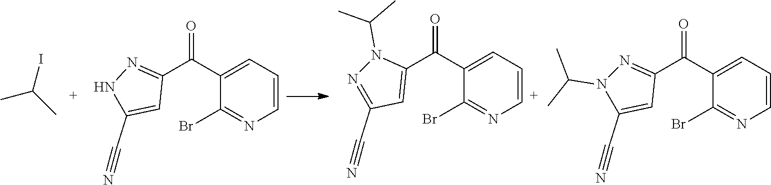 | 319 [M + H]⁺ |
| 3-(2-bromonicotinoyl)-1-ethyl-1H-pyrazole-5-carbonitrile | |
| 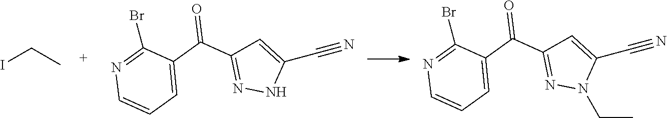 | 305 [M + H]⁺ |
| 3-(2-bromonicotinoyl)-1-(2-methoxyethyl)-1H-pyrazole-5-carbonitrile | |
| 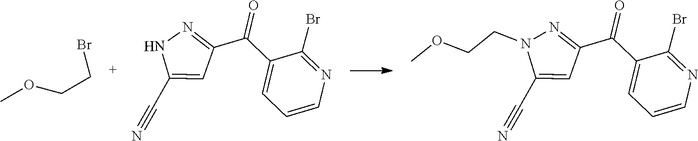 | 335 [M + H]⁺ |

### Synthesis of (1-(cyclopropylmethyl)-3-(methylthio)-1H-pyrazol-4-yl)methanol

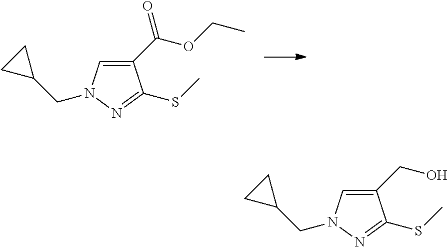 [SMILES: CCOC(=O)c1cn(CC2CC2)nc1SC ; CSc1nn(CC2CC2)cc1CO]

To a solution of ethyl 1-(cyclopropylmethyl)-3-(methylthio)-1H-pyrazole-4-carboxylate (1.00 g, 4.16 mmol) in THF (19 mL) was added DIBAL-H (1 M in THF, 12.48 mL, 12.48 mmol) at −70° C. The reaction was stirred at 0° C. for 1 h. The mixture was quenched with sat. aq. NH₄Cl (30 mL) and extracted with EtOAc (30 mL×3). The combined organic phase was washed with brine (25 mL×2), dried with anhydrous Na₂SO₄ and concentrated. The residue was purified by flash column chromatography on silica gel (0→100% EtOAc in PE) to afford (1-(cyclopropylmethyl)-3-(methylthio)-1H-pyrazol-4-yl)methanol (800 mg, 97.0% yield) as a white solid. LC/MS (ESI) (m/z): 199 [M+H]⁺.

The following intermediate was synthesized using a similar protocol (with m/z (ESI) values:

(3-(cyclopropylmethyl)-1-((2-(trimethylsilyl)ethoxy)methyl)-1H-pyrazol-5-yl)methanol

| | |
|---|---|
| 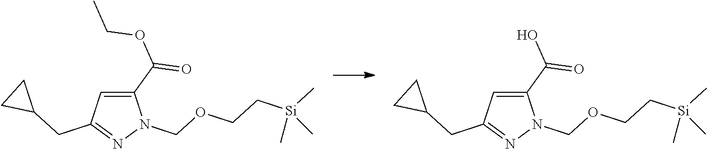 [SMILES: CCOC(=O)c1cc(CC2CC2)nn1COCC[Si](C)(C)C ; C[Si](C)(C)CCOCn1nc(CC2CC2)cc1C(=O)O] | 283 [M + H]⁺ |

### Synthesis of 4-((2-chloropyridin-3-yl)(hydroxy)methyl)-1-ethyl-1H-pyrazole-3-carbonitrile

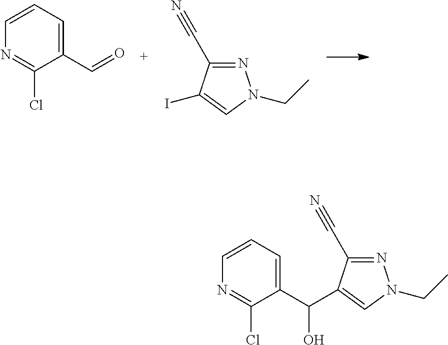

To a stirred solution of 1-ethyl-4-iodo-1H-pyrazole-3-carbonitrile (2.00 g, 8.10 mmol) in THF (20 mL) was added i-PrMgCl (1 M in THF, 6.20 mL, 8.10 mmol) at 0° C. After stirring at 0° C. for 0.5 h, a solution of 2-chloronicotinaldehyde (2.23 g, 16.2 mmol) in THE (20 mL) was added dropwise at 0° C. The reaction was stirred at 25° C. for 0.5 h. The mixture was quenched with sat. aq. NH₄Cl (20 mL) and extracted with EtOAc (50 mL×3). The combined organic layers were washed with brine (20 mL×3), dried by anhydrous Na₂SO₄, and concentrated. The residue was purified by flash column chromatography on silica gel (40-50% EtOAc in PE) to give 4-((2-chloropyridin-3-yl)(hydroxy)methyl)-1-ethyl-1H-pyrazole-3-carbonitrile (930 mg, yield: 44.0%) as white solid. LC/MS (ESI) m/z: 263 [M+H]⁺.

The following intermediates were synthesized using a similar protocol (with m/z (ESI) values):

4-((3-bromo-1-methyl-1H-pyrazol-4-yl)(hydroxy)methyl)-1-cyclobutyl-1H-pyrazole-3-
carbonitrile

| | |
|---|---|
| 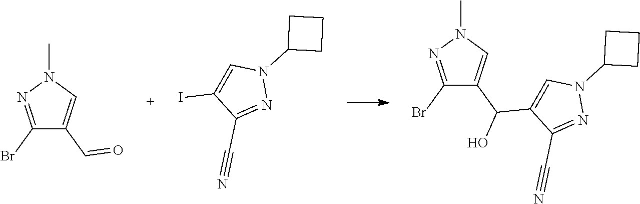 | 336 [M + H]⁺ |

4-((2-chloropyridin-3-yl)(hydroxy)methyl)-1-(cyclopropylmethyl)-1H-pyrazole-3-carbonitrile

| | |
|---|---|
| 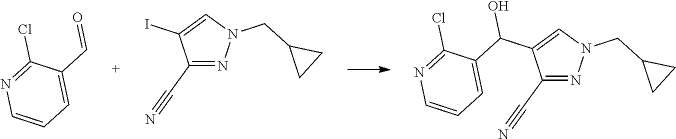 [SMILES: O=Cc1cccnc1Cl ; N#Cc1nn(CC2CC2)cc1I ; N#Cc1nn(CC2CC2)cc1C(O)c1cccnc1Cl] | 289 [M + H]⁺ |

4-{[1-(2-bromo-4,6-difluorophenyl)-3-methyl-1H-pyrazol-5-yl](hydroxy)methyl}-1-
(cyclopropylmethyl)-1H-pyrazole-3-carbonitrile

| | |
|---|---|
| 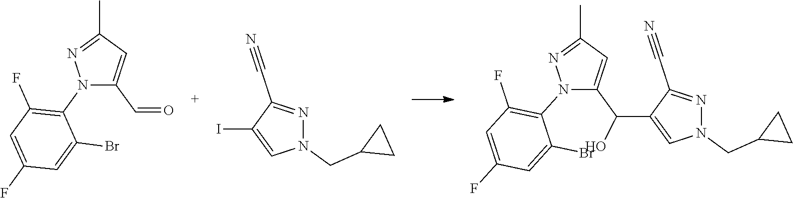 [SMILES: Cc1cc(C=O)n(-c2c(F)cc(F)cc2Br)n1 ; N#Cc1nn(CC2CC2)cc1I ; Cc1cc(C(O)c2cn(CC3CC3)nc2C#N)n(-c2c(F)cc(F)cc2Br)n1] | 448 [M + H]⁺ |

4-((2-bromopyridin-3-yl)(hydroxy)methyl)-1-ethyl-1H-pyrazole-3-carbonitrile

| | |
|---|---|
| 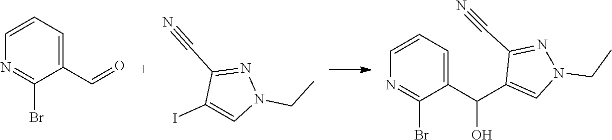 | 307 [M + H]⁺ |

(2-bromopyridin-3-yl)(5-cyclobutyl-1-((2-(trimethylsilyl)ethoxy)methyl)-1H-pyrazol-3-
yl)methanol

| | |
|---|---|
| 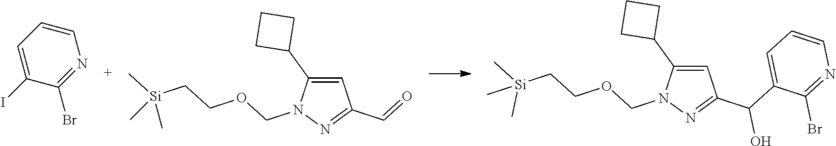 [SMILES: Brc1ncccc1I ; C[Si](C)(C)CCOCn1nc(C=O)cc1C1CCC1 ; C[Si](C)(C)CCOCn1nc(C(O)c2cccnc2Br)cc1C1CCC1] | 438 [M + H]⁺ |

4-((1-(2-bromo-4-fluorophenyl)-3-methyl-1H-pyrazol-5-yl)(hydroxy)methyl)-1-cyclobutyl-1H-
pyrazole-3-carbonitrile

| | |
|---|---|
| 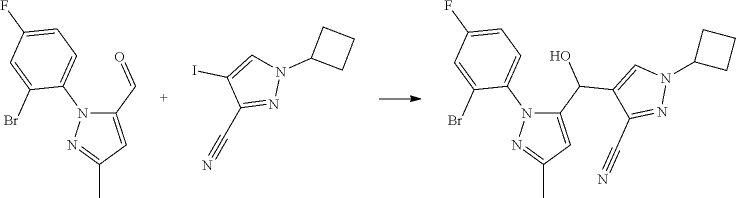 [SMILES: Cc1cc(C=O)n(-c2ccc(F)cc2Br)n1 ; N#Cc1nn(C2CCC2)cc1I ; Cc1cc(C(O)c2cn(C3CCC3)nc2C#N)n(-c2ccc(F)cc2Br)n1] | 430 [M+ H]⁺ |

(2-bromopyridin-3-yl)(1-(cyclopropylmethyl)-1H-pyrazol-4-yl)methanol

| | |
|---|---|
| 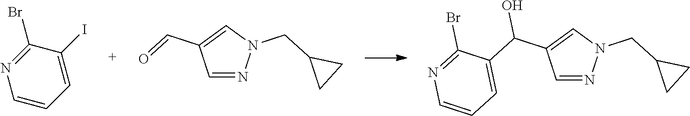 [SMILES: Brc1ncccc1I ; O=Cc1cnn(CC2CC2)c1 ; OC(c1cnn(CC2CC2)c1)c1cccnc1Br] | 308 [M + H]⁺ |
| (2-chloropyridin-3-yl)(3-ethyl-1-((2-(trimethylsilyl)ethoxy)methyl)-1H-pyrazol-5-yl)methanol | |
| 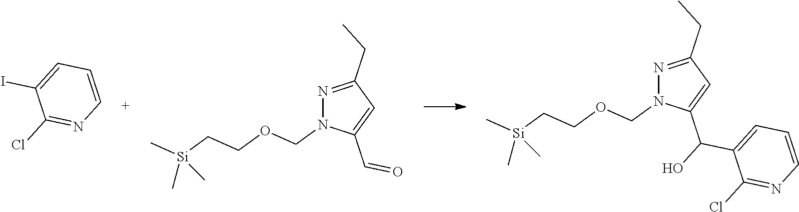 [SMILES: Clc1ncccc1I ; CCc1cc(C=O)n(COCC[Si](C)(C)C)n1 ; CCc1cc(C(O)c2cccnc2Cl)n(COCC[Si](C)(C)C)n1] | 368 [M + H]⁺ |

### Synthesis of (2-bromopyridin-3-yl)(5-ethyl-1-methyl-1H-pyrazol-3-yl)methanone

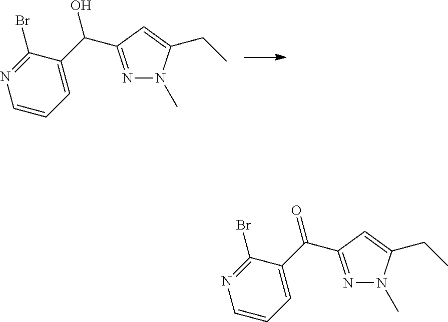 [SMILES: CCc1cc(C(O)c2cccnc2Br)nn1C ; CCc1cc(C(=O)c2cccnc2Br)nn1C]

To a solution of (2-bromopyridin-3-yl)(5-ethyl-1-methyl-1H-pyrazol-3-yl)methanol (1.8 g, 6.08 mmol) in DCM (10 mL) and MeOH (1 mL), was added MnO₂ (2.64 g, 30.4 mmol). The reaction was stirred at 45° C. for 12 h. The mixture was filtered, and the filtrate was concentrated. The residue was purified by column chromatography on silica gel (0→5% MeOH in DCM) to afford (2-bromopyridin-3-yl)(5-ethyl-1-methyl-1H-pyrazol-3-yl)methanone (1.1 g, 61.1% yield) as a yellow solid. LC/MS (ESI) (m/z): 294.1 [M+H]⁺.

The following intermediates were synthesized using a similar protocol (with m/z (ESI) values):

(2-bromopyridin-3-yl)(1-(cyclopropylmethyl)-1H-pyrazol-4-yl)methanone

| | |
|---|---|
| 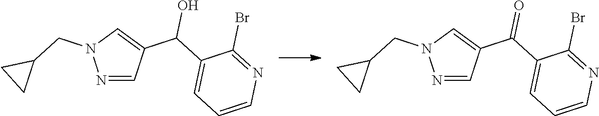 [SMILES: OC(c1cnn(CC2CC2)c1)c1cccnc1Br ; O=C(c1cnn(CC2CC2)c1)c1cccnc1Br] | 306 [M + H]⁺ |

3-(2,4-dibromothiazole-5-carbonyl)-1-((2-(trimethylsilyl)ethoxy)methyl)-1H-pyrazole-5-
carbonitrile

| | |
|---|---|
| 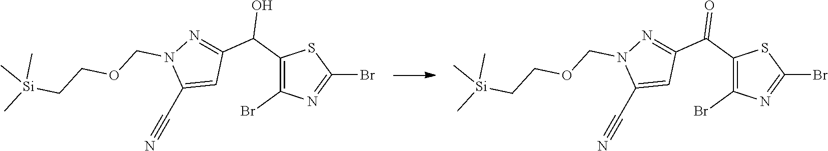 [SMILES: C[Si](C)(C)CCOCn1nc(C(O)c2sc(Br)nc2Br)cc1C#N ; C[Si](C)(C)CCOCn1nc(C(=O)c2sc(Br)nc2Br)cc1C#N] | 491 [M + H]⁺ |

### 3-(2,4-dibromothiazole-5-carbonyl)-1-((2-(trimethylsilyl)ethoxy)methyl)-1H-pyrazole-5-carbonitrile

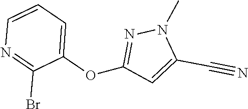 [SMILES: Cn1nc(Oc2cccnc2Br)cc1C#N]

To a mixture of 3-((2-bromopyridin-3-yl)oxy)-1-methyl-1H-pyrazole-5-carboxylic acid (730 mg, 2.45 mmol) in THF (20 mL) was added oxalyl chloride (1.84 mL, 3.67 mmol) and DMF (0.020 mL, 0.245 mmol) at 0° C. under N₂, the mixture was stirred at 60° C. for 2.5 h. Then NH₄OH (4 mL, 8.57 mmol) was added at 0° C. and the mixture was stirred at r.t. under N₂ for 12 h. The reaction was quenched with sat. aq. NH₄Cl (20 mL) and extracted with EtOAc (20 mL×3). The combined organic phases were washed with brine (30 mL×2), dried with anhydrous Na₂SO₄ and concentrated to give 3-((2-bromopyridin-3-yl)oxy)-1-methyl-1H-pyrazole-5-carboxamide (710 mg, crude) as a yellow oil. LC/MS (ESI) (m/z): 297 [M+H]⁻.

To a mixture of 3-((2-bromopyridin-3-yl)oxy)-1-methyl-1H-pyrazole-5-carboxamide (710 mg, 2.39 mmol) in THE (20 mL) was added TEA (0.830 mL, 5.97 mmol) and TFAA (1.16 mL, 8.36 mmol). The reaction was stirred at 25° C. for 1 h. The mixture was quenched with sat. aq. NH₄Cl (20 mL) and extracted with EtOAc (30 mL×2). The combined organic phases were washed with brine (20 mL×2), dried over anhydrous Na₂SO₄, filtered, and concentrated. The residue was purified by flash column chromatography on silica gel (0→30% EtOAc in PE) to give 3-((2-bromopyridin-3-yl)oxy)-1-methyl-1H-pyrazole-5-carbonitrile (365 mg, yield: 54.7%) as a colorless oil. LC/MS (ESI) (m/z): 279 [M+H]⁺

### Synthesis of 4-((2-bromopyridin-3-yl)(hydroxy)methyl)-1-ethyl-1H-pyrazole-3-carboxylic acid

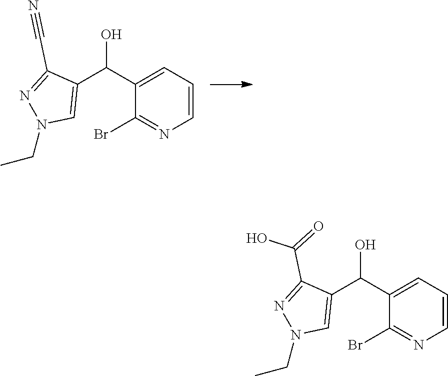 [SMILES: CCn1cc(C(O)c2cccnc2Br)c(C#N)n1 ; CCn1cc(C(O)c2cccnc2Br)c(C(=O)O)n1]

A mixture of 4-((2-bromopyridin-3-yl)(hydroxy)methyl)-1-ethyl-1H-pyrazole-3-carbonitrile (1.00 g, 3.27 mmol) and conc. HCl (10 mL) was stirred at 80° C. for 4 h. The mixture was diluted with H₂O (20 mL) and extracted with EtOAc (30 mL×3). The combined organic layers were dried over anhydrous Na₂SO₄, filtered and concentrated to afford 4-((2-bromopyridin-3-yl)(hydroxy)methyl)-1-ethyl-1H-pyrazole-3-carboxylic acid (900 mg, crude) as yellow oil. LC/MS (ESI) m/z: 326 [M+H]⁺.

The following intermediates were synthesized using a similar protocol (with m/z (ESI) values):

4-((2-bromopyridin-3-yl)methyl)-1-(cyclopropylmethyl)-1H-pyrazole-3-carboxylic acid

| | |
|---|---|
| 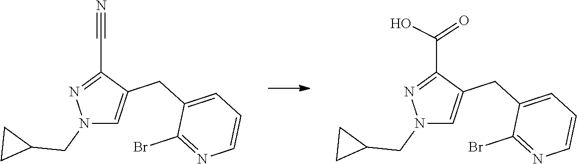 [SMILES: N#Cc1nn(CC2CC2)cc1Cc1cccnc1Br ; O=C(O)c1nn(CC2CC2)cc1Cc1cccnc1Br] | 336 [M + H]⁺ |

4-((1-(2-bromo-4,6-difluorophenyl)-3-methyl-1H-pyrazol-5-yl)methyl)-1-(cyclopropylmethyl)-

| | |
|---|---|
| 1H-pyrazole-3-carboxylic acid | |
| 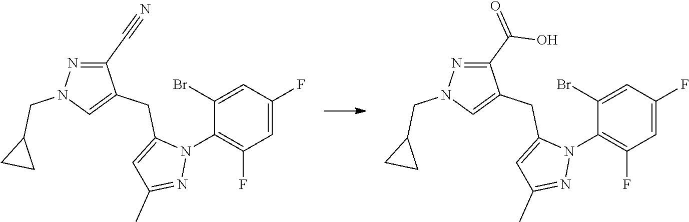 [SMILES: Cc1cc(Cc2cn(CC3CC3)nc2C#N)n(-c2c(F)cc(F)cc2Br)n1 ; Cc1cc(Cc2cn(CC3CC3)nc2C(=O)O)n(-c2c(F)cc(F)cc2Br)n1] | 451 [M + H]⁺ |

4-((2-bromopyridin-3-yl)methyl)-1-ethyl-1H-pyrazole-3-carboxylic acid

| | |
|---|---|
| 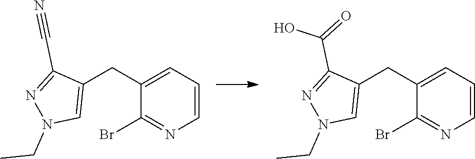 [SMILES: CCn1cc(Cc2cccnc2Br)c(C#N)n1 ; CCn1cc(Cc2cccnc2Br)c(C(=O)O)n1] | 310 [M + H]⁺ |

4-((2-chloropyridin-3-yl)methyl)-1-cyclobutyl-1H-pyrazole-3-carboxylic acid

| | |
|---|---|
| 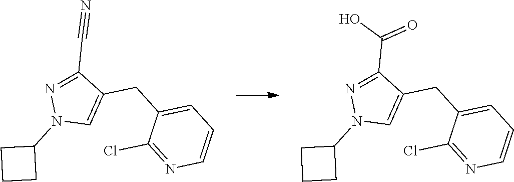 [SMILES: N#Cc1nn(C2CCC2)cc1Cc1cccnc1Cl ; O=C(O)c1nn(C2CCC2)cc1Cc1cccnc1Cl] | 292 [M + H]⁺ |

4-((1-(2-bromo-4-fluorophenyl)-3-methyl-1H-pyrazol-5-yl)methyl)-1-cyclobutyl-1H-pyrazole-
3-carboxylic acid

| | |
|---|---|
| 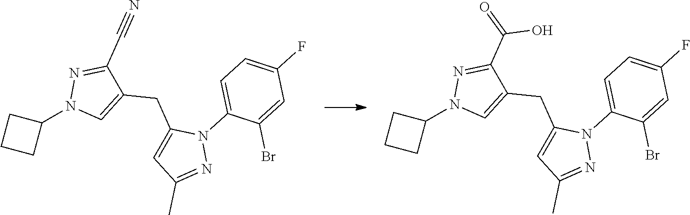 [SMILES: Cc1cc(Cc2cn(C3CCC3)nc2C#N)n(-c2ccc(F)cc2Br)n1 ; Cc1cc(Cc2cn(C3CCC3)nc2C(=O)O)n(-c2ccc(F)cc2Br)n1] | 433 [M + H]⁺ |

### Synthesis of 4-((2-chloropyridin-3-yl)(hydroxy)methyl)-1-cyclobutyl-1H-pyrazole-3-carbonitrile

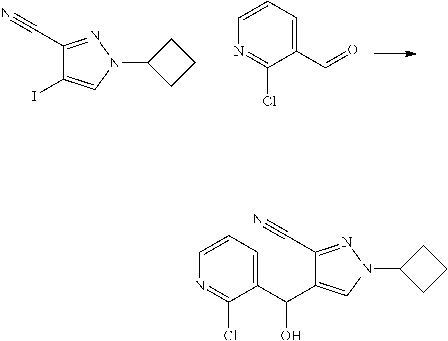

To a solution of 1-cyclobutyl-4-iodo-1H-pyrazole-3-carbonitrile (1.70 g, 6.23 mmol) in THF (50 mL) was added i-PrMgCl·LiCl (1.3 M in THF 4.80 mL, 6.23 mmol) at 0° C. After stirring at 0° C. for 1 h a solution of 2-chloronicotinaldehyde (1.77 g, 12.45 mmol) in THF (50 mL) was added at 20° C. The reaction was stirred at 20° C. for 1 h. The mixture was quenched with sat. aq. NH₄Cl (25 mL) and extracted with EtOAc (45 mL×3). The combined organic layers were dried over anhydrous Na₂SO₄, filtered, and concentrated. The residue was purified by flash column chromatography on silica gel (50% EtOAc in PE) to afford 4-((2-chloropyridin-3-yl)(hydroxy)methyl)-1-cyclobutyl-1H-pyrazole-3-carbonitrile (1.10 g, 49.0% yield) as white oil. LC/MS (ESI) (m/z): 289 [M+H]⁺.

The following intermediates were synthesized using a similar protocol (with m/z (ESI) values):

1-(cyclopropylmethyl)-4-((1-(4-fluoro-2-iodophenyl)-3-methyl-1H-pyrazol-5-
yl)(hydroxy)methyl)-1H-pyrazole-3-carbonitrile

| | |
|---|---|
| 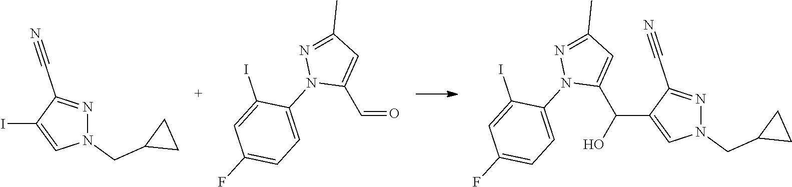 [SMILES: N#Cc1nn(CC2CC2)cc1I ; Cc1cc(C=O)n(-c2ccc(F)cc2I)n1 ; Cc1cc(C(O)c2cn(CC3CC3)nc2C#N)n(-c2ccc(F)cc2I)n1] | 478 [M + H]⁺ |

4-((2-bromopyridin-3-yl)(hydroxy)methyl)-1-(cyclopropylmethyl)-1H-pyrazole-3-carbonitrile

| | |
|---|---|
| 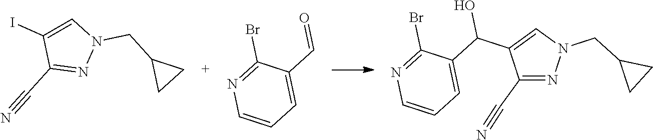 [SMILES: N#Cc1nn(CC2CC2)cc1I ; O=Cc1cccnc1Br ; N#Cc1nn(CC2CC2)cc1C(O)c1cccnc1Br] | 333 [M + H]⁺ |

(2-chloropyridin-3-yl)(3-(cyclopropylmethyl)-1-((2-(trimethylsilyl)ethoxy)methyl)-1H-pyrazol-
5-yl)methanol

| | |
|---|---|
| 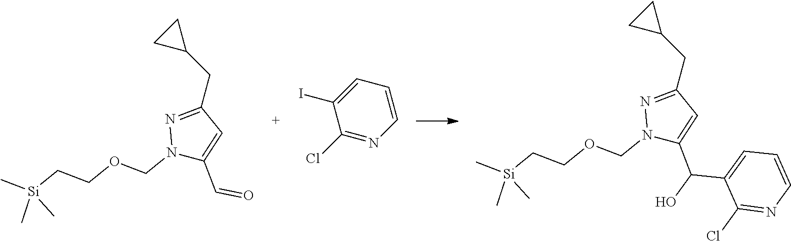 [SMILES: C[Si](C)(C)CCOCn1nc(CC2CC2)cc1C=O ; Clc1ncccc1I ; C[Si](C)(C)CCOCn1nc(CC2CC2)cc1C(O)c1cccnc1Cl] | 394 [M + H]⁺ |

(3-bromo-1-methyl-1H-pyrazol-4-y1)(3-(cyclopropylmethyl)-1-((2-
(trimethylsilyl)ethoxy)methyl)-1H-pyrazol-5-yl)methanol

| | |
|---|---|
| 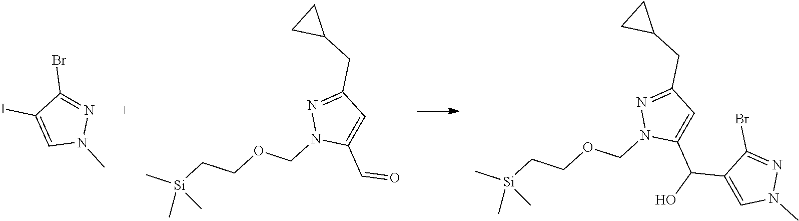 | 441 [M + H]⁺ |

### Synthesis of 3-((2-bromopyridin-3-yl)(methoxymethoxy)methyl)-1-methyl-1H-pyrazole-5-carbonitrile

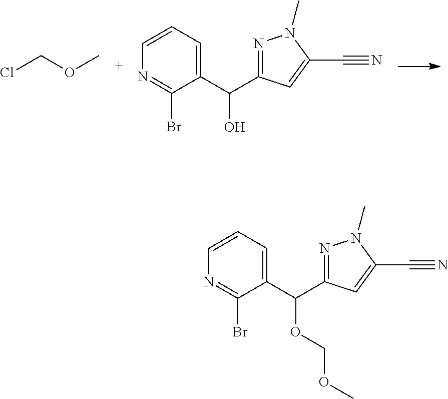

To a stirred solution of 3-((2-bromopyridin-3-yl)(hydroxy)methyl)-1-methyl-1H-pyrazole-5-carbonitrile (1.00 g, 3.42 mmol) in THF (10 mL) was added NaH (120 mg, 5.13 mmol, 60% in mineral oil) at 0° C. and the mixture was stirred at 0° C. for 30 min. Then MOMCl (410 mg, 5.13 mmol) was added dropwise at 0° C. and the mixture was stirred at 25° C. for 16 h. The reaction was quenched with sat. aq. NH₄Cl (100 mL) and extracted with DCM (30 mL). The organic phase was dried over anhydrous Na₂SO₄ and concentrated. The residue was purified by flash column chromatography on silica gel (0→25% EtOAc in PE) to give 3-((2-bromopyridin-3-yl)(methoxymethoxy)methyl)-1-methyl-1H-pyrazole-5-carbonitrile (761 mg, yield: 65.9%) as a yellow oil. LC/MS (ESI) (m/z): 337 [M+H]⁺.

### Synthesis of ethyl 1-(2-acetyl-4-fluorophenyl)-3-methyl-1H-pyrazole-5-carboxylate

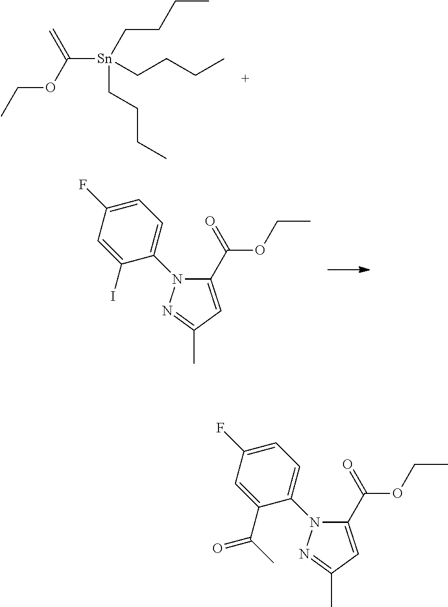 [SMILES: C=[C](OCC)[Sn]([CH2]CCC)([CH2]CCC)[CH2]CCC ; CCOC(=O)c1cc(C)nn1-c1ccc(F)cc1I ; CCOC(=O)c1cc(C)nn1-c1ccc(F)cc1C(C)=O]

To a solution of ethyl 1-(4-fluoro-2-iodophenyl)-3-methyl-1H-pyrazole-5-carboxylate (5.30 g, 14.16 mmol) and tributyl(1-ethoxyvinyl)stannane (7.67 g, 21.3 mmol) in toluene (30 mL) was added Pd(PPh₃)₄ (820 mg, 0.710 mmol). The reaction was degassed with N₂ three times and stirred at 100° C. for 16 h. The mixture was diluted with sat. aq. KF (20 mL) and ethyl acetate (20 mL) and stirring was continued for 1 h at r.t. The mixture was filtered, and the filtrate was extracted with ethyl acetate (20 mL×3). The combined organic phases were washed with brine (20 mL×2), dried over anhydrous Na₂SO₄, filtered, and concentrated. The residue was diluted with THF (20 mL) and aq. HCl (1 M, 20 mL) and the mixture was stirred at r.t. for 1 h (this acid hydrolysis step can be skipped in order to isolate the enol ether intermediate). The mixture was extracted with EtOAc (30 mL×3). The combined organic phases were washed with brine (20 mL×2), dried over anhydrous Na₂SO₄, filtered and concentrated. The residue was purified by flash column chromatography on silica gel (0→8% MeOH in DCM) to give ethyl 1-(2-acetyl-4-fluorophenyl)-3-methyl-1H-pyrazole-5-carboxylate (1.60 g, yield: 39%) as a yellow oil. LC/MS ESI (m/z): 291 [M+H]⁺.

The following intermediates were synthesized using a similar protocol (with m/z (ESI) values):

1-(cyclopropylmethyl)-4-((1-(2-(1-ethoxyvinyl)-4-fluorophenyl)-3-methyl-1H-pyrazol-5-
yl)methyl)-1H-pyrazole-3-carbonitrile

| | |
|---|---|
| 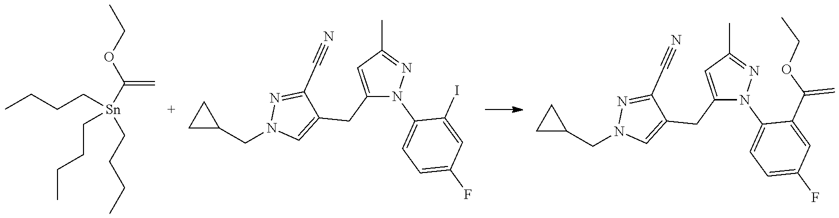 [SMILES: C=[C](OCC)[Sn]([CH2]CCC)([CH2]CCC)[CH2]CCC ; Cc1cc(Cc2cn(CC3CC3)nc2C#N)n(-c2ccc(F)cc2I)n1 ; C=C(OCC)c1cc(F)ccc1-n1nc(C)cc1Cc1cn(CC2CC2)nc1C#N] | 406 [M + H]⁺ |
| 1-(cyclopropylmethyl)-4-({1-[2-(1-ethoxyethenyl)-4,6-difluorophenyl]-3-methyl-1H-pyrazol-5- | |
| yl}methyl)-N,N-dimethyl-1H-pyrazole-3-carboxamide | |
| 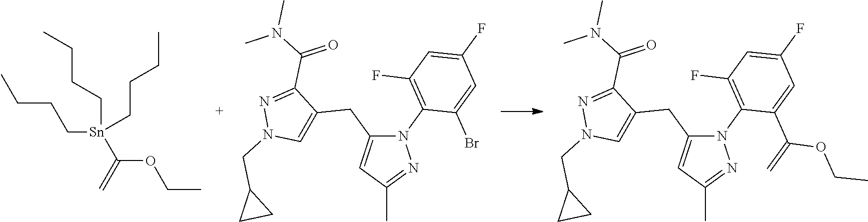 [SMILES: C=[C](OCC)[Sn]([CH2]CCC)([CH2]CCC)[CH2]CCC ; Cc1cc(Cc2cn(CC3CC3)nc2C(=O)N(C)C)n(-c2c(F)cc(F)cc2Br)n1 ; C=C(OCC)c1cc(F)cc(F)c1-n1nc(C)cc1Cc1cn(CC2CC2)nc1C(=O)N(C)C] | 470 [M + H]⁺ |
| 4-((1-(2-acetyl-4-fluorophenyl)-3-methyl-1H-pyrazol-5-yl)methyl)-1-cyclobutyl-N,N-dimethyl- | |
| 1H-pyrazole-3-carboxamide | |
| 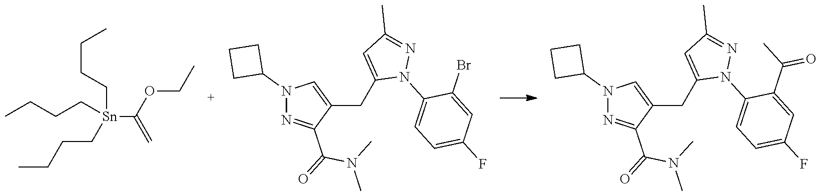 | 424 [M + H]⁺ |

### Synthesis of (3-ethyl-1-((2-(trimethylsilyl)ethoxy)methyl)-1H-pyrazol-5-yl)methanol

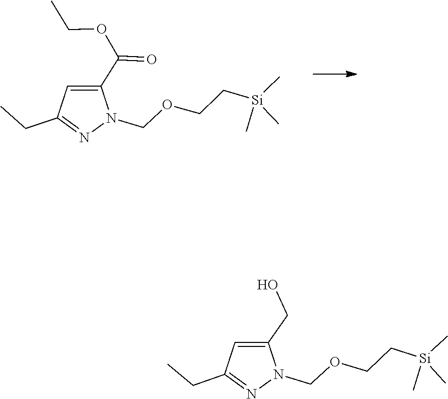

To a solution of ethyl 3-ethyl-1-((2-(trimethylsilyl)ethoxy)methyl)-1H-pyrazole-5-carboxylate (5.30 g, 17.8 mmol) in THF (70 mL) was added diisobutylaluminium hydride (1 M in hexane, 19.5 mL, 19.5 mmol) slowly at −78° C. under N₂. The reaction was stirred at 25° C. for 16 h. The mixture was quenched with sat. aq. NH₄Cl (30 mL) and potassium sodium tartrate (30 mL) and then extracted with EtOAc (80 mL×3). The combined organic layers were washed with brine (50 mL×2), dried over anhydrous Na₂SO₄ and concentrated. The residue was purified by flash column chromatography on silica gel (0→100% EtOAc in PE) to give (3-ethyl-1-((2-(trimethylsilyl)ethoxy)methyl)-1H-pyrazol-5-yl)methanol (4.50 g, yield: 90.3%) as a colorless oil. LC/MS ESI (m/z): 257 [M+H]⁺.

The following intermediates were synthesized using a similar protocol (with m/z (ESI) values):

(1-(2-(1-((tert-butyldimethylsilyl)oxy)ethyl)-4-fluorophenyl)-3-methyl-1H-pyrazol-5-
yl)methanol

| | |
|---|---|
| 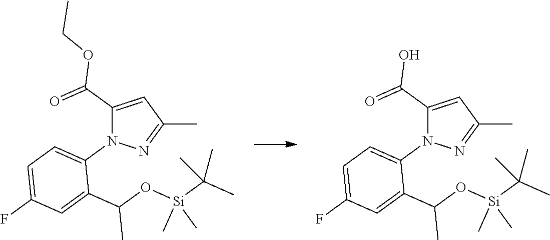 [SMILES: CCOC(=O)c1cc(C)nn1-c1ccc(F)cc1C(C)O[Si](C)(C)C(C)(C)C ; Cc1cc(C(=O)O)n(-c2ccc(F)cc2C(C)O[Si](C)(C)C(C)(C)C)n1] | 365 [M + H]⁺ |

(5-cyclobutyl-1-((2-(trimethylsilyl)ethoxy)methyl)-1H-pyrazol-3-yl)methanol

| | |
|---|---|
| 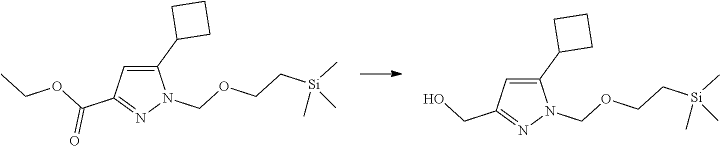 [SMILES: CCOC(=O)c1cc(C2CCC2)n(COCC[Si](C)(C)C)n1 ; C[Si](C)(C)CCOCn1nc(CO)cc1C1CCC1] | 283 [M + H]⁺ |

### Synthesis of 4-((3-bromo-1-methyl-1H-pyrazol-4-yl)methyl)-1-cyclobutyl-1H-pyrazole-3-carbonitrile

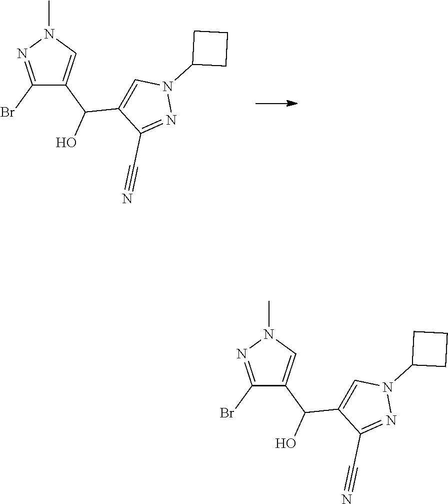 [SMILES: Cn1cc(C(O)c2cn(C3CCC3)nc2C#N)c(Br)n1 ; Cn1cc(C(O)c2cn(C3CCC3)nc2C#N)c(Br)n1]

To a solution of 4-((3-bromo-1-methyl-1H-pyrazol-4-yl)(hydroxy)methyl)-1-cyclobutyl-1H-pyrazole-3-carbonitrile (872 mg, 2.59 mmol) in DCM (10 mL) was added Et₃SiH (1.21 g, 10.4 mmol) and TFA (887 mg, 7.78 mmol) at 0° C. The mixture was stirred at 0° C. for 1 h and then concentrated. The residue was dissolved by DCM (20 mL) and washed with sat. aq. NaHCO₃ (15 mL×2). The combined organic layers were dried over anhydrous Na₂SO₄, filtered and concentrated. The residue was purified by flash column chromatography on silica gel (50→100% EtOAc in PE) to afford 4-((3-bromo-1-methyl-1H-pyrazol-4-yl)methyl)-1-cyclobutyl-1H-pyrazole-3-carbonitrile (562 mg, 67.7% yield) as a yellow oil. LC/MS (ESI) (m/z): 320 [M+H]⁺.

The following intermediate was synthesized using a similar protocol (with m/z (ESI) values):

1-(cyclopropylmethyl)-4-((3-iodo-1-methyl-1H-pyrazol-4-yl)methyl)-1H-pyrazole-3-
carbonitrile

| | |
|---|---|
| 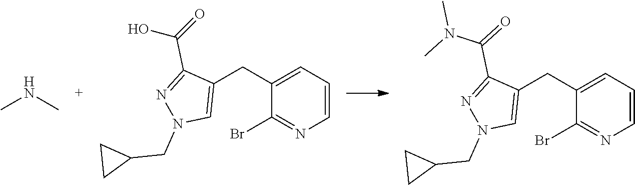 | 368 [M + H]⁺ |

### Synthesis of 4-((2-bromopyridin-3-yl)methyl)-1-ethyl-N,N-dimethyl-1H-pyrazole-3-carboxamide

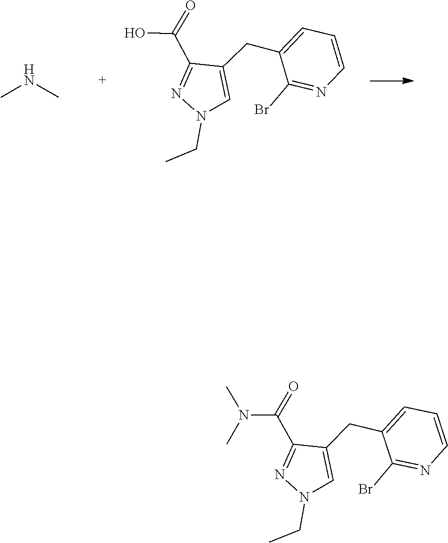

To solutions of 4-((2-bromopyridin-3-yl)methyl)-1-ethyl-1H-pyrazole-3-carboxylic acid (447 mg, 1.44 mmol) in DMF (10 mL) was added dimethylamine hydrochloride (353 mg, 4.32 mmol), HATU (877 mg, 2.31 mmol) and DIEA (0.310 mL, 1.87 mmol). The mixture was stirred at 20° C. for 16 h. The reaction was diluted with EtOAc (30 mL) and washed with water (25 mL×3). The organic layer was washed with brine (25 mL×2), dried over anhydrous Na₂SO₄ and concentrated. The residue was purified by flash column chromatography on silica gel (80→100% EtOAc in PE) to afford 4-((2-bromopyridin-3-yl)methyl)-1-ethyl-N,N-dimethyl-1H-pyrazole-3-carboxamide (250 mg, yield: 51.4%) as a colorless oil. LC/MS (ESI) (m/z): 337 [M+H]⁺.

The following intermediates were synthesized using a similar protocol (with m/z (ESI) values):

4-((2-bromopyridin-3-yl)methyl)-1-(cyclopropylmethyl)-N,N-dimethyl-1H-pyrazole-3-
carboxamide

| | |
|---|---|
| 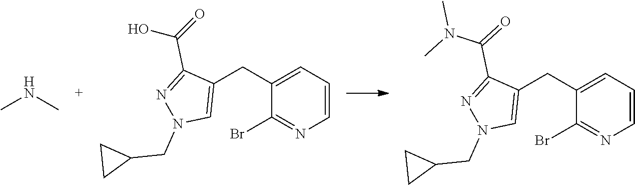 | 363 [M + H]⁺ |
| 4-{[1-(2-bromo-4,6-difluorophenyl)-3-methyl-1H-pyrazol-5-yl]methyl}-1-(cyclopropylmethyl)- | |
| N,N-dimethyl-1H-pyrazole-3-carboxamide | |
| 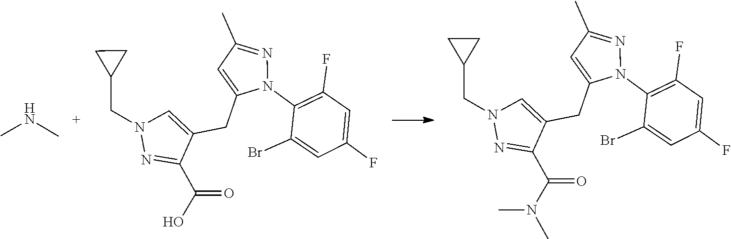 | 478 [M + H]⁺ |
| 4-((2-chloropyridin-3-yl)methyl)-1-cyclobutyl-N,N-dimethyl-1H-pyrazole-3-carboxamide | |
| 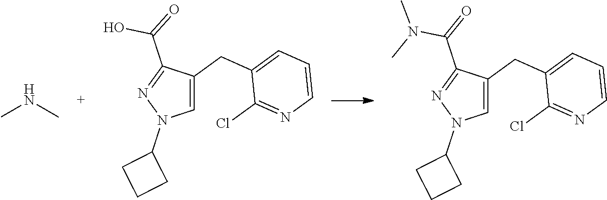 | 319 [M + H]⁺ |
| 4-((2-bromopyridin-3-yl)(hydroxy)methyl)-1-ethyl-N,N-dimethyl-1H-pyrazole-3-carboxamide | |
| 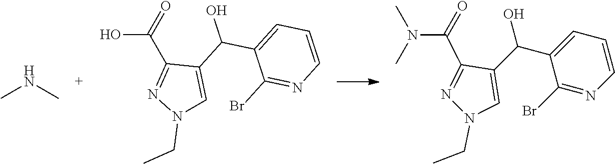 | 353 [M + H]⁺ |
| 4-(cyclopropylmethyl)-1-((3-iodo-1-methyl-1H-pyrazol-4-yl)methyl)-N,N-dimethyl-1H- | |
| imidazole-2-carboxamide | |
| 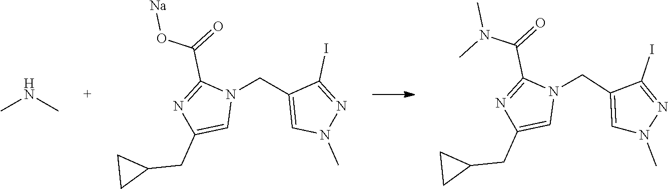 | 414 [M + H]⁺ |

### Synthesis of Sodium 4-(cyclopropylmethyl)-1-((3-iodo-1-methyl-1H-pyrazol-4-yl)methyl)-1H-imidazole-2-carboxylate

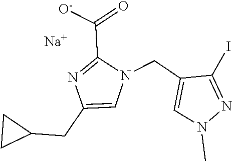 [SMILES: Cn1cc(Cn2cc(CC3CC3)nc2C(=O)[O-])c(I)n1 ; [Na+]]

To a solution of 4-(cyclopropylmethyl)-N,N-dimethyl-1H-imidazole-1-sulfonamide (1.14 g, 4.97 mol) in THE (30 mL) was added LDA (2.0 M in THF, 4.97 mL, 9.94 mol) at −78° C. After stirring at −78° C. for 0.5 h, a solution of dimethyl carbonate (670 mg, 7.46 mol) in THE (10 mL) was added dropwise at −78° C. and reaction was stirred at r.t. for 4 h. The mixture was quenched with sat. aq. NH₄Cl (50 mL) and extracted with EtOAc (30 mL×3). The combined organic phases were washed with brine (30 mL×3), dried over anhydrous Na₂SO₄, filtered and concentrated. The residue was purified by flash column chromatography on silica gel (0→5% MeOH in DCM) to give methyl 4-(cyclopropylmethyl)-1-(N,N-dimethylsulfamoyl)-1H-imidazole-2-carboxylate (410 mg, yield: 28.7%) as a yellow gum. LC/MS ESI (m/z): 288 [M+H]⁺.

To a solution of methyl 4-(cyclopropylmethyl)-1-(N,N-dimethylsulfamoyl)-1H-imidazole-2-carboxylate (400 mg, 1.39 mmol) in DCM (5 mL) was added HCl (4 M in dioxane, 5 mL, 20 mmol). The reaction was stirred at 50° C. for 2 h. The mixture was concentrated to directly give methyl 4-(cyclopropylmethyl)-1H-imidazole-2-carboxylate (250 mg, crude) as a yellow gum. LC/MS ESI (m/z): 181 [M+H]⁺.

To a solution of methyl 4-(cyclopropylmethyl)-1H-imidazole-2-carboxylate (250 mg, 1.39 mmol) and 4-(chloromethyl)-3-iodo-1-methyl-1H-pyrazole (580 mg, 2.78 mmol) in DMF (5 mL) was added Cs₂CO₃ (1.81 g, 5.57 mmol). The mixture was stirred at 80° C. for 2 h. The reaction mixture was quenched with water (30 mL) and extracted with EtOAc (30 mL×3). The combined organic phases were washed with H₂O (25 mL×3), brine (30 mL×2), dried over anhydrous Na₂SO₄, filtered and concentrated. The residue was purified by flash column chromatography on silica gel (0→50% EtOAc in PE) to give methyl 4-(cyclopropylmethyl)-1-((3-iodo-1-methyl-1H-pyrazol-4-yl)methyl)-1H-imidazole-2-carboxylate (540 mg, 97.2%, over two steps) as a yellow solid. LC/MS (ESI) (m/z): 401 [M+H]⁺.

To a solution of methyl 4-(cyclopropylmethyl)-1-((3-iodo-1-methyl-1H-pyrazol-4-yl)methyl)-1H-imidazole-2-carboxylate (360 mg, 0.900 mmol) in MeOH (5 mL) was added aq. NaOH (2 M in water, 1.80 mL, 3.60 mmol) at r.t. The mixture was stirred at 60° C. for 1 h. The reaction mixture was concentrated to give sodium 4-(cyclopropylmethyl)-1-((3-iodo-1-methyl-1H-pyrazol-4-yl)methyl)-1H-imidazole-2-carboxylate (340 mg, crude) as a white solid. LC/MS (ESI) (m/z): 387 [M+H]⁺.

### Synthesis of ethyl 3-(cyclopropylmethyl)-1-((2-(trimethylsilyl)ethoxy)methyl)-1H-pyrazole-5-carboxylate

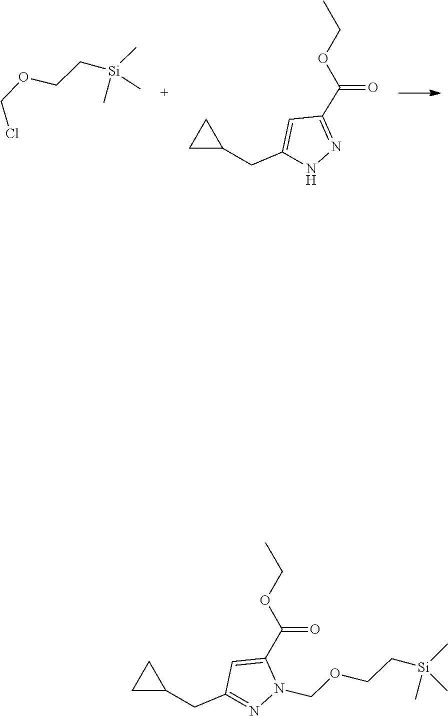

To a stirred solution of ethyl 5-(cyclopropylmethyl)-1H-pyrazole-3-carboxylate (1.30 g, 6.63 mmol) in THF (15 mL) was added NaH (401 mg, 10.0 mmol, 60% in mineral oil) at 0° C. under N₂. After stirring at 0° C. for 1 h, a solution of SEMCl (1.34 g, 8.03 mmol) in THF (5 mL) was added dropwise. The reaction was stirred at 0° C. for 2 h. The mixture was quenched with sat. aq. NH₄Cl (15 mL) and extracted with EtOAc (25 mL×3). The combined organic layers were washed with brine (20 mL×2), dried over anhydrous Na₂SO₄ and concentrated. The residue was purified by flash column chromatography on silica gel (10→25% EtOAc in PE) to give ethyl 3-(cyclopropylmethyl)-1-((2-(trimethylsilyl)ethoxy)methyl)-1H-pyrazole-5-carboxylate (1.77 g, 81.0% yield) as a white solid. LC/MS (ESI) (m/z): 325 [M+H]⁺.

### Synthesis of (R)-5-bromo-3-(1-(5-fluoro-2-(trimethylstannyl)phenyl)ethoxy)pyrazin-2-amine

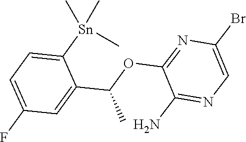 [SMILES: C[C@@H](Oc1nc(Br)cnc1N)c1cc(F)cc[c]1[Sn]([CH3])([CH3])[CH3]]

To a solution of (R)-1-(5-fluoro-2-iodophenyl)ethan-1-ol (500 mg, 1.88 mmol) and 3,5-dibromopyrazin-2-anine (470 mg, 2.26 mmol) in THF (5 mL) was added NaHMDS (1.4 mL, 2.82 mmol, 2.0 M in THF) at 0° C. under N₂ and the mixture was stirred at 70° C. overnight. The reaction was quenched with sat. aq. NH₄Cl (10 mL) and extracted with EtOAc (15 mL×3). The combined organic phase dried over anhydrous Na₂SO₄ and concentrated. The residue was purified by flash column chromatography on silica gel (0→50% EtOAc in PE) to give (R)-5-bromo-3-(1-(5-fluoro-2-iodophenyl)ethoxy)pyrazin-2-amine (570 mg, yield: 69.2%) as a yellow oil. LC/MS (ESI) m/z: 438 [M+H]⁺.

To a solution of (R)-5-bromo-3-(1-(5-fluoro-2-iodophenyl)ethoxy)pyrazin-2-amine (550 mg, 1.26 mmol) in toluene (5 mL) was added hexamethyldistannane (494 mg, 1.51 mmol) and Pd(PPh₃)₄ (72.5 mg, 0.0630 mmol) stirred at 25° C. The mixture was degassed and purged with N₂ three times, and the mixture was stirred at 100° C. for 16 h. The mixture was diluted with water (10 mL) and extracted with EtOAc (20 mL×3). The combined organic phases were washed with brine (20 mL×2), dried over anhydrous Na₂SO₄ and concentrated. The residue was purified by flash column chromatography on silica gel (0→20% EtOAc in PE) to give (R)-5-bromo-3-(1-(5-fluoro-2-(trimethylstannyl)phenyl)ethoxy)pyrazin-2-amine (570 mg, yield: 95.6%) as a yellow solid. LC/MS (ESI) m/z: 476 [M+H]⁺.

### Synthesis of (R)-1-(2-(3-((1-ethyl-1H-pyrazol-4-yl)oxy)pyridin-2-yl)-5-fluorophenyl)ethan-1-ol

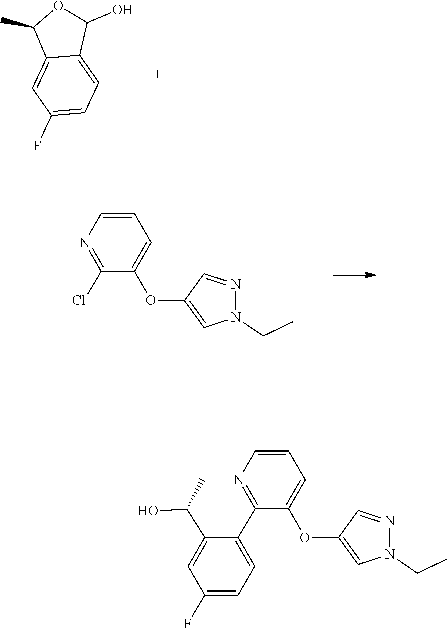 [SMILES: C[C@H]1OC(O)c2ccc(F)cc21 ; CCn1cc(Oc2cccnc2Cl)cn1 ; CCn1cc(Oc2cccnc2-c2ccc(F)cc2[C@@H](C)O)cn1]

To a solution of 2-chloro-3-((1-ethyl-1H-pyrazol-4-yl)oxy)pyridine (35.0 mg, 0.160 mmol), (3R)-5-fluoro-3-methyl 1,3-dihydro-2,1-benzoxaborol-1-ol (31.0 mg, 0.180 mmol), and Na₂CO₃ (33.0 mg, 0.310 mmol) in 1,4-dioxane (8 mL) and water (2 mL) was added Pd(dppf)Cl₂ (11.5 mg, 0.0160 mmol). The mixture was thrice degassed and purged with N₂ and then stirred at 80° C. for 16 h. The mixture was filtered, and the filtrate was concentrated. The residue was purified by flash column chromatography on silica gel (0→50% EtOAc in PE) to give (R)-1-(2-(3-((1-ethyl-1H-pyrazol-4-yl)oxy)pyridin-2-yl)-5-fluorophenyl)ethan-1-ol (20.0 mg, 39.0% yield) as a yellow oil. LCMS (ESI): m/z: 328 [M+H]⁺.

The following intermediates were synthesized using a similar protocol (with m/z (ESI) values):

(R)-1-ethyl-4-((2-(4-fluoro-2-(1-hydroxyethyl)phenyl)pyridin-3-yl)methyl)-1H-pyrazole-3-
carbonitrile

| | |
|---|---|
| 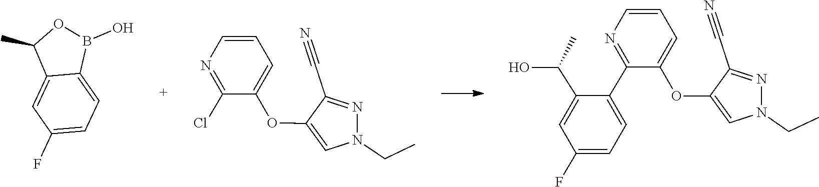 [SMILES: C[C@H]1OB(O)c2ccc(F)cc21 ; CCn1cc(Oc2cccnc2Cl)c(C#N)n1 ; CCn1cc(Oc2cccnc2-c2ccc(F)cc2[C@@H](C)O)c(C#N)n1] | 351 [M + H]⁺ |

(R)-1-cyclobutyl-4-((3-(4-fluoro-2-(1-hydroxyethyl)phenyl)-1-methyl-1H-pyrazol-4-
yl)methyl)-1H-pyrazole-3-carbonitrile

| | |
|---|---|
| 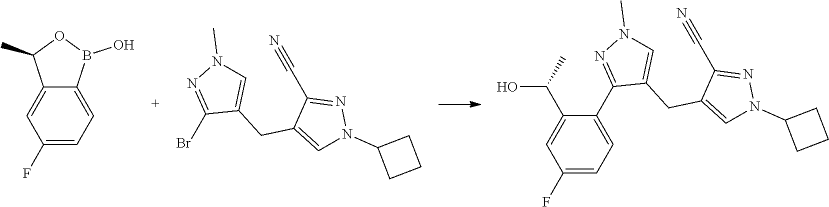 [SMILES: C[C@H]1OB(O)c2ccc(F)cc21 ; Cn1cc(Cc2cn(C3CCC3)nc2C#N)c(Br)n1 ; C[C@@H](O)c1cc(F)ccc1-c1nn(C)cc1Cc1cn(C2CCC2)nc1C#N] | 380 [M + H]⁺ |

(R)-1-(cyclobutylmethyl)-4-((3-(4-fluoro-2-(1-hydroxyethyl)phenyl)-1-methyl-1H-pyrazol-4-
yl)methyl)-1H-pyrazole-3-carbonitrile

| | |
|---|---|
| 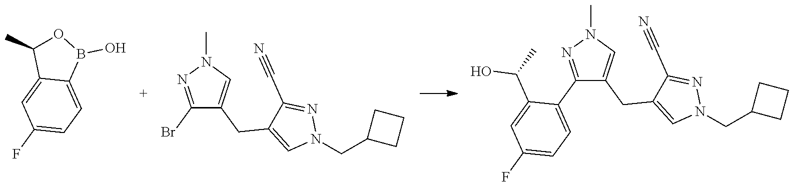 [SMILES: C[C@H]1OB(O)c2ccc(F)cc21 ; Cn1cc(Cc2cn(CC3CCC3)nc2C#N)c(Br)n1 ; C[C@@H](O)c1cc(F)ccc1-c1nn(C)cc1Cc1cn(CC2CCC2)nc1C#N] | 394 [M + H]⁺ |

(R)-1-(cyclopropylmethyl)-4-((2-(4-fluoro-2-(1-hydroxyethyl)phenyl)pyridin-3-yl)methyl)-1H-
pyrazole-3-carbonitrile

| | |
|---|---|
| 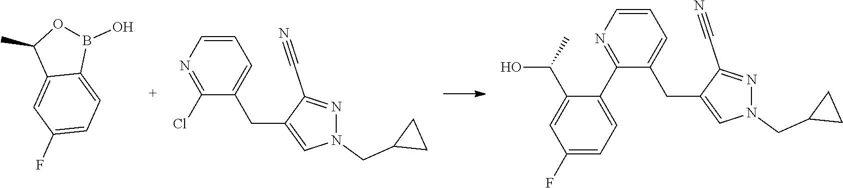 [SMILES: C[C@H]1OB(O)c2ccc(F)cc21 ; N#Cc1nn(CC2CC2)cc1Cc1cccnc1Cl ; C[C@@H](O)c1cc(F)ccc1-c1ncccc1Cc1cn(CC2CC2)nc1C#N] | 377 [M + H]⁺ |

(R)-(1-ethyl-1H-pyrazol-4-yl)(3-(4-fluoro-2-(1-hydroxyethyl)phenyl)-1-methyl-1H-pyrazol-4-
yl)methanone

| | |
|---|---|
| 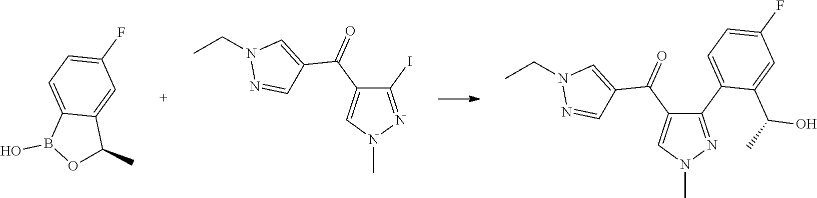 [SMILES: C[C@H]1OB(O)c2ccc(F)cc21 ; CCn1cc(C(=O)c2cn(C)nc2I)cn1 ; CCn1cc(C(=O)c2cn(C)nc2-c2ccc(F)cc2[C@@H](C)O)cn1] | 343 [M + H]⁺ |

(R)-1-ethy1-4-((3-(4-fluoro-2-(1-hydroxyethyl)phenyl)-1-methyl-1H-pyrazol-4-yl)methyl)-1H-
pyrazole-3-carbonitrile

| | |
|---|---|
| 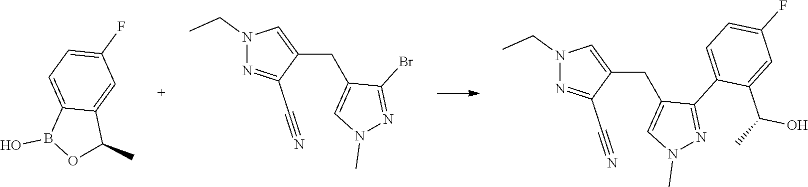 [SMILES: C[C@H]1OB(O)c2ccc(F)cc21 ; CCn1cc(Cc2cn(C)nc2Br)c(C#N)n1 ; CCn1cc(Cc2cn(C)nc2-c2ccc(F)cc2[C@@H](C)O)c(C#N)n1] | 354 [M + H]⁺ |

(R)-1-(cyclopropylmethyl)-4-((3-(4-fluoro-2-(1-hydroxyethyl)phenyl)-1-methyl-1H-pyrazol-4-
yl)methyl)-1H-pyrazole-3-carbonitrile

| | |
|---|---|
| 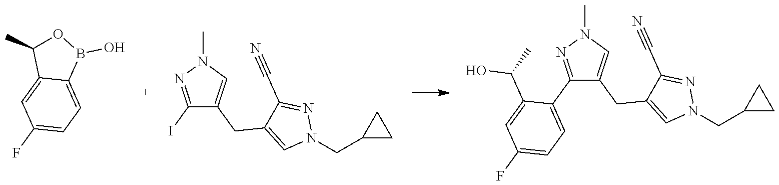 [SMILES: C[C@H]1OB(O)c2ccc(F)cc21 ; Cn1cc(Cc2cn(CC3CC3)nc2C#N)c(I)n1 ; C[C@@H](O)c1cc(F)ccc1-c1nn(C)cc1Cc1cn(CC2CC2)nc1C#N] | 380 [M + H]⁺ |

ethyl (R)-1-(cyclopropylmethyl)-4-((2-(4-fluoro-2-(1-hydroxyethyl)phenyl)pyridin-3-yl)oxy)-
1H-pyrazole-3-carboxylate

| | |
|---|---|
| 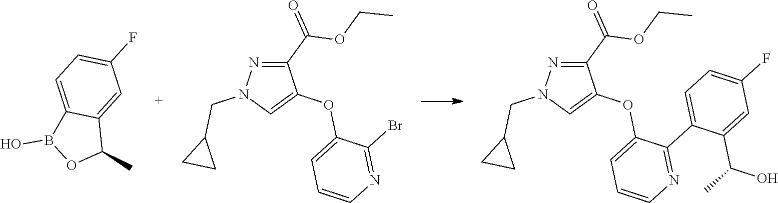 [SMILES: C[C@H]1OB(O)c2ccc(F)cc21 ; CCOC(=O)c1nn(CC2CC2)cc1Oc1cccnc1Br ; CCOC(=O)c1nn(CC2CC2)cc1Oc1cccnc1-c1ccc(F)cc1[C@@H](C)O] | 426 [M + H]⁺ |

(R)-1-(2-(4-((1-(cyclopropylmethyl)-3-(methylthio)-1H-pyrazol-4-yl)methyl)-1-methyl-1H-
pyrazol-3-yl)-5-fluorophenyl)ethan-1-ol

| | |
|---|---|
| 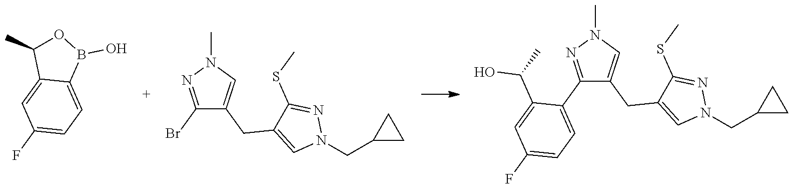 [SMILES: C[C@H]1OB(O)c2ccc(F)cc21 ; CSc1nn(CC2CC2)cc1Cc1cn(C)nc1Br ; CSc1nn(CC2CC2)cc1Cc1cn(C)nc1-c1ccc(F)cc1[C@@H](C)O] | 401 [M + H]⁺ |

1-(cyclopropylmethyl)-4-((2-(2,4-difluoro-6-((R)-1-hydroxyethyl)phenyl)pyridin-3-yl)methyl)-
N,N-dimethyl-1H-pyrazole-3-carboxamide

| | |
|---|---|
| 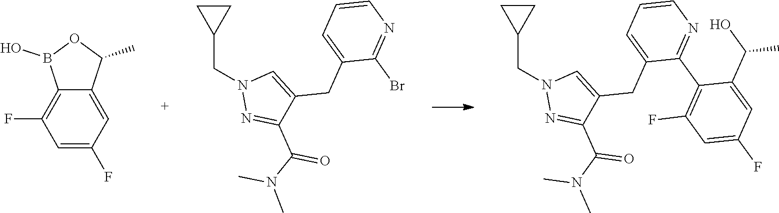 | 441 [M + H]⁺ |

4-((2-(2,4-difluoro-6-((R)-1-hydroxyethyl)phenyl)pyridin-3-yl)methyl)-1-ethyl-N,N-dimethyl-
1H-pyrazole-3-carboxamide

| | |
|---|---|
| 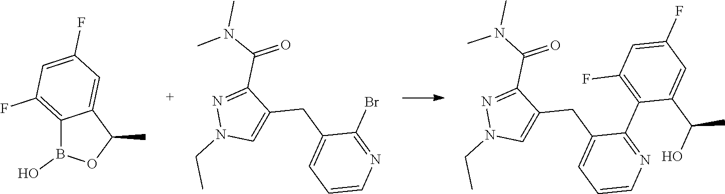 | 415 [M + H]⁺ |

(R)-1-cyclobutyl-4-((2-(4-fluoro-2-(1-hydroxyethyl)phenyl)pyridin-3-yl)methyl)-N,N-
dimethyl-1H-pyrazole-3-carboxamide

| | |
|---|---|
| 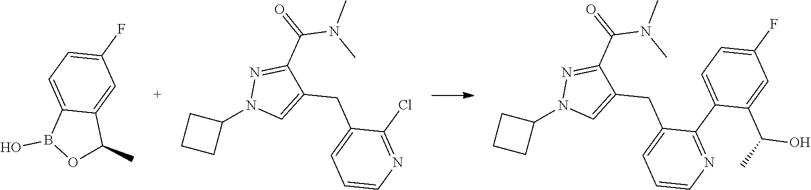 [SMILES: C[C@H]1OB(O)c2ccc(F)cc21 ; CN(C)C(=O)c1nn(C2CCC2)cc1Cc1cccnc1Cl ; C[C@@H](O)c1cc(F)ccc1-c1ncccc1Cc1cn(C2CCC2)nc1C(=O)N(C)C] | 423 [M + H]⁺ |

(R)-3-cyano-5-((2-(4-fluoro-2-(1-hydroxyethyl)phenyl)pyridin-3-yl)methyl)-N,N-dimethyl-1H-
pyrazole-1-carboxamide

| | |
|---|---|
| 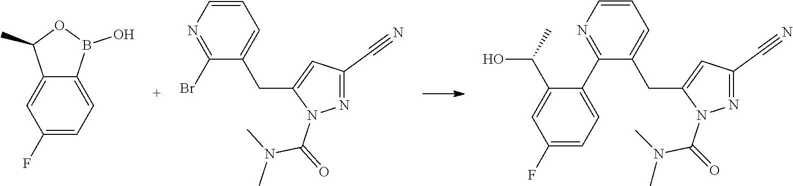 [SMILES: C[C@H]1OB(O)c2ccc(F)cc21 ; CN(C)C(=O)n1nc(C#N)cc1Cc1cccnc1Br ; C[C@@H](O)c1cc(F)ccc1-c1ncccc1Cc1cc(C#N)nn1C(=O)N(C)C] | 394 [M + H]⁺ |

(R)-3-cyclobutyl-5-((2-(4-fluoro-2-(1-hydroxyethyl)phenyl)pyridin-3-yl)methyl)-N,N-
dimethyl-1H-pyrazole-1-carboxamide

| | |
|---|---|
| 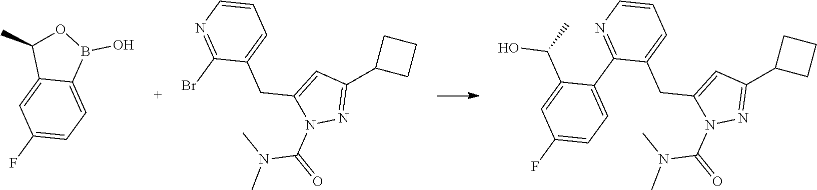 [SMILES: C[C@H]1OB(O)c2ccc(F)cc21 ; CN(C)C(=O)n1nc(C2CCC2)cc1Cc1cccnc1Br ; C[C@@H](O)c1cc(F)ccc1-c1ncccc1Cc1cc(C2CCC2)nn1C(=O)N(C)C] | 423 [M + H]⁺ |

(R)-3-(cyclopropylmethyl)-5-((2-(4-fluoro-2-(1-hydroxyethyl)phenyl)pyridin-3-yl)methyl)-
N,N-dimethyl-1H-pyrazole-1-carboxamide

| | |
|---|---|
| 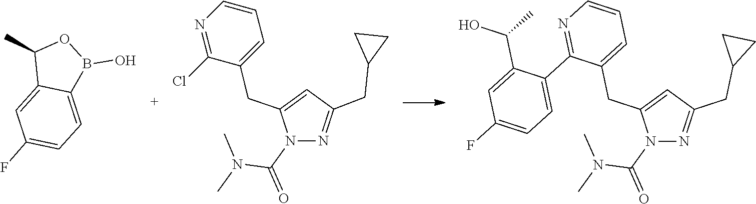 [SMILES: C[C@H]1OB(O)c2ccc(F)cc21 ; CN(C)C(=O)n1nc(CC2CC2)cc1Cc1cccnc1Cl ; C[C@@H](O)c1cc(F)ccc1-c1ncccc1Cc1cc(CC2CC2)nn1C(=O)N(C)C] | 423 [M + H]⁺ |

(R)-3-(cyclopropylmethyl)-5-((3-(2,4-difluoro-6-(1-hydroxyethyl)phenyl)-1-methyl-1H-
pyrazol-4-yl)methyl)-N,N-dimethyl-1H-pyrazole-1-carboxamide

| | |
|---|---|
| 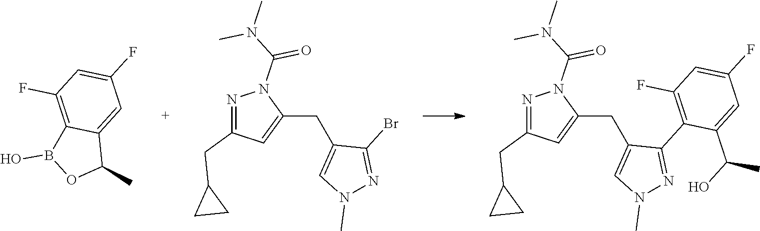 [SMILES: C[C@H]1OB(O)c2c(F)cc(F)cc21 ; CN(C)C(=O)n1nc(CC2CC2)cc1Cc1cn(C)nc1Br ; C[C@@H](O)c1cc(F)cc(F)c1-c1nn(C)cc1Cc1cc(CC2CC2)nn1C(=O)N(C)C] | 444 [M + H]⁺ |
| (R)-3-ethyl-5-((2-(4-fluoro-2-(1-hydroxyethyl)phenyl)pyridin-3-yl)methyl)-N,N-dimethyl-1H- | |

pyrazole-1-carboxamide

| | |
|---|---|
| 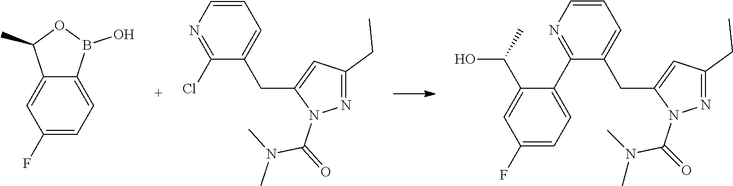 [SMILES: C[C@H]1OB(O)c2ccc(F)cc21 ; CCc1cc(Cc2cccnc2Cl)n(C(=O)N(C)C)n1 ; CCc1cc(Cc2cccnc2-c2ccc(F)cc2[C@@H](C)O)n(C(=O)N(C)C)n1] | 397 [M + H]⁺ |
| (R)-4-(cyclopropylmethyl)-1-((3-(4-fluoro-2-(1-hydroxyethyl)phenyl)-1-methyl-1H-pyrazol-4- | |
| yl)methyl)-N,N-dimethyl-1H-imidazole-2-carboxamide | |
| 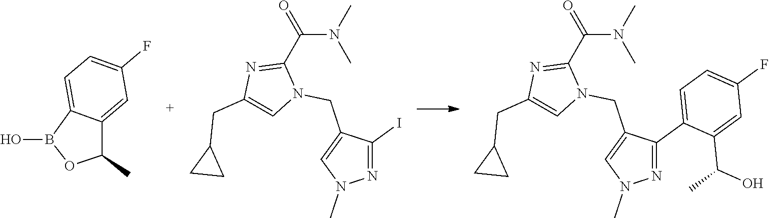 [SMILES: C[C@H]1OB(O)c2ccc(F)cc21 ; CN(C)C(=O)c1nc(CC2CC2)cn1Cc1cn(C)nc1I ; C[C@@H](O)c1cc(F)ccc1-c1nn(C)cc1Cn1cc(CC2CC2)nc1C(=O)N(C)C] | 426 [M + H]⁺ |
| 3-((2-(2,4-difluoro-6-((R)-1-hydroxyethyl)phenyl)pyridin-3-yl)(methoxymethoxy)methyl)-1- | |
| methyl-1H-pyrazole-5-carbonitrile | |
| 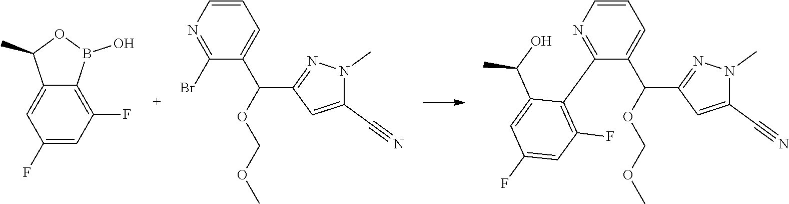 [SMILES: C[C@H]1OB(O)c2c(F)cc(F)cc21 ; COCOC(c1cc(C#N)n(C)n1)c1cccnc1Br ; COCOC(c1cc(C#N)n(C)n1)c1cccnc1-c1c(F)cc(F)cc1[C@@H](C)O] | 415 [M + H]⁺ |

### Synthesis of 5-((2-chloro-6-methylpyridin-3-yl)methyl)-1-((2-(trimethylsilyl)ethoxy)methyl)-1H-pyrazole-3-carbonitrile

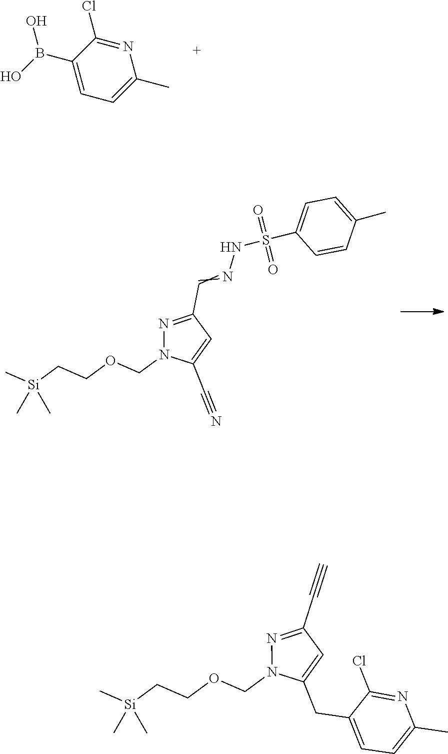

To a solution of N′-((5-cyano-1-((2-(trimethylsilyl)ethoxy)methyl)-1H-pyrazol-3-yl)methylene)-4-methylbenzenesulfonohydrazide (400 mg, 0.95 mmol) in dioxane (5 mL) was added (2-chloro-6-methylpyridin-3-yl)boronic acid (250 mg, 1.43 mmol) and K₂CO₃ (72.5 mg, 0.0630 mmol), then degassed and purged with N₂ three times. The mixture was stirred at 100° C. for 16 h. The mixture was diluted with water (10 mL) and extracted with EtOAc (20 mL×3). The combined organic phases were washed with brine (20 mL×3), dried over anhydrous Na₂SO₄ and concentrated. The residue was purified by flash column chromatography on silica gel (0→20% EtOAc in PE) to give 5-((2-chloro-6-methylpyridin-3-yl)methyl)-1-((2-(trimethylsilyl)ethoxy)methyl)-1H-pyrazole-3-carbonitrile (120 mg, yield: 34.8%) as a yellow oil. LC/MS (ESI) m/z: 363 [M+H]⁺.

The following intermediate was synthesized using a similar protocol (with m/z (ESI) values):

3-((2-bromopyridin-3-yl)methyl)-1-((2-(trimethylsilyl)ethoxy)methyl)-1H-pyrazole-5-
carbonitrile

| | |
|---|---|
| 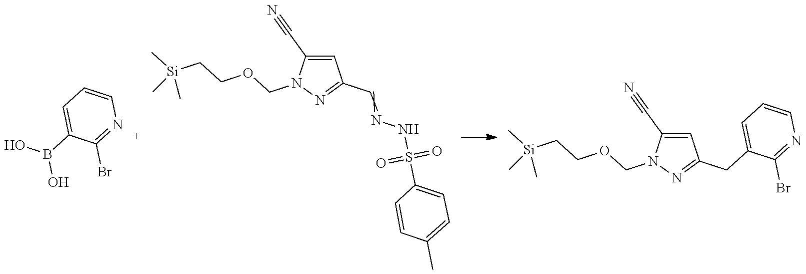 [SMILES: OB(O)c1cccnc1Br ; Cc1ccc(S(=O)(=O)NN=Cc2cc(C#N)n(COCC[Si](C)(C)C)n2)cc1 ; C[Si](C)(C)CCOCn1nc(Cc2cccnc2Br)cc1C#N] | 393 [M + H]⁺ |

### Synthesis of 3-((2-bromopyridin-3-yl)(hydroxy)methyl)-1-((2-(trimethylsilyl)ethoxy)methyl)-1H-pyrazole-5-carbonitrile

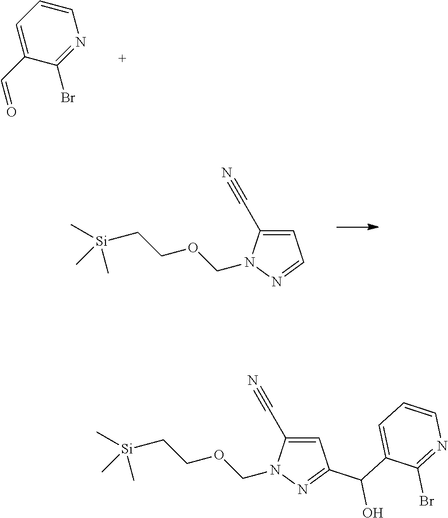 [SMILES: O=Cc1cccnc1Br ; C[Si](C)(C)CCOCn1nccc1C#N ; C[Si](C)(C)CCOCn1nc(C(O)c2cccnc2Br)cc1C#N]

To a mixture of 1-((2-(trimethylsilyl)ethoxy)methyl)-1H-pyrazole-5-carbonitrile (3.00 g, 15.1 mmol) in THF (30 mL) was added TMPMgCl·LiCl (1 M in heptane, 21.5 mL, 21.5 mmol) under N₂ at −20° C. and the mixture was stirred at −20° C. for 1 h. Then a solution of 2-bromopyridine-3-carbaldehyde (3.30 g, 17.9 mmol) in THE (10 mL) was added at −20° C. The mixture was stirred for 1 h at −20° C. The mixture was quenched with sat. aq. NH₄Cl (50 mL) and extracted with EtOAc (100 mL×3). The combined organic phase was washed with brine (40 mL×2), dried over anhydrous Na₂SO₄, and concentrated. The residue was purified by flash column chromatography on silica gel (0→50% EtOAc in PE) to give 3-((2-bromopyridin-3-yl)(hydroxy)methyl)-1-((2-(trimethylsilyl)ethoxy)methyl)-1H-pyrazole-5-carbonitrile (4.00 g, 54.0% yield) as a colorless oil. LCMS (ESI): m/z: 409 [M+H]⁺.

The following intermediate was synthesized using a similar protocol (with m/z (ESI) values):

3-((2,4-dibromothiazol-5-yl)(hydroxy)methyl)-1-((2-(trimethylsilyl)ethoxy)methyl)-1H-
pyrazole-5-carbonitrile

| | |
|---|---|
| 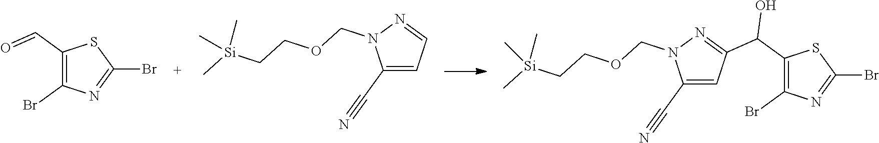 [SMILES: O=Cc1sc(Br)nc1Br ; C[Si](C)(C)CCOCn1nccc1C#N ; C[Si](C)(C)CCOCn1nc(C(O)c2sc(Br)nc2Br)cc1C#N] | 493 [M + H]⁺ |

### Synthesis of (R)-3-((2-(4-fluoro-2-(1-hydroxyethyl)phenyl)pyridin-3-yl)oxy)-1-methyl-1H-pyrazole-5-carbonitrile

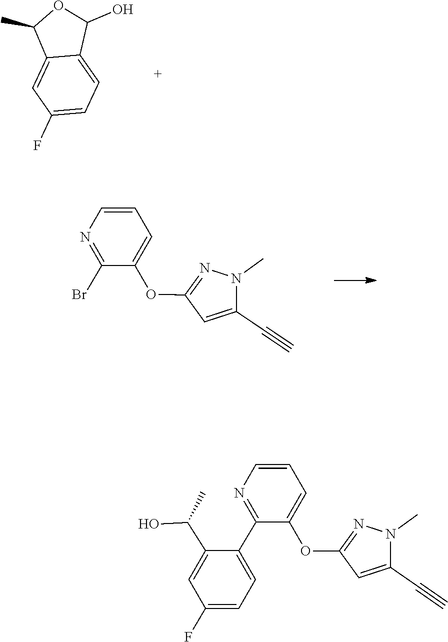 [SMILES: C[C@H]1OC(O)c2ccc(F)cc21 ; C#Cc1cc(Oc2cccnc2Br)nn1C ; C#Cc1cc(Oc2cccnc2-c2ccc(F)cc2[C@@H](C)O)nn1C]

To a solution of 3-((2-bromopyridin-3-yl)oxy)-1-methyl-1H-pyrazole-5-carbonitrile (165 mg, 0.591 mmol), (R)-5-fluoro-3-methylbenzo[c][1,2]oxaborol-1(3H)-ol (147 mg, 0.887 mmol), K₃PO₄ (376 mg, 1.77 mmol) and BI-DIME (39.1 mg, 0.118 mmol) in toluene (10 mL) and H₂O (2 mL) were added Pd₂(dba)₃ (54.1 mg, 0.0590 mmol). The mixture was degassed with N₂ three times and stirred at 100° C. for 3.5 h. The mixture was filtered, and the filtrate was concentrated. The residue was purified by flash column chromatography on silica gel (0-8% MeOH in DCM) to give (R)-3-((2-(4-fluoro-2-(1-hydroxyethyl)phenyl)pyridin-3-yl)oxy)-1-methyl-1H-pyrazole-5-carbonitrile (155 mg, yield: 77.5%) as a yellow solid. LC/MS ESI (m/z): 339 [M+H]⁺.

The following intermediates were synthesized using a similar protocol (with m/z (ESI) values):

3-(2-(2,4-difluoro-6-((R)-1-hydroxyethyl)phenyl)nicotinoyl)-1-((2-
(trimethylsilyl)ethoxy)methyl)-1H-pyrazole-5-carbonitrile

| | |
|---|---|
| 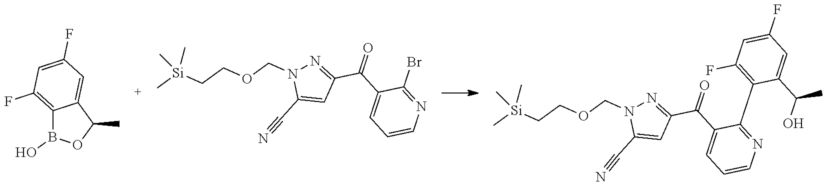 [SMILES: C[C@H]1OB(O)c2c(F)cc(F)cc21 ; C[Si](C)(C)CCOCn1nc(C(=O)c2cccnc2Br)cc1C#N ; C[C@@H](O)c1cc(F)cc(F)c1-c1ncccc1C(=O)c1cc(C#N)n(COCC[Si](C)(C)C)n1] | 485 [M + H]⁺ |
| (R)-3-(3-(2,4-difluoro-6-(1-hydroxyethyl)phenyl)-1-methyl-1H-pyrazole-4-carbonyl)-1-methyl- | |
| 1H-pyrazole-5-carbonitrile | |
| 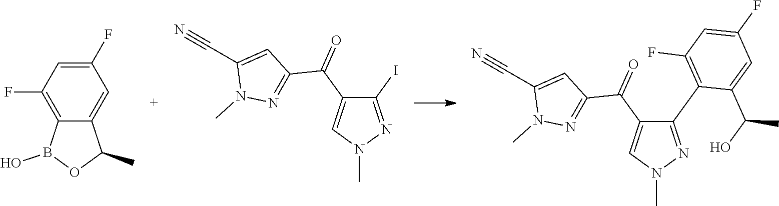 [SMILES: C[C@H]1OB(O)c2c(F)cc(F)cc21 ; Cn1cc(C(=O)c2cc(C#N)n(C)n2)c(I)n1 ; C[C@@H](O)c1cc(F)cc(F)c1-c1nn(C)cc1C(=O)c1cc(C#N)n(C)n1] | 372 [M + H]⁺ |
| (R)-3-cyano-5-((4-(4-fluoro-2-(1-hydroxyethyl)phenyl)-2-methylthiazol-5-yl)methyl)-N,N- | |
| dimethyl-1H-pyrazole-1-carboxamide | |
| 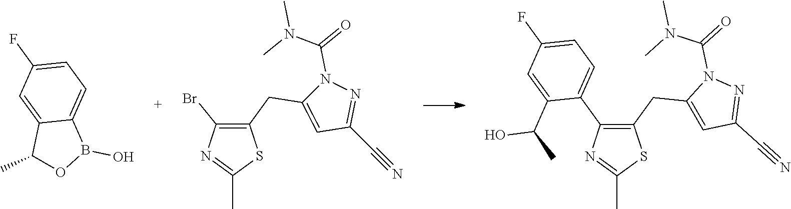 [SMILES: C[C@H]1OB(O)c2ccc(F)cc21 ; Cc1nc(Br)c(Cc2cc(C#N)nn2C(=O)N(C)C)s1 ; Cc1nc(-c2ccc(F)cc2[C@@H](C)O)c(Cc2cc(C#N)nn2C(=O)N(C)C)s1] | 414 [M + H]⁺ |
| 3-((2-(2,4-difluoro-6-((R)-1-hydroxyethyl)phenyl)pyridin-3-yl)oxy)-1-methyl-1H-pyrazole-5- | |
| carbonitrile | |
| 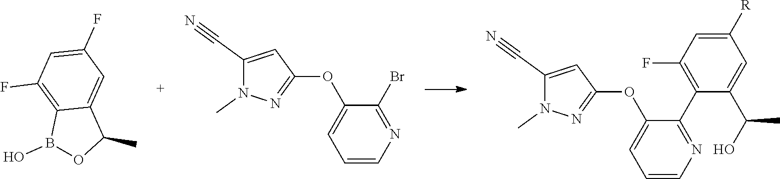 | 357 [M + H]⁺ |
| (R)-5-((2-(4-fluoro-2-(1-hydroxyethyl)phenyl)-6-methylpyridin-3-yl)methyl)-1-((2- | |
| (trimethylsilyl)ethoxy)methyl)-1H-pyrazole-3-carbonitrile | |
| 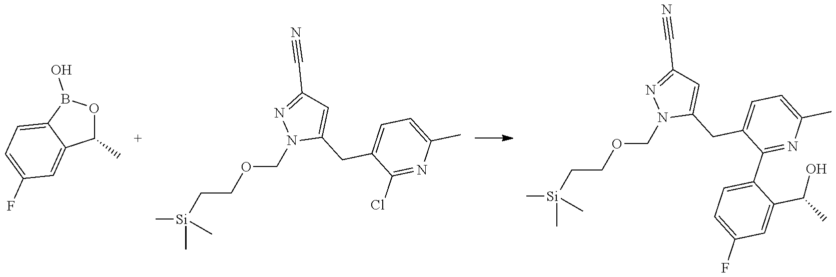 | 467 [M + H]⁺ |

### Synthesis of 3-(3-iodo-1-methyl-1H-pyrazole-4-carbonyl)-1-((2-(trimethylsilyl)ethoxy)methyl)-1H-pyrazole-5-carbonitrile

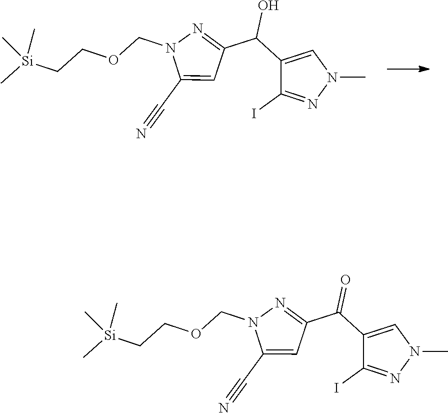

To a solution of 3-(hydroxy(3-iodo-1-methyl-1H-pyrazol-4-yl)methyl)-1-((2-(trimethylsilyl)ethoxy)methyl)-1H-pyrazole-5-carbonitrile (1.29 g, 2.80 mmol) in DCM (10 mL) was added Dess-Martin periodinane (2.38 g, 5.61 mmol) at 0° C. and stirred for 1 h at 0° C. The reaction was quenched with sat. aq. NaHCO₃ (5 mL) and sat. aq. Na₂S₂O₃ (5 mL). The combined organic phase was washed with brine (20 mL×2), dried with anhydrous Na₂SO₄ and concentrated. The residue was purified by flash column chromatography on silica gel (30→50% EtOAc in PE) to afford 3-(3-iodo-1-methyl-1H-pyrazole-4-carbonyl)-1-((2-(trimethylsilyl)ethoxy)methyl)-1H-pyrazole-5-carbonitrile (878 mg, yield: 68.6%) as a yellow oil. LC/MS (ESI) (m/z): 458 [M+H]⁺.

### Synthesis of 3-(4-bromo-1,3-thiazole-5-carbonyl)-1-{[2-(trimethylsilyl)ethoxy]methyl}-1H-pyrazole-5-carbonitrile and 3-(4-bromo-2-methylthiazole-5-carbonyl)-1-((2-(trimethylsilyl)ethoxy)methyl)-1H-pyrazole-5-carbonitrile

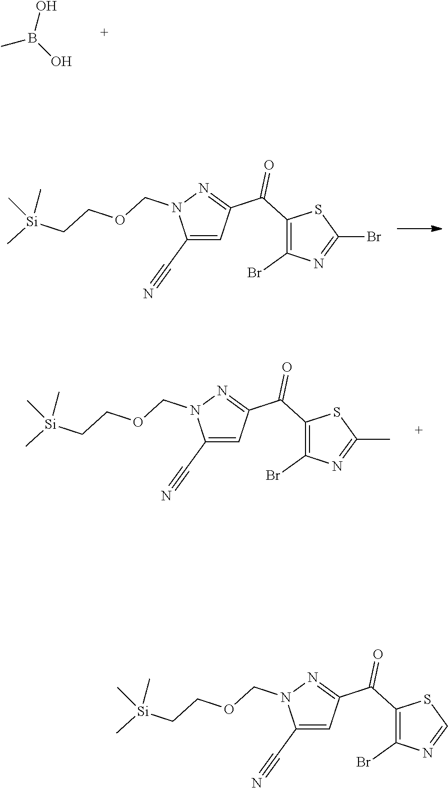

To a stirred solution of 3-(2,4-dibromothiazole-5-carbonyl)-1-((2-(trimethylsilyl)ethoxy)methyl)-1H-pyrazole-5-carbonitrile (500 mg, 1.01 mmol) and methylboronic acid (92 mg, 1.52 mmol) in dioxane (20 mL) were added K₂CO₃ (420 mg, 3.04 mmol) and Pd(PPh₃)₄ (117 mg, 0.10 mmol). The mixture was degassed and purged with N₂ three times, then stirred at 100° C. for 12 h. The reaction was filtered, and the filter cake was washed with EtOAc (30 mL). The filtrate was washed with water (15 mL) and brine (15 mL). The combined organic phase was dried over anhydrous Na₂SO₄ and concentrated. The residue was purified by flash column chromatography on silica gel (0→30% EtOAc in PE) to give 3-(4-bromo-2-methylthiazole-5-carbonyl)-1-((2-(trimethylsilyl)ethoxy)methyl)-1H-pyrazole-5-carbonitrile and 3-(4-bromo-1,3-thiazole-5-carbonyl)-1-{[2-(trimethylsilyl)ethoxy]methyl}-1H-pyrazole-5-carbonitrile (100 mg, yield: 23%) as a white solid mixture. LC/MS (ESI) (m/z): 427 [M+H]⁺.

The following intermediate was synthesized using a similar protocol (with m/z (ESI) values):

5-((4-bromo-2-methylthiazol-5-yl)methyl)-3-cyano-N,N-dimethyl-1H-pyrazole-1-carboxamide

| | |
|---|---|
| 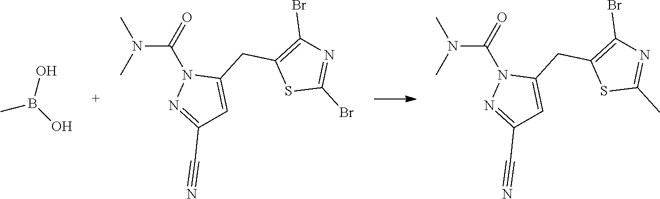 | 354 [M + H]⁺ |

### Synthesis of 3-(2-chloronicotinoyl)-1H-pyrazole-5-carbonitrile

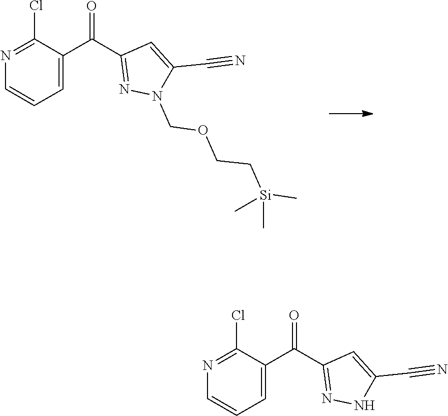 [SMILES: C[Si](C)(C)CCOCn1nc(C(=O)c2cccnc2Cl)cc1C#N ; N#Cc1cc(C(=O)c2cccnc2Cl)n[nH]1]

To a solution of 3-(2-chloronicotinoyl)-1-((2-(trimethylsilyl)ethoxy)methyl)-1H-pyrazole-5-carbonitrile (960 mg, 2.65 mmol) in DCM (6 mL) was added TFA (3 mL, 40.4 mmol). The mixture was stirred at r.t. for 2 h and then concentrated. The residue was diluted with EtOAc (10 mL) and sat. aq. NaHCO₃ (20 mL). The mixture extracted with EtOAc (30 mL×3). The combined organic phases were washed with brine (20 mL×2), dried over anhydrous Na₂SO₄, filtered and concentrated. The residue was purified by flash column chromatography on silica gel (0→10% MeOH in DCM) to 3-(2-chloronicotinoyl)-1H-pyrazole-5-carbonitrile (583 mg, yield: 94.7%) as a yellow solid. LC/MS ESI (m/z): 233 [M+H]⁺.

The following intermediates were synthesized using a similar protocol (with m/z (ESI) values):

3-((2-bromopyridin-3-yl)methyl)-1H-pyrazole-5-carbonitrile

| | |
|---|---|
| 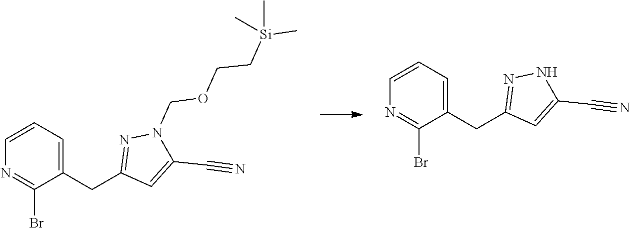 [SMILES: C[Si](C)(C)CCOCn1nc(Cc2cccnc2Br)cc1C#N ; N#Cc1cc(Cc2cccnc2Br)n[nH]1] | 263 [M + H]⁺ |

3-(3-iodo-1-methyl-1H-pyrazole-4-carbonyl)-1H-pyrazole-5-carbonitrile

| | |
|---|---|
| 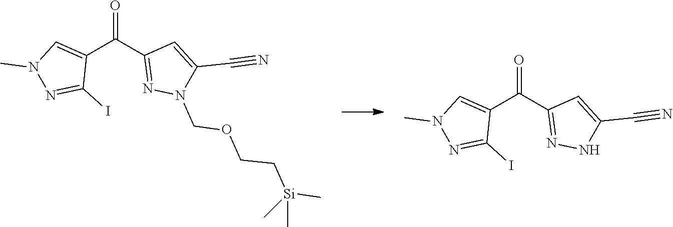 | 328 [M + H]⁺ |
| (R)-5-((2-(2-(1-((5-bromo-2-nitropyridin-3-yl)oxy)ethyl)-4-fluorophenyl)-6-methylpyridin-3- | |
| yl)methyl)-1H-pyrazole-3-carbonitrile | |
| 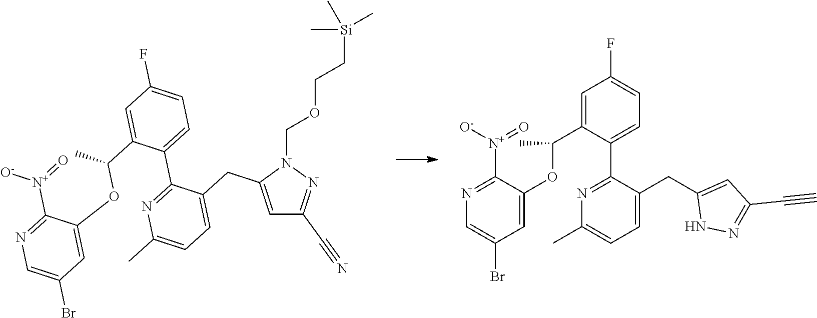 [SMILES: Cc1ccc(Cc2cc(C#N)nn2COCC[Si](C)(C)C)c(-c2ccc(F)cc2[C@@H](C)Oc2cc(Br)cnc2[N+](=O)[O-])n1 ; C#Cc1cc(Cc2ccc(C)nc2-c2ccc(F)cc2[C@@H](C)Oc2cc(Br)cnc2[N+](=O)[O-])[nH]n1] | 537 [M + H]⁺ |
| 3-(2-bromonicotinoyl)-1H-pyrazole-5-carbonitrile | |
| 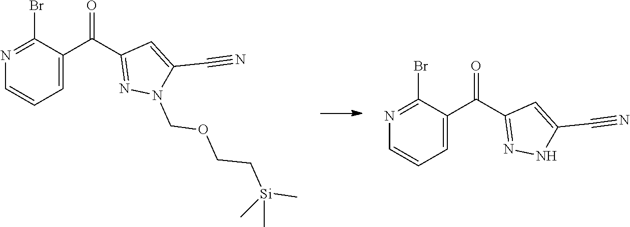 [SMILES: C[Si](C)(C)CCOCn1nc(C(=O)c2cccnc2Br)cc1C#N ; N#Cc1cc(C(=O)c2cccnc2Br)n[nH]1] | 277 [M + H]⁺ |

### Synthesis of 5-((2-chloropyridin-3-yl)methyl)-3-ethyl-N,N-dimethyl-1H-pyrazole-1-carboxamide

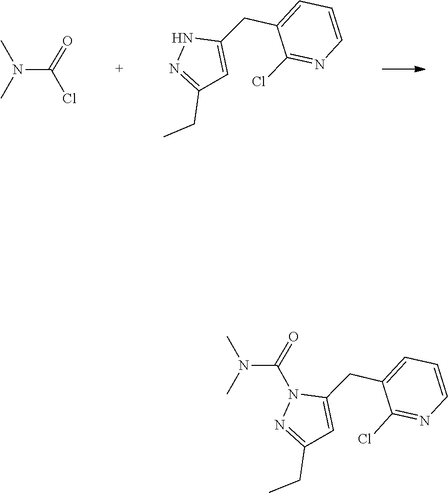

To a solution of 2-chloro-3-((3-ethyl-1H-pyrazol-5-yl)methyl)pyridine (165 mg, 0.744 mmol) and TEA (0.0600 mL, 0.433 mmol) in toluene (10 mL) was added dimethylazanecarbonyl chloride (0.0690 mL, 0.744 mmol). The reaction mixture was stirred at 100° C. for 16 h. The reaction mixture was added to water (10 mL) and extracted with EtOAc (30 mL×3). The combined organic layers were washed with brine (20 mL×2), dried by anhydrous Na₂SO₄ and concentrated. The residue was purified by flash column chromatography on silica gel (0→100% EtOAc in PE, then 0→10% MeOH in DCM) to give a mixture of regioisomers. The regioisomers were separated by SFC (ChiralPak IB, 100×4.6 mm ID., 5 μm; 40% MeOH+0.05% DEA in C02) to give 5-[(2-chloropyridin-3-yl)methyl]-3-ethyl-N,N-dimethylpyrazole-1-carboxamide (100 mg, yield: 45.9%) as a white solid. LC/MS ESI (m/z): 293 [M+H]⁺.

The following intermediates were synthesized using a similar protocol (with m/z (ESI) values):

5-((2-chloropyridin-3-yl)methyl)-3-(cyclopropylmethyl)-N,N-dimethyl-1H-pyrazole-1-
carboxamide

| | |
|---|---|
| 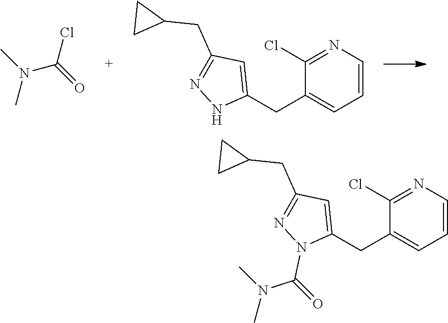 | 319 [M + H]⁺ |

5-((3-bromo-1-methyl-1H-pyrazol-4-yl)methyl)-3-(cyclopropylmethyl)-N,N-dimethyl-1H-
pyrazole-1-carboxamide

| | |
|---|---|
| 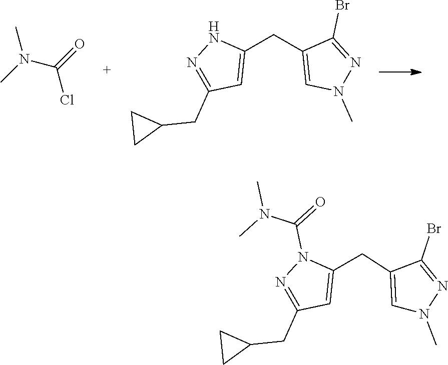 | 366 [M + H]⁺ |

### Synthesis of 3-(4-bromo-1,3-thiazole-5-carbonyl)-1-methyl-1H-pyrazole-5-carbonitrile and 3-(4-bromo-2-methylthiazole-5-carbonyl)-1-methyl-1H-pyrazole-5-carbonitrile

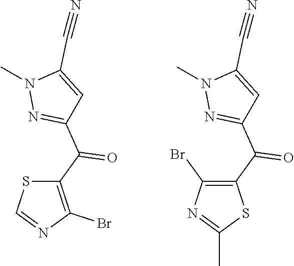 [SMILES: Cn1nc(C(=O)c2scnc2Br)cc1C#N ; Cc1nc(Br)c(C(=O)c2cc(C#N)n(C)n2)s1]

A mixture of 3-(4-bromo-2-methylthiazole-5-carbonyl)-1-((2-(trimethylsilyl)ethoxy)methyl)-1H-pyrazole-5-carbonitrile and 3-(4-bromo-1,3-thiazole-5-carbonyl)-1-{[2-(trimethylsilyl)ethoxy]methyl}-1H-pyrazole-5-carbonitrile (100 mg, 0.20 mmol) and TFA (5 mL) was stirred at 25° C. for 3 h. The reaction mixture was concentrated. The residue was dissolved in MeCN (3 mL) and aq. NH₃ (1 mL). The solution was stirred at 25° C. for 0.5 h. The mixture was diluted with H₂O (20 mL) and extracted with EtOAc (15 mL×3). The combined organic phases were washed with brine (30 mL×3), dried over anhydrous Na₂SO₄ and concentrated. The residue was purified by column chromatography on silica gel (0→30% EtOAc in PE) to give 5-(4-bromo-2-methylthiazole-5-carbonyl)-1H-pyrazole-3-carbonitrile and 3-(4-bromo-1,3-thiazole-5-carbonyl)-1H-pyrazole-5-carbonitrile (30 mg, crude) as a yellow oil mixture. LC/MS (ESI) (m/z): 297 [M+H]⁺.

To a mixture of 5-(4-bromo-2-methylthiazole-5-carbonyl)-1H-pyrazole-3-carbonitrile and 3-(4-bromo-1,3-thiazole-5-carbonyl)-1H-pyrazole-5-carbonitrile (90 mg, 0.25 mmol) and K₂CO₃ (69 mg, 0.49 mmol) in DMF (1 mL) was added iodomethane (39 mg, 0.27 mmol) dropwise at 0° C. The reaction was stirred at 25° C. for 16 h and then concentrated. The residue was diluted with EtOAc (15 mL), washed with brine (10 mL), dried over Na₂SO₄ and concentrated. The residue was purified by column chromatography on silica gel (0→25% EtOAc in PE) to give 3-(4-bromo-2-methylthiazole-5-carbonyl)-1-methyl-1H-pyrazole-5-carbonitrile and 3-(4-bromo-1,3-thiazole-5-carbonyl)-1-methyl-1H-pyrazole-5-carbonitrile (60 mg, yield: 64.5%) as a yellow oil mixture. LC/MS (ESI) (m/z): 311 [M+H]⁺.

### Synthesis of 3-cyano-5-((2,4-dibromothiazol-5-yl)methyl)-N,N-dimethyl-1H-pyrazole-1-carboxamide

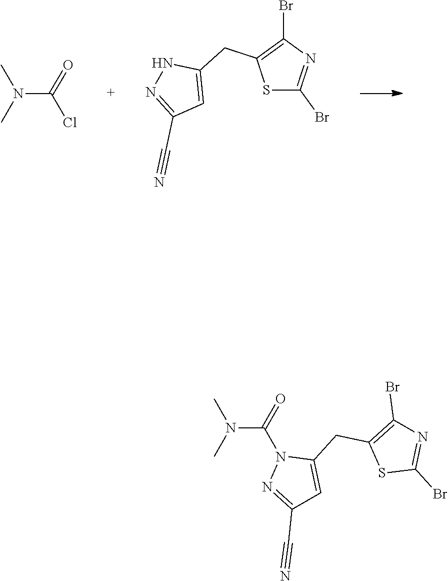

To a solution of 5-((2,4-dibromothiazol-5-yl)methyl)-1H-pyrazole-3-carbonitrile (550 mg, 1.58 mmol) in THF (10 mL) was added dimethylcarbamic chloride (340 mg, 3.16 mmol), TEA (0.659 mL, 4.74 mmol) and DMAP (96.5 mg, 0.790 mmol). The mixture was degassed and purged with N₂ three times, then stirred at 70° C. for 3 h. The mixture was quenched with H₂O (10 mL) and extracted with EtOAc (30 mL×3). The combined organic layer was dried over anhydrous Na₂SO₄ and concentrated. The residue was purified by flash column chromatography on silica gel (10→50% EtOAc in PE) to afford 3-cyano-5-((2,4-dibromothiazol-5-yl)methyl)-N,N-dimethyl-1H-pyrazole-1-carboxamide (600 mg, yield: 90.6%) as a yellow oil. LC/MS (ESI) m/z: 418 [M+H]⁺.

The following intermediates were synthesized using a similar protocol (with m/z (ESI) values):

3-((2-bromopyridin-3-yl)methyl)-5-cyano-N,N-dimethyl-1H-pyrazole-1-carboxamide and 5-
((2-bromopyridin-3-yl)methyl)-3-cyano-N,N-dimethyl-1H-pyrazole-1-carboxamide

| | |
|---|---|
| 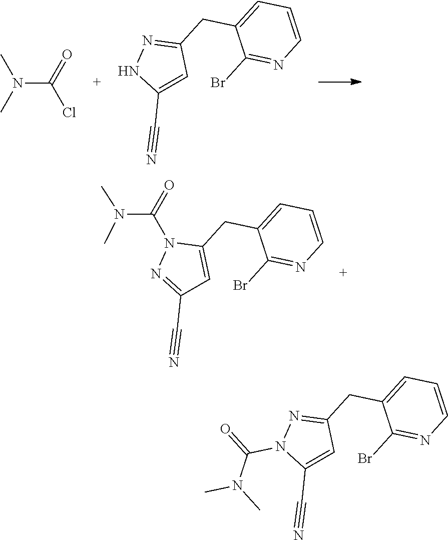 | 334 [M + H]⁺ |

5-((2-bromopyridin-3-yl)methyl)-3-cyano-N,N-dimethyl-1H-pyrazole-1-carboxamide

| | |
|---|---|
| 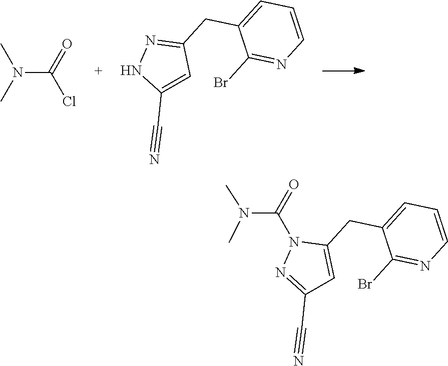 | 334 [M + H]⁺ |

5-((2-bromopyridin-3-yl)methyl)-3-cyclobutyl-N,N-dimethyl-1H-pyrazole-1-carboxamide

| | |
|---|---|
| 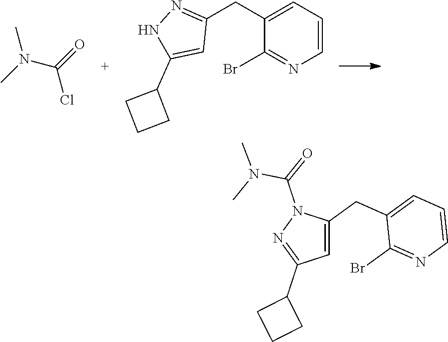 | 363 [M + H]⁺ |

(R)-5-((2-(2-(1-((5-bromo-2-nitropyridin-3-yl)oxy)ethyl)-4-fluorophenyl)-6-methylpyridin-3-
yl)methyl)-3-cyano-N,N-dimethyl-1H-pyrazole-1-carboxamide

| | |
|---|---|
| 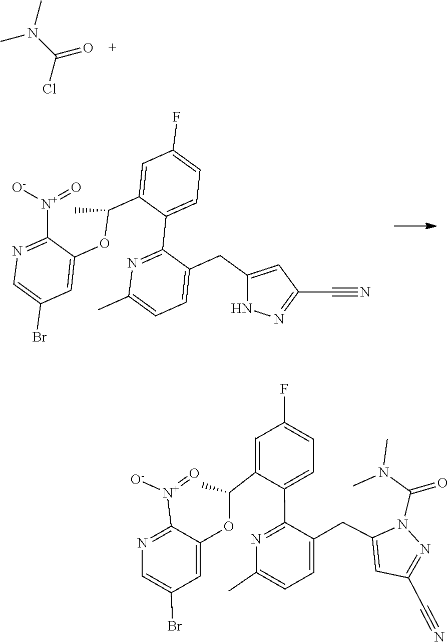 | 608 [M + H]⁺ |

### Synthesis of 3-(2-(2,4-difluoro-6-((R)-1-hydroxyethyl)phenyl)nicotinoyl)-1-methyl-1H-pyrazole-5-carbonitrile and 5-(2-{2,4-difluoro-6-[(1R)-1-hydroxyethyl]phenyl}pyridine-3-carbonyl)-1-methyl-1H-pyrazole-3-carbonitrile

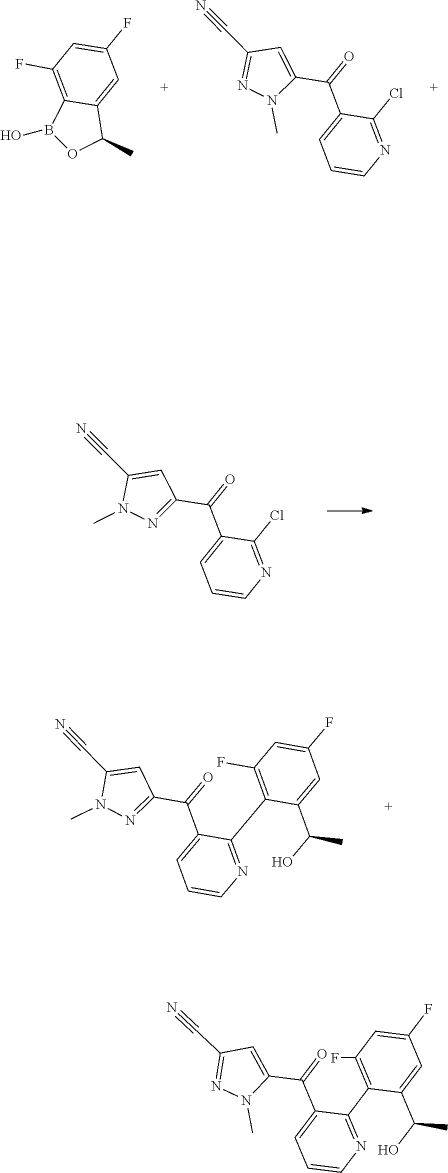 [SMILES: C[C@H]1OB(O)c2c(F)cc(F)cc21 ; Cn1nc(C#N)cc1C(=O)c1cccnc1Cl ; Cn1nc(C(=O)c2cccnc2Cl)cc1C#N ; C[C@@H](O)c1cc(F)cc(F)c1-c1ncccc1C(=O)c1cc(C#N)n(C)n1 ; C[C@@H](O)c1cc(F)cc(F)c1-c1ncccc1C(=O)c1cc(C#N)nn1C]

To a mixture of 3-(2-chloronicotinoyl)-1-methyl-1H-pyrazole-5-carbonitrile and 5-(2-chloronicotinoyl)-1-methyl-1H-pyrazole-3-carbonitrile (300 mg, 0.810 mmol), (R)-5,7-difluoro-3-methylbenzo[c][1,2]oxaborol-1(3H)-ol (270 mg, 1.22 mmol) and Na₂CO₃ (387 mg, 3.65 mmol) in 1,4-dioxane (9 mL) and water (3 mL) was added Pd(dppf)Cl₂ (89.0 mg, 0.120 mmol). The reaction was degassed with N₂ three times and stirred at 100° C. for 16 h. The mixture was filtered, and the filtrate was concentrated. The residue was purified by flash column chromatography on silica gel (0→8% MeOH in DCM) to give 3-(2-(2,4-difluoro-6-((R)-1-hydroxyethyl)phenyl)nicotinoyl)-1-methyl-1H-pyrazole-5-carbonitrile and 5-(2-{2,4-difluoro-6-[(1R)-1-hydroxyethyl]phenyl}pyridine-3-carbonyl)-1-methyl-1H-pyrazole-3-carbonitrile (70.0 mg, yield: 15.1%) as a white solid mixture. LC/MS ESI (m/z): 369 [M+H]⁺.

The following intermediates were synthesized using a similar protocol (with m/z (ESI) values):

3-cyano-5-((2-(2,4-difluoro-6-((R)-1-hydroxyethyl)phenyl)pyridin-3-yl)methyl)-N,N-dimethyl-
1H-pyrazole-1-carboxamide and 5-cyano-3-((2-(2,4-difluoro-6-((R)-1-
hydroxyethyl)phenyl)pyridin-3-yl)methyl)-N,N-dimethyl-1H-pyrazole-1-carboxamide

| | |
|---|---|
| 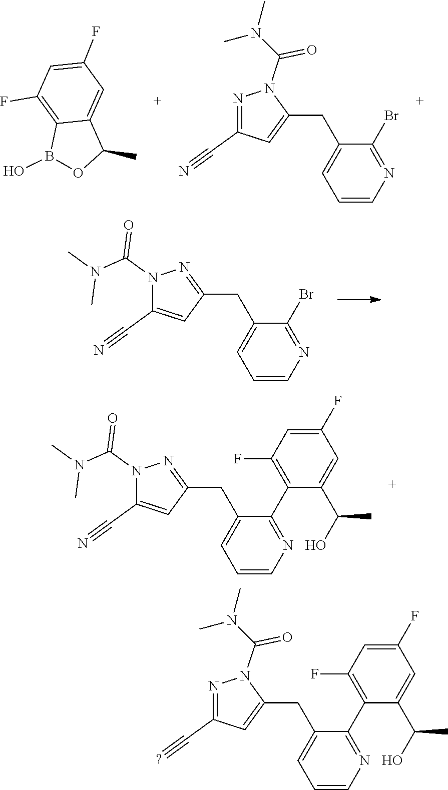 | 412 [M + H]⁺ |

### Synthesis of ethyl 1-(2-(1-((tert-butyldimethylsilyl)oxy)ethyl)-4-fluorophenyl)-3-methyl-1H-pyrazole-5-carboxylate

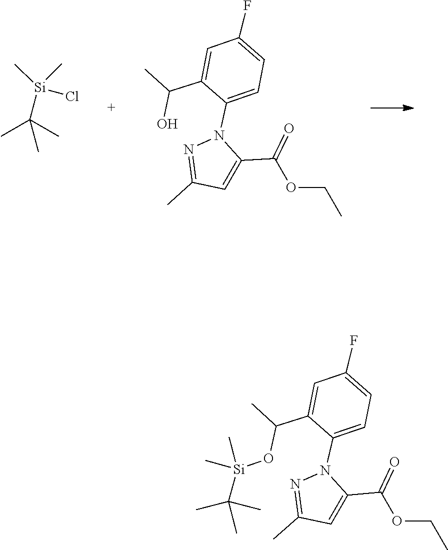

To a solution of ethyl 1-(4-fluoro-2-(1-hydroxyethyl)phenyl)-3-methyl-1H-pyrazole-5-carboxylate (2.60 g, 8.90 mmol) in DMF (15 mL) were added imidazole (2.72 g, 39.9 mmol) and tert-butylchlorodimethylsilane (4.02 g, 26.7 mmol) at 0° C. The reaction was stirred at r.t. for 1 h. The mixture was poured into water (50 mL) and extracted with EtOAc (30 mL). The organic phases were washed with brine (30 mL×2), dried over anhydrous Na₂SO₄, filtered and concentrated. The residue was purified by flash column chromatography on silica gel (0-20% EtOAc in PE) to give ethyl 1-(2-(1-((tert-butyldimethylsilyl)oxy)ethyl)-4-fluorophenyl)-3-methyl-1H-pyrazole-5-carboxylate (3.30 g, yield: 91.3%) as a transparent oil. LC/MS ESI (m/z): 407 [M+H]⁺.

### Synthesis of (Z)—N′-((5-cyano-1-((2-(trimethylsilyl)ethoxy)methyl)-1H-pyrazol-3-yl)methylene)-4-methylbenzenesulfonohydrazide

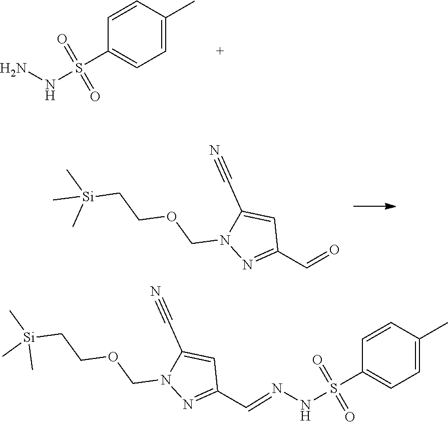

To a solution of 3-formyl-1-((2-(trimethylsilyl)ethoxy)methyl)-1H-pyrazole-5-carbonitrile (1.50 g, 5.97 mmol) in dioxane (50 mL) was added 4-methylbenzene-1-sulfonohydrazide (1.10 g, 5.91 mmol) and the reaction was stirred at 80° C. for 3 h. The reaction was concentrated. The residue was purified by flash column chromatography on silica gel (20→40% EtOAc in PE) to give (Z)—N′-((5-cyano-1-((2-(trimethylsilyl)ethoxy)methyl)-1H-pyrazol-3-yl)methylene)-4-methylbenzenesulfonohydrazide (500 mg, 99.8% yield) as a yellow oil. LC/MS (ESI) (m/z): 420 [M+H]⁺.

The following intermediate was synthesized using a similar protocol (with m/z (ESI) values):

N′-((5-cyano-1-((2-(trimethylsilyl)ethoxy)methyl)-1H-pyrazol-3-yl)methylene)-4-
methylbenzenesulfonohydrazide

| | |
|---|---|
| 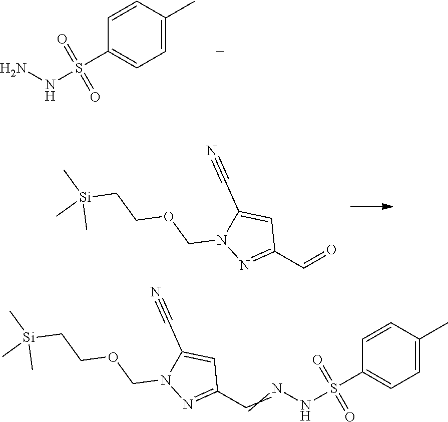 | 420 [M + H]⁺ |

### Synthesis of 1-(cyclopropylmethyl)-4-((3-(2,4-difluoro-6-(1-hydroxyethyl)phenyl)-1-methyl-1H-pyrazol-4-yl)methyl)-1H-pyrazole-3-carbonitrile

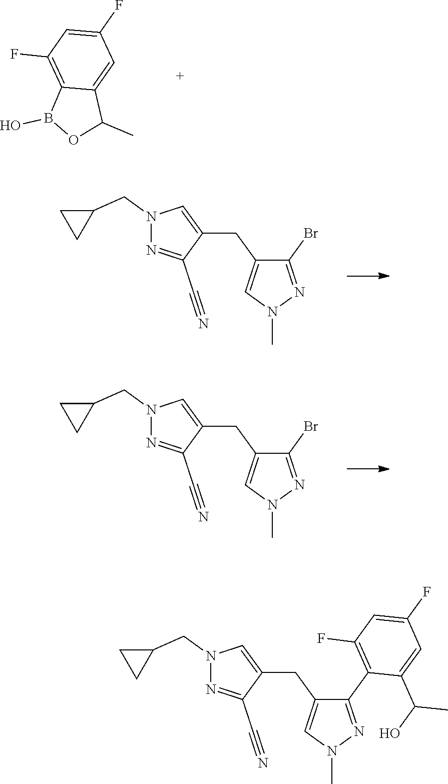 [SMILES: CC1OB(O)c2c(F)cc(F)cc21 ; Cn1cc(Cc2cn(CC3CC3)nc2C#N)c(Br)n1 ; Cn1cc(Cc2cn(CC3CC3)nc2C#N)c(Br)n1 ; CC(O)c1cc(F)cc(F)c1-c1nn(C)cc1Cc1cn(CC2CC2)nc1C#N]

To a solution of 4-((3-bromo-1-methyl-1H-pyrazol-4-yl)methyl)-1-(cyclopropylmethyl)-1H-pyrazole-3-carbonitrile (850 mg, 2.66 mmol) in 1,4-dioxane (9 mL) and water (3 mL) were added 5,7-difluoro-3-methylbenzo[c][1,2]oxaborol-1(3H)-ol (732 mg, 3.98 mmol), Na₂CO₃ (844 mg, 7.96 mmol) and X-phos G₃Pd (112 mg, 0.130 mmol). The reaction was degassed with N₂ three times and stirred at 100° C. for 2 h. The reaction was diluted with H₂O (30 mL) and extracted with EtOAc (40 mL×3). The combined organic phases were washed with brine (30 mL×2), dried over anhydrous Na₂SO₄ and concentrated. The residue was purified by flash column chromatography on silica gel (30→100% EtOAc in PE) to 1-(cyclopropylmethyl)-4-((3-(2,4-difluoro-6-(1-hydroxyethyl)phenyl)-1-methyl-1H-pyrazol-4-yl)methyl)-1H-pyrazole-3-carbonitrile (950 mg, 90.0% yield) as a black oil. LC/MS (ESI) (m/z): 398 [M+H]⁺.

### Synthesis of 4-((1-(2-bromo-4-fluorophenyl)-3-methyl-1H-pyrazol-5-yl)methyl)-1-cyclobutyl-N,N-dimethyl-1H-pyrazole-3-carboxamide

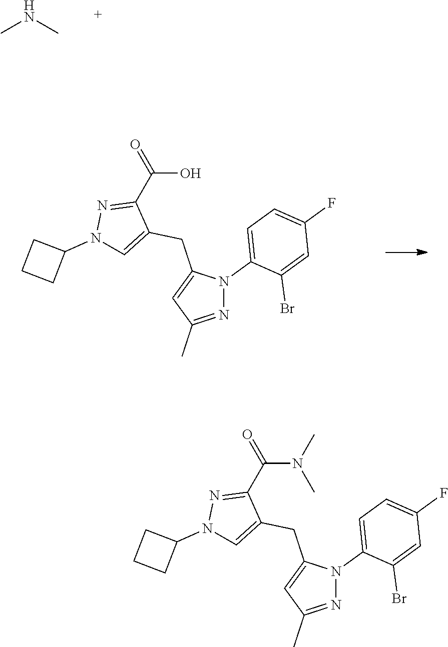

To a solution of 4-((1-(2-bromo-4-fluorophenyl)-3-methyl-1H-pyrazol-5-yl)methyl)-1-cyclobutyl-1H-pyrazole-3-carboxylic acid (330 mg, 0.762 mmol), dimethylamine (93.42 mg, 1.142 mmol) and DIEA (344.54 mg, 2.666 mmol) in DMF (4 mL) were added HATU (347.52 mg, 0.914 mmol) at 0° C. and the reaction was stirred at 25° C. for 2 h under N₂ atmosphere. The reaction was diluted with H₂O (15 mL) and extracted with EtOAc (20 mL×3). The combined organic layers were dried over anhydrous Na₂SO₄, filtered, and concentrated to give a residue. The residue was purified by column chromatography on silica gel (0→3% MeOH in DCM) to give 4-((1-(2-bromo-4-fluorophenyl)-3-methyl-1H-pyrazol-5-yl)methyl)-1-cyclobutyl-N,N-dimethyl-1H-pyrazole-3-carboxamide (312 mg, yield: 89%) as a yellow oil. LC/MS (ESI) (m/z): 460 [M+H]⁺.

### Synthesis of (R)-4-bromo-3-ethyl-5-((2-(4-fluoro-2-(1-hydroxyethyl)phenyl)pyridin-3-yl)methyl)-N,N-dimethyl-1H-pyrazole-1-carboxamide

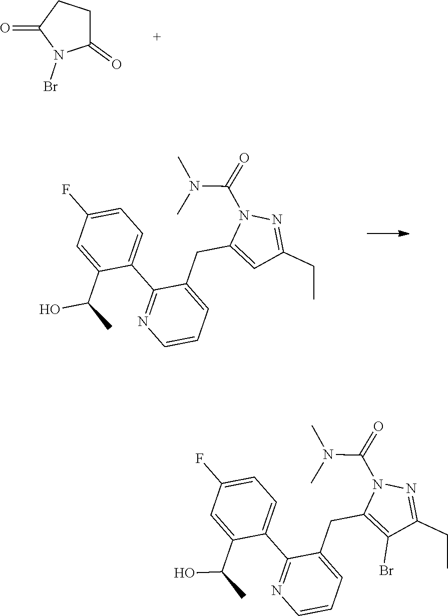 [SMILES: O=C1CCC(=O)N1Br ; CCc1cc(Cc2cccnc2-c2ccc(F)cc2[C@@H](C)O)n(C(=O)N(C)C)n1 ; CCc1nn(C(=O)N(C)C)c(Cc2cccnc2-c2ccc(F)cc2[C@@H](C)O)c1Br]

To a solution of (R)-3-ethyl-5-((2-(4-fluoro-2-(1-hydroxyethyl)phenyl)pyridin-3-yl)methyl)-N,N-dimethyl-1H-pyrazole-1-carboxamide (88.0 mg, 0.222 mmol) in THF (4 mL) was added dropwise a solution of NBS (39.5 mg, 0.222 mmol) in THF (1 mL) at 0° C. and the mixture was stirred at 0° C. for 30 min. The reaction mixture was quenched with H₂O (10 mL) and extracted with EtOAc (20 mL×2). The combined organic layers were washed with brine (10 mL), dried over anhydrous Na₂SO₄ and concentrated. The residue was purified by preparative TLC (100% EtOAc) to afford (R)-4-bromo-3-ethyl-5-((2-(4-fluoro-2-(1-hydroxyethyl)phenyl)pyridin-3-yl)methyl)-N,N-dimethyl-1H-pyrazole-1-carboxamide (100 mg, yield: 95.0%) as a yellow solid. LC/MS ESI (m/z): 475 [M+H]⁺.

The following intermediates were synthesized using a similar protocol (with m/z (ESI) values):

(R)-4-bromo-3-(cyclopropylmethyl)-5-((2-(4-fluoro-2-(1-hydroxyethyl)phenyl)pyridin-3-
yl)methyl)-N,N-dimethyl-1H-pyrazole-1-carboxamide

| | |
|---|---|
| 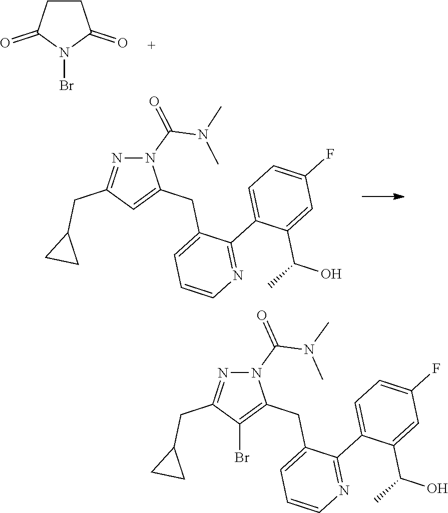 | 501 [M + H]⁺ |

(R)-4-bromo-3-(cyclopropylmethyl)-5-((3-(2,4-difluoro-6-(1-hydroxyethyl)phenyl)-1-methyl-
1H-pyrazol-4-yl)methyl)-N,N-dimethyl-1H-pyrazole-1-carboxamide

| | |
|---|---|
| 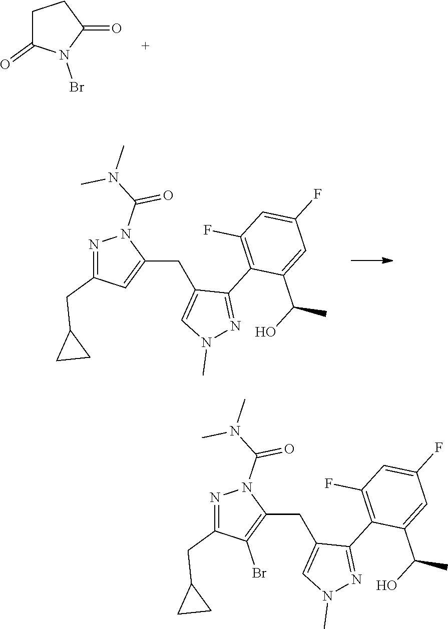 [SMILES: O=C1CCC(=O)N1Br ; C[C@@H](O)c1cc(F)cc(F)c1-c1nn(C)cc1Cc1cc(CC2CC2)nn1C(=O)N(C)C ; C[C@@H](O)c1cc(F)cc(F)c1-c1nn(C)cc1Cc1c(Br)c(CC2CC2)nn1C(=O)N(C)C] | 522 [M + H]⁺ |

### Synthesis of 4-((1-(2-acetyl-4-fluorophenyl)-3-methyl-1H-pyrazol-5-yl)methyl)-1-(cyclopropylmethyl)-1H-pyrazole-3-carbonitrile

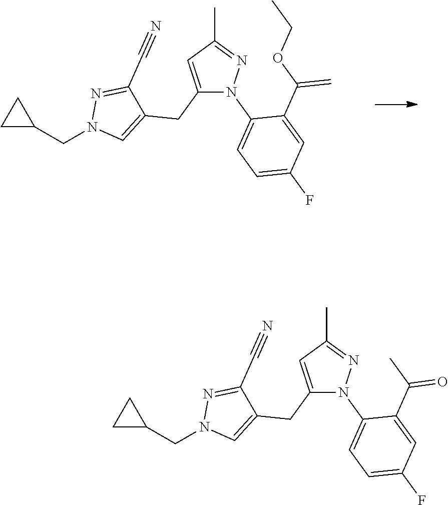 [SMILES: C=C(OCC)c1cc(F)ccc1-n1nc(C)cc1Cc1cn(CC2CC2)nc1C#N ; CC(=O)c1cc(F)ccc1-n1nc(C)cc1Cc1cn(CC2CC2)nc1C#N]

To a solution of 1-(cyclopropylmethyl)-4-((1-(2-(1-ethoxyvinyl)-4-fluorophenyl)-3-methyl-1H-pyrazol-5-yl)methyl)-1H-pyrazole-3-carbonitrile (264 mg, 0.650 mmol) in THF (9 mL) was added HCl (3 mL). The reaction was stirred at 20° C. for 1 h. The mixture was diluted with sat. aq. NaHCO₃ and extracted with EtOAc (30 mL×2). The combined organic layers were washed with brine (15 mL×2), dried over anhydrous Na₂SO₄, and concentrated. The residue was purified by flash column chromatography on silica gel (0→100% EtOAc in PE) to afford 4-((1-(2-acetyl-4-fluorophenyl)-3-methyl-1H-pyrazol-5-yl)methyl)-1-(cyclopropylmethyl)-1H-pyrazole-3-carbonitrile (160 mg, yield: 65.0%) as a yellow solid. LC/MS (ESI): m/z=378 [M+H]⁺.

The following intermediate was synthesized using a similar protocol (with m/z (ESI) values):

4-{[1-(2-acetyl-4,6-difluorophenyl)-3-methyl-1H-pyrazol-5-yl]methyl}-1-(cyclopropylmethyl)-
N,N-dimethyl-1H-pyrazole-3-carboxamide

| | |
|---|---|
| 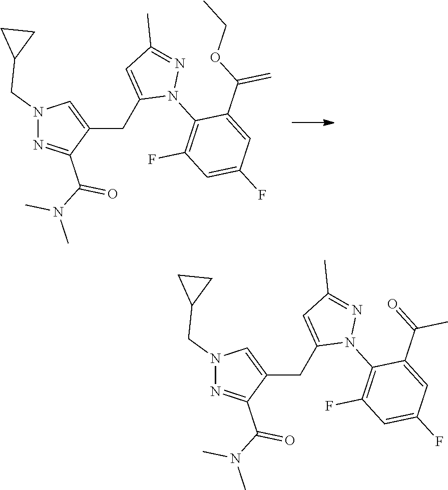 [SMILES: C=C(OCC)c1cc(F)cc(F)c1-n1nc(C)cc1Cc1cn(CC2CC2)nc1C(=O)N(C)C ; CC(=O)c1cc(F)cc(F)c1-n1nc(C)cc1Cc1cn(CC2CC2)nc1C(=O)N(C)C] | 442 [M + H]⁺ |

### Synthesis of 1-cyclobutyl-4-((1-(4-fluoro-2-(1-hydroxyethyl)phenyl)-3-methyl-1H-pyrazol-5-yl)methyl)-N N-dimethyl-1H-pyrazole-3-carboxamide

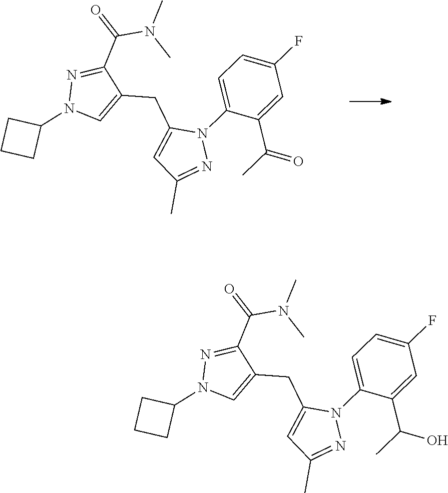 [SMILES: CC(=O)c1cc(F)ccc1-n1nc(C)cc1Cc1cn(C2CCC2)nc1C(=O)N(C)C ; Cc1cc(Cc2cn(C3CCC3)nc2C(=O)N(C)C)n(-c2ccc(F)cc2C(C)O)n1]

To a solution of 4-(1-(2-acetyl-4-fluorophenyl)-3-methyl-1H-pyrazol-5-yl)methyl)-1-cyclobutyl-N,N-dimethyl-1H-pyrazole-3-carboxamide (240 mg, 0.567 mmol) in MeOH (5 mL) was added NaBH₄ (23.58 mg, 0.623 mmol) at 0° C. and the reaction was stirred at 0° C. for 0.5 h under N₂. The reaction was quenched with aq. HCl (1 M, 5 mL) and extracted with EtOAc (15 mL×3). The combined organic layers were dried over anhydrous Na₂SO₄, filtered, and concentrated under vacuum to give a residue. The residue was purified by column chromatography on silica gel (0→2% MeOH in DCM) to give 1-cyclobutyl-4-((1-(4-fluoro-2-(1-hydroxyethyl)phenyl)-3-methyl-1H-pyrazol-5-yl)methyl)-N,N-dimethyl-1H-pyrazole-3-carboxamide (181 mg, yield: 75.1%) as a yellow oil. LC/MS (ESI) (m/z): 426 [M+H]⁺.

The following intermediates were synthesized using a similar protocol (with m/z (ESI) values):

1-(cyclopropylmethyl)-4-((1-(4-fluoro-2-(1-hydroxyethyl)phenyl)-3-methyl-1H-pyrazol-5-
yl)methyl)-1H-pyrazole-3-carbonitrile

| | |
|---|---|
| 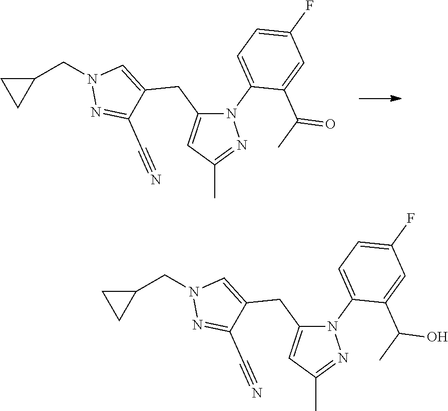 [SMILES: CC(=O)c1cc(F)ccc1-n1nc(C)cc1Cc1cn(CC2CC2)nc1C#N ; Cc1cc(Cc2cn(CC3CC3)nc2C#N)n(-c2ccc(F)cc2C(C)O)n1] | 380 [M + H]⁺ |

3-((3-(2-((R)-1-((5-bromo-2-nitropyridin-3-yl)oxy)ethyl)-4,6-difluorophenyl)-1-methyl-1H-
pyrazol-4-yl)(hydroxy)methyl)-1-methyl-1H-pyrazole-5-carbonitrile

| | |
|---|---|
| 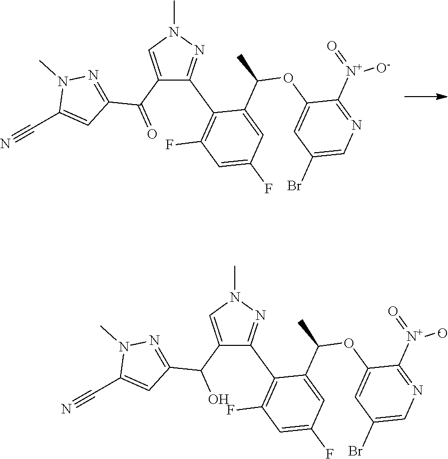 [SMILES: C[C@@H](Oc1cc(Br)cnc1[N+](=O)[O-])c1cc(F)cc(F)c1-c1nn(C)cc1C(=O)c1cc(C#N)n(C)n1 ; C[C@@H](Oc1cc(Br)cnc1[N+](=O)[O-])c1cc(F)cc(F)c1-c1nn(C)cc1C(O)c1cc(C#N)n(C)n1] | 574 [M + H]⁺ |

### Synthesis of (R)-5-bromo-3-(1-(2-(3-((1-ethyl-1H-pyrazol-4-yl)oxy)pyridin-2-yl)-5-fluorophenyl)ethoxy)-2-nitropyridine

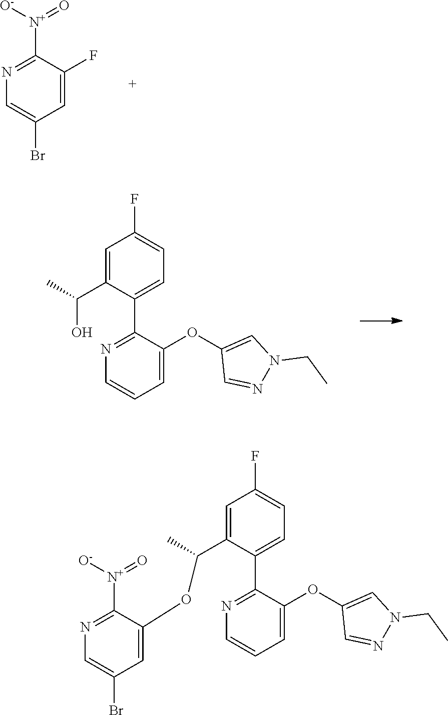 [SMILES: O=[N+]([O-])c1ncc(Br)cc1F ; CCn1cc(Oc2cccnc2-c2ccc(F)cc2[C@@H](C)O)cn1 ; CCn1cc(Oc2cccnc2-c2ccc(F)cc2[C@@H](C)Oc2cc(Br)cnc2[N+](=O)[O-])cn1]

To a solution of (R)-1-(2-(3-((1-ethyl-1H-pyrazol-4-yl)oxy)pyridin-2-yl)-5-fluorophenyl)ethan-1-ol (25.0 mg, 0.0760 mmol) in THF (15 mL) was added NaH (4.60 mg, 0.120 mmol, 60% mineral oil) and the mixture was stirred for 0.5 h. Then 5-bromo-3-fluoro-2-nitropyridine (20.0 mg, 0.0900 mmol) was added. The reaction was stirred for 18 h at 25° C. The mixture was quenched with water (5 mL) and extracted with EtOAc (15 mL×3). The combined organic layers were dried over anhydrous Na₂SO₄, filtered, and concentrated. The residue was purified by flash column chromatography on silica gel (0→50% EtOAc in PE) to give (R)-5-bromo-3-(1-(2-(3-((1-ethyl-1H-pyrazol-4-yl)oxy)pyridin-2-yl)-5-fluorophenyl)ethoxy)-2-nitropyridine (26.0 mg, 64.0% yield) as a yellow solid. LCMS (ESI): m/z: 528 [M+H]⁺.

The following intermediates were synthesized using a similar protocol (with m/z (ESI) values):

(R)-3-((2-(2-(1-((5-bromo-2-nitropyridin-3-yl)oxy)ethyl)-4-fluorophenyl)pyridin-3-yl)oxy)-1-
methyl-1H-pyrazole-5-carbonitrile

| | |
|---|---|
| 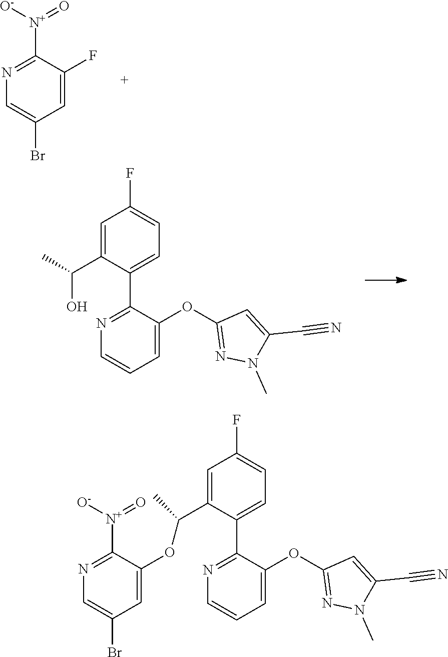 | 539 [M + H]⁺ |

(R)-(3-(2-(1-((5-bromo-2-nitropyridin-3-yl)oxy)ethyl)-4-fluorophenyl)-1-methyl-1H-pyrazol-
4-yl)(1-ethyl-1H-pyrazol-4-yl)methanone

| | |
|---|---|
| 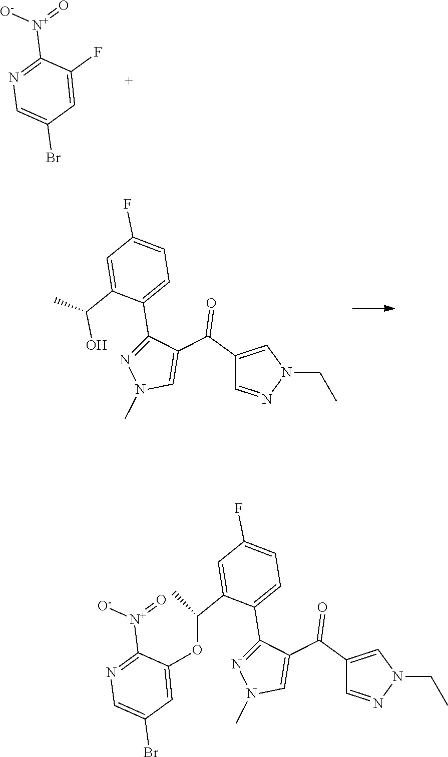 [SMILES: O=[N+]([O-])c1ncc(Br)cc1F ; CCn1cc(C(=O)c2cn(C)nc2-c2ccc(F)cc2[C@@H](C)O)cn1 ; CCn1cc(C(=O)c2cn(C)nc2-c2ccc(F)cc2[C@@H](C)Oc2cc(Br)cnc2[N+](=O)[O-])cn1] | 543 [M + H]⁺ |

4-((3-(2-(1-((5-bromo-2-nitropyridin-3-yl)oxy)ethyl)-4,6-difluorophenyl)-1-methyl-1H-
pyrazol-4-yl)methyl)-1-(cyclopropylmethyl)-1H-pyrazole-3-carbonitrile

| | |
|---|---|
| 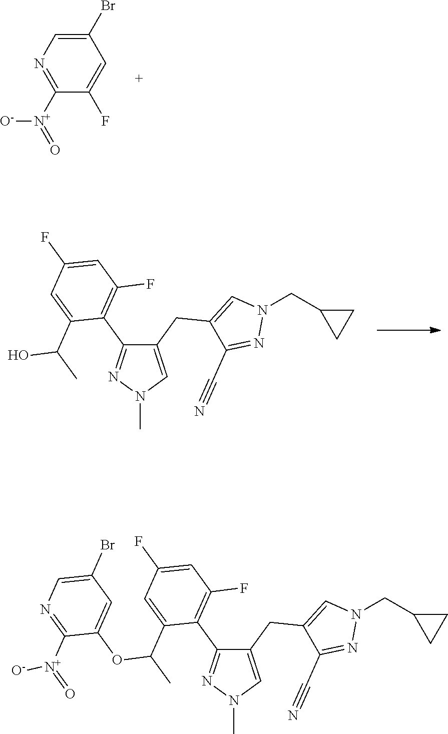 [SMILES: O=[N+]([O-])c1ncc(Br)cc1F ; CC(O)c1cc(F)cc(F)c1-c1nn(C)cc1Cc1cn(CC2CC2)nc1C#N ; CC(Oc1cc(Br)cnc1[N+](=O)[O-])c1cc(F)cc(F)c1-c1nn(C)cc1Cc1cn(CC2CC2)nc1C#N] | 598 [M + H]⁺ |

4-((2-(2-((R)-1-((5-bromo-2-nitropyridin-3-yl)oxy)ethyl)-4,6-difluorophenyl)pyridin-3-
yl)methyl)-1-(cyclopropylmethyl)-N,N-dimethyl-1H-pyrazole-3-carboxamide

| | |
|---|---|
| 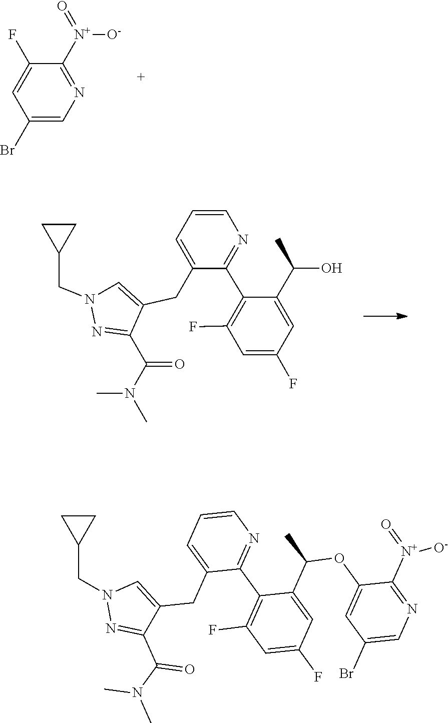 [SMILES: O=[N+]([O-])c1ncc(Br)cc1F ; C[C@@H](O)c1cc(F)cc(F)c1-c1ncccc1Cc1cn(CC2CC2)nc1C(=O)N(C)C ; C[C@@H](Oc1cc(Br)cnc1[N+](=O)[O-])c1cc(F)cc(F)c1-c1ncccc1Cc1cn(CC2CC2)nc1C(=O)N(C)C] | 641 [M + H]⁺ |

4-((2-(2-((R)-1-((5-bromo-2-nitropyridin-3-yl)oxy)ethyl)-4,6-difluorophenyl)pyridin-3-
yl)methyl)-1-ethyl-N,N-dimethyl-1H-pyrazole-3-carboxamide

| | |
|---|---|
| 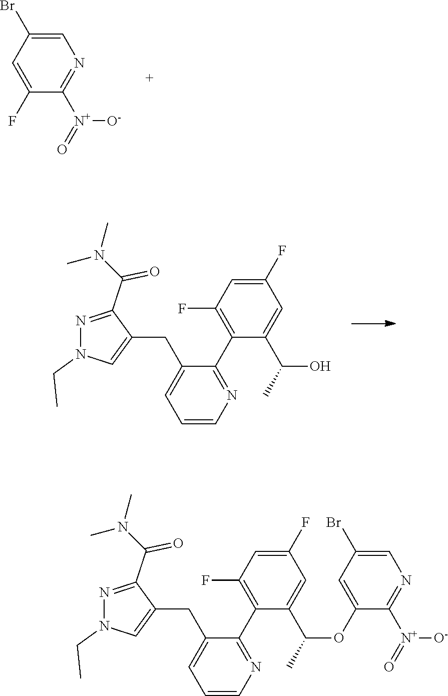 [SMILES: O=[N+]([O-])c1ncc(Br)cc1F ; CCn1cc(Cc2cccnc2-c2c(F)cc(F)cc2[C@@H](C)O)c(C(=O)N(C)C)n1 ; CCn1cc(Cc2cccnc2-c2c(F)cc(F)cc2[C@@H](C)Oc2cc(Br)cnc2[N+](=O)[O-])c(C(=O)N(C)C)n1] | 615 [M + H]⁺ |

(R)-4-((2-(2-(1-((5-bromo-2-nitropyridin-3-yl)oxy)ethyl)-4-fluorophenyl)pyridin-3-yl)
methyl)-1-cyclobutyl-N,N-dimethyl-1H-pyrazole-3-carboxamide

| | |
|---|---|
| 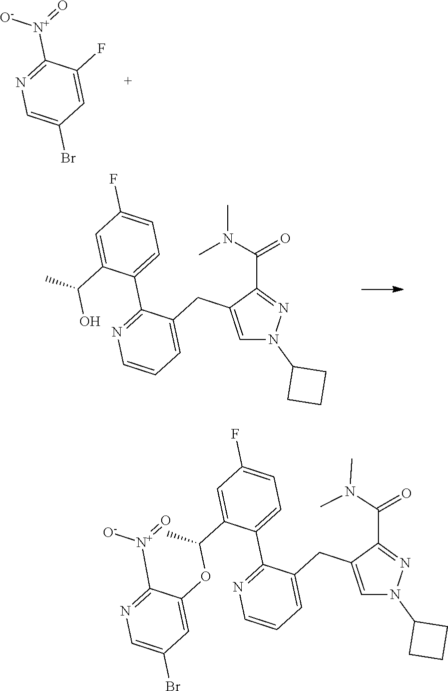 [SMILES: O=[N+]([O-])c1ncc(Br)cc1F ; C[C@@H](O)c1cc(F)ccc1-c1ncccc1Cc1cn(C2CCC2)nc1C(=O)N(C)C ; C[C@@H](Oc1cc(Br)cnc1[N+](=O)[O-])c1cc(F)ccc1-c1ncccc1Cc1cn(C2CCC2)nc1C(=O)N(C)C] | 623 [M + H]⁺ |

(R)-5-((2-(2-(1-((5-bromo-2-nitropyridin-3-yl)oxy)ethyl)-4-fluorophenyl)pyridin-3-yl)
methyl)-3-cyano-N,N-dimethyl-1H-pyrazole-1-carboxamide

| | |
|---|---|
| 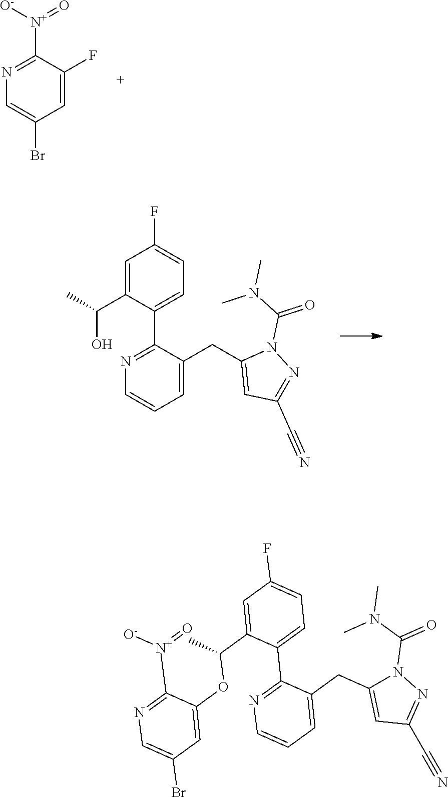 [SMILES: O=[N+]([O-])c1ncc(Br)cc1F ; C[C@@H](O)c1cc(F)ccc1-c1ncccc1Cc1cc(C#N)nn1C(=O)N(C)C ; C[C@@H](Oc1cc(Br)cnc1[N+](=O)[O-])c1cc(F)ccc1-c1ncccc1Cc1cc(C#N)nn1C(=O)N(C)C] | 594 [M + H]⁺ |

(R)-5-((4-(2-(1-((5-bromo-2-nitropyridin-3-yl)oxy)ethyl)-4-fluorophenyl)-2-methylthiazol-5-
yl)methyl)-3-cyano-N,N-dimethyl-1H-pyrazole-1-carboxamide

| | |
|---|---|
| 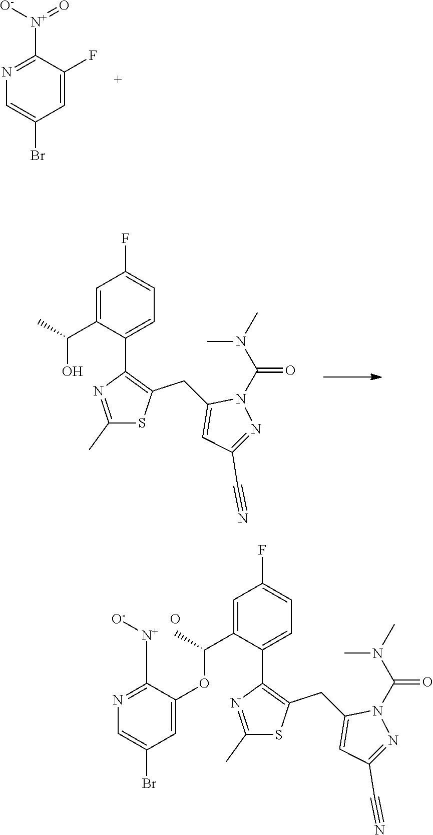 | 614 [M + H]⁺ |

3-((2-(2-((R)-1-((5-bromo-2-nitropyridin-3-yl)oxy)ethyl)-4,6-difluorophenyl)pyridin-3-yl)
oxy)-1-methyl-1H-pyrazole-5-carbonitrile

| | |
|---|---|
| 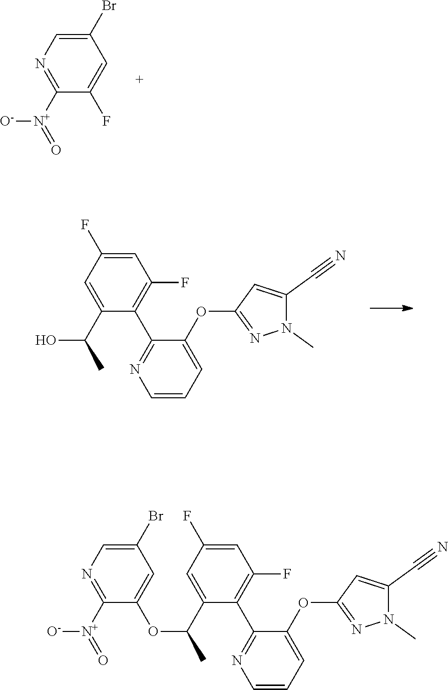 [SMILES: O=[N+]([O-])c1ncc(Br)cc1F ; C[C@@H](O)c1cc(F)cc(F)c1-c1ncccc1Oc1cc(C#N)n(C)n1 ; C[C@@H](Oc1cc(Br)cnc1[N+](=O)[O-])c1cc(F)cc(F)c1-c1ncccc1Oc1cc(C#N)n(C)n1] | 557 [M + H]⁺ |

3-(1-(2-(1-((5-bromo-2-nitropyridin-3-yl)oxy)ethyl)-4,6-difluorophenyl)-3-methyl-1H-
pyrazole-5-carbonyl)-1-methyl-1H-pyrazole-5-carbonitrile

| | |
|---|---|
| 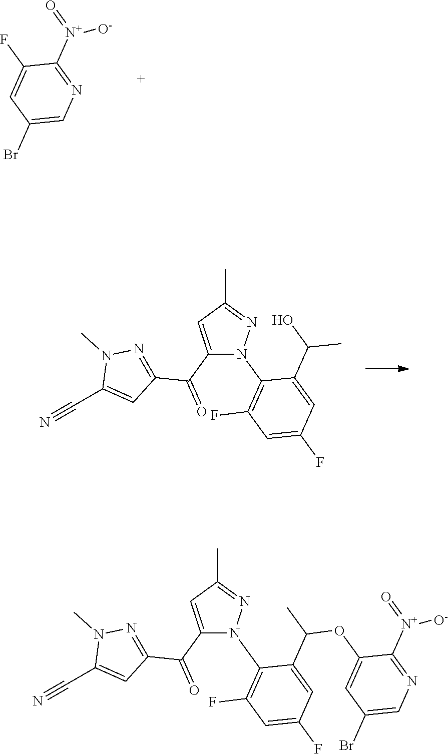 [SMILES: O=[N+]([O-])c1ncc(Br)cc1F ; Cc1cc(C(=O)c2cc(C#N)n(C)n2)n(-c2c(F)cc(F)cc2C(C)O)n1 ; Cc1cc(C(=O)c2cc(C#N)n(C)n2)n(-c2c(F)cc(F)cc2C(C)Oc2cc(Br)cnc2[N+](=O)[O-])n1] | 572 [M + H]⁺ |

(R)-5-((2-(2-(1-((5-bromo-2-nitropyridin-3-yl)oxy)ethyl)-4-fluorophenyl)-6-methylpyridin-3-
yl)methyl)-1-((2-(trimethylsilyl)ethoxy)methyl)-1H-pyrazole-3-carbonitrile

| | |
|---|---|
| 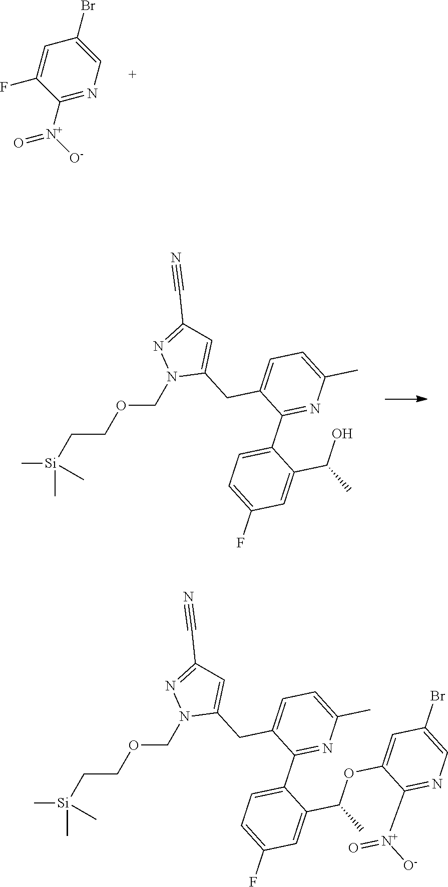 | 667 [M + H]⁺ |

4-((1-(2-(1-((5-bromo-2-nitropyridin-3-yl)oxy)ethyl)-4-fluorophenyl)-3-methyl-1H-pyrazol-5-
yl)methyl)-1-cyclobutyl-N,N-dimethyl-1H-pyrazole-3-carboxamide

| | |
|---|---|
| 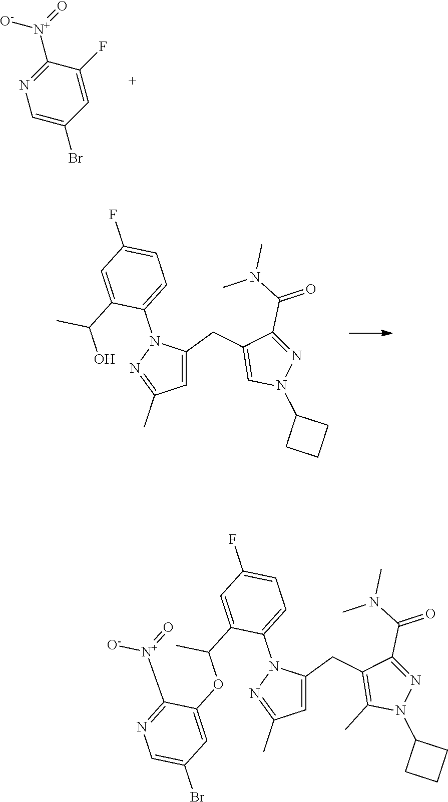 [SMILES: O=[N+]([O-])c1ncc(Br)cc1F ; Cc1cc(Cc2cn(C3CCC3)nc2C(=O)N(C)C)n(-c2ccc(F)cc2C(C)O)n1 ; Cc1cc(Cc2c(C(=O)N(C)C)nn(C3CCC3)c2C)n(-c2ccc(F)cc2C(C)Oc2cc(Br)cnc2[N+](=O)[O-])n1] | 626 [M + H]⁺ |

### Synthesis of (R)-4-((2-(2-(1-((5-bromo-2-nitropyridin-3-yl)oxy)ethyl)-4-fluorophenyl)pyridin-3-yl)methyl)-1-ethyl-1H-pyrazole-3-carbonitrile

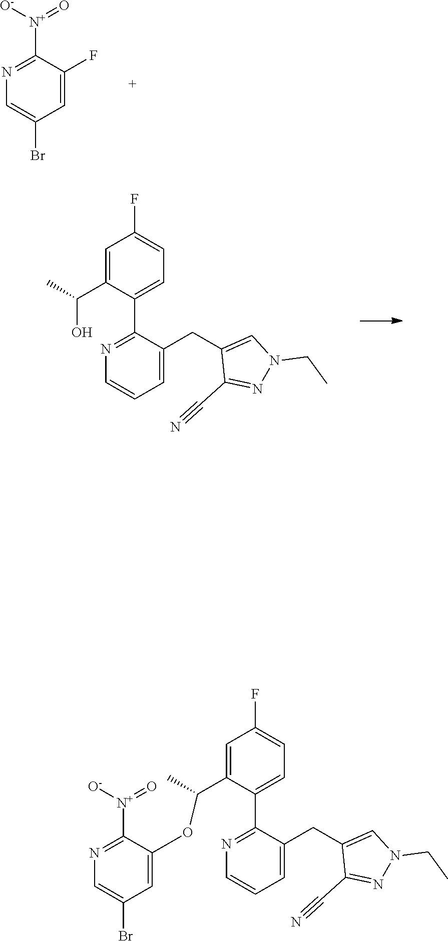 [SMILES: O=[N+]([O-])c1ncc(Br)cc1F ; CCn1cc(Cc2cccnc2-c2ccc(F)cc2[C@@H](C)O)c(C#N)n1 ; CCn1cc(Cc2cccnc2-c2ccc(F)cc2[C@@H](C)Oc2cc(Br)cnc2[N+](=O)[O-])c(C#N)n1]

To a solution of (R)-1-ethyl-4-((2-(4-fluoro-2-(1-hydroxyethyl)phenyl)pyridin-3-yl)methyl)-1H-pyrazole-3-carbonitrile (320 mg, 0.910 mmol) and 5-bromo-3-fluoro-2-nitropyridine (303 mg, 1.37 mmol) in THE (10 mL) was added t-BuOK (1 M in THE, 1.37 mL, 1.37 mmol) at −70° C. The reaction was stirred for 10 min at −70° C. The mixture was quenched with sat. aq. NH₄Cl (15 mL) and extracted with EtOAc (30 mL×3). The combined organic layers were washed with brine (20 mL×2), dried over anhydrous Na₂SO₄ and concentrated to give (R)-4-((2-(2-(1-((5-bromo-2-nitropyridin-3-yl)oxy)ethyl)-4-fluorophenyl)pyridin-3-yl)methyl)-1-ethyl-1H-pyrazole-3-carbonitrile (500 mg, crude) as white solid. LC/MS (ESI) (m/z): 551 [M+H]⁺.

The following intermediates were synthesized using a similar protocol (with m/z (ESI) values):

4-((1-(2-(1-((5-bromo-2-nitropyridin-3-yl)oxy)ethyl)-4-fluorophenyl)-3-methyl-1H-pyrazol-5-
yl)methyl)-1-(cyclopropylmethyl)-1H-pyrazole-3-carbonitrile

| | |
|---|---|
| 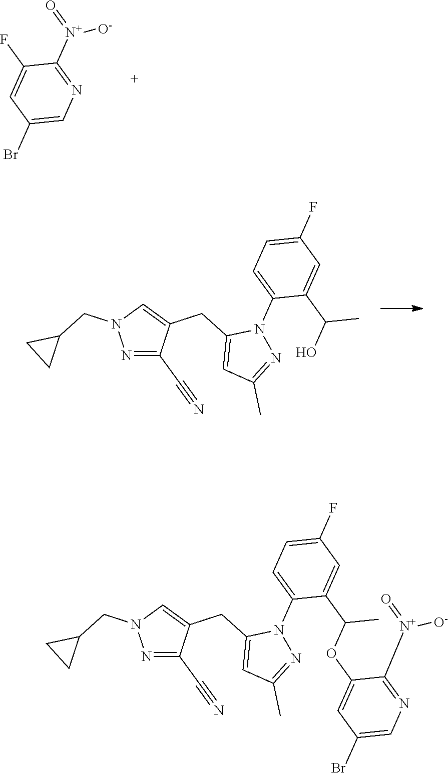 | 580 [M + H]⁺ |

(R)-4-((3-(2-(1-((5-bromo-2-nitropyridin-3-yl)oxy)ethyl)-4-fluorophenyl)-1-methyl-1H-
pyrazol-4-yl)methyl)-1-(cyclobutylmethyl)-1H-pyrazole-3-carbonitrile

| | |
|---|---|
| 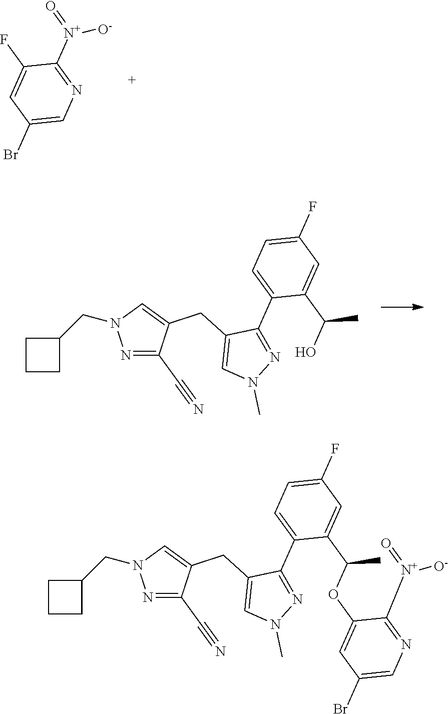 [SMILES: O=[N+]([O-])c1ncc(Br)cc1F ; C[C@@H](O)c1cc(F)ccc1-c1nn(C)cc1Cc1cn(CC2CCC2)nc1C#N ; C[C@@H](Oc1cc(Br)cnc1[N+](=O)[O-])c1cc(F)ccc1-c1nn(C)cc1Cc1cn(CC2CCC2)nc1C#N] | 594 [M + H]⁺ |

(R)-4-((2-(2-(1-((5-bromo-2-nitropyridin-3-yl)oxy)ethyl)-4-fluorophenyl)pyridin-3-yl)methyl)-
1-(cyclopropylmethyl)-1H-pyrazole-3-carbonitrile

| | |
|---|---|
| 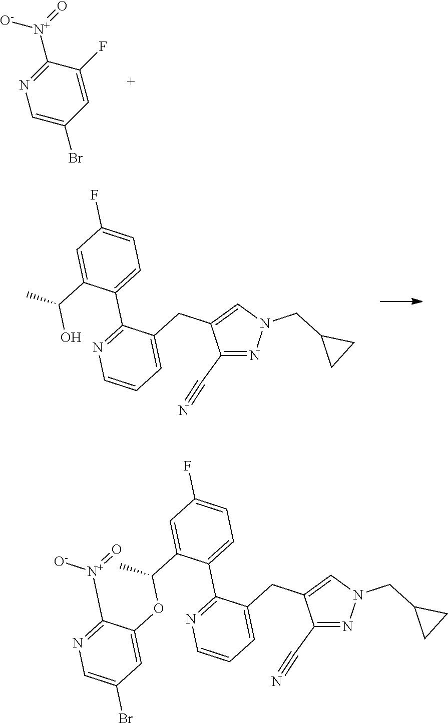 | 577 [M + H]⁺ |

(R)-5-bromo-3-(1-(2-(4-((1-(cyclopropylmethyl)-3-(methylthio)-1H-pyrazol-4-yl)methyl)-1-
methyl-1H-pyrazol-3-yl)-5-fluorophenyl)ethoxy)-2-nitropyridine

| | |
|---|---|
| 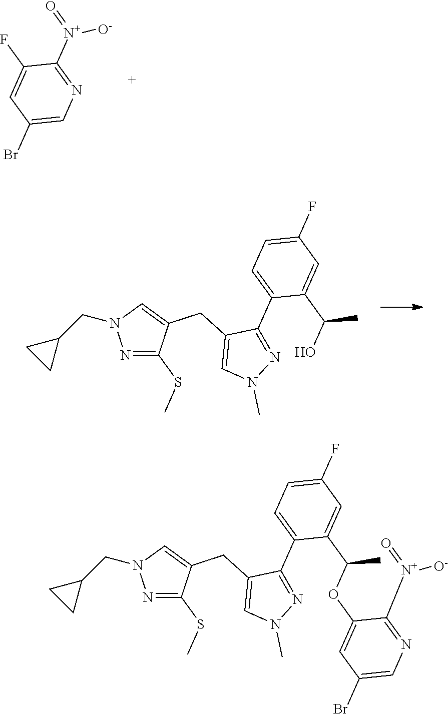 [SMILES: O=[N+]([O-])c1ncc(Br)cc1F ; CSc1nn(CC2CC2)cc1Cc1cn(C)nc1-c1ccc(F)cc1[C@@H](C)O ; CSc1nn(CC2CC2)cc1Cc1cn(C)nc1-c1ccc(F)cc1[C@@H](C)Oc1cc(Br)cnc1[N+](=O)[O-]] | 601 [M + H]⁺ |

(R)-5-((2-(2-(1-((5-bromo-2-nitropyridin-3-yl)oxy)ethyl)-4-fluorophenyl)pyridin-3-yl)methyl)-
3-cyclobutyl-N,N-dimethyl-1H-pyrazole-1-carboxamide

| | |
|---|---|
| 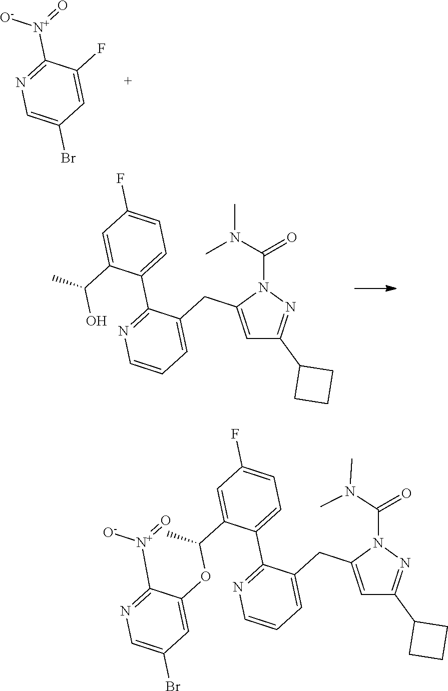 [SMILES: O=[N+]([O-])c1ncc(Br)cc1F ; C[C@@H](O)c1cc(F)ccc1-c1ncccc1Cc1cc(C2CCC2)nn1C(=O)N(C)C ; C[C@@H](Oc1cc(Br)cnc1[N+](=O)[O-])c1cc(F)ccc1-c1ncccc1Cc1cc(C2CCC2)nn1C(=O)N(C)C] | 623 [M + H]⁺ |

(R)-4-bromo-5-((2-(2-(1-((5-bromo-2-nitropyridin-3-yl)oxy)ethyl)-4-fluorophenyl)pyridin-3-
yl)methyl)-3-(cyclopropylmethyl)-N,N-dimethyl-1H-pyrazole-1-carboxamide

| | |
|---|---|
| 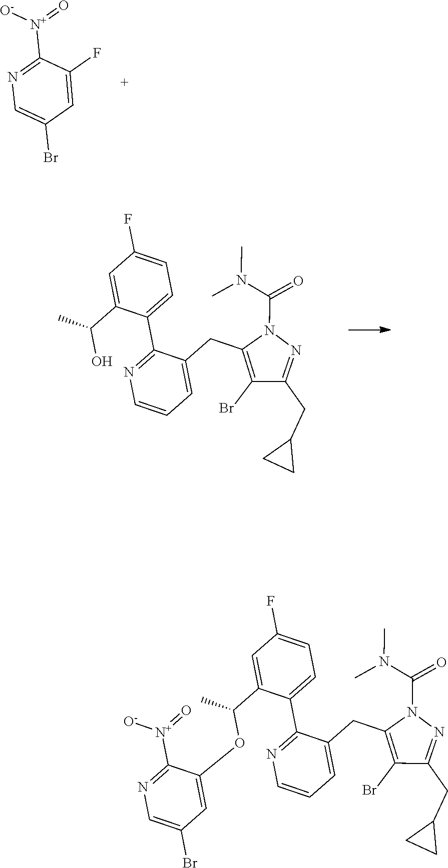 [SMILES: O=[N+]([O-])c1ncc(Br)cc1F ; C[C@@H](O)c1cc(F)ccc1-c1ncccc1Cc1c(Br)c(CC2CC2)nn1C(=O)N(C)C ; C[C@@H](Oc1cc(Br)cnc1[N+](=O)[O-])c1cc(F)ccc1-c1ncccc1Cc1c(Br)c(CC2CC2)nn1C(=O)N(C)C] | 701 [M + H]⁺ |

(R)-4-bromo-5-((3-(2-(1-((5-bromo-2-nitropyridin-3-yl)oxy)ethyl)-4,6-difluorophenyl)-1-
methyl-1H-pyrazol-4-yl)methyl)-3-(cyclopropylmethyl)-N,N-dimethyl-1H-pyrazole-1-
carboxamide

| | |
|---|---|
| 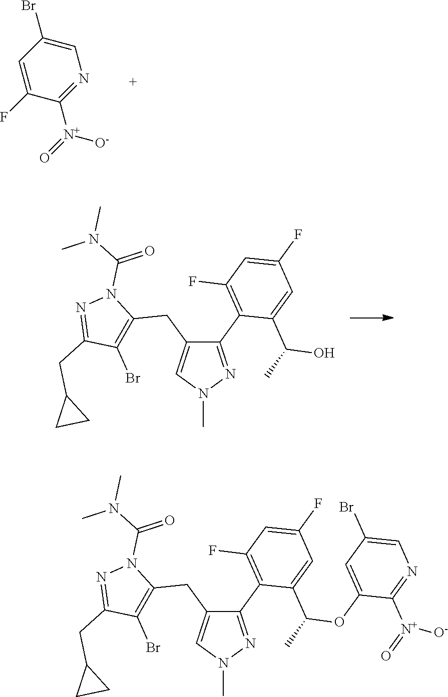 [SMILES: O=[N+]([O-])c1ncc(Br)cc1F ; C[C@@H](O)c1cc(F)cc(F)c1-c1nn(C)cc1Cc1c(Br)c(CC2CC2)nn1C(=O)N(C)C ; C[C@@H](Oc1cc(Br)cnc1[N+](=O)[O-])c1cc(F)cc(F)c1-c1nn(C)cc1Cc1c(Br)c(CC2CC2)nn1C(=O)N(C)C] | 722 [M + H]⁺ |

(R)-4-bromo-5-((2-(2-(1-((5-bromo-2-nitropyridin-3-yl)oxy)ethyl)-4-fluorophenyl)pyridin-3-
yl)methyl)-3-ethyl-N,N-dimethyl-1H-pyrazole-1-carboxamide

| | |
|---|---|
| 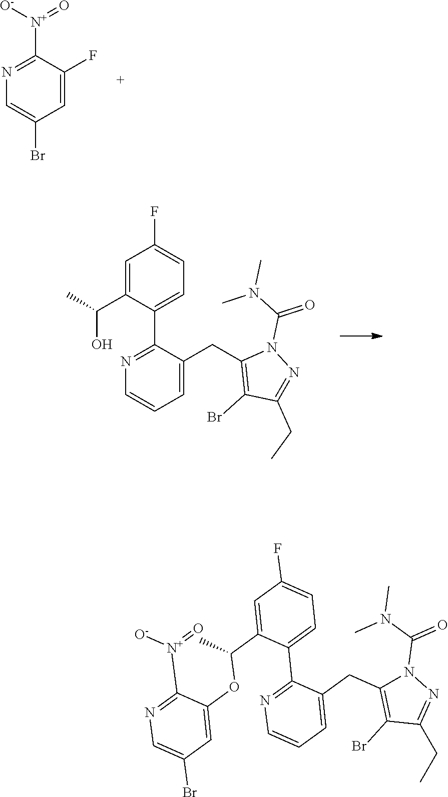 [SMILES: O=[N+]([O-])c1ncc(Br)cc1F ; CCc1nn(C(=O)N(C)C)c(Cc2cccnc2-c2ccc(F)cc2[C@@H](C)O)c1Br ; CCc1nn(C(=O)N(C)C)c(Cc2cccnc2-c2ccc(F)cc2[C@@H](C)Oc2cc(Br)cnc2[N+](=O)[O-])c1Br] | 675 [M + H]⁺ |

(R)-1-((3-(2-(1-((5-bromo-2-nitropyridin-3-yl)oxy)ethyl)-4-fluorophenyl)-1-methyl-1H-
pyrazol-4-yl)methyl)-4-(cyclopropylmethyl)-N,N-dimethyl-1H-imidazole-2-carboxamide

| | |
|---|---|
| 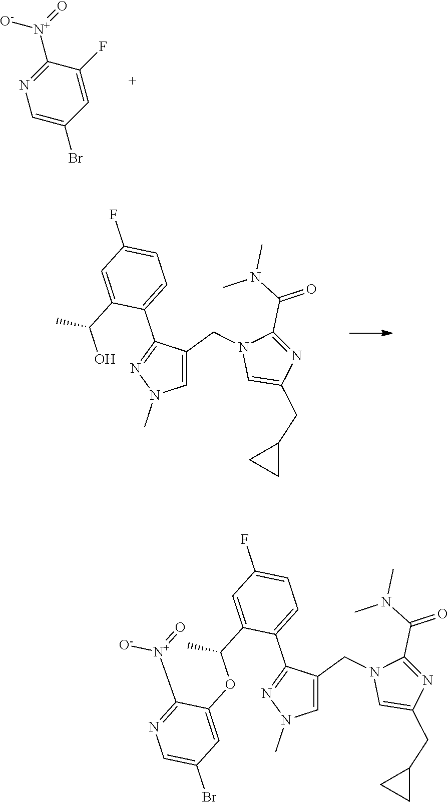 [SMILES: O=[N+]([O-])c1ncc(Br)cc1F ; C[C@@H](O)c1cc(F)ccc1-c1nn(C)cc1Cn1cc(CC2CC2)nc1C(=O)N(C)C ; C[C@@H](Oc1cc(Br)cnc1[N+](=O)[O-])c1cc(F)ccc1-c1nn(C)cc1Cn1cc(CC2CC2)nc1C(=O)N(C)C] | 626 [M + H]⁺ |

### Synthesis of (R)-4-((3-(2-(1-((5-bromo-2-nitropyridin-3-yl)oxy)ethyl)-4-fluorophenyl)-1-methyl-1H-pyrazol-4-yl)methyl)-1-ethyl-1H-pyrazole-3-carbonitrile

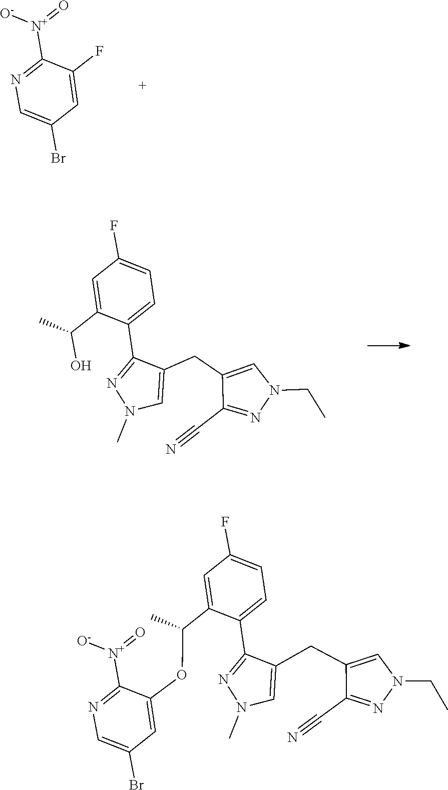 [SMILES: O=[N+]([O-])c1ncc(Br)cc1F ; CCn1cc(Cc2cn(C)nc2-c2ccc(F)cc2[C@@H](C)O)c(C#N)n1 ; CCn1cc(Cc2cn(C)nc2-c2ccc(F)cc2[C@@H](C)Oc2cc(Br)cnc2[N+](=O)[O-])c(C#N)n1]

To a stirred solution of (R)-1-ethyl-4-((3-(4-fluoro-2-(1-hydroxyethyl)phenyl)-1-methyl-1H-pyrazol-4-yl)methyl)-1H-pyrazole-3-carbonitrile (584 mg, 1.65 mmol) in THF (10 mL) was added NaH (198 mg, 4.96 mmol, 60% in mineral oil) at 0° C. under N₂. After stirring at 0° C. for 1 h, a solution of 5-bromo-3-fluoro-2-nitropyridine (365 mg, 1.65 mmol) in THF (1 mL) was added dropwise. The reaction was stirred at 0° C. for 1 h, quenched with sat. aq. NH₄Cl (10 mL) and extracted with EtOAc (10 mL). The organic layer was dried over anhydrous Na₂SO₄ and the filtrate was concentrated to give crude (R)-4-((3-(2-(1-((5-bromo-2-nitropyridin-3-yl)oxy)ethyl)-4-fluorophenyl)-1-methyl-1H-pyrazol-4-yl)methyl)-1-ethyl-1H-pyrazole-3-carbonitrile (916 mg) as a yellow oil. LC/MS (ESI) (m/z): 554.1 [M+H]⁺.

The following intermediates were synthesized using a similar protocol (with m/z (ESI) values):

(R)-4-((3-(2-(1-((5-bromo-2-nitropyridin-3-yl)oxy)ethyl)-4-fluorophenyl)-1-methyl-1H-
pyrazol-4-yl)methyl)-1-cyclobutyl-1H-pyrazole-3-carbonitrile

| | |
|---|---|
| 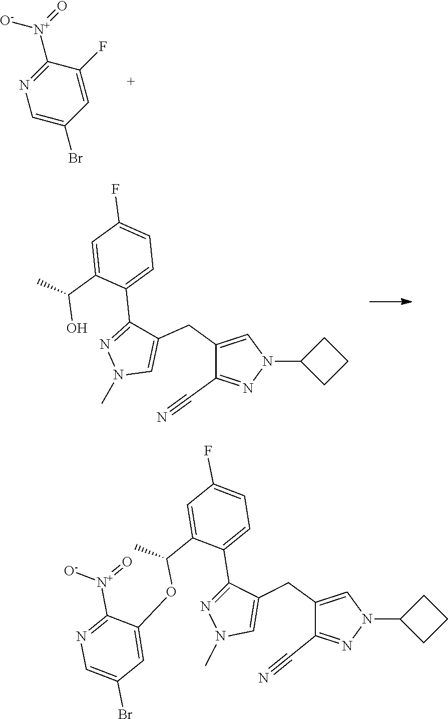 [SMILES: O=[N+]([O-])c1ncc(Br)cc1F ; C[C@@H](O)c1cc(F)ccc1-c1nn(C)cc1Cc1cn(C2CCC2)nc1C#N ; C[C@@H](Oc1cc(Br)cnc1[N+](=O)[O-])c1cc(F)ccc1-c1nn(C)cc1Cc1cn(C2CCC2)nc1C#N] | 580 [M + H]⁺ |

3-(2-(2-((R)-1-((5-bromo-2-nitropyridin-3-yl)oxy)ethyl)-4,6-difluorophenyl)nicotinoyl)-1-((2-
(trimethylsilyl)ethoxy)methyl)-1H-pyrazole-5-carbonitrile

| | |
|---|---|
| 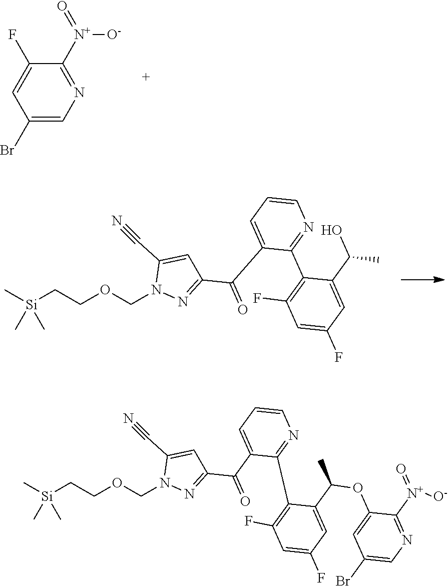 | 685 [M + H]⁺ |

(R)-4-((3-(2-(1-((5-bromo-2-nitropyridin-3-yl)oxy)ethyl)-4-fluorophenyl)-1-methyl-1H-
pyrazol-4-yl)methyl)-1-(cyclopropylmethyl)-1H-pyrazole-3-carbonitrile

| | |
|---|---|
| 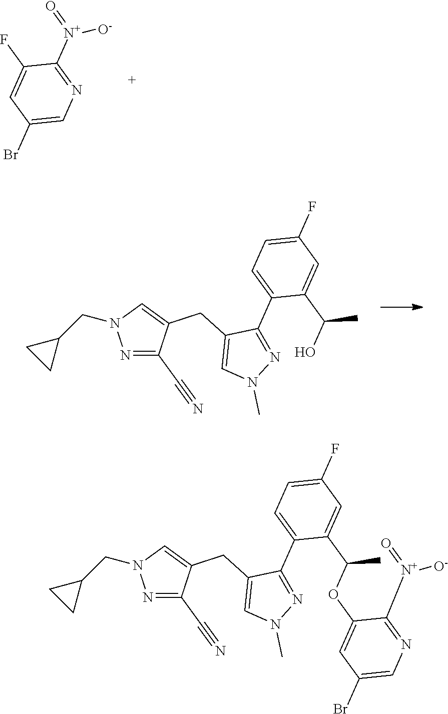 [SMILES: O=[N+]([O-])c1ncc(Br)cc1F ; C[C@@H](O)c1cc(F)ccc1-c1nn(C)cc1Cc1cn(CC2CC2)nc1C#N ; C[C@@H](Oc1cc(Br)cnc1[N+](=O)[O-])c1cc(F)ccc1-c1nn(C)cc1Cc1cn(CC2CC2)nc1C#N] | 580 [M + H]⁺ |

(R)-3-(3-(2-(1-((5-bromo-2-nitropyridin-3-yl)oxy)ethyl)-4,6-difluorophenyl)-1-methyl-1H-
pyrazole-4-carbonyl)-1-methyl-1H-pyrazole-5-carbonitrile

| | |
|---|---|
| 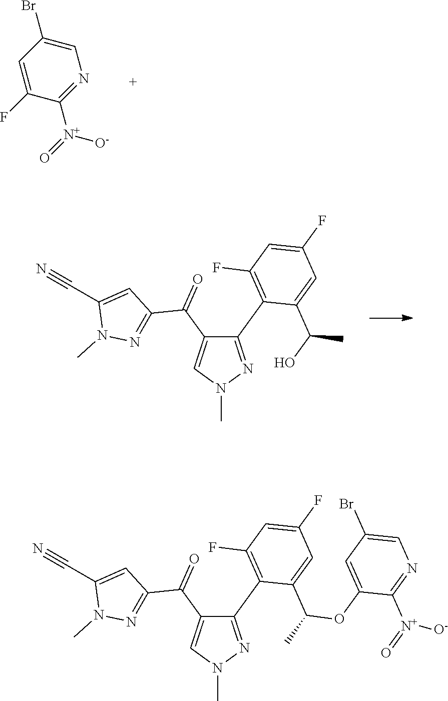 [SMILES: O=[N+]([O-])c1ncc(Br)cc1F ; C[C@@H](O)c1cc(F)cc(F)c1-c1nn(C)cc1C(=O)c1cc(C#N)n(C)n1 ; C[C@@H](Oc1cc(Br)cnc1[N+](=O)[O-])c1cc(F)cc(F)c1-c1nn(C)cc1C(=O)c1cc(C#N)n(C)n1] | 572 [M + H]⁺ |

### Synthesis of 3-(2-(2-((R)-1-((5-bromo-2-nitropyridin-3-yl)oxy)ethyl)-4,6-difluorophenyl)nicotinoyl)-1-methyl-1H-pyrazole-5-carbonitrile and 5-(2-{2-[(1R)-1-[(5-bromo-2-nitropyridin-3-yl)oxy]ethyl]-4,6-difluorophenyl}pyridine-3-carbonyl)-1-methyl-1H-pyrazole-3-carbonitrile

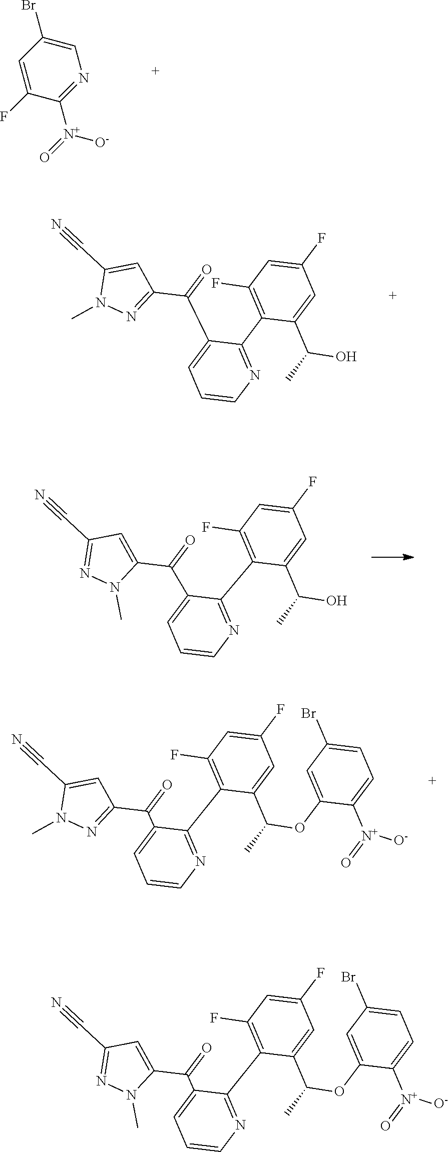 [SMILES: O=[N+]([O-])c1ncc(Br)cc1F ; C[C@@H](O)c1cc(F)cc(F)c1-c1ncccc1C(=O)c1cc(C#N)n(C)n1 ; C[C@@H](O)c1cc(F)cc(F)c1-c1ncccc1C(=O)c1cc(C#N)nn1C ; C[C@@H](Oc1cc(Br)ccc1[N+](=O)[O-])c1cc(F)cc(F)c1-c1ncccc1C(=O)c1cc(C#N)n(C)n1 ; C[C@@H](Oc1cc(Br)ccc1[N+](=O)[O-])c1cc(F)cc(F)c1-c1ncccc1C(=O)c1cc(C#N)nn1C]

To a solution of 3-(2-(2,4-difluoro-6-((R)-1-hydroxyethyl)phenyl)nicotinoyl)-1-methyl-1H-pyrazole-5-carbonitrile and 5-(2-{2,4-difluoro-6-[(1R)-1-hydroxyethyl]phenyl}pyridine-3-carbonyl)-1-methyl-1H-pyrazole-3-carbonitrile (70 mg, 0.190 mmol) in THF (5 mL) was added NaH (15.2 mg, 0.380 mmol, 60% in mineral oil) at 0° C. under N₂ and the mixture was stirred at 0° C. for 0.5 h. 5-bromo-3-fluoro-2-nitropyridine (83.9 mg, 0.380 mmol) was then added and the reaction was stirred at r.t. for 12 h under N₂. The mixture was quenched with sat. aq. NH₄Cl (10 mL) and extracted with EtOAc (15 mL×3). The combined organic phases were washed with brine (20 mL), dried over anhydrous Na₂SO₄, filtered and concentrated. The residue was purified by flash column chromatography on silica gel (0→10% MeOH in DCM) to give 3-(2-(2-((R)-1-((5-bromo-2-nitropyridin-3-yl)oxy)ethyl)-4,6-difluorophenyl) nicotinoyl)-1-methyl-1H-pyrazole-5-carbonitrile and 5-(2-{2-[(1R)-1-[(5-bromo-2-nitropyridin-3-yl)oxy]ethyl]-4,6-difluorophenyl}pyridine-3-carbonyl)-1-methyl-1H-pyrazole-3-carbonitrile (60.0 mg, yield:55.5%) as a yellow solid mixture. LC/MS ESI (m/z): 569 [M+H]⁺.

### Synthesis of 3-((2-(2-((R)-1-((3-amino-6-bromopyrazin-2-yl)oxy)ethyl)-4,6-difluorophenyl)pyridin-3-yl)(methoxymethoxy)methyl)-1-methyl-1H-pyrazole-5-carbonitrile

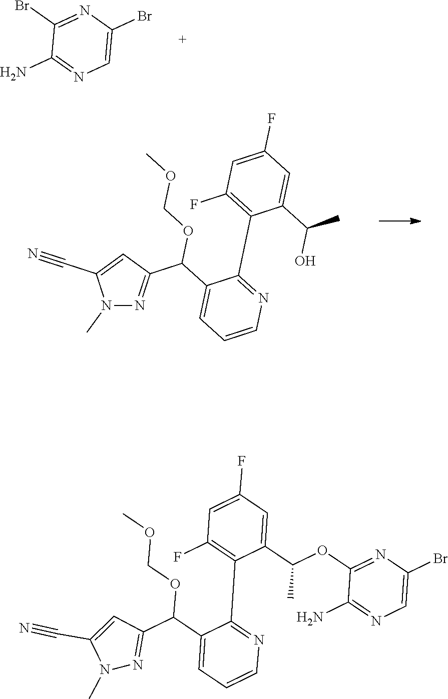 [SMILES: Nc1ncc(Br)nc1Br ; COCOC(c1cc(C#N)n(C)n1)c1cccnc1-c1c(F)cc(F)cc1[C@@H](C)O ; COCOC(c1cc(C#N)n(C)n1)c1cccnc1-c1c(F)cc(F)cc1[C@@H](C)Oc1nc(Br)cnc1N]

To a stirred solution of 3-((2-(2,4-difluoro-6-((R)-1-hydroxyethyl)phenyl)pyridin-3-yl)(methoxymethoxy)methyl)-1-methyl-1H-pyrazole-5-carbonitrile (1.67 g, 4.03 mmol) in THF (30 mL) was added NaH (242 mg, 6.04 mmol, 60% in mineral oil) at 0° C. under N₂. After stirring at 0° C. for 1 h, a solution of 3,5-dibromopyrazin-2-amine (1.20 g, 4.84 mmol) in THF (10 mL) was added dropwise. The reaction was stirred at 70° C. for 1 h. The reaction was quenched with sat. aq. NH₄Cl (100 mL), extracted with EtOAc (100 mL). The organic phase was dried over Na₂SO₄ and concentrated to dryness to give 3-((2-(2-((R)-1-((3-amino-6-bromopyrazin-2-yl)oxy)ethyl)-4,6-difluorophenyl)pyridin-3-yl)(methoxymethoxy)methyl)-1-methyl-1H-pyrazole-5-carbonitrile (800 mg, 34% yield) as a yellow oil. LC/MS (ESI) (m/z): 586 [M+H]⁺.

### Synthesis of 3-((2-(2-((R)-1-((5-bromo-2-nitropyridin-3-yl)oxy)ethyl)-4,6-difluorophenyl)pyridin-3-yl)methyl)-5-cyano-N,N-dimethyl-1H-pyrazole-1-carboxamide and 5-((2-(2-((R)-1-((5-bromo-2-nitropyridin-3-yl)oxy)ethyl)-4,6-difluorophenyl)pyridin-3-yl)methyl)-3-cyano-N,N-dimethyl-1H-pyrazole-1-carboxamide

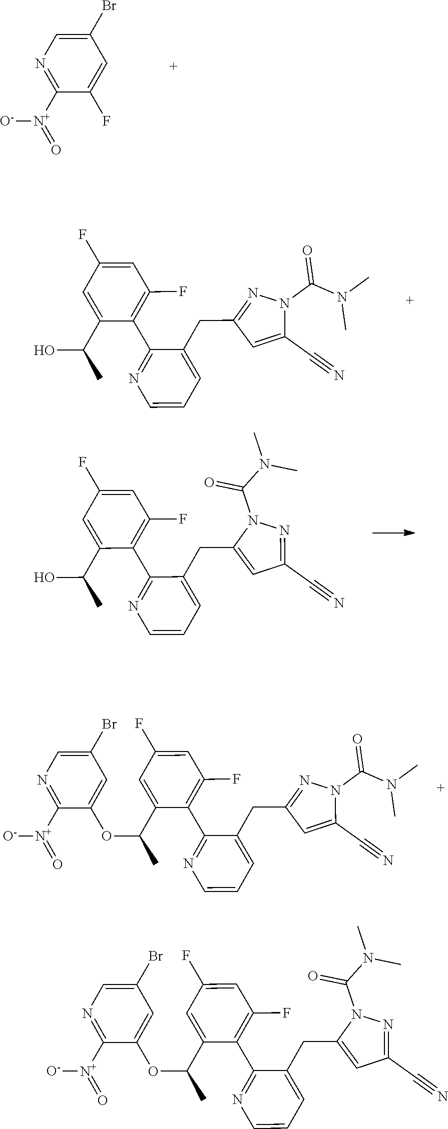 [SMILES: O=[N+]([O-])c1ncc(Br)cc1F ; C[C@@H](O)c1cc(F)cc(F)c1-c1ncccc1Cc1cc(C#N)n(C(=O)N(C)C)n1 ; C[C@@H](O)c1cc(F)cc(F)c1-c1ncccc1Cc1cc(C#N)nn1C(=O)N(C)C ; C[C@@H](Oc1cc(Br)cnc1[N+](=O)[O-])c1cc(F)cc(F)c1-c1ncccc1Cc1cc(C#N)n(C(=O)N(C)C)n1 ; C[C@@H](Oc1cc(Br)cnc1[N+](=O)[O-])c1cc(F)cc(F)c1-c1ncccc1Cc1cc(C#N)nn1C(=O)N(C)C]

To a mixture of 3-cyano-5-((2-(2,4-difluoro-6-((R)-1-hydroxyethyl)phenyl)pyridin-3-yl)methyl)-N,N-dimethyl-1H-pyrazole-1-carboxamide and 5-cyano-3-((2-(2,4-difluoro-6-((R)-1-hydroxyethyl)phenyl)pyridin-3-yl)methyl)-N,N-dimethyl-1H-pyrazole-1-carboxamide (110 mg, 0.267 mmol) and 5-bromo-3-fluoro-2-nitropyridine (70.9 mg, 0.321 mmol) in THF was added potassium tert-butoxide (1 M in THF, 0.401 mL, 0.401 mmol) at −78° C. under N₂. The reaction was stirred at −78° C. for 1 h. The reaction mixture was quenched with sat. aq. NH₄Cl (7 mL) and extracted with EtOAc (10 mL×3). The combined organic layers were washed with brine (10 mL×2), dried over anhydrous Na₂SO₄ and concentrated. The residue was purified by flash column chromatography on silica gel (0→100% EtOAc in PE) to give 5-((2-(2-((R)-1-((5-bromo-2-nitropyridin-3-yl)oxy)ethyl)-4,6-difluorophenyl)pyridin-3-yl)methyl)-3-cyano-N,N-dimethyl-1H-pyrazole-1-carboxamide and 3-((2-(2-((R)-1-((5-bromo-2-nitropyridin-3-yl)oxy)ethyl)-4,6-difluorophenyl)pyridin-3-yl)methyl)-5-cyano-N,N-dimethyl-1H-pyrazole-1-carboxamide (110 mg, yield: 67.2%) as yellow oil mixture. LC/MS (ESI) (m/z): 612 [M+H]⁺.

### Synthesis of ethyl (R)-4-((2-(2-(1-((5-bromo-2-nitropyridin-3-yl)oxy)ethyl)-4-fluorophenyl)pyridin-3-yl)oxy)-1-(cyclopropylmethyl)-1H-pyrazole-3-carboxylate

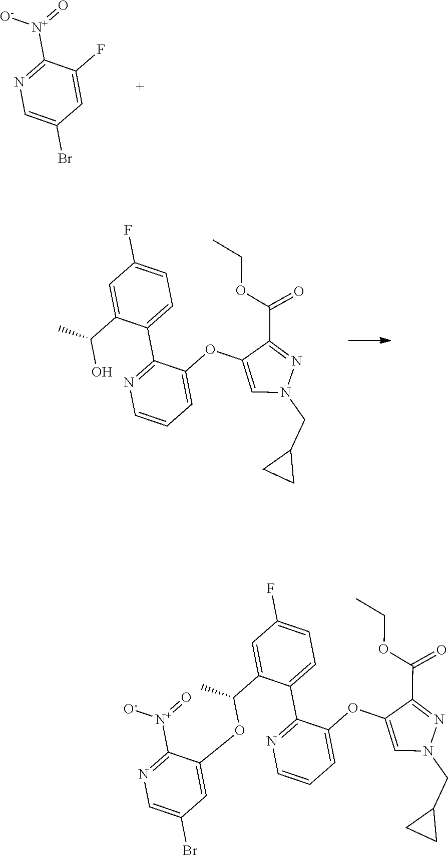 [SMILES: O=[N+]([O-])c1ncc(Br)cc1F ; CCOC(=O)c1nn(CC2CC2)cc1Oc1cccnc1-c1ccc(F)cc1[C@@H](C)O ; CCOC(=O)c1nn(CC2CC2)cc1Oc1cccnc1-c1ccc(F)cc1[C@@H](C)Oc1cc(Br)cnc1[N+](=O)[O-]]

To a solution of ethyl (R)-1-(cyclopropylmethyl)-4-((2-(4-fluoro-2-(1-hydroxyethyl)phenyl)pyridin-3-yl)oxy)-1H-pyrazole-3-carboxylate (550 mg, 1.29 mmol) and 5-bromo-3-fluoro-2-nitropyridine (314 mg, 1.42 mmol) in THF (22 mL) was added t-BuOK (1 M in THF, 2.59 mL) at −70° C. under N₂. After stirring for 15 min, the mixture was quenched with sat. aq. NH₄Cl solution (20 mL) and extracted with EtOAc (20 mL×3). The combined organic phase was washed with brine (25 mL×2), dried with anhydrous Na₂SO₄ and concentrated. The residue was purified by flash column chromatography on silica gel (0→100% of EtOAc in PE, V/V) to give ethyl (R)-4-((2-(2-(1-((5-bromo-2-nitropyridin-3-yl)oxy)ethyl)-4-fluorophenyl)pyridin-3-yl)oxy)-1-(cyclopropylmethyl)-1H-pyrazole-3-carboxylate (400 mg, 49.0% yield) as a white solid. LC/MS (ESI) (m/z): 626 [M+H]⁺.

The following intermediate was synthesized using a similar protocol (with m/z (ESI) values):

4-{[1-(2-{1-[(5-bromo-2-nitropyridin-3-yl)oxy]ethyl}-4,6-difluorophenyl)-3-methyl-1H-
pyrazol-5-yl]methyl}-1-(cyclopropylmethyl)-N,N-dimethyl-1H-pyrazole-3-carboxamide

| | |
|---|---|
| 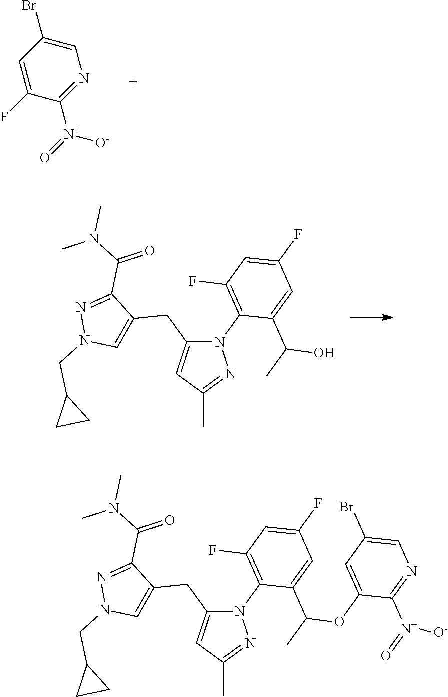 [SMILES: O=[N+]([O-])c1ncc(Br)cc1F ; Cc1cc(Cc2cn(CC3CC3)nc2C(=O)N(C)C)n(-c2c(F)cc(F)cc2C(C)O)n1 ; Cc1cc(Cc2cn(CC3CC3)nc2C(=O)N(C)C)n(-c2c(F)cc(F)cc2C(C)Oc2cc(Br)cnc2[N+](=O)[O-])n1] | 644 [M + H]⁺ |

### Synthesis of 3-{1-[4-fluoro-2-(1-hydroxyethyl)phenyl]-3-methyl-1H-pyrazole-5-carbonyl}-1H-pyrazole-5-carbonitrile

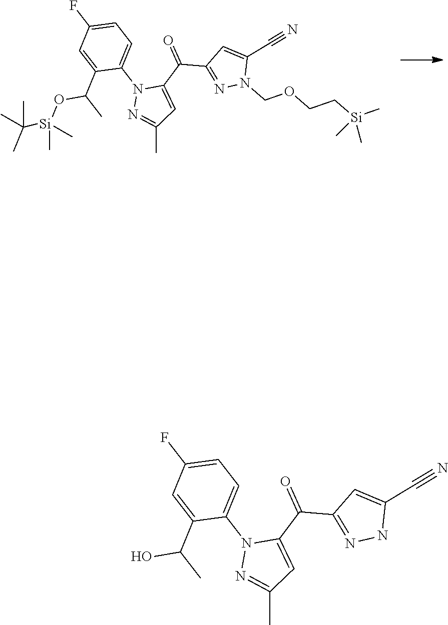 [SMILES: Cc1cc(C(=O)c2cc(C#N)n(COCC[Si](C)(C)C)n2)n(-c2ccc(F)cc2C(C)O[Si](C)(C)C(C)(C)C)n1 ; Cc1cc(C(=O)c2cc(C#N)[nH]n2)n(-c2ccc(F)cc2C(C)O)n1]

To a solution of 3-(1-(2-(1-((tert-butyldimethylsilyl)oxy)ethyl)-4-fluorophenyl)-3-methyl-1H-pyrazole-5-carbonyl)-1-((2-(trimethylsilyl)ethoxy)methyl)-1H-pyrazole-5-carbonitrile (584 mg, 1.00 mmol) in THF (8 mL) was added TBAF (2.00 mL, 4.00 mmol) at r.t. The reaction was stirred at 40° C. for 2 h. The mixture was diluted with water (15 mL) and extracted with EtOAc (20 mL×2) The combined organic phases were washed with brine (20 mL×3), dried over anhydrous Na₂SO₄, filtered and concentrated. The residue was purified by flash column chromatography on silica gel (0→8% MeOH in DCM) to give 3-{1-[4-fluoro-2-(1-hydroxyethyl)phenyl]-3-methyl-1H-pyrazole-5-carbonyl}-1H-pyrazole-5-carbonitrile (223 mg, yield:65.7%) as a white solid. LC/MS ESI (m/z): 340 [M+H]⁺.

### Synthesis of 3-(1-(2-(1-((5-bromo-2-nitropyridin-3-yl)oxy)ethyl)-4-fluorophenyl)-3-methyl-1H-pyrazole-5-carbonyl)-1-methyl-1H-pyrazole-5-carbonitrile and 5-[1-(2-{1-[(5-bromo-2-nitropyridin-3-yl)oxy]ethyl}-4-fluorophenyl)-3-methyl-1H-pyrazole-5-carbonyl]-1-methyl-1H-pyrazole-3-carbonitrile

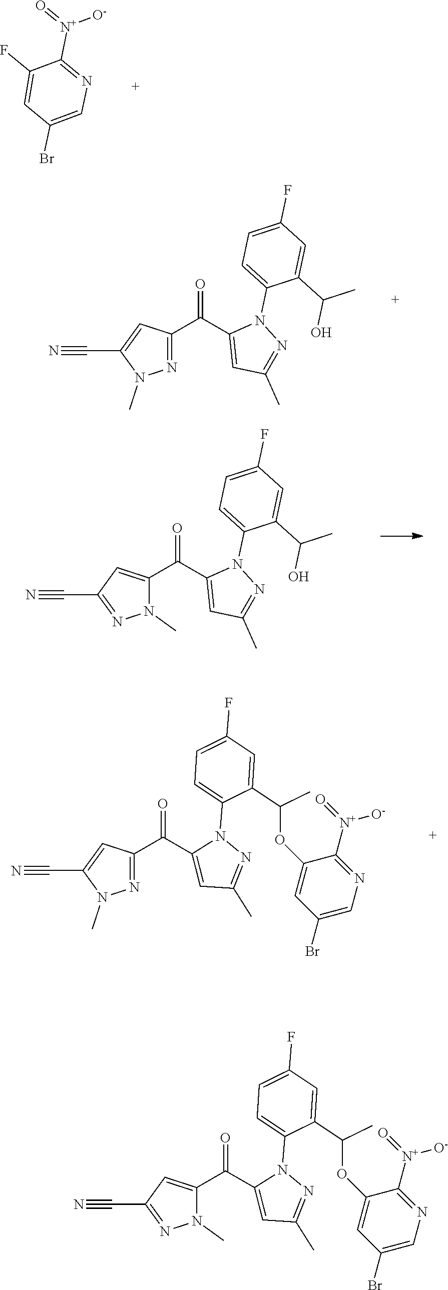

To a stirred solution of 5-(1-(4-fluoro-2-(1-hydroxyethyl)phenyl)-3-methyl-1H-pyrazole-5-carbonyl)-1-methyl-1H-pyrazole-3-carbonitrile and 3-(1-(4-fluoro-2-(1-hydroxyethyl)phenyl)-3-methyl-1H-pyrazole-5-carbonyl)-1-methyl-1H-pyrazole-5-carbonitrile (76.0 mg, mixture, 0.220 mmol) in THF (5 mL) was added NaH (10.3 mg, 0.260 mmol, 60% in mineral oil) at 0° C. and the mixture was stirred at 0° C. for 0.5 h under N₂. A solution of 5-bromo-3-fluoro-2-nitropyridine (61.8 mg, 0.280 mmol) in THE (5 mL) was then added and the reaction was stirred at 50° C. for 12 h. The mixture was quenched with sat. aq. NH₄C1 (10 mL) and extracted with EtOAc (15 mL×3). The combined organic phases were washed with brine (10 mL×2), dried over anhydrous Na₂SO₄, filtered, and concentrated. The residue was purified by flash column chromatography on silica gel (0→10% MeOH in DCM) to give 3-(1-(2-(1-((5-bromo-2-nitropyridin-3-yl)oxy)ethyl)-4-fluorophenyl)-3-methyl-1H-pyrazole-5-carbonyl)-1-methyl-1H-pyrazole-5-carbonitrile (45.0 mg, yield:37.7%) as a yellow solid. 5-[1-(2-{1-[(5-bromo-2-nitropyridin-3-yl)oxy]ethyl}-4-fluorophenyl)-3-methyl-1H-pyrazole-5-carbonyl]-1-methyl-1H-pyrazole-3-carbonitrile was not isolated. LC/MS ESI (m/z): 554 [M+H]⁺.

### Compounds

### Example 1 (Method A)

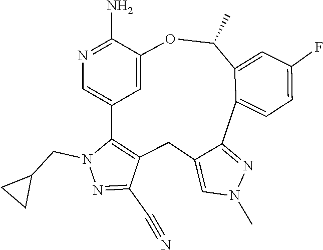 [SMILES: C[C@H]1Oc2cc(cnc2N)-c2c(c(C#N)nn2CC2CC2)Cc2cn(C)nc2-c2ccc(F)cc21]

Name: (19R)-22-amino-3-(cyclopropylmethyl)-16-fluoro-10,19-dimethyl-20-oxa-3,4,10,11,23-pentaazapentacyclo[19.3.1.0^{2,6}.0^{8,12}.0^{13,18}]pentacosa-1(24),2(6),4,8,11,13,15,17,21(25),22-decaene-5-carbonitrile

NMR: 1H NMR (400 MHz, MeOD) δ 7.60 (s, 1H), 7.55 (dd, J=10.1, 2.5 Hz, 1H). 7.49 (d. J=1.6 Hz. 1H), 7.29-7.16 (m, 1H). 7.08 (d, J=2.6 Hz. 1H), 6.46 (s, 1H), 5.45 (d, J=6.3 Hz, 1H), 4.09 (d, J=6.3 Hz, 1H), 4.02 (s, 1H). 3.94 (s, 3H), 3.80 (d. J=15.8 Hz, 1H), 3.04 (d, J=15.8 Hz, 1H), 1.82 (d, J=6.2 Hz, 3H), 1.17-1.07 (m, 1H), 0.57-0.41 (m, 2H), 0.31 (d. J=4.8 Hz, 1H), 0.20-0.07 (m, 1H).

LCMS: Method N; t_{R}: 1.19 min; m/z: 470 [M+H]⁺

To a solution of (R)-4-((3-(2-(1-((5-bromo-2-nitropyridin-3-yl)oxy)ethyl)-4-fluorophenyl)-1-methyl-1H-pyrazol-4-yl)methyl)-1-(cyclopropylmethyl)-1H-pyrazole-3-carbonitrile (100 mg, 0.17 mmol) in EtOH (5 mL) and H₂O (1.5 mL) was added iron powder (96.2 mg, 1.72 mmol) and NH₄Cl (184 mg, 3.44 mmol). The reaction was stirred at 70° C. for 1 h. The mixture was diluted with water (5 mL) and extracted with EtOAc (20 mL×2). The combined organic layers were dried over anhydrous Na₂SO₄, filtered, and concentrated. The residue was purified by column chromatography (SiO₂, 50% EtOAc in PE) to give (R)-4-((3-(2-(1-((2-amino-5-bromopyridin-3-yl)oxy)ethyl)-4-fluorophenyl)-1-methyl-1H-pyrazol-4-yl)methyl)-1-(cyclopropylmethyl)-1H-pyrazole-3-carbonitrile (60 mg, 63% yield) as yellow solid. LC/MS (ESI) (m/z): 550 [M+H]⁺.

To a solution of (R)-4-((3-(2-(1-((2-amino-5-bromopyridin-3-yl)oxy)ethyl)-4-fluorophenyl)-1-methyl-1H-pyrazol-4-yl)methyl)-1-(cyclopropylmethyl)-1H-pyrazole-3-carbonitrile (60 mg, 0.11 mmol) in 2-methyl-2-butanol (2 mL) was added KOAc (27 mg, 0.27 mmol), Pd(OAc)₂ (6.0 mg, 0.026 mmol) and cataCXium A (19 mg, 0.052 mmol) and the reaction was stirred at 120° C. for 12 h under N₂. The reaction was diluted with water (5 mL) and extracted with EtOAc (20 mL×2). The combined organic layers were dried over anhydrous Na₂SO₄, filtered, and concentrated. The residue was purified by preparative HPLC (Column: Gemini Sum C18 250×21.2 mm; 20→95% MeCN in H₂O+0.1% FA) to afford the target compound (22 mg, 43% yield). LC/MS (ESI) (m/z): 470 [M+H]⁺.

The following compounds were prepared in a similar manner:

| | | |
|---|---|---|
| Example 3 | 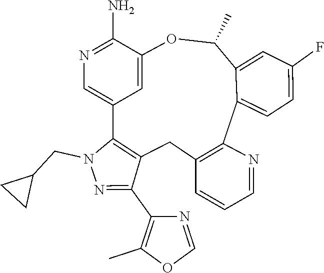 [SMILES: Cc1ocnc1-c1nn(CC2CC2)c2c1Cc1cccnc1-c1ccc(F)cc1[C@@H](C)Oc1cc-2cnc1N] | (20R)-3-(cyclopropylmethyl)-17-fluoro-20-methyl-5-(5- methyl-1,3-oxazol-4-yl)-21-oxa-3,4,12,24- tetraazapentacyclo[20.3.1.0²,⁶.0⁸,¹³.0¹⁴,¹⁹]hexacosa- 1(25),2(6),4,8(13),9,11,14,16,18,22(26),23-undecaen-23- amine 1H NMR (400 MHz, MeOD) δ 8.47 (d, J = 3.9 Hz, 1H), 8.08 (s, 1H), 7.97 (d, J = 7.9 Hz, 1H), 7.57 (d, J = 2.6 Hz, 1H), 7.50 (d, J = 1.6 Hz, 1H), 7.37 (dd, J = 8.0, 4.8 Hz, 1H), 7.26 (dd, J = 8.5, 5.7 Hz, 1H), 7.09 (td, J = 8.3, 2.7 Hz, 1H), 6.30 (d, J = 1.6 Hz, 1H), 5.24-5.10 (m, 1H), 4.52 (d, J = 15.7 Hz, 1H), 4.17 (dd, J = 14.4, 6.1 Hz, 1H), 4.00 (dd, J = 14.4, 7.4 Hz, 1H), 3.26 (d, J = 15.7 Hz, 1H), 2.66 (s, 3H), 1.85 (d, J = 6.3 Hz, 3H), 1.23- 1.12 (m, 1H), 0.61-0.40 (m, 2H), 0.37-0.28 (m, 1H), 0.17 (dt, J = 9.8, 4.8 Hz, 1H). LCMS Method U; t_{R}: 1.63 min; m/z: 523 [M + H]⁺ |
| Example 4 | 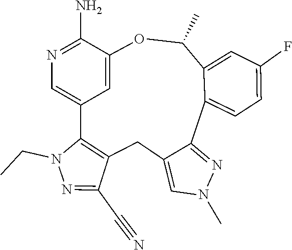 [SMILES: CCn1nc(C#N)c2c1-c1cnc(N)c(c1)O[C@H](C)c1cc(F)ccc1-c1nn(C)cc1C2] | (R)-22-amino-3-ethyl-16-fluoro-10-methyl-19-methyl-20- oxa-3,4,10,11,23- pentaazapentacyclo[19.3.1.0²,⁶.0⁸,¹².0¹³,¹⁸]pentacosa- 1(24),2(6),4,8,11,13,15,17,21(25),22-decaene-5-carbonitrile 1H NMR (400 MHz, DMSO) δ 7.71 (dd, J = 10.4, 2.6 Hz, 1H), 7.64 (s, 1H), 7.53 (d, J = 1.8 Hz, 1H), 7.27-7.10 (m, 2H), 6.34- 6.20 (m, 3H), 5.38-5.27 (m, 1H), 4.20-4.10 (m, 2H), 3.88 (s, 3H), 3.71 (d, J = 15.9 Hz, 1H), 2.83 (d, J = 15.7 Hz, 1H), 1.72 (d, J = 6.2 Hz, 3H), 1.31 (t, J = 7.2 Hz, 3H). LCMS Method F; t_{R}: 1.13 min; m/z: 444 [M + H]⁺ |
| Example 14 | 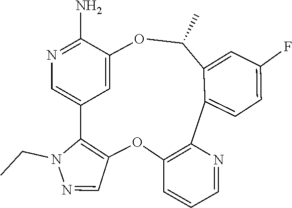 [SMILES: CCn1ncc2c1-c1cnc(N)c(c1)O[C@H](C)c1cc(F)ccc1-c1ncccc1O2] | (R)-3-ethyl-17-fluoro-20-methyl-7,21-dioxa-3,4,12,24- tetraazapentacyclo[20.3.1.0²,⁶.0⁸,¹³.0¹⁴,¹⁹]hexacosa- 1(25),2(6),4,8(13),9,11,14,16,18,22(26),23-undecaen-23- ylamine 1H NMR (400 MHz, DMSO) δ 8.44 (dd, J = 4.6, 1.1 Hz, 1H), 7.93 (s, 1H), 7.80 (dd, J = 8.5, 1.2 Hz, 1H), 7.58 (dd, J = 10.4, 2.7 Hz, 1H), 7.52 (dd, J = 8.5, 4.6 Hz, 1H), 7.49 (d, J = 1.8 Hz, 1H), 7.26 (dd, J = 8.5, 5.8 Hz, 1H), 7.10 (td, J = 8.5, 2.7 Hz, 1H), 6.26 (d, J = 1.6 Hz, 1H), 6.16 (s, 2H), 5.10 (d, J = 4.7 Hz, 1H), 4.16 (ddd, J = 14.1, 8.7, 5.2 Hz, 2H), 1.73 (d, J = 6.2 Hz, 3H), 1.32 (t, J = 7.2 Hz, 3H). LCMS Method F; t_{R}: 0.95 min; m/z: 418 [M + H]⁺ |
| Example 15 | 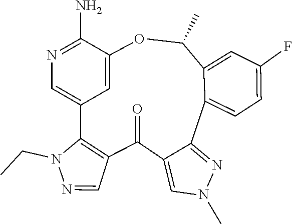 [SMILES: CCn1ncc2c1-c1cnc(N)c(c1)O[C@H](C)c1cc(F)ccc1-c1nn(C)cc1C2=O] | (R)-22-amino-3-ethyl-16-fluoro-10-methyl-19-methyl-20- oxa-3,4,10,11,23- pentaazapentacyclo[19.3.1.0²,⁶.0⁸,¹².0¹³,¹⁸]pentacosa- 1(24),2(6),4,8,11,13,15,17,21(25),22-decaen-7-one 1H NMR (400 MHz, DMSO) δ 7.85 (s, 2H), 7.68-7.58 (m, 2H), 7.57 (d, J = 2.0 Hz, 1H), 7.24 (td, J = 8.5, 2.7 Hz, 1H), 6.19 (s, 2H), 6.14 (d, J = 2.0 Hz, 1H), 5.48-5.41 (m, 1H), 4.08 (q, J = 7.2 Hz, 2H), 3.76 (s, 3H), 1.71 (d, J = 6.5 Hz, 3H), 1.29 (t, J = 7.2 Hz, 3H). LCMS Method N; t_{R}: 0.86 min; m/z: 433 [M + H]⁺ |
| Example 18 | 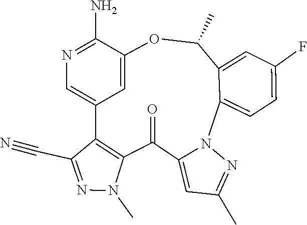 [SMILES: Cc1cc2n(n1)-c1ccc(F)cc1[C@@H](C)Oc1cc(cnc1N)-c1c(C#N)nn(C)c1C2=O] | (R)-22-amino-16-fluoro-5-methyl-10,19-dimethyl-7-oxo-20- oxa-4,5,11,12,23- pentaazapentacyclo[19.3.1.0²,⁶.0⁸,¹².0¹³,¹⁸]pentacosa- 1(24),2(6),3,8,10,13,15,17,21(25),22-decaene-3-carbonitrile 1H NMR (400 MHz, DMSO) δ 7.83 (dd, J = 8.9, 5.3 Hz, 1H), 7.74 (d, J = 2.0 Hz, 1H), 7.69 (dd, J = 9.7, 2.9 Hz, 1H), 7.37 (td, 1H), 7.06 (s, 1H), 6.30 (d, J = 1.6 Hz, 3H), 4.54-4.46 (m, 1H), 4.06 (s, 3H), 2.28 (s, 3H), 1.65 (d, J = 6.3 Hz, 3H). LCMS Method U; t_{R}: 1.41 min; m/z: 444 [M + H]⁺ |
| Example 19 | 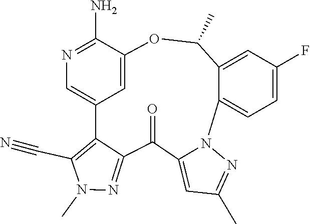 [SMILES: Cc1cc2n(n1)-c1ccc(F)cc1[C@@H](C)Oc1cc(cnc1N)-c1c(nn(C)c1C#N)C2=O] | (R)-22-amino-16-fluoro-4-methyl-10,19-dimethyl-7-oxo-20- oxa-4,5,11,12,23- pentaazapentacyclo[19.3.1.0²,⁶.0⁸,¹².0¹³,¹⁸]pentacosa- 1(24),2,5,8, 10,13,15,17,21(25),22-decaene-3-carbonitrile 1H NMR (400 MHz, DMSO) δ 7.77 (dd, J = 8.9, 5.3 Hz, 1H), 7.69 (dd, J = 9.7, 2.9 Hz, 1H), 7.64 (d, J = 1.9 Hz, 1H), 7.33 (td, J = 8.5, 3.0 Hz, 1H), 6.82 (s, 1H), 6.36 (s, 2H), 6.32 (d, J = 1.9 Hz, 1H), 4.66-4.57 (m, 1H), 4.10 (s, 3H), 2.30 (s, 3H), 1.62 (d, J = 6.2 Hz, 3H). LCMS Method U; t_{R}: 1.36 min; m/z: 444 [M + H]⁺ |
| Example 21 | 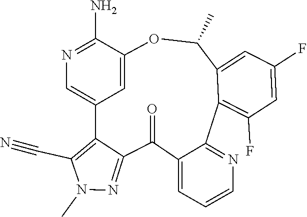 [SMILES: C[C@H]1Oc2cc(cnc2N)-c2c(nn(C)c2C#N)C(=O)c2cccnc2-c2c(F)cc(F)cc21] | (R)-23-amino-15,17-difluoro-4-methyl-20-methyl-7-oxo-21- oxa-4,5,12,24- tetraazapentacyclo[20.3.1.0²,⁶.0⁸,¹³.0¹⁴,¹⁹]hexacosa- 1(25),2,5,8,10,12,14,16,18,22(26),23-undecaene-3- carbonitrile 1H NMR (400 MHz, DMSO) δ 8.97 (dd, J = 4.9, 1.6 Hz, 1H), 8.47 (dd, J = 8.0, 1.6 Hz, 1H), 7.78 (dd, J = 8.0, 4.9 Hz, 1H), 7.65 (d, J = 1.9 Hz, 1H), 7.45 (dd, J = 9.3, 1.0 Hz, 1H), 7.29 (td, J = 9.0, 2.3 Hz, 1H), 6.33 (s, 2H), 6.14 (d, J = 1.7 Hz, 1H), 4.75- 4.69 (m, 1H), 4.12 (s, 3H), 1.59 (d, J = 6.1 Hz, 3H). LCMS Method U; t_{R}: 1.30 min; m/z: 459 [M + H]⁺ |
| Example 22 | 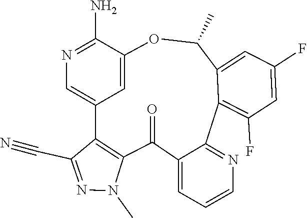 [SMILES: C[C@H]1Oc2cc(cnc2N)-c2c(C#N)nn(C)c2C(=O)c2cccnc2-c2c(F)cc(F)cc21] | (R)-23-amino-15,17-difluoro-5-methyl-20-methyl-7-oxo-21- oxa-4,5,12,24- tetraazapentacyclo[20.3.1.0²,⁶.0⁸,¹³.0¹⁴,¹⁹]hexacosa- 1(25),2(6),3,8,10,12,14,16,18,22(26),23-undecaene-3- carbonitrile 1H NMR (400 MHz, DMSO) δ 8.95 (dd, J = 4.9, 1.5 Hz, 1H), 8.30 (dd, J = 8.0, 1.5 Hz, 1H), 7.77 (d, J = 2.0 Hz, 1H), 7.69 (dd, J = 8.0, 4.9 Hz, 1H), 7.43 (dd, J = 9.7, 1.7 Hz, 1H), 7.36 (td, J = 9.2, 2.4 Hz, 1H), 6.27 (s, 2H), 6.19 (d, J = 2.0 Hz, 1H), 4.45-4.36 (m, 1H), 4.07 (s, 3H), 1.58 (d, J = 6.3 Hz, 3H). LCMS Method U; t_{R}: 1.11 min; m/z: 459 [M + H]⁺ |
| Example 27 | 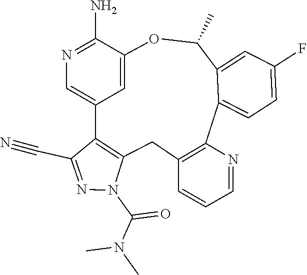 [SMILES: C[C@H]1Oc2cc(cnc2N)-c2c(C#N)nn(C(=O)N(C)C)c2Cc2cccnc2-c2ccc(F)cc21] | N,N-dimethyl-(R)-23-amino-3-cyano-17-fluoro-20-methyl- 21-oxa-4,5,12,24- tetraazapentacyclo[20.3.1.0²,⁶.0⁸,¹³.0¹⁴,¹⁹]hexacosa- 1(25),2(6),3,8(13),9,11,14,16,18,22(26),23-undecaene-5- carboxamide 1H NMR (400 MHz, DMSO) δ 8.66-8.59 (m, 1H), 8.14-8.08 (m, 1H), 7.63 (td, J = 5.6, 2.7 Hz, 2H), 7.51 (dd, J = 8.0, 4.7 Hz, 1H), 7.40 (dd, J = 8.5, 5.8 Hz, 1H), 7.19 (td, J = 8.4, 2.7 Hz, 1H), 6.19 (s, 2H), 6.07 (s, 1H), 4.98 (q, J = 5.8 Hz, 1H), 4.41 (d, J = 16.3 Hz, 1H), 3.48 (d, J = 16.2 Hz, 1H), 3.17 (s, 3H), 2.84 (s, 3H), 1.74 (d, J = 6.2 Hz, 3H). LCMS Method Q; t_{R}: 1.21 min; m/z: 484 [M + H]⁺ |
| Example 28 | 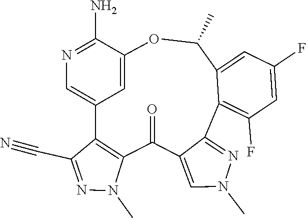 [SMILES: C[C@H]1Oc2cc(cnc2N)-c2c(C#N)nn(C)c2C(=O)c2cn(C)nc2-c2c(F)cc(F)cc21] | (R)-22-amino-14,16-difluoro-5,10-dimethyl-19-methyl-7-oxo- 20-oxa-4,5,10,11,23- pentaazapentacyclo[19.3.1.0²,⁶.0⁸,¹².0¹³,¹⁸]pentacosa- 1(24),2(6),3,8,11,13,15,17,21(25),22-decaene-3-carbonitrile 1H NMR (400 MHz, MeOD) δ 8.52 (s, 1H), 7.62 (d, J = 1.8 Hz, 1H), 7.32 (d, J = 8.4 Hz, 1H), 7.12-7.07 (m, 1H), 6.78 (s, 1H), 5.14 (d, J = 6.2 Hz, 1H), 4.14 (s, 3H), 4.06 (s, 3H), 1.74 (d, J = 6.4 Hz, 3H). LCMS Method U; t_{R}: 0.87 min; m/z: 462 [M + H]⁺ |
| Example 44 | 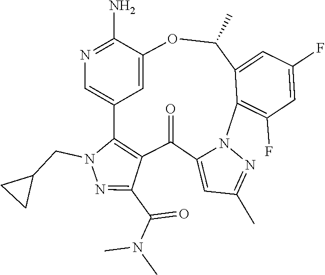 | N,N-dimethyl-(R)-22-amino-3-(cyclopropylmethyl)-14,16- difluoro-10,19-dimethyl-20-oxa-3,4,11,12,23- pentaazapentacyclo[19.3.1.0²,^{6.}0⁸,¹².0¹³,¹⁸]pentacosa- 1(24),2(6),4,8, 10,13,15,17,21(25),22-decaene-5-carboxamide 1H NMR (400 MHz, CDCl3) δ 7.61 (s, 1H), 7.14 (d, J = 9.3 Hz, 1H), 6.90-6.80 (m, 1H), 6.46 (d, J = 8.7 Hz, 2H), 5.42 (d, J = 6.3 Hz, 1H), 4.93 (s, 2H), 4.17 (dd, J = 14.5, 5.9 Hz, 2H), 3.91 (dd, J = 14.2, 7.3 Hz, 1H), 3.33 (s, 3H), 3.18 (s, 3H), 3.02- 2.89 (m, 1H), 2.29 (s, 3H), 1.81 (d, J = 6.3 Hz, 3H), 1.25- 1.17 (m, 1H), 0.59-0.47 (m, 2H), 0.38-0.32 (m, 1H), 0.27- 0.20 (m, 1H). LCMS Method U; t_{R}: 1.25 min; m/z: 534 [M + H]⁺ |
| Example 45 | 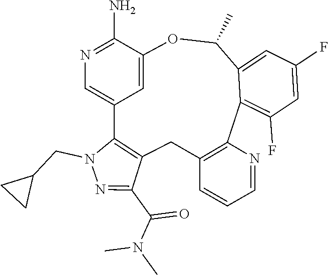 [SMILES: C[C@H]1Oc2cc(cnc2N)-c2c(c(C(=O)N(C)C)nn2CC2CC2)Cc2cccnc2-c2c(F)cc(F)cc21] | N,N-dimethyl-(R)-23-amino-3-(cyclopropylmethyl)-15,17- difluoro-20-methyl-21-oxa-3,4,12,24- tetraazapentacyclo[20.3.1.0²,⁶.0⁸,¹³.0¹⁴,¹⁹]hexacosa- 1(25),2(6),4,8(13),9,11,14,16, 18,22(26),23-undecaene-5- carboxamide 1H NMR (400 MHz, MeOD) δ 8.57 (d, J = 4.1 Hz, 1H), 8.22 (d, J = 7.7 Hz, 1H), 7.54-7.47 (m, 2H), 7.43 (d, J = 9.5 Hz, 1H), 7.03-6.95 (m, 1H), 6.27 (d, J = 1.7 Hz, 1H), 5.13 (d, J = 5.3 Hz, 1H), 4.22-4.10 (m, 2H), 4.02 (dd, J = 14.4, 7.4 Hz, 1H), 3.24 -3.14 (m, 6H), 1.82 (d, J = 6.2 Hz, 3H), 1.29 (s, 1H), 1.16 (s, 1H), 0.48 (ddd, J = 19.5, 9.1, 4.6 Hz, 2H), 0.32 (dd, J = 9.3, 4.5 Hz, 1H), 0.15 (dd, J = 9.8, 4.6 Hz, 1H). LCMS Method U; t_{R}: 1.14 min; m/z: 531 [M + H]⁺ |
| Example 46 | 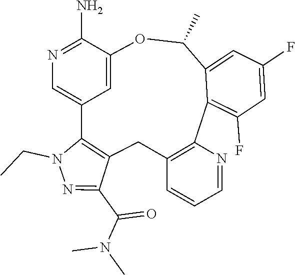 [SMILES: CCn1nc(C(=O)N(C)C)c2c1-c1cnc(N)c(c1)O[C@H](C)c1cc(F)cc(F)c1-c1ncccc1C2] | N,N-dimethyl-(R)-23-amino-3-ethyl-15,17-difluoro-20- methyl-21-oxa-3,4,12,24- tetraazapentacyclo[20.3.1.0²,⁶.0⁸,¹³.0¹⁴,¹⁹]hexacosa- 1(25),2(6),4,8(13),9,11,14,16,18,22(26),23-undecaene-5- carboxamide 1H NMR (400 MHz, MeOD) δ 8.56 (d, J = 3.9 Hz, 1H), 8.21 (d, J = 7.9 Hz, 1H), 7.52-7.48 (m, 2H), 7.43 (dd, J = 9.5, 1.3 Hz, 1H), 7.03-6.97 (m, 1H), 6.27 (d, J = 1.7 Hz, 1H), 5.15- 5.09 (m, 1H), 4.26-4.20 (m, 2H), 4.13 (d, J = 15.6 Hz, 1H), 3.30 (s, 3H), 3.18-3.14 (m, 4H), 1.82 (d, J = 6.3 Hz, 3H), 1.40 (t, J = 7.2 Hz, 3H). LCMS Method R; t_{R}: 0.72 min; m/z: 505 [M + H]⁺ |
| Example 48 | 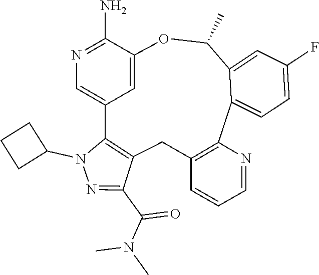 [SMILES: C[C@H]1Oc2cc(cnc2N)-c2c(c(C(=O)N(C)C)nn2C2CCC2)Cc2cccnc2-c2ccc(F)cc21] | N,N-dimethyl-(R)-23-amino-3-cyclobutyl-17-fluoro-20- methyl-21-oxa-3,4,12,24- tetraazapentacyclo[20.3.1.0²,⁶.0⁸,¹³.0¹⁴,¹⁹]hexacosa- 1(25),2(6),4,8(13),9,11,14,16,18,22(26),23-undecaene-5- carboxamide 1H NMR (400 MHz, DMSO) δ 8.52 (d, J = 3.4 Hz, 1H), 8.09 (d, J = 7.6 Hz, 1H), 7.68 (dd, J = 10.4, 2.6 Hz, 1H), 7.43 (dd, J = 8.0, 4.7 Hz, 1H), 7.35 (d, J = 1.7 Hz, 1H), 7.26 (dd, J = 8.6, 5.8 Hz, 1H), 7.12 (td, J = 8.4, 2.6 Hz, 1H), 6.21 (s, 2H), 6.05 (s, 1H), 5.11 (d, J = 6.4 Hz, 1H), 4.94-4.81 (m, 1H), 4.09 (d, J = 15.4 Hz, 1H), 3.23 (s, 3H), 3.08 (s, 3H), 3.04 (d, J = 15.5 Hz, 1H), 2.66-2.57 (m, 1H), 2.46 (d, J = 8.3 Hz, 1H), 2.13 (d, J = 11.1 Hz, 1H), 2.02-1.97 (m, 1H), 1.74 (d, J = 6.1 Hz, 3H), 1.56-1.38 (m, 1H), 1.27 (s, 1H). LCMS Method R; t_{R}: 1.18 min; m/z: 513 [M + H]⁺ |
| Example 55 | 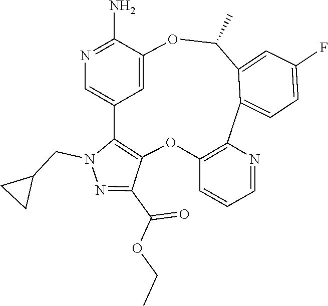 [SMILES: CCOC(=O)c1nn(CC2CC2)c2c1Oc1cccnc1-c1ccc(F)cc1[C@@H](C)Oc1cc-2cnc1N] | ethyl (20R)-23-amino-3-(cyclopropylmethyl)-17-fluoro-20- methyl-7,21-dioxa-3,4,12,24- tetraazapentacyclo[20.3.1.0²,⁶.0⁸,¹³.0¹⁴,¹⁹]hexacosa- 1(25),2(6),4,8(13),9,11,14,16,18,22(26),23-undecaene-5- carboxylate 1H NMR (400 MHz, DMSO) δ 8.44 (d, J = 4.4 Hz, 1H), 7.60 (d, J = 9.3 Hz, 3H), 7.53-7.44 (m, 1H), 7.28-7.19 (t, 1H), 7.14 (t, J = 7.2 Hz, 1H), 6.28 (d, J = 11.5 Hz, 3H), 5.04 (d, J = 6.2 Hz, 1H), 4.40-4.11 (m, 4H), 1.72 (d, J = 5.8 Hz, 3H), 1.24 (t, J = 7.1 Hz, 3H), 1.21-1.12 (m, 1H), 0.44 (m, 2H), 0.30 (m, 1H), 0.16 (m, J = 4.8 Hz, 1H). LCMS Method C1; t_{R}: 1.57 min; m/z: 516 [M + H]⁺ |
| Example 56 | 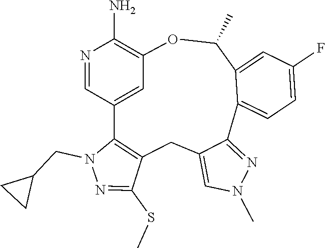 [SMILES: CSc1nn(CC2CC2)c2c1Cc1cn(C)nc1-c1ccc(F)cc1[C@@H](C)Oc1cc-2cnc1N] | (R)-3-(cyclopropylmethyl)-16-fluoro-10-methyl-19-methyl-5- (methylthio)-20-oxa-3,4,10,11,23- pentaazapentacyclo[19.3.1.0²,⁶.0⁸,¹².0¹³,¹⁸]pentacosa- 1(25),2(6),4,8,11,13,15,17,21,23-decaen-22-ylamine 1H NMR (400 MHz, DMSO) δ 7.69 (d, J = 10.1 Hz, 1H), 7.53 (s, 1H), 7.46 (s, 1H), 7.28-6.98 (m, 2H), 6.27 (s, 1H), 6.13 (s, 2H), 5.31 (d, J = 6.3 Hz, 1H), 3.95 (dd, J = 14.5, 6.0 Hz, 1H), 3.87 (s, 3H), 3.81 (dd, J = 14.4, 7.3 Hz, 1H), 3.51 (d, J = 15.6 Hz, 1H), 2.69 (d, J = 15.6 Hz, 1H), 2.53 (s, 3H), 1.70 (d, J = 6.1 Hz, 3H), 1.09 (m, 1H), 0.39 (m, J = 12.6, 8.7, 4.6 Hz, 2H), 0.24 (m, J = 9.2, 4.5 Hz, 1H), 0.06 (m, J = 9.1, 4.7 Hz, 1H). LCMS Method C1; t_{R}: 1.69 min; m/z: 491 [M + H]⁺ |
| Example 57 | 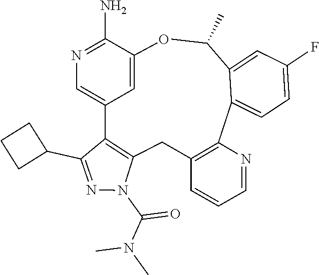 [SMILES: C[C@H]1Oc2cc(cnc2N)-c2c(C3CCC3)nn(C(=O)N(C)C)c2Cc2cccnc2-c2ccc(F)cc21] | (R)-5-(N,N-dimethylcarbamoyl)-3-cyclobutyl-17-fluoro-20- methyl-21-oxa-4,5,12,24- tetraazapentacyclo[20.3.1.0²,⁶.0⁸,¹³.0¹⁴,¹⁹]hexacosa- 1(25),2(6),3,8(13),9,11,14,16,18,22(26),23-undecaen-23- ylamine 1H NMR (400 MHz, DMSO) δ 8.59 (d, J = 4.5 Hz, 1H), 7.98 (d, J = 8.0 Hz, 1H), 7.65 (dd, J = 10.3, 2.6 Hz, 1H), 7.48 (dd, J = 8.0, 4.7 Hz, 1H), 7.33 (dd, J = 8.5, 5.8 Hz, 1H), 7.26 (d, J = 1.5 Hz, 1H), 7.15 (td, J = 8.4, 2.7 Hz, 1H), 6.00 (s, 1H), 5.87 (s, 2H), 5.04-4.93 (m, 1H), 4.41 (d, J = 15.8 Hz, 1H), 3.61-3.49 (m, 1H), 3.38 (d, J = 15.8 Hz, 1H), 3.13 (s, 3H), 3.01 (s, 3H), 2.37-2.31 (m, 1H), 2.22 (ddd, J = 13.0, 9.9, 4.1 Hz, 2H), 2.11- 2.01 (m, 1H), 1.95 (dt, J = 17.8, 8.7 Hz, 1H), 1.83 (td, J = 9.4, 4.4 Hz, 1H), 1.72 (d, J = 6.1 Hz, 3H). LCMS Method R; t_{R}: 1.15 min; m/z: 513 [M + H]⁺ |
| Example 58 | 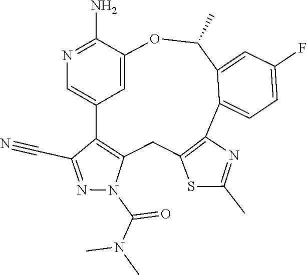 [SMILES: Cc1nc2c(s1)Cc1c(c(C#N)nn1C(=O)N(C)C)-c1cnc(N)c(c1)O[C@H](C)c1cc(F)ccc1-2] | N,N-dimethyl-(R)-22-amino-3-cyano-16-fluoro-10,19- dimethyl-20-oxa-9-thia-4,5,11,23- tetraazapentacyclo[19.3.1.0²,⁶.0⁸,¹².0¹³,¹⁸]pentacosa- 1(24),2(6),3,8(12),10,13,15,17,21(25),22-decaene-5- carboxamide 1H NMR (400 MHz, McOD) δ 7.64 (d, J = 1.8 Hz, 1H), 7.53 (dd, J = 10.0, 2.6 Hz, 1H), 7.27-7.23 (m, 1H), 7.11 (td, J = 8.4, 2.8 Hz, 1H), 6.57 (d, J = 1.8 Hz, 1H), 5.22 (d, J = 6.4 Hz, 1H), 4.46 (d, J = 15.8 Hz, 1H), 3.76 (d, J = 16.0 Hz, 1H), 3.23 (s, 3H), 3.08 (s, 3H), 2.72 (s, 3H), 1.84 (d, J = 6.3 Hz, 3H). LCMS Method N; t_{R}: 1.24 min; m/z: 504 [M + H]⁺ |
| Example 59 | 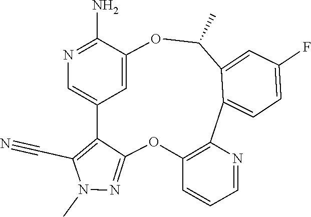 [SMILES: C[C@H]1Oc2cc(cnc2N)-c2c(nn(C)c2C#N)Oc2cccnc2-c2ccc(F)cc21] | (R)-23-amino-17-fluoro-4-methyl-20-methyl-7,21-dioxa- 4,5,12,24-tetraazapentacyclo[20.3.1.0²,⁶.0⁸,¹³.0¹⁴,¹⁹]hexacosa- 1(25),2,5,8(13),9,11,14,16,18,22(26),23-undecaene-3- carbonitrile 1H NMR (400 MHz, DMSO) δ 8.53 (dd, J = 4.6, 1.3 Hz, 1H), 7.83 (dd, J = 8.5, 1.3 Hz, 1H), 7.61 (d, J = 1.9 Hz, 1H), 7.58- 7.50 (m, 2H), 7.37 (dd, J = 8.6, 5.8 Hz, 1H), 7.15 (td, J = 8.5, 2.7 Hz, 1H), 6.35 (d, J = 1.9 Hz, 1H), 6.15 (s, 1H), 5.09-4.92 (m, 1H), 4.02 (s, 3H), 1.75 (d, J = 6.3 Hz, 3H). LCMS Method U; t_{R}: 0.94 min; m/z: 429 [M + H]⁺ |
| Example 60 | 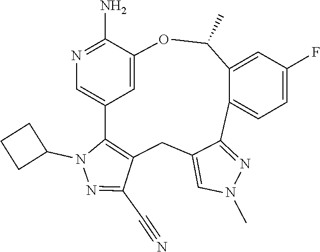 [SMILES: C[C@H]1Oc2cc(cnc2N)-c2c(c(C#N)nn2C2CCC2)Cc2cn(C)nc2-c2ccc(F)cc21] | (R)-22-amino-3-cyclobutyl-16-fluoro-10-methyl-19-methyl- 20-oxa-3,4,10,11,23- pentaazapentacyclo[19.3.1.0²,⁶.0⁸,¹².0¹³,¹⁸]pentacosa- 1(24),2(6),4,8, 11,13,15,17,21(25),22-decaene-5-carbonitrile LCMS Method W1; t_{R}: 1.04 min; m/z: 470 [M + H]⁺ |
| Example 61 | 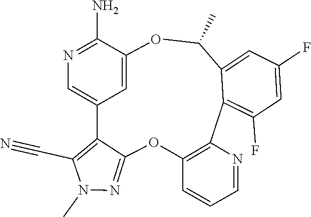 [SMILES: C[C@H]1Oc2cc(cnc2N)-c2c(nn(C)c2C#N)Oc2cccnc2-c2c(F)cc(F)cc21] | (R)-23-amino-15,17-difluoro-4-methyl-20-methyl-7,21-dioxa- 4,5,12,24-tetraazapentacyclo[20.3.1.0²,⁶.0⁸,¹³.0¹⁴,¹⁹]hexacosa- 1(25),2,5,8(13),9,11,14,16,18,22(26),23-undecaene-3- carbonitrile 1H NMR (400 MHz, DMSO) δ 8.60 (dd, J = 4.6, 1.3 Hz, 1H), 7.85 (dd, J = 8.5, 1.3 Hz, 1H), 7.65-7.59 (m, 2H), 7.46 (dd, J = 9.7, 1.8 Hz, 1H), 7.28 (td, J = 9.6, 2.5 Hz, 1H), 6.33 (d, J = 1.9 Hz, 1H), 6.20 (s, 1H), 5.05-4.95 (m, 1H), 4.03 (s, 3H), 1.73 (d, J = 6.3 Hz, 3H). LCMS Method U; t_{R}: 1.03 min; m/z: 447 [M + H]⁺ |
| Example 62 | 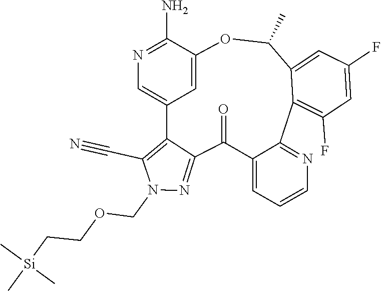 [SMILES: C[C@H]1Oc2cc(cnc2N)-c2c(nn(COCC[Si](C)(C)C)c2C#N)C(=O)c2cccnc2-c2c(F)cc(F)cc21] | (R)-23-amino-15,17-difluoro-20-methyl-7-oxo-4-{[2- (trimethylsilyl)ethoxy]methyl}-21-oxa-4,5,12,24- tetraazapentacyclo[20.3.1.0²,⁶.0⁸,¹³.0¹⁴,¹⁹]hexacosa- 1(25),2,5,8,10,12,14,16,18,22(26),23-undecaene-3- carbonitrile LCMS Method R; t_{R}: 2.00 min; m/z: 575 [M + H]⁺ |
| Example 63 | 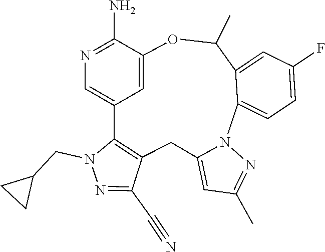 [SMILES: Cc1cc2n(n1)-c1ccc(F)cc1C(C)Oc1cc(cnc1N)-c1c(c(C#N)nn1CC1CC1)C2] | 22-amino-3-(cyclopropylmethyl)-16-fluoro-10,19-dimethyl- 20-oxa-3,4,11,12,23- pentaazapentacyclo[19.3.1.0²,⁶.0⁸,¹².0¹³,¹⁸]pentacosa- 1(24),2(6),4,8,10,13,15,17,21(25),22-decaene-5-carbonitrile LCMS Method W; t_{R}: 1.11 min; m/z: 470 [M + H]⁺ |
| Example 64 | 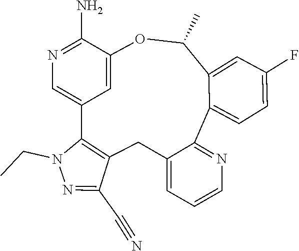 [SMILES: CCn1nc(C#N)c2c1-c1cnc(N)c(c1)O[C@H](C)c1cc(F)ccc1-c1ncccc1C2] | (R)-23-amino-3-ethyl-17-fluoro-20-methyl-21-oxa-3,4,12,24- tetraazapentacyclo[20.3.1.0²,⁶.0⁸,¹³.0¹⁴,¹⁹]hexacosa- 1(25),2(6),4,8,10,12,14,16,18,22(26),23-undecaene-5- carbonitrile LCMS Method R; t_{R}: 1.21 min; m/z: 441 [M + H]⁺ |
| Example 65 | 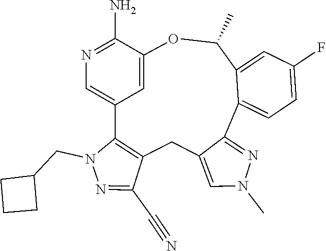 [SMILES: C[C@H]1Oc2cc(cnc2N)-c2c(c(C#N)nn2CC2CCC2)Cc2cn(C)nc2-c2ccc(F)cc21] | (R)-22-amino-3-(cyclobutylmethyl)-16-fluoro-10-methyl-19- methyl-20-oxa-3,4,10,11,23- pentaazapentacyclo[19.3.1.0²,⁶.0⁸,¹².0¹³,¹⁸]pentacosa- 1(24),2(6),4,8, 11,13,15,17,21(25),22-decaene-5-carbonitrile LCMS Method R; t_{R}: 1.20 min; m/z: 484 [M + H]⁺ |
| Example 66 | 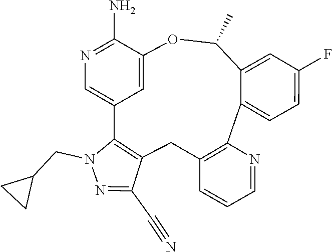 [SMILES: C[C@H]1Oc2cc(cnc2N)-c2c(c(C#N)nn2CC2CC2)Cc2cccnc2-c2ccc(F)cc21] | (R)-23-amino-3-(cyclopropylmethyl)-17-fluoro-20-methyl- 21-oxa-3,4,12,24- tetraazapentacyclo[20.3.1.0²,⁶.0⁸,¹³.0¹⁴,¹⁹]hexacosa- 1(25),2(6),4,8(13),9,11,14,16,18,22(26),23-undecaene-5- carbonitrile 1H NMR (400 MHz, DMSO) δ 8.63 (dd, J = 4.6, 1.2 Hz, 1H), 7.65 (dd, J = 10.3, 2.7 Hz, 1H), 7.61 (d, J = 7.3 Hz, 1H), 7.57 (d, J = 1.8 Hz, 1H), 7.48 (dd, J = 8.0, 4.7 Hz, 1H), 7.35 (dd, J = 8.5, 5.8 Hz, 1H), 7.24 (td, J = 8.4, 2.7 Hz, 1H), 6.07 (s, 2H), 6.01 (d, J = 1.7 Hz, 1H), 4.97-4.90 (m, 1H), 4.45 (d, J = 16.8 Hz, 1H), 4.40 (d, J = 7.1 Hz, 2H), 3.38 (d, J = 16.7 Hz, 1H), 1.73 (d, J = 6.2 Hz, 3H), 1.43-1.36 (m, 1H), 0.63-0.41 (m, 4H). LCMS Method F; t_{R}: 1.06 min; m/z: 467 [M + H]⁺ |
| Example 67 | 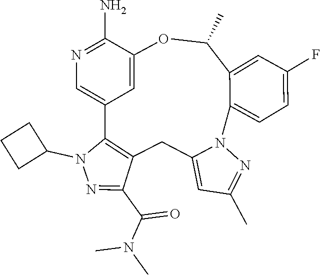 [SMILES: Cc1cc2n(n1)-c1ccc(F)cc1[C@@H](C)Oc1cc(cnc1N)-c1c(c(C(=O)N(C)C)nn1C1CCC1)C2] | N,N-dimethyl-(R)-22-amino-3-cyclobutyl-16-fluoro-10,19- dimethyl-20-oxa-3,4,11,12,23- pentaazapentacyclo[19.3.1.0²,⁶.0⁸,¹².0¹³,¹⁸]pentacosa- 1(24),2(6),4,8,10,13,15,17,21(25),22-decaene-5-carboxamide 1H NMR (400 MHz, DMSO) δ 7.79 (dd, J = 9.8, 2.9 Hz, 1H), 7.40 (d, J = 1.7 Hz, 1H), 7.32 (dd, J = 8.7, 5.2 Hz, 1H), 7.21 (td, J = 8.3, 3.0 Hz, 1H), 6.26 (s, 1H), 6.13 (s, 1H), 6.12 (d, J = 1.6 Hz, 1H), 5.25 (d, J = 4.8 Hz, 1H), 4.90-4.79 (m, 1H), 4.06 (d, J = 15.9 Hz, 1H), 3.24 (s, 3H), 3.06 (s, 3H), 2.79 (d, J = 15.7 Hz, 1H), 2.64-2.42 (m, 3H), 2.19 (s, 3H), 2.16-2.09 (m, 1H), 1.80-1.73 (m, 2H), 1.71 (d, J = 6.2 Hz, 3H). LCMS Method W; t_{R}: 1.10 min; m/z: 516 [M + H]⁺ |
| Example 68 | 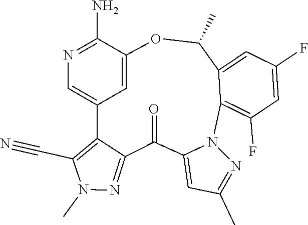 [SMILES: Cc1cc2n(n1)-c1c(F)cc(F)cc1[C@@H](C)Oc1cc(cnc1N)-c1c(nn(C)c1C#N)C2=O] | (R)-22-amino-14,16-difluoro-4-methyl-10,19-dimethyl-7-oxo- 20-oxa-4,5,11,12,23- pentaazapentacyclo[19.3.1.0²,⁶.0⁸,¹².0¹³,¹⁸]pentacosa- 1(24),2,5,8,10,13,15,17,21(25),22-decaene-3-carbonitrile 1H NMR (400 MHz, MeOD) δ 7.84 (d, J = 1.9 Hz, 1H), 7.39 (d, J = 8.9 Hz, 1H), 7.22 (dd, J = 13.6, 7.0 Hz, 1H), 6.95 (s, 1H), 6.46 (d, J = 1.8 Hz, 1H), 4.50-4.44 (m, 1H), 4.06 (s, 3H), 2.36 (s, 3H), 1.68 (d, J = 6.4 Hz, 3H). LCMS Method R; t_{R}: 1.32 min; m/z: 462 [M + H]⁺ |
| Example 69 | 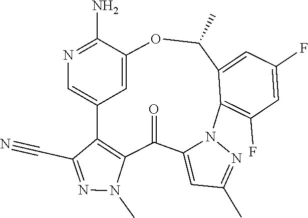 [SMILES: Cc1cc2n(n1)-c1c(F)cc(F)cc1[C@@H](C)Oc1cc(cnc1N)-c1c(C#N)nn(C)c1C2=O] | (R)-22-amino-14,16-difluoro-5-methyl-10,19-dimethyl-7-oxo- 20-oxa-4,5,11,12,23- pentaazapentacyclo[19.3.1.0²,⁶.0⁸,¹².0¹³,¹⁸]pentacosa- 1(24),2(6),3,8,10,13,15, 17,21(25),22-decaene-3-carbonitrile 1H NMR (400 MHz, MeOD) δ 7.70 (d, J = 1.9 Hz, 1H), 7.43- 7.35 (m, 1H), 7.25-7.12 (m, 1H), 6.90 (s, 1H), 6.47 (d, J = 1.8 Hz, 1H), 4.79-4.73 (m, 1H), 4.13 (s, 3H), 2.39 (s, 3H), 1.69 (d, J = 6.3 Hz, 3H). LCMS Method U; t_{R}: 1.37 min; m/z: 462 [M + H]⁺ |
| Example 70 | 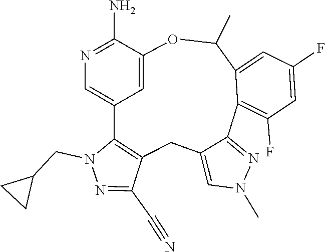 [SMILES: CC1Oc2cc(cnc2N)-c2c(c(C#N)nn2CC2CC2)Cc2cn(C)nc2-c2c(F)cc(F)cc21] | 22-amino-3-(cyclopropylmethyl)-14,16-difluoro-10,19- dimethyl-20-oxa-3,4,10,11,23- pentaazapentacyclo[19.3.1.0²,⁶.0⁸,¹².0¹³,¹⁸]pentacosa- 1(24),2(6),4,8,11,13,15,17,21(25),22-decaene-5-carbonitrile 1H NMR (400 MHz, MeOD) δ 7.62 (s, 1H), 7.53 (s, 1H), 7.45 (d, J = 8.6 Hz, 1H), 7.00 (td, J = 9.3, 2.5 Hz, 1H), 6.49 (d, J = 1.7 Hz, 1H), 5.42 (q, J = 6.2 Hz, 1H), 4.12 (dd, J = 14.4, 6.4 Hz, 1H), 4.01 (dd, J = 13.8, 7.2 Hz, 1H), 3.97 (s, 3H), 3.76 (d, J = 16.1 Hz, 1H), 2.94 (dd, J = 15.8, 1.7 Hz, 1H), 1.80 (d, J = 6.3 Hz, 3H), 1.13 (s, 1H), 0.48 (ddt, J = 17.2, 13.5, 6.7 Hz, 2H), 0.32 (td, J = 9.5, 4.7 Hz, 1H), 0.13 (td, J = 9.7, 5.0 Hz, 1H). LCMS Method U; t_{R}: 1.46 min; m/z: 488 [M + H]⁺ |
| Example 71 | 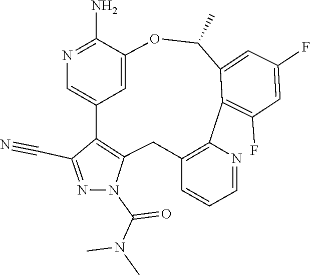 [SMILES: C[C@H]1Oc2cc(cnc2N)-c2c(C#N)nn(C(=O)N(C)C)c2Cc2cccnc2-c2c(F)cc(F)cc21] | N,N-dimethyl-(R)-23-amino-3-cyano-15,17-difluoro-20- methyl-21-oxa-4,5,12,24- tetraazapentacyclo[20.3.1.0²,⁶.0⁸,¹³.0¹⁴,¹⁹]hexacosa- 1(25),2(6),3,8(13),9,11,14,16,18,22(26),23-undecaene-5- carboxamide 1H NMR (400 MHz, MeOD) δ 8.66 (d, J = 3.6 Hz, 1H), 8.27 (d, J = 7.8 Hz, 1H), 7.72 (s, 1H), 7.55 (dd, J = 8.1, 4.8 Hz, 1H), 7.42 (d, J = 9.6 Hz, 1H), 7.05 (dd, J = 14.7, 6.0 Hz, 1H), 6.28 (d, J = 1.6 Hz, 1H), 5.02 (d, J = 4.8 Hz, 1H), 4.44 (d, J = 16.6 Hz, 1H), 3.62-3.56 (m, 1H), 3.24 (s, 3H), 3.00 (s, 3H), 1.83 (d, J = 6.3 Hz, 3H). LCMS Method R; t_{R}: 1.31 min; m/z: 502 [M + H]⁺ |
| Example 72 | 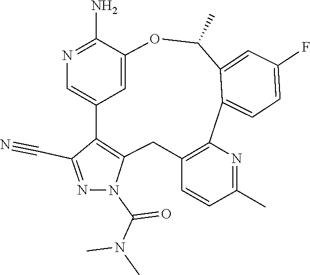 [SMILES: Cc1ccc2c(n1)-c1ccc(F)cc1[C@@H](C)Oc1cc(cnc1N)-c1c(C#N)nn(C(=O)N(C)C)c1C2] | N,N-dimethyl-(R)-23-amino-3-cyano-17-fluoro-11,20- dimethyl-21-oxa-4,5,12,24- tetraazapentacyclo[20.3.1.0²,⁶.0⁸,¹³.0¹⁴,¹⁹]hexacosa- 1(25),2(6),3,8(13),9,11,14,16,18,22(26),23-undecaene-5- carboxamide 1H NMR (400 MHz, MeOD) δ 8.13 (d, J = 8.0 Hz, 1H), 7.69 (s, 1H), 7.55-7.49 (m, 1H), 7.36 (d, J = 8.4 Hz, 1H), 7.34- 7.29 (m, 1H), 7.16-7.09 (m, 1H), 6.33 (s, 1H), 5.08-5.02 (m, 1H), 4.39 (d, J = 16.5 Hz, 1H), 3.64 (d, J = 16.1 Hz, 1H), 3.23 (s, 3H), 2.96 (s, 3H), 2.57 (s, 3H), 1.85 (d, J = 6.3 Hz, 3H). LCMS Method R; t_{R}: 1.36 min; m/z: 498 [M + H]⁺ |

### Example 5 (Method B)

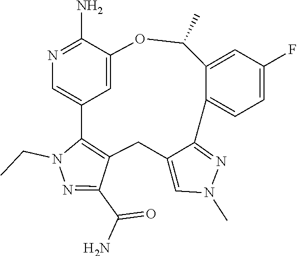 [SMILES: CCn1nc(C(N)=O)c2c1-c1cnc(N)c(c1)O[C@H](C)c1cc(F)ccc1-c1nn(C)cc1C2]

Name: (R)-22-amino-3-ethyl-16-fluoro-10-methyl-19-methyl-20-oxa-3,4,10,11,23-pentaazapentacyclo[19.3.1.0^{2,6}.0^{8,12}.0^{13,18}]pentacosa-1(24),2(6),4,8,11.13,15,17,21(25),22-decaene-5-carboxamide

NMR: 1H NMR (400 MHz, DMSO) δ 7.70 (d, J=10.0 Hz, 1H), 7.52-7.45 (m, 2H), 7.42 (s, 1H), 7.24 (s, 1H), 7.16-7.10 (m, 2H), 6.31 (d, J=1.6 Hz, 1H), 6.16 (s, 2H), 5.39-5.29 (m, 1H), 4.23 (d. J=14.9 Hz, 1H), 4.14-3.98 (m, 2H), 3.85 (s, 3H), 2.62 (d, J=14.5 Hz, 1H), 1.71 (d, J=6.2 Hz, 3H), 1.29 (t, J=7.2 Hz, 3H).

LCMS: Method F; t_{R}: 0.71 min; m/z: 462 [M+H]⁻

To a solution of (19R)-22-amino-3-ethyl-16-fluoro-10,19-dimethyl-20-oxa-3,4,10,11,23-pentaazapentacyclo[19.3.1.0^{2,6}.0^{8,12}.0^{13,18}]pentacosa-1(24),2(6),4,8,11,13,15,17,21(25),22-decaene-5-carbonitrile (20 mg, 0.045 mmol) in THF (1.0 mL) was added a solution of NaOH (5.0 mg, 0.14 mmol) in water (2.0 mL) at r.t. The reaction was stirred at 100° C. for 12 h in a sealed tube. The mixture was extracted with EtOAc (2 mL). The combined organic layers were dried over anhydrous Na₂SO₄ and concentrated in vacuo. The residue was purified by prep-HPLC (Column: Gemini 5 um C18 250×21.2 mm; 5→95% MeCN in H₂O+0.1% FA) to give the target product (10 mg, 48% yield). LC/MS (ESI) (m/z): 462 [M+H]⁺.

The following compounds were prepared in a similar manner:

| | | |
|---|---|---|
| Example 6 | 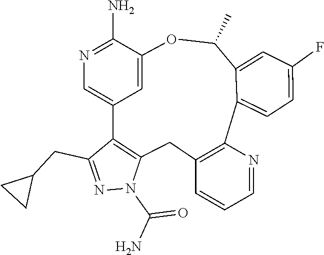 [SMILES: C[C@H]1Oc2cc(cnc2N)-c2c(CC3CC3)nn(C(N)=O)c2Cc2cccnc2-c2ccc(F)cc21] | (R)-23-amino-3-(cyclopropylmethyl)-17-fluoro-20-methyl-21-oxa- 3,4,12,24-tetraazapentacyclo[20.3.1.0²,⁶.0⁸,¹³.0¹⁴,¹⁹]hexacosa- 1(25),2(6),4,8(13),9,11,14,16, 18,22(26),23-undecaene-5- carboxamide 1H NMR (400 MHz, MeOD) δ 8.48 (d, J = 4.8 Hz, 1H), 8.09 (d, J = 7.9 Hz, 1H), 7.55 (dd, J = 10.1, 2.7 Hz, 1H), 7.49 (d, J = 1.8 Hz, 1H), 7.43 (dd, J = 8.0, 4.8 Hz, 1H), 7.25 (dd, J = 8.5, 5.6 Hz, 1H), 7.09 (td, J = 8.3, 2.7 Hz, 1H), 6.26 (d, J = 1.7 Hz, 1H), 5.24-5.14 (m, 1H), 4.59 (d, J = 15.3 Hz, 1H), 4.17 (dd, J = 14.2, 6.2 Hz, 1H), 4.03 (dd, J = 14.5, 7.5 Hz, 1H), 3.21 (d, J = 15.3 Hz, 1H), 1.85 (d, J = 6.3 Hz, 3H), 1.22-1.14 (m, 1H), 0.56-0.37 (m, 2H), 0.31 (dd, J = 9.5, 4.6 Hz, 1H), 0.13 (dd, J = 9.7, 4.8 Hz, 1H). LCMS Method W; t_{R}: 0.38 min; m/z: 485 [M + H]⁺ |
| Example 30 | 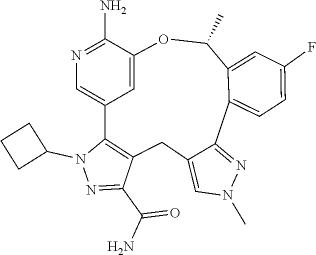 [SMILES: C[C@H]1Oc2cc(cnc2N)-c2c(c(C(N)=O)nn2C2CCC2)Cc2cn(C)nc2-c2ccc(F)cc21] | (R)-22-amino-3-cyclobutyl-16-fluoro-10-methyl-19-methyl-20-oxa- 3,4,10,11,23-pentaazapentacyclo[19.3.1.0²,⁶.0⁸,¹².0¹³,¹⁸] pentacosa- 1(24),2(6),4,8,11,13,15,17,21(25),22-decaene-5-carboxamide 1H NMR (400 MHz, MeOD) δ 7.55-7.51 (m, 2H), 7.33 (d, J = 1.5 Hz, 1H), 7.17 (dd, J = 8.5, 5.7 Hz, 1H), 7.06 (td, J = 8.4, 2.7 Hz, 1H), 6.52 (d, J = 1.7 Hz, 1H), 5.47-5.42 (m, 1H), 4.81 (d, J = 8.0 Hz, 1H), 4.31 (d, J = 15.1 Hz, 1H), 3.89 (s, 3H), 2.80 (dd, J = 16.3, 6.6 Hz, 2H), 2.64 (dd, J = 19.8, 10.0 Hz, 1H), 2.46 (dd, J = 10.7, 7.6 Hz, 1H), 2.19-2.10 (m, 1H), 1.84 (m, 5H). LCMS Method W; t_{R}: 0.87 min; m/z: 488 [M + H]⁺ |

### Example 7 (Method C)

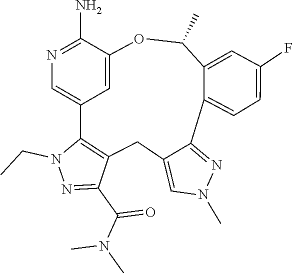 [SMILES: CCn1nc(C(=O)N(C)C)c2c1-c1cnc(N)c(c1)O[C@H](C)c1cc(F)ccc1-c1nn(C)cc1C2]

Name: N,N-dimethyl-(R)-22-amino-3-ethyl-16-fluoro-10-methyl-19-methyl-20-oxa-3,4,10,11,23-pentaazapentacyclo[19.3.1.0^{2,6}.0^{8,12}.0^{13,18}]pentacosa-1(24),2(6),4,8,11,13,15,17.21(25),22-decaene-5-carboxamide

NMR: 1H NMR (400 MHz, DMSO) δ 7.73 (s, 1H), 7.69 (d, J=10.6 Hz, 1H), 7.46 (s, 1H), 7.12 (d, J=7.0 Hz, 2H), 6.32 (s, 1H), 6.14 (s, 2H), 5.35 (d, J=6.0 Hz, 1H), 4.05 (q, J=7.2 Hz, 2H), 3.86 (s, 3H), 3.72 (d, J=15.0 Hz, 1H), 3.20 (s, 3H), 3.05 (s, 3H), 2.67 (s, 1H), 1.71 (d, J=6.1 Hz, 3H), 1.28 (t, J=7.2 Hz, 3H).

LCMS: Method F; t_{R}: 1.10 min; m/z: 490 [M+H]⁺

To a solution of (19R)-22-amino-3-ethyl-16-fluoro-10,19-dimethyl-20-oxa-3,4,10,11,23-pentaazapentacyclo[19.3.1.0^{2,6}.0^{8,12}.0^{13,18}]pentacosa-1(24),2(6),4,8,11,13,15,17,21(25),22-decaene-5-carbonitrile (10 mg, 0.023 mmol) in MeOH (2 mL) was added NaOH (2 M in water, 0.015 mL, 0.09 mmol) and the reaction was stirred at 80° C. for 12 h. The mixture was directly concentrated to give crude (19R)-22-amino-3-ethyl-16-fluoro-10,19-dimethyl-20-oxa-3,4,10,11,23-pentaazapentacyclo[19.3.1.0^{2,6}.0^{8,12}.0^{13,18}]pentacosa-1(24),2(6),4,8,11,13,15,17,21(25),22-decaene-5-carboxylic acid (8 mg, 76% yield) as yellow oil. LC/MS (ESI) (m/z): 463 [M+H]⁺.

To a solution of crude (19R)-22-amino-3-ethyl-16-fluoro-10,19-dimethyl-20-oxa-3,4,10,11,23-pentaazapentacyclo[19.3.1.0^{2,6}.0^{8,12}.0^{13,18}]pentacosa-1(24),2(6),4,8,11,13,15,17,21(25),22-decaene-5-carboxylic acid (8.0 mg, 0.017 mmol) in DMF (1.0 mL) was added dimethylamine hydrochloride (0.003 mL, 0.04 mmol), DIEA (0.009 mL, 0.05 mmol) and HATU (11 mg, 0.028 mmol). The reaction was stirred at r.t. for 2 h. The reaction mixture was diluted with water (10 mL) and extracted with EtOAc (20 mL×2). The combined organic layers were dried over Na₂SO₄, filtered, and concentrated in vacuo. The residue was purified by preparative HPLC (Column: Gemini Sum C18 250×21.2 mm; 5→95% MeCN in H₂O+0.1% FA) to afford the target product (1.6 mg, 19% yield). LC/MS (ESI) (m/z): 490 [M+H]⁺.

The following compounds were prepared in a similar manner:

| | | |
|---|---|---|
| Example 8 | 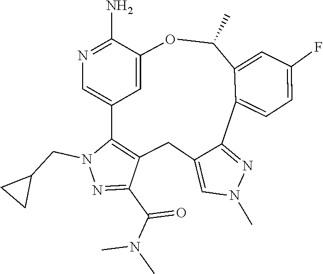 [SMILES: C[C@H]1Oc2cc(cnc2N)-c2c(c(C(=O)N(C)C)nn2CC2CC2)Cc2cn(C)nc2-c2ccc(F)cc21] | N,N-dimethyl-(R)-22-amino-3-(cyclopropylmethyl)-16-fluoro- 10-methyl-19-methyl-20-oxa-3,4,10,11,23- pentaazapentacyclo[19.3.1.0²,⁶.0⁸,¹².0¹³,¹⁸]pentacosa- 1(24),2(6),4,8,11,13,15, 17,21(25),22-decaene-5-carboxamide 1H NMR (400 MHz, DMSO) δ 7.76 (s, 1H), 7.69 (d, J = 10.2 Hz, 1H), 7.50 (s, 1H), 7.12 (d, J = 6.2 Hz, 2H), 6.32 (s, 1H), 6.17 (s, 2H), 5.36 (d, J = 5.9 Hz, 1H), 4.01 (dd, J = 14.3, 5.9 Hz, 1H), 3.84 (d, J = 14.8 Hz, 4H), 3.72 (d, J = 15.0 Hz, 1H), 3.21 (s, 3H), 3.06 (s, 3H), 2.69 (s, 1H), 1.71 (d, J = 6.2 Hz, 3H), 1.11 (dd, J = 17.3, 8.6 Hz, 1H), 0.48-0.35 (m, 2H), 0.31-0.23 (m, 1H), 0.10 (dd, J = 8.7, 4.0 Hz, 1H). LCMS Method N; t_{R}: 0.78 min; m/z: 516 [M + H]⁺ |
| Example 9 | 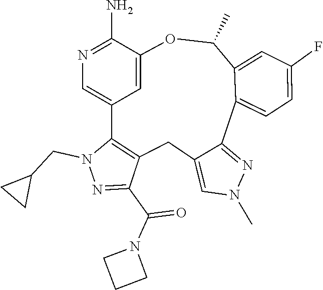 [SMILES: C[C@H]1Oc2cc(cnc2N)-c2c(c(C(=O)N3CCC3)nn2CC2CC2)Cc2cn(C)nc2-c2ccc(F)cc21] | {(R)-22-amino-3-(cyclopropylmethyl)-16-fluoro-10-methyl-19- methyl-20-oxa-3,4,10,11,23- pentaazapentacyclo[19.3.1.0²,⁶.0⁸,¹².0¹³,¹⁸]pentacosa- 1(24),2(6),4,8,11,13,15,17,21(25),22-decaen-5-yl}(1- azetidinyl)methanone 1H NMR (400 MHz, DMSO) δ 7.69 (d, 1H), 7.57 (s, 1H), 7.52 (d, J = 1.5 Hz, 1H), 7.16-7.10 (m, 2H), 6.76 (br s, 2H), 6.40 (s, 1H), 5.39 (d, J = 5.6 Hz, 1H), 4.62 (dd, J = 16.2, 9.2 Hz, 1H), 4.43 (dd, J = 16.1, 8.7 Hz, 1H), 4.17 (d, J = 14.7 Hz, 1H), 4.08 (t, J = 7.7 Hz, 2H), 4.00 (dd, J = 14.3, 6.1 Hz, 1H), 3.86-3.80 (m, 4H), 2.63 (d, J = 14.6 Hz, 1H), 2.34-2.26 (m, 2H), 1.73 (d, J = 6.2 Hz, 3H), 1.11 (s, 1H), 0.50-0.38 (m, 2H), 0.32-0.24 (m, 1H), 0.18-0.11 (m, 1H). LCMS Method U; t_{R}: 1.13 min; m/z: 528 [M + H]⁺ |
| Example 10 | 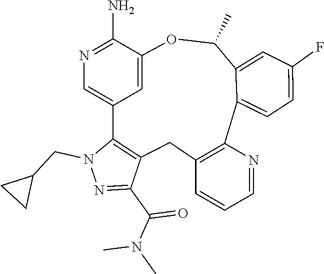 [SMILES: C[C@H]1Oc2cc(cnc2N)-c2c(c(C(=O)N(C)C)nn2CC2CC2)Cc2cccnc2-c2ccc(F)cc21] | N,N-dimethyl-(R)-23-amino-3-(cyclopropylmethyl)-17-fluoro- 20-methyl-21-oxa-3,4,12,24- tetraazapentacyclo[20.3.1.0²,⁶.0⁸,¹³.0¹⁴,¹⁹]hexacosa- 1(25),2(6),4,8(13),9,11,14,16,18,22(26),23-undecaene-5- carboxamide 1H NMR (400 MHz, DMSO) δ 8.46-8.37 (m, 1H), 8.01 (d, J = 7.6 Hz, 1H), 7.57 (dd, J = 10.4, 2.7 Hz, 1H), 7.42 (d, J = 1.7 Hz, 1H), 7.33 (d, J = 3.3 Hz, 1H), 7.15 (s, 1H), 7.02 (td, J = 8.4, 2.7 Hz, 1H), 6.11 (s, 2H), 5.95 (d, J = 1.4 Hz, 1H), 4.99 (dt, J = 10.5, 5.1 Hz, 1H), 3.99 (dd, J = 14.6, 5.8 Hz, 2H), 3.83 (dd, J = 14.4, 7.3 Hz, 1H), 3.10 (s, 3H), 2.95 (d, J = 12.1 Hz, 4H), 1.69-1.53 (m, 3H), 1.02 (dd, J = 9.1, 4.4 Hz, 1H), 0.38-0.22 (m, 2H), 0.20- 0.11 (m, 1H),-0.00 (dt, J = 9.6, 4.9 Hz, 1H). LCMS Method U; t_{R}: 1.11 min; m/z: 513 [M + H]⁺ |
| Example 11 | 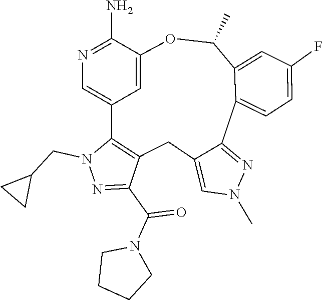 [SMILES: C[C@H]1Oc2cc(cnc2N)-c2c(c(C(=O)N3CCCC3)nn2CC2CC2)Cc2cn(C)nc2-c2ccc(F)cc21] | {(R)-22-amino-3-(cyclopropylmethyl)-16-fluoro-10-methyl-19- methyl-20-oxa-3,4,10,11,23- pentaazapentacyclo[19.3.1.0²,⁶.0⁸,¹².0¹³,¹⁸]pentacosa- 1(24),2(6),4,8,11,13,15,17,21(25),22-decaen-5-yl}(1- pyrrolidinyl)methanone 1H NMR (400 MHz, MeOD) δ 7.80 (s, 1H), 7.54 (dd, J = 10.0, 2.7 Hz, 1H), 7.46 (d, J = 1.6 Hz, 1H), 7.18 (dd, J = 8.5, 5.8 Hz, 1H), 7.08 (td, J = 8.4, 2.7 Hz, 1H), 6.63 (s, 1H), 5.51 (d, J = 5.2 Hz, 1H), 4.09 (dd, J = 14.4, 6.3 Hz, 1H), 4.06-3.99 (m, 2H), 3.97-3.89 (m, 4H), 3.82-3.71 (m, 1H), 3.67 (t, J = 6.2 Hz, 2H), 2.87 (d, J = 15.1 Hz, 1H), 1.99 (dd, J = 11.9, 6.1 Hz, 4H), 1.84 (d, J = 6.3 Hz, 3H), 1.18-1.05 (m, 1H), 0.56-0.37 (m, 2H), 0.31 (dd, J = 9.5, 4.7 Hz, 1H), 0.13 (dd, J = 9.3, 4.5 Hz, 1H). LCMS Method R; t_{R}: 1.04 min; m/z: 542 [M + H]⁺ |
| Example 12 | 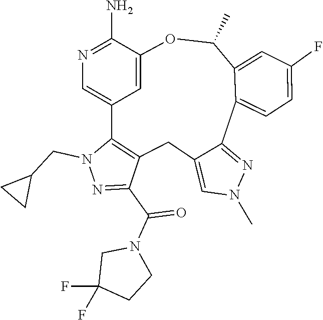 [SMILES: C[C@H]1Oc2cc(cnc2N)-c2c(c(C(=O)N3CCC(F)(F)C3)nn2CC2CC2)Cc2cn(C)nc2-c2ccc(F)cc21] | {(R)-22-amino-3-(cyclopropylmethyl)-16-fluoro-10-methyl-19- methyl-20-oxa-3,4,10,11,23- pentaazapentacyclo[19.3.1.0²,⁶.0⁸,¹².013,18]pentacosa- 1(24),2(6),4,8,11,13,15,17,21(25),22-decaen-5-yl}(3,3-difluoro- 1-pyrrolidinyl)methanone 1H NMR (400 MHz, MeOD) δ 7.74 (d, J = 16.5 Hz, 1H), 7.54 (dd, J = 10.1, 2.6 Hz, 1H), 7.46 (d, J = 1.8 Hz, 1H), 7.17 (dd, J = 8.6, 5.8 Hz, 1H), 7.07 (td, J = 8.4, 2.8 Hz, 1H), 6.57 (s, 1H), 5.48 (d, J = 6.6 Hz, 1H), 4.57-4.13 (m, 2H), 4.11 (d, J = 14.5 Hz, 3H), 3.96-3.89 (m, 5H), 2.86 (d, J = 15.0 Hz, 1H), 2.48 (d, J = 19.8 Hz, 2H), 1.83 (d, J = 6.3 Hz, 3H), 1.12 (dd, J = 12.1, 6.7 Hz, 1H), 0.49 (ddd, J = 18.1, 11.4, 4.6 Hz, 2H), 0.30 (s, 1H), 0.14 (dd, J = 9.2, 4.9 Hz, 1H). LCMS Method U; t_{R}: 1.20 min; m/z: 578 [M + H]⁺ |
| Example 13 | 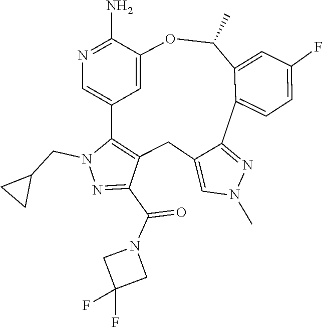 [SMILES: C[C@H]1Oc2cc(cnc2N)-c2c(c(C(=O)N3CC(F)(F)C3)nn2CC2CC2)Cc2cn(C)nc2-c2ccc(F)cc21] | {(R)-22-amino-3-(cyclopropylmethyl)-16-fluoro-10-methyl-19- methyl-20-oxa-3,4,10,11,23- pentaazapentacyclo[19.3.1.0²,⁶.0⁸,¹².0¹³,¹⁸]pentacosa- 1(24),2(6),4,8,11,13,15, 17,21(25),22-decaen-5-yl}(3,3-difluoro- 1-azetidinyl)methanone 1H NMR (400 MHz, MeOD) δ 7.61 (s, 1H), 7.54 (dd, J = 10.2, 2.6 Hz, 1H), 7.45 (d, J = 1.8 Hz, 1H), 7.17 (dd, J = 8.5, 5.8 Hz, 1H), 7.06 (td, J = 8.3, 2.7 Hz, 1H), 6.52 (d, J = 1.7 Hz, 1H), 5.45 (d, J = 4.6 Hz, 1H), 5.12-4.99 (m, 1H), 4.91 (d, J = 12.1 Hz, 1H), 4.53 (t, J = 12.3 Hz, 2H), 4.26 (d, J = 15.4 Hz, 1H), 4.12- 4.05 (m, 1H), 3.97-3.91 (m, 1H), 3.90 (s, 3H), 2.84 (d, J = 14.9 Hz, 1H), 1.82 (d, J = 6.3 Hz, 3H), 1.18-1.08 (m, 1H), 0.49 (ddd, J = 17.8, 8.8, 4.9 Hz, 2H), 0.31 (dd, J = 9.5, 4.7 Hz, 1H), 0.14 (dd, J = 9.5, 4.7 Hz, 1H). LCMS Method U; t_{R}: 1.10 min; m/z: 564 [M + H]⁺ |
| Example 31 | 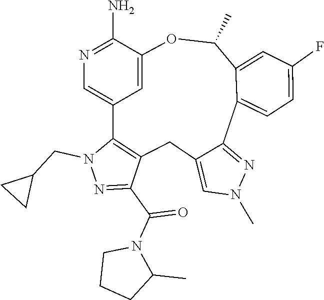 [SMILES: CC1CCCN1C(=O)c1nn(CC2CC2)c2c1Cc1cn(C)nc1-c1ccc(F)cc1[C@@H](C)Oc1cc-2cnc1N] | {(R)-22-amino-3-(cyclopropylmethyl)-16-fluoro-10-methyl-19- methyl-20-oxa-3,4,10,11,23- pentaazapentacyclo[19.3.1.0²,⁶.0⁸,¹².0¹³,¹⁸]pentacosa- 1(24),2(6),4,8,11, 13,15,17,21(25),22-decaen-5-yl}(2-methyl-1- pyrrolidinyl)methanone 1H NMR (400 MHz, MeOD) δ 7.90 (s, 0.5H), 7.70 (d, J = 16.9 Hz, 0.5H), 7.54 (dd, J = 9.9, 2.5 Hz, 1H), 7.49-7.43 (m, 1H), 7.16 (dd, J = 9.3, 5.0 Hz, 1H), 7.06 (dd, J = 11.0, 5.4 Hz, 1H), 6.54 (dd, J = 7.4, 1.8 Hz, 1H), 5.46 (t, J = 8.2 Hz, 1H), 4.62 (d, J = 10.7 Hz, 0.5H), 4.43-4.34 (m, 0.5H), 4.13-4.03 (m, 1H), 3.96 (s, 1H), 3.93-3.91 (m, 3H), 3.86 (d, J = 15.3 Hz, 1H), 3.83- 3.54 (m, 2H), 2.87 (d, J = 15.2 Hz, 1H), 2.08 (dd, J = 62.4, 17.4 Hz, 3H), 1.83 (d, J = 6.3 Hz, 3H), 1.77-1.64 (m, 1H), 1.42- 1.34 (m, 2.5H), 1.12 (t, J = 8.7 Hz, 1H), 0.99 (d, J = 6.3 Hz, 0.5H), 0.53-0.40 (m, 2H), 0.31 (t, J = 8.1 Hz, 1H), 0.16-0.08 (m, 1H). LCMS Method U; t_{R}: 1.01 min; m/z: 556 [M + H]⁺ |
| Example 32 | 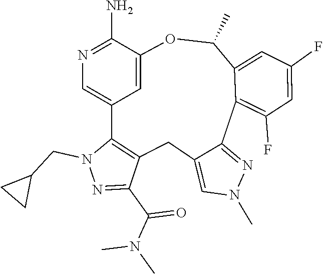 [SMILES: C[C@H]1Oc2cc(cnc2N)-c2c(c(C(=O)N(C)C)nn2CC2CC2)Cc2cn(C)nc2-c2c(F)cc(F)cc21] | N,N-dimethyl-(R)-22-amino-3-(cyclopropylmethyl)-14,16- difluoro-10-methyl-19-methyl-20-oxa-3,4,10,11,23- pentaazapentacyclo[19.3.1.0²,⁶.0⁸,¹².0¹³,¹⁸]pentacosa- 1(24),2(6),4,8,11,13,15,17,21(25),22-decaene-5-carboxamide 1H NMR (400 MHz, MeOD) 8 7.86 (s, 1H), 7.50 (d, J = 1.8 Hz, 1H), 7.43 (d, J = 9.7 Hz, 1H), 6.96 (td, J = 9.2, 2.5 Hz, 1H), 6.57 (d, J = 1.7 Hz, 1H), 5.49-5.29 (m, 1H), 4.10 (dd, J = 14.4, 6.2 Hz, 1H), 3.98-3.90 (m, 4H), 3.74 (d, J = 15.6 Hz, 1H), 3.25 (s, 3H), 3.17 (s, 3H), 2.79 (dd, J = 15.5, 1.9 Hz, 1H), 1.80 (d, J = 6.3 Hz, 3H), 1.16-1.05 (m, 1H), 0.55-0.37 (m, 2H), 0.30 (td, J = 9.5, 4.8 Hz, 1H), 0.17-0.04 (m, 1H). LCMS Method U; t_{R}: 0.70 min; m/z: 534 [M + H]⁺ |
| Example 33 | 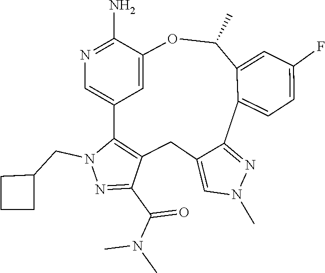 [SMILES: C[C@H]1Oc2cc(cnc2N)-c2c(c(C(=O)N(C)C)nn2CC2CCC2)Cc2cn(C)nc2-c2ccc(F)cc21] | (19R)-22-amino-3-(cyclobutylmcthyl)-16-fluoro-N,N,10,19- tetramethyl-20-oxa-3,4,10,11,23- pentaazapentacyclo[19.3.1.0²,⁶.0⁸,¹².0¹³,¹⁸]pentacosa- 1(24),2(6),4,8,11,13,15,17,21(25),22-decaene-5-carboxamide 1H NMR (400 MHz, MeOD) δ 7.81 (s, 1H), 7.53 (dd, J = 10.1, 2.7 Hz, 1H), 7.43 (d, J = 1.7 Hz, 1H), 7.16 (dd, J = 8.5, 5.8 Hz, 1H), 7.06 (td, J = 8.4, 2.7 Hz, 1H), 6.53 (d, J = 1.7 Hz, 1H), 5.46 (d, J = 4.7 Hz, 1H), 4.18 (dd, J = 13.9, 7.9 Hz, 1H), 4.09 (dd, J = 13.9, 6.6 Hz, 1H), 3.92 (s, 3H), 3.75 (d, J = 15.6 Hz, 1H), 3.20 (d, J = 27.1 Hz, 6H), 2.87 (d, J = 15.3 Hz, 1H), 2.68 (d, J = 7.1 Hz, 1H), 2.01-1.94 (m, 1H), 1.87 (d, J = 10.3 Hz, 2H), 1.83 (d, J = 6.3 Hz, 3H), 1.80-1.72 (m, 2H), 1.66-1.57 (m, 1H). LCMS Method U; t_{R}: 0.84 min; m/z: 530 [M + H]⁺ |
| Example 34 | 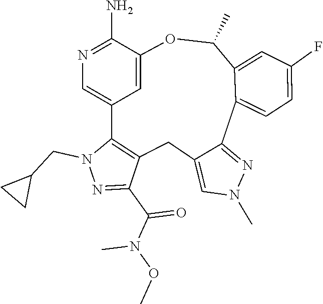 [SMILES: CON(C)C(=O)c1nn(CC2CC2)c2c1Cc1cn(C)nc1-c1ccc(F)cc1[C@@H](C)Oc1cc-2cnc1N] | N-methoxy-N-methyl-(R)-22-amino-3-(cyclopropylmethyl)-16- fluoro-10-methyl-19-methyl-20-oxa-3,4,10,11,23- pentaazapentacyclo[19.3.1.0²,⁶.0⁸,¹².0¹³,¹⁸]pentacosa- 1(24),2(6),4,8,11,13,15,17,21(25),22-decaene-5-carboxamide 1H NMR (400 MHz, MeOD) δ 7.80 (s, 1H), 7.54 (dd, J = 10.1, 2.7 Hz, 1H), 7.47 (d, J = 1.7 Hz, 1H), 7.19 (dd, J = 8.5, 5.7 Hz, 1H), 7.08 (td, J = 8.4, 2.7 Hz, 1H), 6.62 (s, 1H), 5.51 (d, J = 5.3 Hz, 1H), 4.10 (dd, J = 14.4, 6.3 Hz, 1H), 3.99-3.86 (m, 5H), 3.85 (s, 3H), 3.51 (s, 3H), 2.90 (d, J = 15.3 Hz, 1H), 1.84 (d, J = 6.3 Hz, 3H), 1.12 (t, J = 13.5 Hz, 1H), 0.59-0.38 (m, 2H), 0.37- 0.24 (m, 1H), 0.14 (d, J = 4.7 Hz, 1H). LCMS Method U; t_{R}: 0.95 min; m/z: 532 [M + H]⁺ |
| Example 35 | 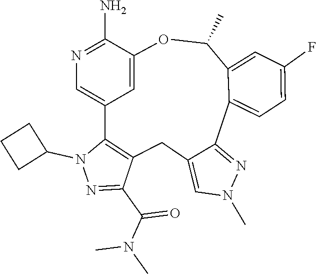 [SMILES: C[C@H]1Oc2cc(cnc2N)-c2c(c(C(=O)N(C)C)nn2C2CCC2)Cc2cn(C)nc2-c2ccc(F)cc21] | N,N-dimethyl-(R)-22-amino-3-cyclobutyl-16-fluoro-10-methyl- 19-methyl-20-oxa-3,4,10,11,23- pentaazapentacyclo[19.3.1.0²,⁶.0⁸,¹².0¹³,¹⁸]pentacosa- 1(24),2(6),4,8,11,13,15,17,21(25),22-decaene-5-carboxamide 1H NMR (400 MHz, MeOD) δ 7.78 (s, IH), 7.53 (dd, J = 10.1, 2.7 Hz, 1H), 7.31 (s, 1H), 7.16 (dd, J = 8.5, 5.6 Hz, 1H), 7.06 (td, J = 8.3, 2.6 Hz, 1H), 6.53 (d, J = 1.6 Hz, 1H), 5.50-5.43 (m, 1H), 4.81 (s, 1H), 3.91 (s, 3H), 3.75 (s, 1H), 3.28 (s, 3H), 3.18 (s, 3H), 2.89-2.83 (m, 1H), 2.74-2.66 (m, 1H), 2.62-2.54 (m, 1H), 2.50-2.42 (m, 1H), 2.20-2.08 (m, 1H), 1.84-1.76 (m, 5H). LCMS Method W; t_{R}: 0.46 min; m/z: 516 [M + H]⁺ |
| Example 36 | 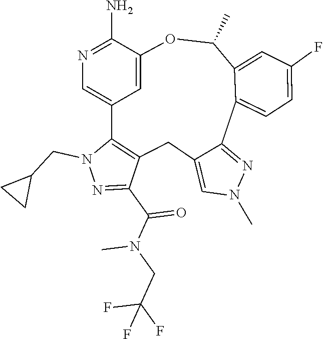 [SMILES: C[C@H]1Oc2cc(cnc2N)-c2c(c(C(=O)N(C)CC(F)(F)F)nn2CC2CC2)Cc2cn(C)nc2-c2ccc(F)cc21] | (19R)-22-amino-3-(cyclopropylmethyl)-16-fluoro-N,10,19- trimethyl-N-(2,2,2-trifluoroethyl)-20-oxa-3,4,10,11,23- pentaazapentacyclo[19.3.1.0²,⁶.0⁸,¹².0¹³,¹⁸]pentacosa- 1(24),2(6),4,8,11,13,15,17,21(25),22-decaene-5-carboxamide 1H NMR (400 MHz, MeOD) δ 7.82 (s, 1H), 7.54 (dd, J = 10.1, 2.7 Hz, 1H), 7.47 (s, 1H), 7.21-7.12 (m, 1H), 7.07 (td, J = 8.3, 2.6 Hz, 1H), 6.56 (s, 1H), 5.48 (d, J = 6.5 Hz, 1H), 5.09 (dd, J = 16.3, 9.0 Hz, 0.5H), 4.59 (ddd, J = 39.9, 15.9, 9.2 Hz, 1H), 4.22 (dd, J = 14.8, 9.2 Hz, 0.5H), 4.10 (dd, J = 12.3, 6.3 Hz, 1H), 3.92 (m, 4H), 3.80 (d, J = 16 Hz, 1H), 3.40 (s, 1.5H), 3.24 (s, 1.5H), 2.88 (dd, J = 15.0, 11.6 Hz, 1H), 1.83 (d, J = 6.3 Hz, 3H), 1.11 (t, J = 10.1 Hz, 1H), 0.46 (dd, J = 17.8, 9.5 Hz, 2H), 0.36-0.26 (m, 1H), 0.17-0.08 (m, 1H). LCMS Method U; t_{R}: 1.13 min; m/z: 584 [M + H]⁺ |
| Example 37 | 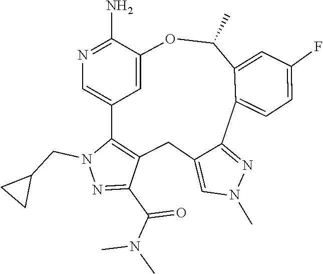 | N-isopropyl-N-methyl-(R)-22-amino-3-(cyclopropylmethyl)-16- fluoro-10-methyl-19-methyl-20-oxa-3,4,10,11,23- pentaazapentacyclo[19.3.1.0²,⁶.0⁸,¹².0¹³,¹⁸]pentacosa- 1(24),2(6),4,8,11,13,15,17,21(25),22-decaene-5-carboxamide 1H NMR (400 MHz, MeOD) δ 7.88 (d, J = 27.7 Hz, 1H), 7.54 (dd, J = 10.1, 2.7 Hz, 1H), 7.47 (s, 1H), 7.17 (dd, J = 8.5, 5.8 Hz, 1H), 7.06 (d, J = 2.7 Hz, 1H), 6.55 (s, 1H), 5.47 (d, J = 4.9 Hz, 1H), 4.97-4.90 (m, 0.5H), 4.42-4.36 (m, 0.5H), 4.09 (dt, J = 12.2, 6.1 Hz, 1H), 3.96-3.86 (m, 4H), 3.67 (dd, J = 15.2, 6.4 Hz, 1H), 3.02 (s, 3H), 2.91 (d, J = 15.4 Hz, 1H), 1.83 (d, J = 6.3 Hz, 3H), 1.31 (m, 4H), 1.17 (d, J = 6.7 Hz, 2H), 1.13-1.05 (m, 1H), 0.54-0.40 (m, 2H), 0.34-0.24 (m, 1H), 0.18-0.06 (m, 1H). LCMS Method U; t_{R}: 0.97 min; m/z: 544 [M + H]⁺ |
| Example 38 | 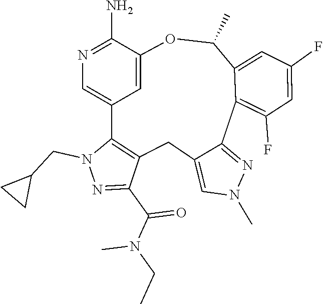 [SMILES: CCN(C)C(=O)c1nn(CC2CC2)c2c1Cc1cn(C)nc1-c1c(F)cc(F)cc1[C@@H](C)Oc1cc-2cnc1N] | N-ethyl-N-methyl-(R)-22-amino-3-(cyclopropylmethyl)-16- fluoro-10-methyl-19-methyl-20-oxa-3,4,10,11,23- pentaazapentacyclo[19.3.1.0²,⁶.0⁸,¹².0¹³,¹⁸]pentacosa- 1(24),2(6),4,8,11,13,15,17,21(25),22-decaene-5-carboxamide 1H NMR (400 MHz, MeOD) δ 7.85 (d, J = 11.0 Hz, 1H), 7.53 (dd, J = 10.1, 2.6 Hz, 1H), 7.46 (d, J = 1.6 Hz, 1H), 7.17 (dd, J = 8.5, 5.7 Hz, 1H), 7.06 (td, J = 8.3, 2.7 Hz, 1H), 6.58 (s, 1H), 5.48 (d, J = 6.5 Hz, 1H), 4.09 (dd, J = 14.5, 6.2 Hz, 1H), 3.96-3.89 (m, 4H), 3.74 (dd, J = 15.1, 4.0 Hz, 1H), 3.62 (dd, J = 18.2, 14.7 Hz, 2H), 3.19 (s, 1H), 3.13 (s, 2H), 2.89 (d, J = 15.2 Hz, 1H), 1.83 (d, J = 6.3 Hz, 3H), 1.28 (q, J = 7.1 Hz, 3H), 1.10 (s, 1H), 0.54- 0.42 (m, 2H), 0.30 (dt, J = 9.4, 4.8 Hz, 1H), 0.16-0.07 (m, 1H). LCMS Method U; t_{R}: 0.90 min; m/z: 530 [M + H]⁺ |
| Example 40 | 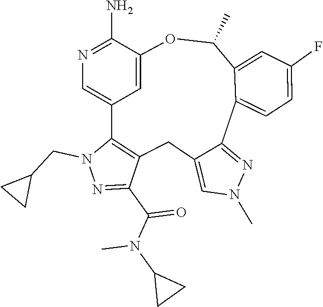 [SMILES: C[C@H]1Oc2cc(cnc2N)-c2c(c(C(=O)N(C)C3CC3)nn2CC2CC2)Cc2cn(C)nc2-c2ccc(F)cc21] | N-cyclopropyl-N-methyl-(R)-22-amino-3-(cyclopropylmethyl)- 16-fluoro-10-methyl-19-methyl-20-oxa-3,4,10,11,23- pontaazapentacyclo[19.3.1.0²,⁶.0⁸,¹².0¹³,¹⁸]pentacosa- 1(24),2(6),4,8,11,13,15,17,21(25),22-decaene-5-carboxamide 1H NMR (400 MHz, MeOD) δ 7.92 (s, 1H), 7.54 (dd, J = 10.1, 2.7 Hz, 1H), 7.47 (s, 1H), 7.17 (dd, J = 8.5, 5.8 Hz, 1H), 7.07 (td, J = 8.4, 2.7 Hz, 1H), 6.58 (s, 1H), 5.49 (d, J = 6.1 Hz, 1H), 4.10 (s, 1H), 3.95-3.86 (m, 4H), 3.70 (d, J = 10.9 Hz, 1H), 3.18- 3.06 (m, 4H), 2.90 (d, J = 15.3 Hz, 1H), 1.84 (d, J = 6.3 Hz, 3H), 1.12 (d, J = 13.9 Hz, 1H), 0.87 (d, J = 17.2 Hz, 1H), 0.70 (d, J = 19.3 Hz, 2H), 0.58-0.35 (m, 3H), 0.30 (d, J = 4.8 Hz, 1H), 0.15- 0.07 (m, 1H). LCMS Method W; t_{R}: 1.16 min; m/z: 542 [M + H]⁺ |
| Example 42 | 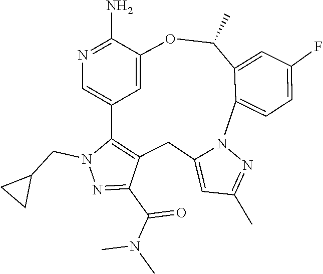 [SMILES: Cc1cc2n(n1)-c1ccc(F)cc1[C@@H](C)Oc1cc(cnc1N)-c1c(c(C(=O)N(C)C)nn1CC1CC1)C2] | N,N-dimethyl-(R)-22-amino-3-(cyclopropylmethyl)-16-fluoro- 10,19-dimethyl-20-oxa-3,4,11,12,23- pentaazapentacyclo[19.3.1.0²,⁶.0⁸,¹².0¹³,¹⁸]pentacosa- 1(24),2(6),4,8,10,13,15,17,21(25),22-decaene-5-carboxamide 1H NMR (400 MHz, DMSO) δ 7.83-7.78 (m, 1H), 7.55 (s, 1H), 7.32 (dd, J = 8.7, 5.4 Hz, 1H), 7.22 (t, J = 8.3 Hz, 1H), 6.26 (s, 2H), 6.17 (s, 1H), 6.14 (s, 1H), 5.27-5.20 (m, 1H), 4.10-4.02 (m, 2H), 3.93 (dd, J = 14.5, 7.3 Hz, 1H), 3.22 (s, 3H), 3.05 (s, 3H), 2.81 (d, J = 15.8 Hz, 1H), 2.20 (s, 3H), 1.71 (d, J = 6.1 Hz, 3H), 0.88-0.82 (m, 1H), 0.49-0.36 (m, 2H), 0.33-0.25 (m, 1H), 0.15-0.08 (m, 1H). LCMS Method R; t_{R}: 0.69 min; m/z: 516 [M + H]⁺ |
| Example 43 | 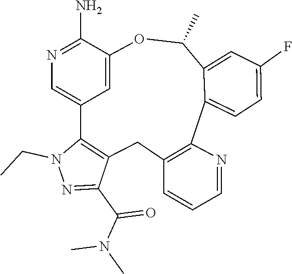 [SMILES: CCn1nc(C(=O)N(C)C)c2c1-c1cnc(N)c(c1)O[C@H](C)c1cc(F)ccc1-c1ncccc1C2] | N,N-dimethyl-(R)-23-amino-3-ethyl-17-fluoro-20-methyl-21- oxa-3,4,12,24- tetraazapentacyclo[20.3.1.0²,⁶.0⁸,¹³.0¹⁴,¹⁹]hexacosa- 1(25),2(6),4,8(13),9,11,14,16,18,22(26),23-undecaene-5- carboxamide 1H NMR (400 MHz, CDC13) δ 8.54 (d, J = 3.6 Hz, IH), 8.39 (d, J = 8.0 Hz, 1H), 7.46 (d, J = 45.9 Hz, 1H), 7.35 (dd, J = 8.0, 4.7 Hz, 1H), 7.29 (dd, J = 9.8, 2.6 Hz, 1H), 7.17 (dd, J = 8.5, 5.6 Hz, 1H), 7.06-6.99 (m, 1H), 6.46 (s, 1H), 5.72 (s, 2H), 5.25 (d, J = 6.6 Hz, 1H), 4.25-4.12 (m, 3H), 3.30 (s, 3H), 3.26 (d, J = 15.6 Hz, 1H), 3.18 (s, 3H), 1.88 (d, J = 6.3 Hz, 3H), 1.46 (t, J = 7.2 Hz, 3H). LCMS Method C1; t_{R}: 1.62 min; m/z: 487 [M + H]⁺ |

### Example 49 (Method D)

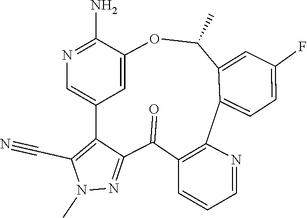 [SMILES: C[C@H]1Oc2cc(cnc2N)-c2c(nn(C)c2C#N)C(=O)c2cccnc2-c2ccc(F)cc21]

Name: (R)-23-amino-17-fluoro-4-methyl-20-methyl-7-oxo-21-oxa-4,5,12,24-tetraazapentacyclo[20.3.1.0^{2,6}.0^{8,13}.0^{14,19}]hexacosa-1(25),2,5,8(13),9,11.14,16,18,22(26),23-undecaene-3-carbonitrile

NMR: 1H NMR (400 MHz, TFA salt, MeOD) δ 8.85 (dd, J=4.9.1.7 Hz, 1H), 8.49 (dd, J=8.0, 1.6 Hz, 1H), 7.66 (ddd, J=8.6, 6.3, 3.8 Hz, 3H), 7.41 (dd, J=10.0, 2.6 Hz, 1H), 7.15 (td, J=8.5, 2.7 Hz, 1H), 6.59 (d, J=1.7 Hz, 1H), 5.00 (q, J=6.3 Hz, 1H), 4.18 (s, 3H), 1.80 (d, J=6.3 Hz, 3H).

LCMS: Method Q; t_{R}: 1.11 min; m/z: 441 [M+H]⁺

To a mixture of 3-(2-bromonicotinoyl)-1-methyl-1H-pyrazole-5-carbonitrile (300 mg, 1.0 mmol), (R)-5-bromo-3-(1-(5-fluoro-2-(trimethylstannyl)phenyl)ethoxy)pyridin-2-amine (635 mg, 1.30 mmol) in DMF (10 mL) was added AsPh₃ (157 mg, 0.510 mmol), CuI (19.6 mg, 0.100 mmol), and Pd₂(dba)₃ (94.0 mg, 0.100 mmol). The mixture was thrice degassed and purged with N₂ and the reaction was stirred at 100° C. for 16 h. The mixture was cooled and filtered. The filtrate was diluted with H₂O (15 mL) and extracted with EtOAc (40 mL×3). The combined organic solutions were washed with water (15 mL×3), brine (20 mL×2), dried over anhydrous Na₂SO₄, filtered, and concentrated. The residue was purified by flash column chromatography on silica gel (0→50% EtOAc in PE) to give (R)-3-(2-(2-(1-((2-amino-5-bromopyridin-3-yl)oxy)ethyl)-4-fluorophenyl)nicotinoyl)-1-methyl-1H-pyrazole-5-carbonitrile (100 mg, 18.6% yield). LCMS (ESI): m/z: 521 [M+H]⁺.

To a mixture of (R)-3-(2-(2-(1-((2-amino-5-bromopyridin-3-yl)oxy)ethyl)-4-fluorophenyl)nicotinoyl)-1-methyl-1H-pyrazole-5-carbonitrile (100 mg, 0.200 mmol) in 2-methyl-2-butanol (5 mL) was added KOAc (56.5 mg, 0.580 mmol), butyl-di-1-adamantylphosphine (27.5 mg, 0.0800 mmol), and palladium acetate (8.60 mg, 0.0400 mmol). The mixture was thrice degassed and purged with N₂ and the reaction tube was sealed. The reaction was stirred at 120° C. for 16 h. The residue was directly purified by flash column chromatography on silica gel (0→60% EtOAc in PE) and further purified by preparative HPLC (Column: YMC-Actus Triart C18 150×20 mm×5 μm; 10→95% MeCN in H₂O+0.1% TFA) to give the target product as the trifluoroacetate salt (26.0 mg, 23% yield). The freebase form was isolated via solid- or liquid-phase acid-base extraction with a basic medium such as aq. NaHCO₃. 1H NMR (400 MHz, freebase, MeOD) δ 8.79 (dd, J=4.9, 1.7 Hz, 1H), 8.28 (dd, J=8.0, 1.7 Hz, 1H), 7.74 (d, J=2.0 Hz, 1H), 7.69 (dd, J=8.6, 5.7 Hz, 1H), 7.57 (dd, J=8.0, 4.9 Hz, 1H), 7.42 (dd, J=10.1, 2.7 Hz, 1H), 7.12 (td, J=8.5, 2.7 Hz, 1H), 6.35 (d, J=1.9 Hz, 1H), 4.71 (q, J=6.3 Hz, 1H), 4.13 (s, 3H), 1.75 (d, J=6.3 Hz, 3H). LCMS (ESI): m/z: 441 [M+H]⁺.

The following compounds were prepared in a similar manner:

| | | |
|---|---|---|
| Example 25 | 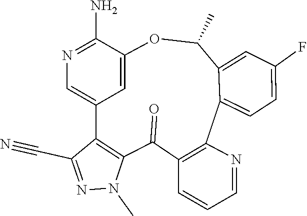 [SMILES: C[C@H]1Oc2cc(cnc2N)-c2c(C#N)nn(C)c2C(=O)c2cccnc2-c2ccc(F)cc21] | (R)-23-amino-17-fluoro-5-methyl-20-methyl-7-oxo-21-oxa- 4,5,12,24-tetraazapentacyclo[20.3.1.0²,⁶.0⁸,¹³.0¹⁴,¹⁹]hexacosa- 1(25),2(6),3,8(13),9,11,14,16,18,22(26),23-undecaene-3- carbonitrile 1H NMR (400 MHz, MeOD) δ 8.71 (dd, J = 4.9, 1.6 Hz, 1H), 8.02-7.87 (m, 2H), 7.86 (d, J = 2.1 Hz, 1H), 7.51 (dd, J = 10.1, 2.7 Hz, 1H), 7.41 (dd, J = 7.9, 4.9 Hz, 1H), 7.20 (td, J = 8.4, 2.7 Hz, 1H), 6.37 (d, J = 2.1 Hz, 1H), 4.54-4.47 (m, 1H), 4.13 (s, 3H), 1.76 (d, J = 6.5 Hz, 3H). LCMS Method W; t_{R}: 1.34 min; m/z: 441 [M + H]⁺ |
| Example 26 | 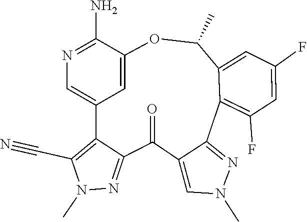 [SMILES: C[C@H]1Oc2cc(cnc2N)-c2c(nn(C)c2C#N)C(=O)c2cn(C)nc2-c2c(F)cc(F)cc21] | (R)-22-amino-14,16-difluoro-4,10-dimethyl-19-methyl-7-oxo- 20-oxa-4,5,10,11,23- pentaazapentacyclo[19.3.1.0²,⁶.0⁸,¹².0¹³,¹⁸]pentacosa- 1(24),2,5,8,11,13,15,17,21(25),22-decaene-3-carbonitrile 1H NMR (400 MHz, MeOD) δ 8.23 (s, 1H), 7.67 (s, 1H), 7.31 (d, J = 8.8 Hz, 1H), 7.00 (dd, J = 11.3, 8.8 Hz, 1H), 6.54 (s, 1H), 5.35-5.32 (m, 1H), 4.10 (s, 3H), 4.03 (s, 3H), 1.68 (d, J = 6.2 Hz, 3H). LCMS Method W; t_{R}: 1.09 min; m/z: 462 [M + H]⁺ |
| Example 29 | 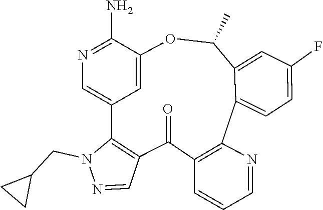 [SMILES: C[C@H]1Oc2cc(cnc2N)-c2c(cnn2CC2CC2)C(=O)c2cccnc2-c2ccc(F)cc21] | (R)-23-amino-3-(cyclopropylmethyl)-17-fluoro-20-methyl-21- oxa-3,4, 12,24- tetraazapentacyclo[20.3.1.0²,⁶.0⁸,¹³.0¹⁴,¹⁹]hexacosa- 1(25),2(6),4,8(13),9,11,14,16,18,22(26),23-undecaen-7-one 1H NMR (400 MHz, MeOD) δ 8.64 (dd, J = 4.9, 1.7 Hz, 1H), 8.06 (s, 1H), 7.91 (dd, J = 7.9, 1.7 Hz, 1H), 7.79 (dd, J = 8.7, 5.8 Hz, 1H), 7.63 (d, J = 1.6 Hz, 1H), 7.56 (dd, J = 10.2, 2.7 Hz, 1H), 7.41 (dd, J = 7.9, 4.9 Hz, IH), 7.17 (td, J = 8.5, 2.7 Hz, 1H), 6.54 (d, J = 1.8 Hz, 1H), 5.17 (qd, J = 6.5, 1.7 Hz, 1H), 4.10-3.97 (m, 2H), 1.84 (d, J = 6.4 Hz, 3H), 1.15 (ddd, J = 11.5, 7.5, 5.0 Hz, 1H), 0.52-0.46 (m, 2H), 0.29-0.19 (m, 2H). LCMS Method R; t_{R}: 1.32 min; m/z: 456 [M + H]⁺ |
| Example 47 | 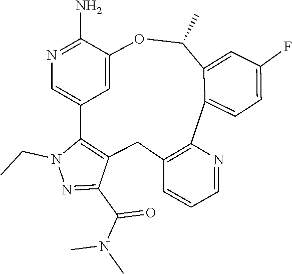 | N,N-dimethyl-(R)-23-amino-3-ethyl-17-fluoro-20-methyl-7- oxo-21-oxa-3,4,12,24- tetraazapentacyclo[20.3.1.0²,⁶.0⁸,¹³.0¹⁴,¹⁹]hexacosa- 1(25),2(6),4,8(13),9,11,14,16,18,22(26),23-undecaene-5- carboxamide 1H NMR (400 MHz, DMSO) δ 8.71 (dd, J = 4.8, 1.5 Hz, 1H), 8.19-8.03 (m, 1H), 7.64-7.56 (m, 3H), 7.49 (dd, J = 7.9, 4.8 Hz, 1H), 7.25-7.11 (m, 1H), 6.31 (s, 2H), 6.18 (d, J = 1.8 Hz, 1H), 4.76 (d, J = 4.4 Hz, 1H), 4.18 (dt, J = 9.7, 7.0 Hz, 2H), 3.27 (s, 3H), 3.01 (s, 3H), 1.68 (d, J = 6.3 Hz, 3H), 1.36 (t, J = 7.2 Hz, 3H). LCMS Method U; t_{R}: 1.16 min; m/z: 501 [M + H]⁺ |
| Example 50 | 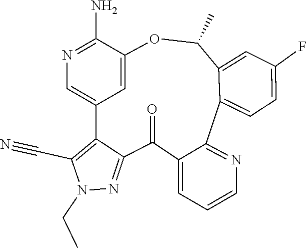 [SMILES: CCn1nc2c(c1C#N)-c1cnc(N)c(c1)O[C@H](C)c1cc(F)ccc1-c1ncccc1C2=O] | (R)-23-amino-4-ethyl-17-fluoro-20-methyl-7-oxo-21-oxa- 4,5,12,24-tetraazapentacyclo[20.3.1.0²,⁶.0⁸,¹³.0¹⁴,¹⁹]hexacosa- 1(25),2,5,8,10,12,14,16,18,22(26),23-undecaene-3- carbonitrile 1H NMR (400 MHz, MeOD) δ 8.86 (dd, J = 4.8, 1.6 Hz, 1H), 8.50 (dd, J = 8.0, 2.0 Hz, 1H), 7.73-7.60 (m, 3H), 7.41 (dd, J = 10.0, 2.4 Hz, 1H), 7.16 (td, J = 8.4, 2.8 Hz, 1H), 6.62 (d, J = 1.6 Hz, 1H), 5.08-4.95 (m, 1H), 4.51 (q, J = 7.2 Hz, 2H), 1.81 (d, J = 6.4 Hz, 3H), 1.63 (t, J = 7.2 Hz, 3H). LCMS Method R; t_{R}: 1.28 min; m/z: 455 [M + H]⁺ |
| Example 51 | 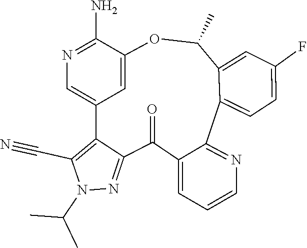 [SMILES: CC(C)n1nc2c(c1C#N)-c1cnc(N)c(c1)O[C@H](C)c1cc(F)ccc1-c1ncccc1C2=O] | (R)-23-amino-17-fluoro-4-isopropyl-20-methyl-7-oxo-21-oxa- 4,5,12,24-tetraazapentacyclo[20.3.1.0²,⁶.0⁸,¹³.0¹⁴,¹⁹]hexacosa- 1(25),2,5,8,10,12,14,16,18,22(26),23-undecaene-3- carbonitrile 1H NMR (400 MHz, MeOD) δ 8.79 (dd, J = 4.9, 1.6 Hz, 1H), 8.26 (dd, J = 8.0, 1.6 Hz, 1H), 7.75 (d, J = 2.0 Hz, 1H), 7.69 (dd, J = 8.6, 5.8 Hz, 1H), 7.60 (dd, J = 8.0, 4.8 Hz, 1H), 7.42 (dd, J = 10.1, 2.8 Hz, 1H), 7.11 (dt, J = 8.4, 4.2 Hz, 1H), 6.36 (d, J = 1.8 Hz, 1H), 4.93-4.89 (m, 1H), 4.76-4.70 (m, 1H), 1.75 (d, J = 6.3 Hz, 3H), 1.67 (d, J = 6.6 Hz, 3H), 1.62 (d, J = 6.6 Hz, 3H). LCMS Method U; t_{R}: 1.24 min; m/z: 469 [M + H]⁺ |
| Example 52 | 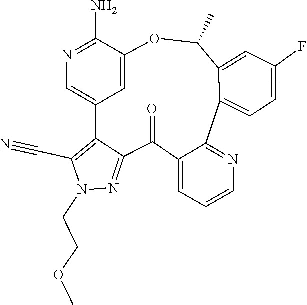 [SMILES: COCCn1nc2c(c1C#N)-c1cnc(N)c(c1)O[C@H](C)c1cc(F)ccc1-c1ncccc1C2=O] | (R)-23-amino-17-fluoro-4-(2-methoxyethyl)-20-methyl-7- oxo-21-oxa-4,5,12,24- tetraazapentacyclo[20.3.1.0²,⁶.0⁸,¹³.0¹⁴,¹⁹]hexacosa- 1(25),2,5,8,10,12,14,16,18,22(26),23-undecaene-3- carbonitrile 1H NMR (400 MHz, DMSO) δ 8.87 (dd, J = 4.8, 1.7 Hz, 1H), 8.22 (dd, J = 8.0, 1.7 Hz, 1H), 7.68-7.64 (m, 2H), 7.59 (dd, J = 8.6, 5.9 Hz, 1H), 7.54 (dd, J = 10.3, 2.7 Hz, 1H), 7.20 (td, J = 8.5, 2.7 Hz, 1H), 6.27 (s, 2H), 6.18 (d, J = 1.9 Hz, 1H), 4.70 (d, J = 6.5 Hz, 1H), 4.55 (t, J = 4.9 Hz, 2H), 3.88-3.75 (m, 2H), 3.29 (s, 3H), 1.65 (d, J = 6.2 Hz, 3H). LCMS Method U; t_{R}: 1.10 min; m/z: 485 [M + H]⁺ |
| Example 53 | 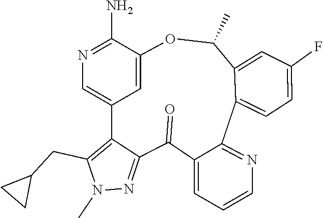 [SMILES: C[C@H]1Oc2cc(cnc2N)-c2c(nn(C)c2CC2CC2)C(=O)c2cccnc2-c2ccc(F)cc21] | (R)-23-amino-3-(cyclopropylmethyl)-17-fluoro-4-methyl-20- methyl-21-oxa-4,5,12,24- tetraazapentacyclo[20.3.1.0²,⁶.0⁸,¹³.0¹⁴,¹⁹]hexacosa- 1(25),2,5,8,10,12,14,16,18,22(26),23-undecaen-7-one 1H NMR (400 MHz, MeOD) δ 8.69 (dd, J = 4.9, 1.6 Hz, 1H), 8.24 (dd, J = 8.0, 1.6 Hz, 1H), 7.72 (dd, J = 8.7, 5.8 Hz, 1H), 7.49 (dd, J = 7.9, 4.9 Hz, 1H), 7.45 (t, J = 5.0 Hz, 2H), 7.11 (td, J = 8.4, 2.7 Hz, 1H), 6.26 (d, J = 1.7 Hz, 1H), 5.34 (t, J = 4.7 Hz, 1H), 3.98 (s, 3H), 2.76 (d, J = 6.3 Hz, 2H), 1.75 (d, J = 6.4 Hz, 3H), 0.90 (t, J = 6.8 Hz, 1H), 0.47 (d, J = 8.0 Hz, 2H), 0.19- 0.06 (m, 2H). LCMS Method R; t_{R}: 1.13 min; m/z: 470 [M + H]⁺ |
| Example 54 | 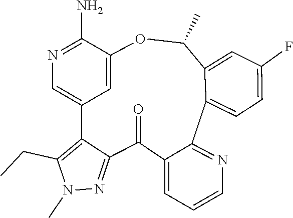 [SMILES: CCc1c2c(nn1C)C(=O)c1cccnc1-c1ccc(F)cc1[C@@H](C)Oc1cc-2cnc1N] | (R)-23-amino-3-ethyl-17-fluoro-4-methyl-20-methyl-21-oxa- 4,5,12,24-tetraazapentacyclo[20.3.1.0²,⁶.0⁸,¹³.0¹⁴,¹⁹]hexacosa- 1(25),2,5,8,10,12,14,16,18,22(26),23-undecaen-7-one 1H NMR (400 MHz, DMSO) δ 8.79 (dd, J = 4.8, 1.7 Hz, 1H), 8.28 (dd, J = 7.9, 1.7 Hz, 1H), 7.61-7.50 (m, 3H), 7.37 (d, J = 1.9 Hz, 1H), 7.16 (td, J = 8.5, 2.8 Hz, 1H), 6.07 (d, J = 1.8 Hz, 1H), 5.88 (s, 2H), 4.79-4.66 (m, 1H), 3.89 (s, 3H), 2.77 (dd, J = 15.0, 7.5 Hz, 1H), 2.63 (dd, J = 14.9, 7.6 Hz, 1H), 1.64 (d, J = 6.3 Hz, 3H), 1.13 (t, J = 7.5 Hz, 3H). LCMS Method U; t_{R}: 0.96 min; m/z: 444 [M + H]⁺ |
| Example 73 | 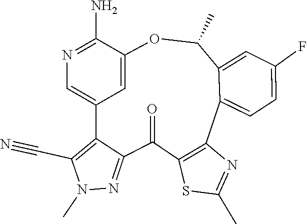 [SMILES: Cc1nc2c(s1)C(=O)c1nn(C)c(C#N)c1-c1cnc(N)c(c1)O[C@H](C)c1cc(F)ccc1-2] | (R)-22-amino-16-fluoro-4-methyl-10,19-dimethyl-7-oxo-20- oxa-9-thia-4,5,11,23- tetraazapentacyclo[19.3.1.0²,⁶.0⁸,¹².0¹³,¹⁸]pentacosa- 1(24),2,5,8(12),10,13,15,17,21(25),22-decaene-3-carbonitrile 1H NMR (400 MHz, MeOD) δ 7.71-7.64 (m, 2H), 7.46 (dd, J = 10.0, 2.7 Hz, 1H), 7.15 (td, J = 8.3, 2.7 Hz, 1H), 6.40 (d, J = 1.9 Hz, 1H), 4.93 (ddd, J = 8.9, 6.9, 1.3 Hz, 1H), 4.11 (s, 3H), 2.79 (s, 3H), 1.76 (d, J = 6.3 Hz, 3H). LCMS Method U; t_{R}: 1.24 min; m/z: 461 [M + H]⁺ |
| Example 74 | 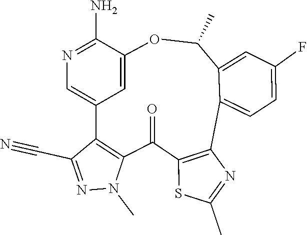 [SMILES: Cc1nc2c(s1)C(=O)c1c(c(C#N)nn1C)-c1cnc(N)c(c1)O[C@H](C)c1cc(F)ccc1-2] | (R)-22-amino-16-fluoro-5-methyl-10,19-dimethyl-7-oxo-20- oxa-9-thia-4,5,11,23- tetraazapentacyclo[19.3.1.0²,⁶.0⁸,¹².0¹³,¹⁸]pentacosa- 1(24),2(6),3,8(12),10,13,15,17,21(25),22-decaene-3- carbonitrile 1H NMR (400 MHz, MeOD) δ 7.89 (dd, J = 8.6, 6.0 Hz, 1H), 7.83 (s, 1H), 7.52 (dd, J = 10.2, 2.6 Hz, 1H), 7.21 (td, J = 8.4, 2.6 Hz, 1H), 6.33 (s, 1H), 5.00-4.98 (m, 1H), 4.10 (s, 3H), 2.69 (s, 3H), 1.79 (d, J = 6.5 Hz, 3H). LCMS Method U; t_{R}: 1.50 min; m/z: 461 [M + H]⁺ |
| Example 75 | 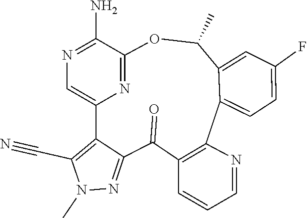 [SMILES: C[C@H]1Oc2nc(cnc2N)-c2c(nn(C)c2C#N)C(=O)c2cccnc2-c2ccc(F)cc21] | (R)-23-amino-17-fluoro-4-methyl-20-methyl-7-oxo-21-oxa- 4,5,12,24,26- pentaazapentacyclo[20.3.1.0²,⁶.0⁸,¹³.0¹⁴,¹⁹]hexacosa- 1(25),2,5,8(13),9,11,14,16,18,22(26),23-undecaene-3- carbonitrile 1H NMR (400 MHz, MeOD) δ 8.76 (dd, J = 5.0, 1.7 Hz, 1H), 8.42 (dd, J = 8.0, 1.7 Hz, 1H), 7.92 (s, 1H), 7.61 (dd, J = 8.0, 5.0 Hz, 1H), 7.52-7.47 (m, 1H), 7.33 (dd, J = 10.0, 2.6 Hz, 1H), 7.12 (td, J = 8.5, 2.7 Hz, 1H), 5.15-5.05 (m, 1H), 4.14 (s, 3H), 1.54 (d, J = 6.6 Hz, 3H). LCMS Method R; t_{R}: 1.46 min; m/z: 442 [M + H]⁺ |
| Example 76 | 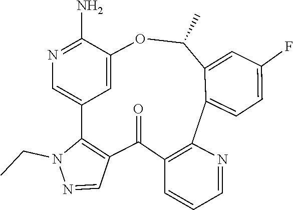 [SMILES: CCn1ncc2c1-c1cnc(N)c(c1)O[C@H](C)c1cc(F)ccc1-c1ncccc1C2=O] | (R)-23-amino-3-ethyl-17-fluoro-20-methyl-21-oxa-3,4,12,24- tetraazapentacyclo[20.3.1.0²,⁶.0⁸,¹³.0¹⁴,¹⁹]hexacosa- 1(25),2(6),4,8(13),9,11,14,16,18,22(26),23-undecaen-7-one 1H NMR (400 MHz, DMSO) δ 8.56 (dd, J = 4.8, 1.7 Hz, 1H), 8.04 (s, 1H), 7.77 (dd, J = 8.7, 6.1 Hz, 1H), 7.67 (dd, J = 10.5, 2.7 Hz, 1H), 7.61 (d, J = 2.0 Hz, 1H), 7.51 (dd, J = 7.8, 1.7 Hz, 1H), 7.27 (td, J = 8.5, 2.7 Hz, 1H), 7.20 (dd, J = 7.8, 4.8 Hz, 1H), 6.20 (s, 2H), 6.02 (d, J = 2.0 Hz, 1H), 5.01 (dd, J = 6.6, 2.0 Hz, 1H), 4.04 (M, 2H), 1.76 (d, J = 6.5 Hz, 3H), 1.25 (t, J = 7.2 Hz, 3H). LCMS Method R; t_{R}: 1.23 min; m/z: 430 [M + H]⁺ |

### Example 23 (Method E)

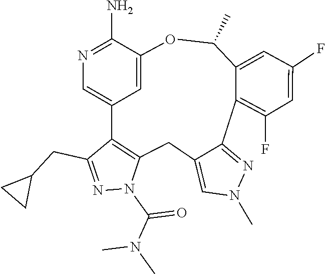 [SMILES: C[C@H]1Oc2cc(cnc2N)-c2c(CC3CC3)nn(C(=O)N(C)C)c2Cc2cn(C)nc2-c2c(F)cc(F)cc21]

Name: (19R)-22-amino-3-(cyclopropylmethyl)-14,16-difluoro-N,N,10,19-tetramethyl-20-oxa-4,5,10,11,23-pentaazapentacyclo[19.3.1.0^{2,6}.0^{8,12}.0^{13,18}]pentacosa-1(24),2(6),3,8,11,13,15,17,21(25),22-decaene-5-carboxamide

NMR: 1H NMR (400 MHz, MeOD) δ 7.99 (s, 1H), 7.41 (d, J=1.6 Hz, 2H), 6.98 (s, 1H), 6.54 (d, J=1.6 Hz, 1H), 5.41 (d, J=5.9 Hz, 1H), 3.97 (m. 4H), 3.12 (m. 7H), 2.59 (dd, J=6.6, 1.9 Hz, 2H). 1.80 (d. J=6.3 Hz, 3H), 0.93 (s, 1H), 0.39 (ddd, J=12.6, 8.6, 4.4 Hz, 2H), 0.14 (s, 1H). 0.03 (d. J=3.8 Hz. 1H).

LCMS: Method U: t_{R}: 1.25 min; m/z: 534 [M+H]⁺

To a mixture of (R)-4-bromo-5-((2-(2-(1-((5-bromo-2-nitropyridin-3-yl)oxy)ethyl)-4-fluorophenyl)pyridin-3-yl)methyl)-3-(cyclopropylmethyl)-N,N-dimethyl-1H-pyrazole-1-carboxamide (65.0 mg, crude) and iron powder (50.0 mg, 0.890 mmol) in EtOH (5 mL) and H₂O (1 mL) was added NH₄C1 (48.0 mg, 0.890 mmol). The reaction was stirred at 80° C. for 2 h. The mixture was filtered, and the filtrate was concentrated. The residue was purified by flash column chromatography on silica gel (0→55% EtOAc in PE) to give (R)-5-((3-(2-(1-((2-amino-5-bromopyridin-3-yl)oxy)ethyl)-4,6-difluorophenyl)-1-methyl-1H-pyrazol-4-yl)methyl)-4-bromo-3-(cyclopropylmethyl)-N,N-dimethyl-1H-pyrazole-1-carboxamide (45.0 mg, 56.0% yield, over two steps) as a light-yellow oil. LC/MS (ESI) (m/z): 692 [M+H]⁺.

To a mixture of (R)-5-((3-(2-(1-((2-amino-5-bromopyridin-3-yl)oxy)ethyl)-4,6-difluorophenyl)-1-methyl-1H-pyrazol-4-yl)methyl)-4-bromo-3-(cyclopropylmethyl)-N,N-dimethyl-1H-pyrazole-1-carboxamide (45.0 mg, 0.0650 mmol), 4,4,4′,4′,5,5,5′,5′-octamethyl-2,2′-bi(1,3,2-dioxaborolane) (33.0 mg, 0.130 mmol), CsF (2 M in H₂O, 0.100 mL, 0.195 mmol) and cataCXium A (9.00 mg, 0.0260 mmol) in MeOH (10 mL) was added Pd(OAc)₂ (3.00 mg, 0.0130 mmol). The suspension was degassed and purged with N₂ several times. The reaction was stirred at 70° C. for 2 h in a sealed tube. The mixture was concentrated, and the residue was purified by preparative HPLC (Column: YMC-TA C18 250×21.2 mm×5 μm; 5→95% MeCN in H₂O+0.10% FA) to give the target product. (1.00 mg, 3.00% yield). LC/MS ESI (m/z):534 [M+H]⁺.

The following compounds were prepared in a similar manner:

| | | |
|---|---|---|
| Example 20 | 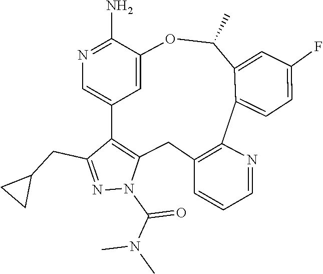 [SMILES: C[C@H]1Oc2cc(cnc2N)-c2c(CC3CC3)nn(C(=O)N(C)C)c2Cc2cccnc2-c2ccc(F)cc21] | (R)-5-(N,N-dimethylcarbamoyl)-3-(cyclopropylmethyl)-17-fluoro- 20-methyl-21-oxa-4,5,12,24- tetraazapentacyclo[20.3.1.0²,⁶.0⁸,¹³.0¹⁴,¹⁹]hexacosa- 1(25),2(6),3,8(13),9,11,14,16,18,22(26),23-undecaen-23-ylamine 1H NMR (400 MHz, MeOD) δ 8.56 (d, J = 4.8 Hz, 1H), 8.19 (d, J = 8.0 Hz, 1H), 7.55-7.48 (m, 2H), 7.44 (d, J = 1.8 Hz, 1H), 7.28 (s, 1H), 7.11 (dd, J = 9.6, 7.1 Hz, 1H), 6.23 (s, 1H), 5.09 (d, J = 6.0 Hz, 1H), 4.41 (d, J = 15.3 Hz, 1H), 3.57 (d, J = 16.0 Hz, 1H), 3.13 (s, 6H), 2.66 (dd, J = 6.6, 3.9 Hz, 2H), 1.83 (d, J = 6.2 Hz, 3H), 0.99 (s, 1H), 0.46-0.38 (m, 2H), 0.15 (s, 1H), 0.10 (s, 1H). LCMS Method U; t_{R}: 1.30 min; m/z: 513 [M + H]⁺ |
| Example 24 | 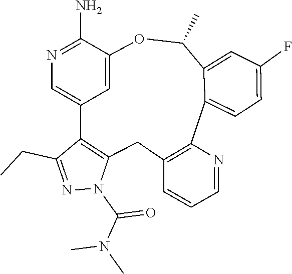 [SMILES: CCc1nn(C(=O)N(C)C)c2c1-c1cnc(N)c(c1)O[C@H](C)c1cc(F)ccc1-c1ncccc1C2] | (R)-5-(N,N-dimethylcarbamoyl)-3-ethyl-17-fluoro-20-methyl-21- oxa-4,5,12,24-tetraazapentacyclo[20.3.1.0²,⁶.0⁸,¹³.0¹⁴,¹⁹]hexacosa- 1(25),2(6),3,8(13),9,11,14,16,18,22(26),23-undecaen-23-ylamine 1H NMR (400 MHz, MeOD) δ 8.55 (d, J = 4.8 Hz, 1H), 8.16 (d, J = 8.0 Hz, 1H), 7.56-7.46 (m, 2H), 7.40 (d, J = 1.6 Hz, 1H), 7.31- 7.24 (m, 1H), 7.14-7.07 (m, 1H), 6.24 (d, J = 1.6 Hz, 1H), 5.11- 5.04 (m, 1H), 4.42 (d, J = 16.0 Hz, 1H), 3.56 (d, J = 16.0 Hz, 1H), 3.13 (s, 6H), 2.74 (q, J = 7.6 Hz, 2H), 1.83 (d, J = 6.4 Hz, 3H), 1.21 (t, J = 7.6 Hz, 3H). LCMS Method U; t_{R}: 1.04 min; m/z: 487 [M + H]⁺ |

### Example 2 (Method F)

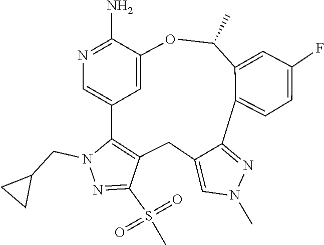 [SMILES: C[C@H]1Oc2cc(cnc2N)-c2c(c(S(C)(=O)=O)nn2CC2CC2)Cc2cn(C)nc2-c2ccc(F)cc21]

Name: (R)-3-(cyclopropylmethyl)-16-fluoro-5-mesyl-10-methyl-19-methyl-20-oxa-3,4,10,11,23-pentaazapentacyclo[19.3.1.0^{2,6}.0^{8,12}.0^{13,18}]pentacosa-1(24),2(6),4,8,11,13,15,17,21(25),22-decaen-22-ylamine

NMR: 1H NMR (400 MHz, MeOD) δ 7.73 (s, 1H), 7.55 (d, J=10.2 Hz, 1H), 7.47 (s, 1H), 7.24-7.12 (m, 1H), 7.07 (t, J=8.0 Hz, 1H). 6.52 (s, 1H), 5.49 (d, J=6.2 Hz, 1H), 4.17-4.08 (m, 2H), 4.06-3.97 (m, 1H), 3.93 (s, 3H), 3.33 (s, 3H). 2.94 (d. J=15.2 Hz, 1H). 1.83 (d, J=6.2 Hz, 3H), 1.12 (m, 1H), 0.47 (m, J=28.5 Hz, 2H), 0.33-0.24 (m, 1H), 0.10 (m, 1H).

LCMS: Method U; t_{R}: 0.88 min; m/z: 523 [M+H]⁺

A solution of (R)-3-(cyclopropylmethyl)-16-fluoro-10-methyl-19-methyl-5-(methylthio)-20-oxa-3,4,10,11,23-pentaazapentacyclo[19.3.1.0^{2,6}.0^{8,12}.0^{13,18}]pentacosa-1(25),2(6),4,8,11,13,15,17,21,23-decaen-22-ylamine (50.0 mg, 0.100 mmol) in DCM (2 mL) was added m-CBPA (21 mg, 0.100 mmol) at 0° C. The reaction was stirred at 0° C. for 30 min. The mixture was quenched with sat. aq. Na₂S₂O₃ (15 mL) and extracted with DCM (15 mL×3). The combined organic layers were dried over anhydrous Na₂SO₄, filtered, and concentrated. The residue was purified by preparative HPLC (Column: YMC-Actus Triart C18 250×20 mm×5 μm; 5→95% MeCN in H₂O+0.1% FA) to give the sulfone product (1.00 mg, 2.0% yield, white solid, LC/MS ESI (m/z): 523 [M+H]⁺) and the sulfoxide product (6.7 mg, 13% yield, LC/MS ESI (m/z): 507 [M+H]⁺).

The following compounds were prepared in a similar manner:

| | | |
|---|---|---|
| Example 17 | 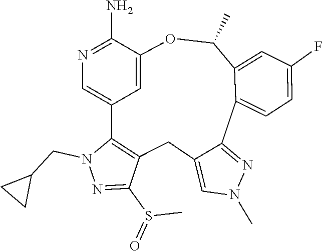 [SMILES: C[C@H]1Oc2cc(cnc2N)-c2c(c(S(C)=O)nn2CC2CC2)Cc2cn(C)nc2-c2ccc(F)cc21] | (R)-3-(cyclopropylmethyl)-16-fluoro-10-methyl-19-methyl-5- (methylsulfinyl)-20-oxa-3,4,10,11,23- pentaazapentacyclo[19.3.1.0²,⁶.0⁸,¹².0¹³,¹⁸]pentacosa- 1(24),2(6),4,8,11,13,15,17,21(25),22-decaen-22-ylamine 1H NMR (400 MHz, DMSO) δ 7.75 (s, 1H), 7.71 (d. J = 12 Hz, 1H),7.50 (s, 1H), 7.17 (m, J = 16.4, 9.4 Hz, 2H), 6.31 (s, 1H), 6.21 (s, 2H), 5.37 (d, J = 6.6 Hz, IH), 4.07 (dd, J = 14.3, 6.1 Hz, 1H), 3.94 (dd, J = 16.7, 9.7 Hz, 2H), 3.85 (s, 3H), 3.08 (s, 3H), 2.80 (d, J = 15.6 Hz, 1H), 1.72 (d, J = 6.0 Hz, 3H), 1.14 (m, 1H), 0.53- 0.35 (m, 2H), 0.28 (m, J = 8.9, 4.1 Hz, 1H), 0.19-0.05 (m, 1H). LCMS Method C1; t_{R}: 0.51 min; m/z: 507 [M + H]⁺ |

### Example 41 (Method G)

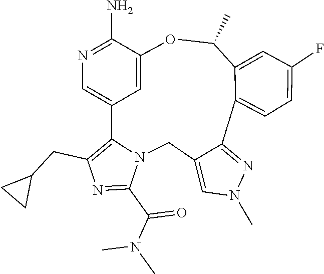 [SMILES: C[C@H]1Oc2cc(cnc2N)-c2c(CC3CC3)nc(C(=O)N(C)C)n2Cc2cn(C)nc2-c2ccc(F)cc21]

Name: N,N-dimethyl-(R)-22-amino-3-(cyclopropylmethyl)-16-fluoro-10-methyl-19-methyl-20-oxa-4,6,10,11,23-pentaazapentacyclo[19.3.1.0^{2,6}.0^{8,12}.0^{13,18}]pentacosa-1(24),2,4,8,11,13,15,17,21(25),22-decaene-5-carboxamide

NMR: 1H NMR (400 MHz, DMSO) δ 7.87 (s, 1H), 7.73 (d, J=10.2 Hz, 1H), 7.33 (s. 1H), 7.11 (d. J=6.5 Hz, 2H), 6.32 (s. 1H), 6.04 (s, 2H), 5.42 (d, J=15.5 Hz, 2H), 3.90 (s, 3H), 3.86 (s, 1H), 3.25 (s, 3H), 3.07 (s, 3H), 2.35 (dd, J=20.5, 6.4 Hz, 2H), 1.73 (d, J=6.2 Hz, 3H), 0.94-0.89 (m, 1H), 0.37-0.28 (m, 2H), 0.14-−0.03 (m, 2H).

LCMS: Method U; t_{R}: 0.80 min; m/z: 516 [M+H]⁺

To a solution of (R)-1-((3-(2-(1-((5-bromo-2-nitropyridin-3-yl)oxy)ethyl)-4-fluorophenyl)-1-methyl-1H-pyrazol-4-yl)methyl)-4-(cyclopropylmethyl)-N,N-dimethyl-1H-imidazole-2-carboxamide (80.0 mg, 0.140 mmol) in EtOH (5 mL) and water (1 mL) was added iron powder (40.0 mg, 0.700 mmol) and NH₄Cl (70.0 mg, 1.40 mmol) at r.t. The reaction was stirred at 80° C. for 1 h. The mixture was filtered, and the filtrate was concentrated. The residue was purified by flash column chromatography on silica gel (0→50% EtOAc in PE) to give (R)-1-((3-(2-(1-((2-amino-5-bromopyridin-3-yl)oxy)ethyl)-4-fluorophenyl)-1-methyl-1H-pyrazol-4-yl)methyl)-4-(cyclopropylmethyl)-N,N-dimethyl-1H-imidazole-2-carboxamide (70.0 mg, yield: 92.1%) as a brown solid. LC/MS (ESI) (m/z): 596 [M+H]⁺.

To a stirred solution of (R)-1-((3-(2-(1-((2-amino-5-bromopyridin-3-yl)oxy)ethyl)-4-fluorophenyl)-1-methyl-1H-pyrazol-4-yl)methyl)-4-(cyclopropylmethyl)-N,N-dimethyl-1H-imidazole-2-carboxamide (40 mg, 0.060 mmol) in MeCN (4 mL) was added a solution of N-bromosuccinimide (10 mg, 0.060 mmol) in MeCN (1 mL) at 0° C. The reaction was stirred at 0° C. for 1 h. The mixture was poured into sat. aq. NaHCO₃ (10 mL) and extracted with EtOAc (10 mL×3). The combined organic phases were washed with brine (10 mL×2), dried over anhydrous Na₂SO₄, filtered, and concentrated. The residue was purified by flash column chromatography on silica gel (0→50% EtOAc in PE) to give (R)-1-((3-(2-(1-((2-amino-5-bromopyridin-3-yl)oxy)ethyl)-4-fluorophenyl)-1-methyl-1H-pyrazol-4-yl)methyl)-5-bromo-4-(cyclopropylmethyl)-N,N-dimethyl-1H-imidazole-2-carboxamide (40.0 mg, yield: 88.9%) as a brown solid. LC/MS (ESI) (m/z): 674 [M+H]⁺.

To a solution of (R)-1-((3-(2-(1-((2-amino-5-bromopyridin-3-yl)oxy)ethyl)-4-fluorophenyl)-1-methyl-1H-pyrazol-4-yl)methyl)-5-bromo-4-(cyclopropylmethyl)-N,N-dimethyl-1H-imidazole-2-carboxamide (50 mg, 0.070 mmol) in MeOH (8 mL) were added B₂Pin₂ (60 mg, 0.22 mmol), cataCXium A (10 mg, 0.040 mmol), CsF (2 M in H₂O, 0.07 mL, 0.150 mmol) and Pd(OAc)₂ (10 mg, 0.020 mmol) at r.t. The reaction was degassed with N₂ three times and stirred at 80° C. overnight. The mixture was filtered, and the filtrate was concentrated. The residue was purified by preparative TLC (5% MeOH in DCM) and further purified by preparative HPLC (Column: YMC TA C18 250×21.2 mm 5 μm, 5-95% MeCN in H₂O with 0.1% FA) to give the target product (1.5 mg, yield: 0.40%). LC/MS (ESI) (m/z): 516 [M+H]⁺.

### Example 39 (Method H)

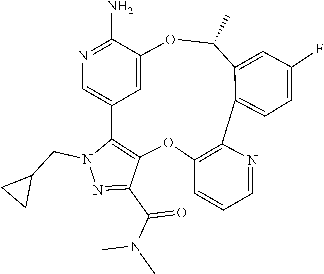 [SMILES: C[C@H]1Oc2cc(cnc2N)-c2c(c(C(=O)N(C)C)nn2CC2CC2)Oc2cccnc2-c2ccc(F)cc21]

Name: N,N-dimethyl-(R)-23-amino-3-(cyclopropylmethyl)-17-fluoro-20-methyl-7,21-dioxa-3,4,12,24-tetraazapentacyclo[20.3.1.0^{2,6}.0^{8,13}.0^{14,19}]hexacosa-1(25),2(6),4,8(13),9,11,14,16,18,22(26),23-undecaene-5-carboxamide

NMR: 1H NMR (400 MHz, DMSO) δ 8.25 (d, J=3.7 Hz, 1H), 7.72 (d, J=7.7 Hz, 1H), 7.37 (ddt, J=13.3, 7.8, 3.6 Hz, 3H), 7.10 (dd, J=8.5, 5.8 Hz, 1H). 6.92 (ddd, J=8.5, 2.7 Hz, 1H), 6.19 (s, 2H). 6.11 (d, J=1.4 Hz, 1H), 4.88 (d, J=4.9 Hz, 1H), 4.00 (dd, J=14.4, 6.2 Hz. 1H), 3.85 (dd, J=14.5, 7.3 Hz, 1H), 2.93 (s, 3H). 2.83 (s, 3H), 1.55 (d, J=6.2 Hz, 3H), 1.01 (m, J=16.7, 9.4 Hz, 1H), 0.26 (m, J=12.4, 8.5, 4.8 Hz, 2H), 0.12 (m. J=9.0, 4.2 Hz, 1H), −0.00 (m, J=9.5, 4.7 Hz, 1H).

LCMS: Method Y2: t_{R}: 1.79 min; m/z: 515 [M+H]⁺

To a solution of ethyl (R)-23-amino-3-(cyclopropylmethyl)-17-fluoro-20-methyl-7,21-dioxa-3,4,12,24-tetraazapentacyclo[20.3.1.0^{2,6}.0^{8,13}.0^{14,19}]hexacosa-1(25),2(6),4,8(13),9,11,14,16,18,22(26),23-undecaene-5-carboxylate (65.0 mg, 0.126 mmol) in THF (2 mL), EtOH (2 mL) and H₂O (2 mL) was added lithium hydroxide hydrate (26.0 mg, 0.630 mmol). The reaction was stirred at r.t. overnight. The mixture was adjusted to pH 5 with aq. HCl (1 M) and extracted with EtOAc (10 mL×3). The combined organic phases were dried over Na₂SO₄, and concentrated to give (20R)-23-amino-3-(cyclopropylmethyl)-17-fluoro-20-methyl-7,21-dioxa-3,4,12,24-tetraazapentacyclo[20.3.1.0^{2,6}.0^{8,13}.0^{14,19}]hexacosa-1(25),2(6),4,8(13),9,11,14,16,18,22(26),23-undecaene-5-carboxylic acid (45.0 mg, crude). LC/MS (ESI) (m/z): 488 [M+H]⁺.

To a solution of (20R)-23-amino-3-(cyclopropylmethyl)-17-fluoro-20-methyl-7,21-dioxa-3,4,12,24-tetraazapentacyclo[20.3.1.0^{2,6}.0^{8,13}.0^{14,19}]hexacosa-1(25),2(6),4,8(13),9,11,14,16,18,22(26),23-undecaene-5-carboxylic acid (25.0 mg, 0.0500 mmol), dimethylamine hydrochloride (4.60 mg, 0.100 mmol) and DIEA (0.03 mL, 0.200 mmol) in DMF (1 mL) was added HATU (23.3 mg, 0.0600 mmol) at 0° C. The reaction was stirred at r.t. for 1 h. The mixture was quenched with water (10 mL) and extracted with EtOAc (10 mL×3). The combined organic phases were washed with brine (10 mL), dried over anhydrous Na₂SO₄, and concentrated. The residue was purified by preparative HPLC (Column: Azzota C18 30×250 mm×10 μm, 15→95% MeCN in H₂O with 0.1% FA) to give the target product (8.00 mg, 30.0% yield, over two steps). LC/MS (ESI) (m/z): 515 [M+H]⁺.

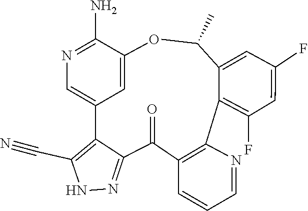 [SMILES: C[C@H]1Oc2cc(cnc2N)-c2c(n[nH]c2C#N)C(=O)c2cccnc2-c2c(F)cc(F)cc21]

### Example 77 (Method I)

Name: (R)-23-amino-15,17-difluoro-20-methyl-7-oxo-21-oxa-4,5,12,24-NH₂ tetraazapentacyclo[20.3.1.0^{2,6}.0^{8,13}.0^{14,19}]hexacosa-1(25),2,5,8,10,12,14,16,18,22(26),23-undecaene-3-carbonitrile

NMR: 1H NMR (400 MHz, DMSO-d6) δ 14.84 (s, 1H), 8.96 (dd, J=4.9, 1.6 Hz, 1H), 8.34 (s, 1H), 7.75 (dd, J=8.0, 4.9 Hz, 1H), 7.71 (s, 1H), 7.44 (d, J=9.8 Hz, 1H), 7.32 (t, J=9.2 Hz, 1H), 6.28 (s, 1H), 6.17 (d, J=1.9 Hz, 1H), 4.64 (s, 1H), 1.58 (d, J=6.2 Hz, 3H).

LCMS: Method U; t_{R}: 0.83 min; m/z: 445 [M+H]⁺

To a solution of (R)-23-amino-15,17-difluoro-20-methyl-7-oxo-4-{[2-(trimethylsilyl)ethoxy]methyl}-21-oxa-4,5,12,24-tetraazapentacyclo[20.3.1.0^{2,6}.0^{8,13}.0^{14,19}]hexacosa-1(25),2,5,8,10,12,14,16,18,22(26),23-undecaene-3-carbonitrile (55 mg, 0.096 mmol) in DCM (3 mL) was added TFA (1.0 mL, 13 mmol) and the reaction was stirred at 25° C. for 4 h. The mixture was quenched with sat. aq. NaHCO₃ and extracted with DCM (20 mL×2). The combined organic layers were dried over anhydrous Na₂SO₄, filtered, and concentrated. The residue was purified by column chromatography on silica gel (0→5% MeOH in DCM) and then further purified by prep-HPLC (Column: YMC Triart C18 250×20 mm I.D, 5 μm, 5→95% MeCN in H₂O with 0.1% FA) to give the target product (9.9 mg, yield: 23.3%). LC/MS ESI (m/z): 445 [M+H]⁺.

### Example 78 (Method J)

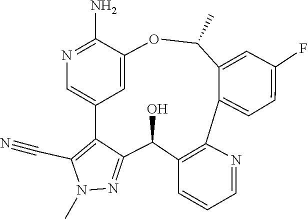 [SMILES: C[C@H]1Oc2cc(cnc2N)-c2c(nn(C)c2C#N)[C@@H](O)c2cccnc2-c2ccc(F)cc21]

Name: (7S,20R)-23-amino-17-fluoro-7-hydroxy-4-methyl-20-methyl-21-oxa-4,5,12,24-tetraazapentacyclo[20.3.1.0^{2,6}.0^{8,13}.0^{14,19}]hexacosa-1(25),2,5,8(13),9,11,14,16,18,22(26),23-undecaene-3-carbonitrile

NMR: 1H NMR (400 MHz, MeOD) δ 8.56-8.48 (m, 1H), 7.96-7.88 (m, 1H), 7.65 (d, J=1.7 Hz, 1H), 7.45-7.30 (m, 3H), 6.96 (td, J=8.5, 2.7 Hz, 1H), 6.72 (d, J=1.6 Hz, 1H), 6.43 (s, 1H), 4.97 (td, J=6.2, 4.4 Hz, 1H), 4.08 (s, 3H), 1.79 (d, J=6.3 Hz, 3H).

LCMS: Method R; t_{R}: 0.76 min; m/z: 443 [M+H]⁺

To a solution of (R)-23-amino-17-fluoro-4-methyl-20-methyl-7-oxo-21-oxa-4,5,12,24-tetraazapentacyclo[20.3.1.0^{2,6}.0^{8,13}.0^{14,19}]hexacosa-1(25),2,5,8(13),9,11,14,16,18,22(26),23-undecaene-3-carbonitrile (100 mg, 0.227 mmol) in MeOH (5 mL) was added NaBH₄ (17.0 mg, 0.454 mmol) at 25° C. After stirring at 25° C. for 1 h, the reaction was quenched with sat. aq. NH₄Cl (10 mL) and extracted with EtOAc (10 mL×3). The combined organic layers were washed with brine, dried over anhydrous Na₂SO₄ and concentrated. The residue was purified by Prep-HPLC (Column: Gemini Sum C18 250×21.2 mm; 5→95% CH₃CN in H₂O+0.1% FA) to give the target product (50 mg, yield: 50%). LC/MS (ESI) (m/z): 443 [M+H]⁺.

The following compounds were prepared in a similar manner:

| | | |
|---|---|---|
| Example 79 | 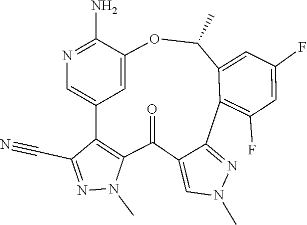 [SMILES: C[C@H]1Oc2cc(cnc2N)-c2c(C#N)nn(C)c2C(=O)c2cn(C)nc2-c2c(F)cc(F)cc21] | (19R)-22-amino-14,16-difluoro-7-hydroxy-5,10-dimethyl-19- methyl-20-oxa-4,5,10,11,23- pentaazapentacyclo[19.3.1.0²,⁶.0⁸,¹².0¹³,¹⁸]pentacosa- 1(24),2(6),3,8,11,13,15,17,21(25),22-decaene-3-carbonitrile 1H NMR (400 MHz, MeOD) δ 8.03 (s, 1H), 7.55 (d, J = 1.7 Hz, 1H), 7.43 (d, J = 8.6 Hz, 1H), 7.11 (dd, J = 10.1, 7.8 Hz, 1H), 6.65 (s, 1H), 5.41 (d, J = 6.6 Hz, 1H), 5.30 (s, 1H), 4.39 (s, 3H), 4.01 (s, 3H), 1.82 (d, J = 6.3 Hz, 3H). LCMS Method Y1; t_{R}: 1.05 min; m/z: 464 [M + H]⁺ |

### Example 80 (Method K)

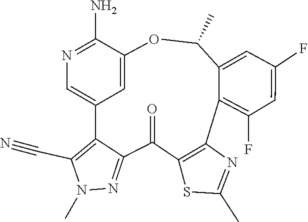 [SMILES: Cc1nc2c(s1)C(=O)c1nn(C)c(C#N)c1-c1cnc(N)c(c1)O[C@H](C)c1cc(F)cc(F)c1-2]

Name: (R)-22-amino-14,16-difluoro-4-methyl-10,19-dimethyl-7-oxo-20-oxa-9-thia-4,5,11,23-tetraazapentacyclo[19.3.1.0^{2,6}.0^{8,12}.0^{13,18}]pentacosa-1(24),2,5,8(12),10,13,15,17,21(25),22-decaene-3-carbonitrile

NMR: 1H NMR (400 MHz, MeOD) δ 7.70 (d, J=1.8 Hz, 1H), 7.34 (ddd, J=9.6, 2.4, 1.2 Hz, 1H), 7.05 (td, J=9.0, 2.5 Hz, 1H), 6.40 (d. J=1.9 Hz, 1H), 4.90 (dd, J=6.1, 1.6 Hz, 1H), 4.13 (s, 3H), 2.86 (s, 3H), 1.72 (d, J=6.3 Hz, 3H).

LCMS: Method U; t_{R}: 1.29 min; m/z: 479 [M+H]⁺

To a mixture of 3-(4-bromo-2-methylthiazole-5-carbonyl)-1-methyl-1H-pyrazole-5-carbonitrile and 3-(4-bromo-1,3-thiazole-5-carbonyl)-1-methyl-1H-pyrazole-5-carbonitrile (50 mg, 0.16 mmol) in toluene (0.6 mL), t-BuOH (0.3 mL), and H₂O (0.2 mL) was added tert-butyl (R)-(3-(1-(3,5-difluoro-2-(4,4,5,5-tetramethyl-1,3,2-dioxaborolan-2-yl)phenyl)ethoxy)pyridin-2-yl)carbamate (115 mg, 0.24 mmol), Pd(dppf)Cl₂ (12 mg, 0.02 mmol), and Na₂CO₃ (51 mg, 0.48 mmol). The reaction was degassed and purged with N₂ three times and stirred at 80° C. for 8 h. The mixture was cooled to 25° C. and filtered using EtOAc (50 mL) to rinse the filter cake. The filtrate was washed with water (15 mL), washed with brine (15 mL), dried over Na₂SO₄, and concentrated to give tert-butyl (R)-(3-(1-(2-(5-(5-cyano-1-methyl-1H-pyrazole-3-carbonyl)-2-methylthiazol-4-yl)-3,5-difluorophenyl)ethoxy)pyridin-2-yl)carbamate and tert-butyl N-{3-[(1R)-1-{2-[5-(5-cyano-1-methyl-1H-pyrazole-3-carbonyl)-1,3-thiazol-4-yl]-3,5-difluorophenyl}ethoxy]pyridin-2-yl}carbamate (80 mg, crude) as a white solid mixture. LC/MS (ESI) (m/z): 581 [M+H]⁺.

To a mixture of tert-butyl (R)-(3-(1-(2-(5-(5-cyano-1-methyl-1H-pyrazole-3-carbonyl)-2-methylthiazol-4-yl)-3,5-difluorophenyl)ethoxy)pyridin-2-yl)carbamate and tert-butyl N-{3-[(1R)-1-{2-[5-(5-cyano-1-methyl-1H-pyrazole-3-carbonyl)-1,3-thiazol-4-yl]-3,5-difluorophenyl}ethoxy]pyridin-2-yl}carbamate (80 mg, crude) in toluene (1 mL) and H₂O (0.2 mL) was added K₂CO₃ (57 mg, 0.41 mmol). The reaction was thrice degassed with N₂ and then stirred at 85° C. for 8 h. The mixture was filtered, and the filter cake was rinsed with EtOAc (10 mL). The filtrate was washed with water (5 mL) and then washed with brine (5 mL). The combined organic phase was dried over anhydrous Na₂SO₄ and concentrated. The residue was purified by flash column chromatography on silica gel (0-80% EtOAc in PE) to give (R)-3-(4-(2-(1-((2-aminopyridin-3-yl)oxy)ethyl)-4,6-difluorophenyl)-2-methylthiazole-5-carbonyl)-1-methyl-1H-pyrazole-5-carbonitrile and 3-(4-{2-[(1R)-1-[(2-aminopyridin-3-yl)oxy]ethyl]-4,6-difluorophenyl}-1,3-thiazole-5-carbonyl)-1-methyl-1H-pyrazole-5-carbonitrile (70 mg, yield: 90.6% over two steps) as a white solid mixture. LC/MS (ESI) (m/z): 481 [M+H]⁺.

To a mixture of (R)-3-(4-(2-(1-((2-aminopyridin-3-yl)oxy)ethyl)-4,6-difluorophenyl)-2-methylthiazole-5-carbonyl)-1-methyl-1H-pyrazole-5-carbonitrile and 3-(4-{2-[(1R)-1-[(2-aminopyridin-3-yl)oxy]ethyl]-4,6-difluorophenyl}-1,3-thiazole-5-carbonyl)-1-methyl-1H-pyrazole-5-carbonitrile (80 mg, 0.17 mmol) in MeCN (2 mL) was added NBS (34 mg, 0.52 mmol) slowly at 0° C. The reaction was stirred at 25° C. for 1 h. The mixture was poured into water (10 mL) and extracted with EtOAc (10 mL×3). The combined organic phases were washed with brine (10 mL), dried over anhydrous Na₂SO₄, and concentrated. The residue was purified by flash column chromatography on silica gel (0→50% EtOAc in PE) to give (R)-3-(4-(2-(1-((2-amino-5-bromopyridin-3-yl)oxy)ethyl)-4,6-difluorophenyl)-2-methylthiazole-5-carbonyl)-1-methyl-1H-pyrazole-5-carbonitrile and 3-(4-{2-[(1R)-1-[(2-amino-5-bromopyridin-3-yl)oxy]ethyl]-4,6-difluorophenyl}-1,3-thiazole-5-carbonyl)-1-methyl-1H-pyrazole-5-carbonitrile (55.0 mg, yield: 59.1%) as a white solid mixture. LC/MS (ESI) (m/z): 559 [M+H]⁺.

To a mixture of (R)-3-(4-(2-(1-((2-amino-5-bromopyridin-3-yl)oxy)ethyl)-4,6-difluorophenyl)-2-methylthiazole-5-carbonyl)-1-methyl-1H-pyrazole-5-carbonitrile and 3-(4-{2-[(1R)-1-[(2-amino-5-bromopyridin-3-yl)oxy]ethyl]-4,6-difluorophenyl}-1,3-thiazole-5-carbonyl)-1-methyl-1H-pyrazole-5-carbonitrile (40 mg, 0.07 mmol) in 2-methyl-2-butanol (5 mL) was added cataCXium A (11 mg, 0.03 mmol), Pd(OAc)₂ (3 mg, 0.02 mmol) and PivOK (40 mg, 0.28 mmol). The mixture was degassed and purged with N₂ three times and stirred at 100° C. for 16 h. The mixture was concentrated, and the residue was purified by preparative TLC (100% EtOAc) and then by preparative HPLC (Column: YMC-Actus Triart C18 150×20 mm; 25→95% MeCN in H₂O+0.1% FA) to give the target product (5.6 mg) and the des-methyl product (4.4 mg). LC/MS ESI (m/z):479 [M+H]⁺.

The following compounds were prepared in a similar manner:

| | | |
|---|---|---|
| Example 81 | 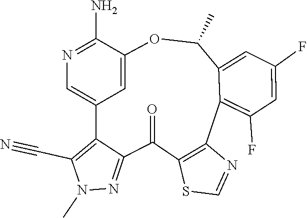 [SMILES: C[C@H]1Oc2cc(cnc2N)-c2c(nn(C)c2C#N)C(=O)c2scnc2-c2c(F)cc(F)cc21] | (R)-22-amino-14,16-difluoro-4-methyl-19-methyl-7-oxo-20- oxa-9-thia-4,5,11,23- tetraazapentacyclo[19.3.1.0²,⁶.0⁸,¹².0¹³,¹⁸]pentacosa- 1(24),2,5,8(12),10,13,15,17,21(25),22-decaene-3-carbonitrile 1H NMR (400 MHz, MeOD) δ 9.38 (s, 1H), 7.69 (d, J = 1.9 Hz, 1H), 7.37-7.31 (m, 1H), 7.05 (td, J = 9.0, 2.5 Hz, 1H), 6.28 (d, J = 1.7 Hz, 1H), 4.80-4.75 (m, 1H), 4.12 (s, 3H), 1.70 (d, J = 6.3 Hz, 3H). LCMS Method U; t_{R}: 1.20 min; m/z: 465 [M + H]⁺ |

### Example 82 (Method L)

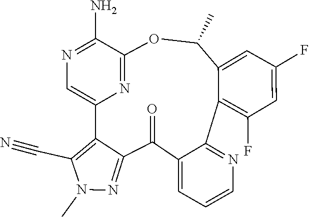 [SMILES: C[C@H]1Oc2nc(cnc2N)-c2c(nn(C)c2C#N)C(=O)c2cccnc2-c2c(F)cc(F)cc21]

Name: (R)-23-amino-15,17-difluoro-4-methyl-20-methyl-7-oxo-21-oxa-4,5,12,24,26-pentaazapentacyclo[20.3.1.0^{2,6}.0^{8,13}.0^{14,19}]hexacosa-1(25),2,5,8(13),9,11,14,16,18,22(26),23-undecaene-3-carbonitrile

NMR: 1H NMR (400 MHz, MeOD) δ 8.83 (dd, J=4.9, 1.7 Hz. 1H), 8.47 (dd, J=8.0, 1.7 Hz, 1H), 7.94 (s, 1H), 7.66 (dd, J=8.0, 5.0 Hz, 1H), 7.20 (dd, J=7.8, 2.4 Hz. 1H), 6.99 (td, J=9.1, 2.5 Hz, 1H). 5.06 (q, J=6.7 Hz, 1H), 4.15 (s, 3H), 1.53 (d, J=6.6 Hz, 3H).

LCMS: Method R; t_{R}: 1.33 min; m/z: 460 [M+H]⁺

A solution of 3-((2-(2-((R)-1-((3-amino-6-bromopyrazin-2-yl)oxy)ethyl)-4,6-difluorophenyl)pyridin-3-yl)(methoxymethoxy)methyl)-1-methyl-1H-pyrazole-5-carbonitrile (400 mg, 0.68 mmol) in MeOH (5 mL) and aq. HCl (1 M, 5 mL) was stirred at 60° C. for 16 h. The mixture was neutralized with sat. aq. NaHCO₃, diluted with EtOAc (50 mL), and washed with brine (50 mL). The organic layer was dried over anhydrous Na₂SO₄ and concentrated. The residue was purified by column chromatography on silica gel (0→30% EtOAc in PE) to give 3-((2-(2-((R)-1-((3-amino-6-bromopyrazin-2-yl)oxy)ethyl)-4,6-difluorophenyl)pyridin-3-yl)(hydroxy)methyl)-1-methyl-1H-pyrazole-5-carbonitrile (320 mg, 86% yield) as a yellow oil. LC/MS (ESI) (m/z): 542 [M+H]⁺.

To a solution of 3-((2-(2-((R)-1-((3-amino-6-bromopyrazin-2-yl)oxy)ethyl)-4,6-difluorophenyl)pyridin-3-yl)(hydroxy)methyl)-1-methyl-1H-pyrazole-5-carbonitrile (50.0 mg, 0.0920 mmol) in DCM (4 mL) was added Dess-Martin periodinane (39.0 mg, 0.138 mmol) slowly at 0° C. The reaction was stirred at 25° C. for 1 h. The mixture was filtered, and the filtrate was concentrated. The residue was purified by column chromatography on silica gel (0→40% EtOAc in PE) to give 3-(2-(2-((R)-1-((3-amino-6-bromopyrazin-2-yl)oxy)ethyl)-4,6-difluorophenyl)nicotinoyl)-1-methyl-1H-pyrazole-5-carbonitrile (20 mg, 40% yield) as a yellow solid. LC/MS (ESI) (m/z): 540 [M+H]⁺.

To a solution of 3-(2-(2-((R)-1-((3-amino-6-bromopyrazin-2-yl)oxy)ethyl)-4,6-difluorophenyl)nicotinoyl)-1-methyl-1H-pyrazole-5-carbonitrile (20.0 mg, 0.0370 mmol) in 2-methyl-2-butanol (5 mL) were added KOAc (10.9 mg, 0.11 mmol), cataCXium A (5.3 mg, 0.015 mmol) and Pd(OAc)₂ (1.7 mg, 0.007 mmol). The mixture was degassed under N₂ three times and stirred at 120° C. for 16 h. The mixture was partitioned between EtOAc (10 mL) and water (10 mL). The organic phase was dried over Na₂SO₄ and concentrated to dryness. The residue was purified by prep-TLC (50% EtOAc in PE) and then by prep-HPLC (Column: Gemini 5 μm C18 250×21.2 mm; 5→95% MeCN in H₂O+0.1% FA) to give the target product (4.0 mg, 24% yield). LC/MS (ESI) (m/z): 460 [M+H]⁺.

### Example 16 (Method M)

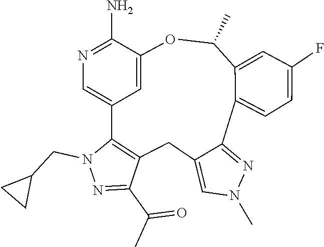 [SMILES: CC(=O)c1nn(CC2CC2)c2c1Cc1cn(C)nc1-c1ccc(F)cc1[C@@H](C)Oc1cc-2cnc1N]

Name: 1-[(19R)-22-amino-3-(cyclopropylmethyl)-16-fluoro-10,19-dimethyl-20-oxa-3.4.10,11,23-pentaazapentacyclo[19.3.1.0^{2,6}.0^{8,12}.0^{13,18}]pentacosa-1(24),2(6),4,8,11.13,15,17,21(25),22-decaen-5-yl]ethan-1-one

NMR: 1H NMR (400 MHz, MeOD) δ 7.54 (dd, J=10.1, 2.7 Hz, 1H). 7.50-7.44 (m, 2H), 7.18 (dd, J=8.5, 5.8 Hz, 1H), 7.07 (td, J=8.3, 2.7 Hz, 1H), 6.54 (s, 1H), 5.46 (d, J=6.3 Hz, 1H), 4.24 (d, J=14.9 Hz, 1H), 4.11 (dd, J=14.3, 6.3 Hz, 1H), 4.03 (dd, J=14.2, 7.4 Hz, 1H), 3.90 (s, 3H), 2.84 (d, J=14.8 Hz, 1H), 2.64 (s, 3H), 1.83 (d, J=6.3 Hz, 3H), 1.12 (t, J=6.8 Hz, 1H), 0.54-0.39 (m, 2H), 0.31 (dd, J=9.6, 4.6 Hz, 1H), 0.09 (dd, J=9.4, 4.9 Hz, 1H).

LCMS: Method W; t_{R}: 0.51 min; m/z: 487 [M+H]⁺

To a solution of N-methoxy-N-methyl-(R)-22-amino-3-(cyclopropylmethyl)-16-fluoro-10-methyl-19-methyl-20-oxa-3,4,10,11,23-pentaazapentacyclo[19.3.1.0^{2,6}.0^{8,12}.0^{13,18}]pentacosa-1(24),2(6),4,8,11,13,15,17,21(25),22-decaene-5-carboxamide (15.0 mg, 0.0280 mmol) in THF (3 mL) was added MeMgBr (3 M in THF, 0.03 mL, 1 mmol) at 0° C. and the reaction was stirred at 0° C. for 2 h. The mixture was quenched with water (5 mL) and extracted with EtOAc (20 mL×2). The combined organic layers were dried over anhydrous Na₂SO₄, filtered, and concentrated. The residue was purified by preparative HPLC (Column: YMC-Actus Triart C18 250×21 mm; 20-95% MeCN in H₂O+0.1% FA) to afford the target product (10.0 mg, 73.0% yield). LC/MS (ESI) (m/z): 487 [M+H]⁺.

### Inhibition Assays

### Biochemical Kinase Assay

First, 250 nL of compound dissolved in DMSO (100-fold of the desired concentration) was dispensed into a 384-well plate. A 12.5 μL substrate solution containing ATP (2 mM) and fluorogenic phosphorylation substrate AQT0101 (26 M for ALK and ROS, AssayQuant) or AQT0104 (26 μM for TRKB, AssayQuant) in buffer (50 mM HEPES pH 7.5, 0.0100 Brij-35, 0.5 mM EGTA, 10 mM MgCl₂) was added and mixed thoroughly. Then, a 12.5 μL kinase solution containing ALK (1.5 nM, Carna, 08-518), ALK I1171N/D1203N (4 nM, SignalChem, custom-made), ROS1 (0.6 nM, Carma, 08-163), ROS1-G2032R (0.5 nM, SignalChem, R14-121BG), or TRKB-wt (1.5 nM, SignalChem, N17-11G) kinase domains in buffer (50 nM HEPES pH 7.5, 0.01% Brij-35,200 glycerol, 0.4 mg/mL BSA, 0.5 mM EGTA, and 10 mM MgCl₂) was added and mixed thoroughly. The plate was sealed and read by SpectraMax Paradigm at λ=485 nm every 2 minutes for 120 minutes at 30° C. Exemplary data is given in Table 3. Initial rates of reaction (v) were calculated from the change in fluorescence intensity over time during the initial, linear portion of the reaction. Finally, apparent inhibitory constants (Kᵢ^{app}) were determined from regression of v and I (inhibitor concentration) to Morrison Equation (E=enzyme concentration):

$$v = 1 - \frac{( E + I + {K}_{i}^{app} ) - \sqrt{{( E + I + {K}_{i}^{app} )}^{2} - 4 EI}}{2 E}$$

TABLE 3
Biochemical Assay Activity Summary

| | | | ALK-I1171N- | |
|---|---|---|---|---|
| Example | ROS1-wt | ROS1-G2032R | D1203N | TRKB-wt |
| 2 | nd | nd | B | C |
| 3 | nd | nd | A | B |
| 4 | A | A | nd | nd |
| 5 | nd | nd | A | A |
| 6 | nd | nd | A | B |
| 7 | A | A | A | A |
| 8 | A | A | A | B |
| 9 | A | A | A | B |
| 10 | nd | nd | A | B |
| 11 | nd | nd | A | B |
| 12 | nd | nd | A | C |
| 13 | nd | nd | A | B |
| 14 | nd | nd | A | A |
| 15 | nd | nd | nd | nd |
| 16 | nd | nd | B | C |
| 17 | nd | nd | A | B |
| 18 | nd | nd | A | B |
| 19 | nd | nd | A | A |
| 20 | nd | nd | A | B |
| 27 | A | A | A | A |
| 30 | nd | nd | A | B |
| 31 | nd | nd | A | C |
| 32 | nd | nd | A | C |
| 33 | nd | nd | A | C |
| 34 | nd | nd | A | B |
| 35 | nd | nd | A | B |
| 36 | nd | nd | A | C |
| 37 | nd | nd | A | C |
| 38 | nd | nd | A | B |
| 39 | nd | nd | A | C |
| 40 | nd | nd | A | C |
| 41 | nd | nd | A | C |
| 42 | nd | nd | A | C |
| 43 | nd | nd | A | A |
| 44 | nd | nd | A | B |
| 45 | nd | nd | A | C |
| 47 | nd | nd | B | nd |
| 49 | A | B | A | B |
| 55 | nd | nd | A | C |
| 56 | nd | nd | B | C |
| 58 | A | A | A | B |
| 66 | A | A | nd | nd |
| 76 | nd | nd | A | nd |

Compound potency can be interpreted by binning Kᵢ^{app} values against the targets: bin A for high potency, Kᵢ^{app}<50 nM; bin B for medium potency, 50 nM≤Kᵢ^{app}<500 nM; and bin C for low potency, Kᵢ^{app}≥500 nM. Compounds are more desirable if they exhibit smaller Kᵢ^{app}values against the on-target kinases (ROS1 or ALK) and larger Kᵢ^{app} values against the off-target kinase (TRKB). Compounds that potently inhibit the on-target kinases (ROS1 or ALK) are also expected to inhibit ROS1 or ALK oncoproteins that are expressed in human cancers, providing support for the potential clinical efficacy of such compounds. Similarly, compounds that do not potently inhibit the off-target kinase (TRKB) are expected to poorly inhibit TRK-family kinases in humans and hence avoid potential clinical toxicity arising from TRKA, TRKB, or TRKC inhibition. “nd” means not determined.

### Generation of Ba/F3 Stable Cell Lines

Genes encoding EML4-ALK v1 (variant 1), EML4-ALK v1 G1202R, EML4-ALK v1 I1171IN, EML4-ALK v1 D1203N, EML4-ALK v1 I1171N/D1203N, and ETV6-TRKB were synthesized at GeneRay, cloned into the retroviral construct pMSCV-puro (Biovector), and packaged into retroviral particles. The virus was used to infect Ba/F3 cells (RIKEN) at multiplicity of infection=1 for 1 day. Infected cells were rescued in media (RPMI-1640 with 10% fetal bovine serum and 1% streptomycin and penicillin) supplemented with mouse IL-3 (10 ng/mL) for 2 days, and stable cell lines were selected by IL-3 withdrawal and puromycin (0.8 μg/mL) for 7 days. Monoclones were selected by single-cell dilution in IL-3-free medium containing puromycin (0.8 μg/mL). Transformation of desired genes was confirmed by Sanger sequencing and western blot using the following antibodies: ALK (CST #3633) and pan-TRK (Abcam #76291).

### Cell Proliferation Assay

Stable cells were plated in a 384-well plate. Test compounds were then added in a 3-fold dilution series and incubated for 72 hours. CellTiter-Glo reagent (Promega) was added. Luminescence was measured on a plate reader. Half-maximal inhibitory concentration (ICso) was calculated from percent inhibition and inhibitor concentration using four-parameter logistic regression. Compound potency can be interpreted by binning IC₅₀ values: bin A for high potency, 0.1 nM IC₅₀<50 nM; bin B for medium potency, 50 nM<IC₅₀<500 nM; and bin C for low potency, IC₅₀>500 nM. Compounds are more desirable if they exhibit smaller IC₅₀ values against the on-target Ba/F3 cells (ALK-fusion) and larger IC₅₀ values against the off-target Ba/F3 cells (TRKB-fusion). Exemplary data is given in Table 4 (nd is not determined).

Ba/F3 proliferation is driven by the transduced oncogenes in the same way that cancer cell proliferation in humans is driven by the expression of equivalent oncogenes. Hence, compounds that potently inhibit the proliferation of the on-target Ba/F3 cells (ALK-fusion) are also expected to inhibit human cancers that express equivalent oncogenes, providing support for the potential clinical efficacy of such compounds. Similarly, compounds that do not potently inhibit the off-target Ba/F3 cells (TRKB-fusion) are expected to poorly inhibit TRK-family kinases in humans and hence avoid the clinical toxicity arising from TRKA, TRKB, or TRKC inhibition.

TRKB selectivity was calculated by dividing a compound's TRKB potency by its primary target potency (e.g., ETV6-TRKB IC50/EML4-ALK v1 IC50). Compound selectivity can be interpreted by binning ratio values: bin A for very high selectivity, ratio≥30-fold; bin B for high selectivity, ratio≥10-fold but<30; bin C for moderate selectivity, ratio≥1 but<10; and bin D for low selectivity, ratio<1. Compounds are more desirable if they exhibit higher selectivity ratios. Exemplary data are given in Table 5. (nd=not determined).

TABLE 4
Cell Assay Activity Summary

| | EML4-ALK | EML4-ALK v1 | |
|---|---|---|---|
| Example | v1 I1171N | I1171N/D1203N | ETV6-TRKB |
| 1 | B | C | C |
| 2 | nd | B | C |
| 3 | A | A | C |
| 4 | B | C | B |
| 5 | A | B | B |
| 6 | nd | A | C |
| 7 | A | A | B |
| 8 | A | A | C |
| 9 | nd | B | C |
| 10 | A | A | C |
| 11 | nd | B | C |
| 12 | nd | B | C |
| 13 | nd | B | C |
| 14 | A | B | A |
| 15 | C | B | B |
| 16 | nd | B | C |
| 17 | nd | B | C |
| 18 | nd | B | C |
| 19 | nd | A | B |
| 20 | A | A | B |
| 21 | A | A | B |
| 22 | nd | A | C |
| 23 | nd | B | C |
| 24 | nd | A | A |
| 25 | nd | A | C |
| 26 | B | A | C |
| 27 | A | A | B |
| 28 | nd | B | C |
| 29 | C | C | C |
| 30 | nd | B | C |
| 31 | nd | A | C |
| 32 | A | A | C |
| 33 | nd | B | C |
| 34 | nd | A | C |
| 35 | A | A | C |
| 36 | nd | B | C |
| 37 | nd | B | C |
| 38 | A | A | C |
| 39 | nd | A | C |
| 40 | nd | B | C |
| 41 | nd | A | C |
| 42 | nd | B | C |
| 43 | nd | A | A |
| 44 | nd | A | C |
| 45 | B | A | C |
| 46 | B | A | B |
| 47 | nd | B | C |
| 48 | A | A | B |
| 49 | A | A | B |
| 50 | A | A | B |
| 51 | A | A | B |
| 52 | A | A | B |
| 53 | B | A | C |
| 54 | A | A | A |
| 55 | nd | B | C |
| 56 | nd | B | C |
| 57 | A | A | B |
| 58 | A | B | C |
| 59 | nd | A | B |
| 61 | nd | A | B |
| 66 | B | C | nd |
| 67 | nd | A | B |
| 68 | nd | B | C |
| 69 | nd | A | B |
| 70 | nd | C | C |
| 71 | B | B | C |
| 72 | B | B | C |
| 73 | A | A | B |
| 74 | nd | B | C |
| 75 | nd | A | A |
| 76 | C | B | C |
| 77 | A | A | B |
| 78 | nd | B | C |
| 79 | nd | B | C |
| 80 | nd | A | B |
| 81 | A | A | C |
| 82 | A | A | B |

TABLE 5
Cell Assay Selectivity Summary

| | ETV6-TRKB / EML4-ALK | ETV6-TRKB / EML4-ALK |
|---|---|---|
| Example | v1 I1171N | v1 I1171N/D1203N |
| 1 | B | C |
| 2 | nd | C |
| 3 | A | B |
| 4 | C | D |
| 5 | C | D |
| 6 | nd | B |
| 7 | B | C |
| 8 | A | A |
| 9 | nd | B |
| 10 | A | A |
| 11 | nd | B |
| 12 | nd | C |
| 13 | nd | C |
| 14 | C | D |
| 15 | D | C |
| 16 | nd | C |
| 17 | nd | C |
| 18 | nd | C |
| 19 | nd | B |
| 20 | A | A |
| 21 | B | A |
| 22 | nd | A |
| 23 | nd | A |
| 24 | nd | C |
| 25 | nd | A |
| 26 | A | A |
| 27 | A | A |
| 28 | nd | A |
| 29 | B | B |
| 30 | nd | B |
| 31 | nd | A |
| 32 | A | A |
| 33 | nd | A |
| 34 | nd | B |
| 35 | A | A |
| 36 | nd | A |
| 37 | nd | B |
| 38 | A | A |
| 39 | nd | A |
| 40 | nd | A |
| 41 | nd | A |
| 42 | nd | A |
| 43 | nd | B |
| 44 | nd | A |
| 45 | B | A |
| 46 | C | B |
| 47 | nd | B |
| 48 | B | A |
| 49 | A | A |
| 50 | A | A |
| 51 | A | A |
| 52 | A | A |
| 53 | B | A |
| 54 | C | B |
| 55 | nd | B |
| 56 | nd | C |
| 57 | A | B |
| 58 | A | C |
| 59 | nd | B |
| 61 | nd | B |
| 66 | nd | nd |
| 67 | nd | B |
| 68 | nd | B |
| 69 | nd | B |
| 70 | nd | C |
| 71 | B | B |
| 72 | A | B |
| 73 | B | B |
| 74 | nd | C |
| 75 | nd | A |
| 76 | C | C |
| 77 | B | A |
| 78 | nd | B |
| 79 | nd | C |
| 80 | nd | B |
| 81 | A | A |
| 82 | C | A |

Additional exemplified compounds were also tested against ALK-INDN and TRKB. The TRKB selectivity was calculated by dividing a compound's TRKB potency by its primary target potency (e.g., ETV6-TRKB IC₅₀/EMIL4-ALK I1171N-D1203N v1 IC₅₀). Compound selectivity can be interpreted by binning ratio values: bin AA for the selectivity with the ratio≥30-fold; bin BB for the selectivity with the ratio≥10-fold but<30; bin CC for the selectivity with the ratio≥5 but<10; bin DD for the selectivity with the ratio≥1 but<5; and bin EE for the selectivity with the ratio<1. Exemplary data are given in Table 6. The structures of compounds E1-E13 are shown in Table 7. Compared to E1-E13, certain compounds provided herein exhibited higher selectivity ratios.

TABLE 6
Cell Assay Selectivity Summary

| Compound | Selectivity for Ba/F3 EML4-ALK v1 I1171N-D1203N |
|---|---|
| No. | IC₅₀ over Ba/F3 ETV6-TRKB IC₅₀ |
| E1 | BB |
| 21 | AA |
| E2 | DD |
| 22 | AA |
| E3 | EE |
| 18 | CC |
| E4 | EE |
| 15 | DD |
| E5 | CC |
| 51 | AA |
| E6 | EE |
| 74 | CC |
| E7 | CC |
| 49 | AA |
| 43 | BB |
| 47 | BB |
| E8 | DD |
| 25 | AA |
| E9 | CC |
| 52 | AA |
| E10 | EE |
| 76 | DD |
| E11 | DD |
| 29 | BB |
| E12 | CC |
| 50 | AA |
| E13 | BB |
| 26 | AA |

TABLE 7
Structures of compounds E1-E13

| | |
|---|---|
| 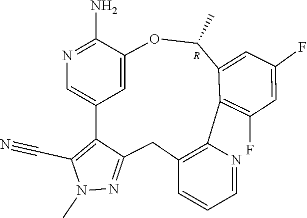 | E1 |
| 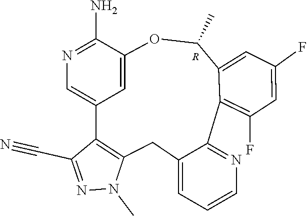 | E2 |
| 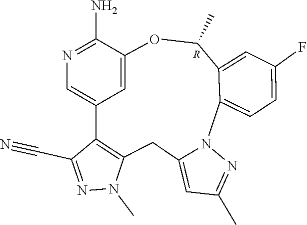 | E3 |
| 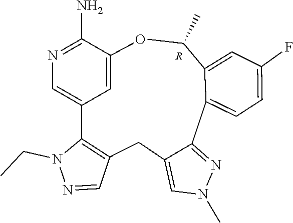 | E4 |
| 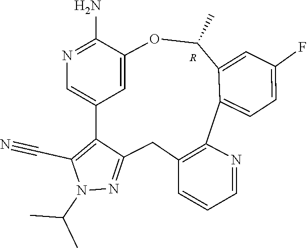 | E5 |
| 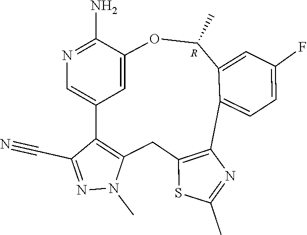 | E6 |
| 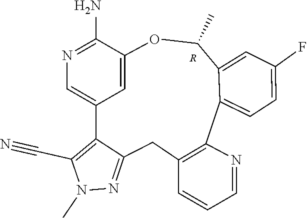 | E7 |
| 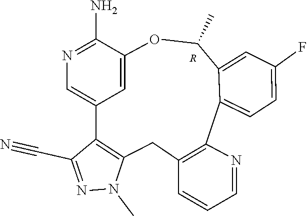 | E8 |
| 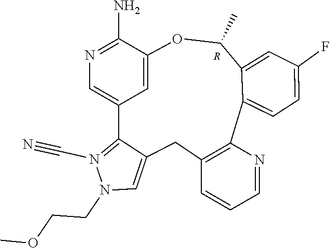 | E9 |
| 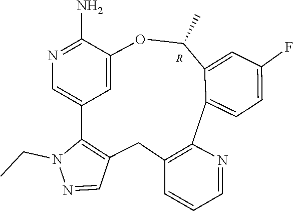 | E10 |
| 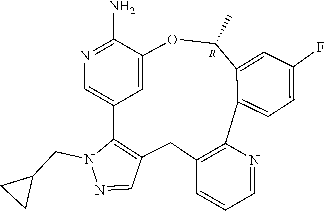 | E11 |
| 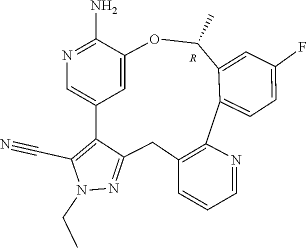 | E12 |
| 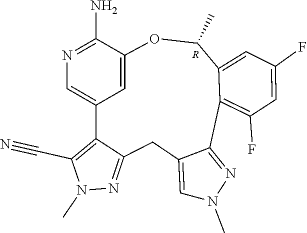 | E13 |

### Generation of Ba/F3 Stable Cell Lines for LTK

Genes encoding CLIP1-LTK are synthesized, cloned into the retroviral construct pMSCV-puro (Biovector), and packaged into retroviral particles. The virus is used to infect Ba/F3 cells (RIKEN) at multiplicity of infection=1 or 10 for 1 day. Infected cells are rescued in media (RPMI-1640 with 10% fetal bovine serum and 1% streptomycin and penicillin) supplemented with mouse IL-3 (10 ng/mL) for 2 days, and stable cell lines are selected by IL-3 withdrawal and puromycin (0.8 μg/mL) for 7 days. Transformation of desired genes is confirmed by Sanger sequencing and western blot.

### Cell Proliferation Assay

Stable cells are plated at 1,000 cells/well (40 μL) in a 384-well plate for 1 day. Test compounds (40 nL) are then added in a 3-fold dilution series using the TECAN EVO200 liquid handler and incubated for 72 hours. Plates are equilibrated at room temperature for 15 minutes followed by addition of 40 μL CellTiter-Glo reagent (Promega). Luminescence is measured on a plate reader. Half-maximal inhibitory concentration (IC₅₀) is calculated from percent inhibition and inhibitor concentration using four-parameter logistic regression.

### Kinetic Solubility Study

The kinetic solubility of compounds in phosphate buffer at pH 6.8 was evaluated using the following protocol. The test compound was dissolved in DMSO at a concentration of 10 mM to make a stock solution. 30 L of this stock solution was diluted with 970 μL of pH 6.8 buffer. This sample was incubated at 25° C. for 2 hours with shaking at 1100 rpm. The shaking was stopped, and the mixture was vacuum filtered. A 10 L aliquot of the filtrate was diluted with DMSO (10 μL), acetonitrile (490 μL), and water (490 μL). This solution was analyzed by HPLC to determine a peak area. The amount in solution was determined by comparison to the peak area measured for a standard sample of the same test compound with the known concentration (3 μL). The standard solution was prepared by diluting 30 μL of the 10 mM DMSO stock solution with 970 μL of DMSO. A 10 μL aliquot of this solution was further diluted with pH 6.8 phosphate buffer (10 μL), acetonitrile (490 μL), and water (490 L) to provide the standard solution.

All publications and patents mentioned herein are hereby incorporated by reference in their entirety as if each individual publication or patent was specifically and individually indicated to be incorporated by reference. In case of conflict, the present application, including any definitions herein, will control.

While specific embodiments of the subject disclosure have been discussed, the above specification is illustrative and not restrictive. Many variations provided herein will become apparent to those skilled in the art upon review of this specification and the claims below. The full scope of the disclosure should be determined by reference to the claims, along with their full scope of equivalents, and the specification, along with such variations.

What is claimed is:

1. A compound of Formula (I): 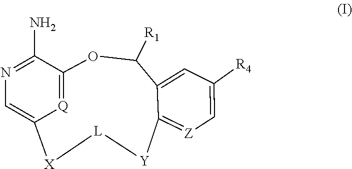
or a stereoisomer, a mixture of stereoisomers, or a pharmaceutically acceptable salt thereof, wherein
Q is CH or N,
Z is CR₅ or N;
L is —CH₂—, C═O, or —O—;
X is a 5-membered heteroarylene, comprising 1 to 3 heteroatoms selected from the group consisting of nitrogen, oxygen and sulfur; wherein the 5-membered heteroarylene is substituted with 1, 2, or 3 occurrences of R₂;
Y is a 5- or 6-membered heteroarylene, comprising 1 to 3 heteroatoms selected from the group consisting of nitrogen, oxygen and sulfur; wherein the 5- or 6-membered heteroarylene is substituted with 0, 1, or 2 occurrences of R₃;
R₁ is selected from the group consisting of H, methyl, and hydroxymethyl;
each instance of R₂ is independently selected from the group consisting of H, CN, halo, 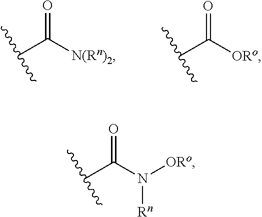
—CO—C₁₋₄ alkyl, 5-membered heteroaryl, C₁₋₄ alkyl-SO—, C₁₋₄ alkyl-SO₂—, C₁₋₄ alkoxy, C₁₋₄ alkyl, halo-C₁₋₄ alkyl, C₁₋₄ alkoxy-Ct-4 alkyl, C₃₋₄ cycloalkylmethyl, C₃₋₆ cycloalkyl, and C₃₋₆ heterocyclyl; provided that, when L is —CH₂—, at least one occurrence of R₂ is 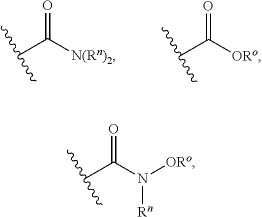
—CO—C₁₋₄ alkyl, 5-membered heteroaryl, C₁₋₄ alkyl-SO—, or C₁₋₄ alkyl-SO₂—; and wherein the heteroaryl, cycloalkyl, heterocyclyl, or alkyl is further substituted with 0, 1, 2, or 3 occurrences of C₁₋₄ alkyl or halogen as valency permits;
each instance of Rⁿ is independently H, C₁₋₄ alkyl, halo-C₁₋₄ alkyl, or C₃₋₆ cycloalkyl, or two Rⁿ groups are taken together with their intervening nitrogen to form a C₃₋₆ heterocycloalkyl optionally substituted with one or more occurrences of C₁₋₄ alkyl or halogen;
each instance of R^{o} is independently H or C₁₋₄ alkyl;
each instance of R₃ is independently selected from the group consisting of H, halo, CN, C₁₋₄ alkoxy, halo-C₁₋₄ alkyl, and C₁₋₄ alkyl; and
each of R₄ and R₅ is independently H or F.

2. A compound of Formula (I): 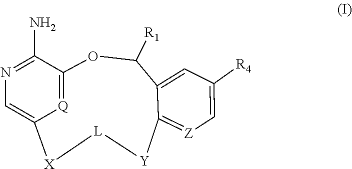
or a stereoisomer, a mixture of stereoisomers, or a pharmaceutically acceptable salt thereof, wherein
Q is CH or N;
Z is CR₅ or N;
L is —CH₂—, C═O, —CH(OH)—, or —O—;
X is a 5-membered heteroarylene, comprising 1 to 3 heteroatoms selected from the group consisting of nitrogen, oxygen and sulfur; wherein the 5-membered heteroarylene is substituted with 1, 2, or 3 occurrences of R₂;
Y is a 5- or 6-membered heteroarylene, comprising 1 to 3 heteroatoms selected from the group consisting of nitrogen, oxygen and sulfur; wherein the 5- or 6-membered heteroarylene is substituted with 0, 1, or 2 occurrences of R₃;
R₁ is selected from the group consisting of H, methyl, and hydroxymethyl;
each instance of R₂ is independently selected from the group consisting of H, CN, halo, 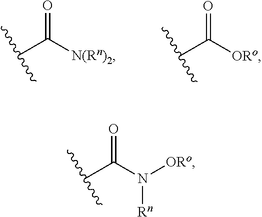
—S—C₁₋₄ alkyl, —CO—C₁₋₄ alkyl, 5-membered heteroaryl, C₁₋₄ alkyl-SO—, C₁₋₄ alkyl-SO₂—, C₁₋₄ alkoxy, C₁₋₄ alkyl, halo-C₁₋₄ alkyl, C₁₋₄ alkoxy-C₁₋₄ alkyl, C₃₋₄ cycloalkylmethyl, C₃₋₆ cycloalkyl, and C₃₋₆ heterocyclyl; provided that, when L is —CH₂—, at least one occurrence of R₂ is 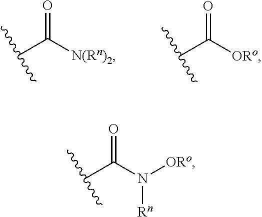
—CO—C₁₋₄ alkyl, 5-membered heteroaryl, —S—C₁₋₄ alkyl, C₁₋₄ alkyl-SO—, or C₁₋₄ alkyl-SO₂—; and wherein the heteroaryl, cycloalkyl, heterocyclyl, alkoxy, or alkyl is further substituted with 0, 1, 2, or 3 occurrences of C₁₋₄ alkyl, Si(C₁₋₄ alkyl)₃, or halogen as valency permits;
each instance of Rⁿ is independently H, C₁₋₄ alkyl, halo-C₁₋₄ alkyl, or C₃₋₆ cycloalkyl, or two Rⁿ groups are taken together with their intervening nitrogen to form a C₃₋₆ heterocycloalkyl optionally substituted with one or more occurrences of C₁₋₄ alkyl or halogen;
each instance of R^{o} is independently H or C₁₋₄ alkyl;
each instance of R₃ is independently selected from the group consisting of H, halo, CN, C₁₋₄ alkoxy, halo-C₁₋₄ alkyl, and C₁₋₄ alkyl; and
each of R₄ and R₅ is independently H or F.

3. The compound of claim 1 or 2, which is a compound of Formula (I-A): 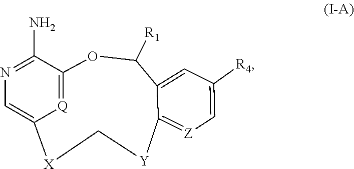
or a stereoisomer, a mixture of stereoisomers, or a pharmaceutically acceptable salt thereof.

4. The compound of claim 1 or 2, which is a compound of Formula (I-A-1): 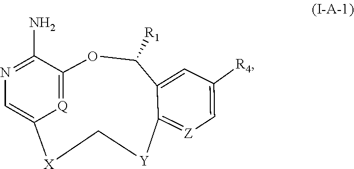
or a pharmaceutically acceptable salt thereof.

5. The compound of claim 1 or 2, which is a compound of Formula (I-B): 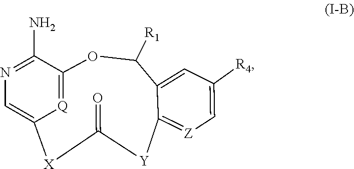
or a stereoisomer, a mixture of stereoisomers, or a pharmaceutically acceptable salt thereof.

6. The compound of claim 1 or 2, which is a compound of Formula (I-B-1): 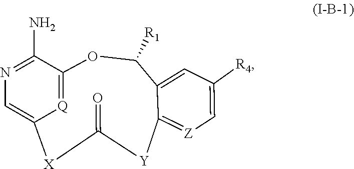
or a pharmaceutically acceptable salt thereof.

7. The compound of claim 1 or 2, which is a compound of Formula (I-C): 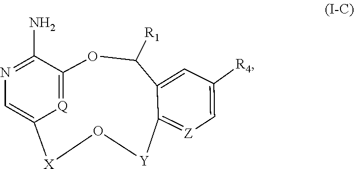
or a stereoisomer, a mixture of stereoisomers, or a pharmaceutically acceptable salt thereof.

8. The compound of claim 1 or 2, which is a compound of Formula (I-C-1): 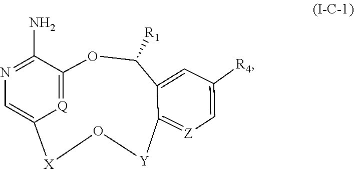
or a pharmaceutically acceptable salt thereof.

9. The compound of claim 1 or 2, which is a compound of Formula (I-D): 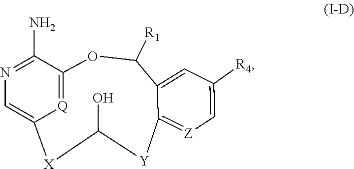
or a stereoisomer, a mixture of stereoisomers, or a pharmaceutically acceptable salt thereof.

10. The compound of claim 1 or 2, which is a compound of Formula (I-D-1): 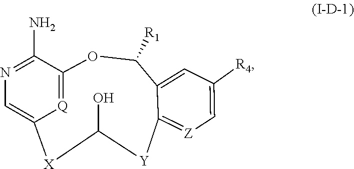
or a pharmaceutically acceptable salt thereof.

11. The compound of claim 1 or 2, which is a compound of Formula (I-D-1-1): 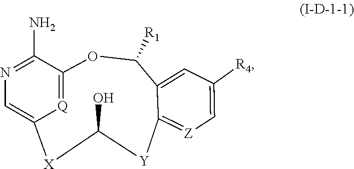
or a pharmaceutically acceptable salt thereof.

12. The compound of any one of claims 1 to 11, wherein X is a 5-membered heteroarylene selected from the group consisting of 3*,4-substituted-pyrazolylene, 4*,5-substituted-pyrazolylene, 4,5*-substituted-pyrazolylene, 1*,5-substituted-pyrazolylene, 4*,5-substituted-imidazolylene, 1*,5-substituted-imidazolylene, or 4*,5-substituted-triazolylene, wherein * indicates the point of attachment of X or Y to the L group bonded to X and Y.

13. The compound of claim 12, wherein X is a 5-membered heteroarylene selected from the group consisting of: 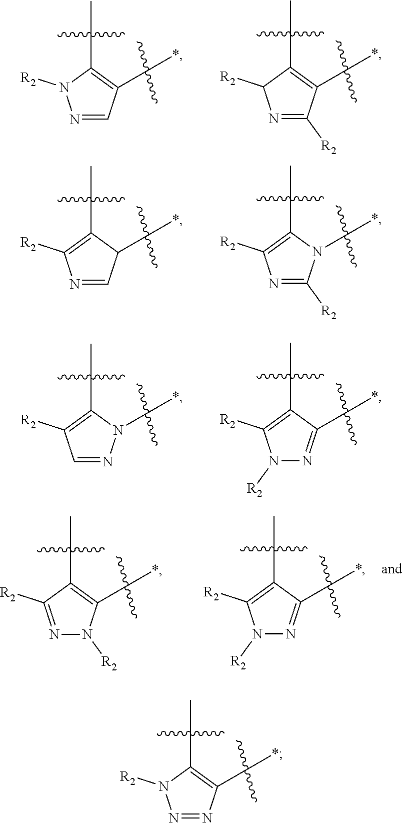
wherein * indicates the point of attachment of X to the L group bonded to X and Y.

14. The compound of claim 13, wherein X is 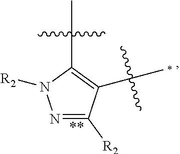
and the R₂ at ** position is 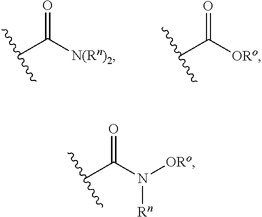
—CO—C₁₋₄ alkyl, —S—C₁₋₄ alkyl, CN, 5-membered heteroaryl, C₁₋₄ alkyl-SO—, or C₁₋₄ alkyl-SO₂—.

15. The compound of claim 13, wherein X is

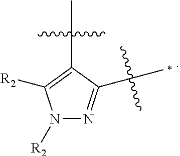

16. The compound of any one of claims 1 to 15, wherein Y is selected from the group consisting of 1*,5-substituted-pyrazolylene, 3*,4-substituted-pyrazolylene, 2,3*-substituted-pyridinylene, 3*,4-substituted-pyridinylene, 3,4*-substituted-pyridinylene, 4,5*-substituted 1,3-thiazolylene, 4*,5-substituted-1,2,3-triazolylene, 1*, 5-substituted-1,2,4-triazolylene, 1,5*-substituted-1,2,4-triazolylene, and 4*,5-substituted 1,3-thiazolylene, wherein * indicates the point of attachment to the L group bonded to X and Y.

17. The compound of claim 16, wherein Y is 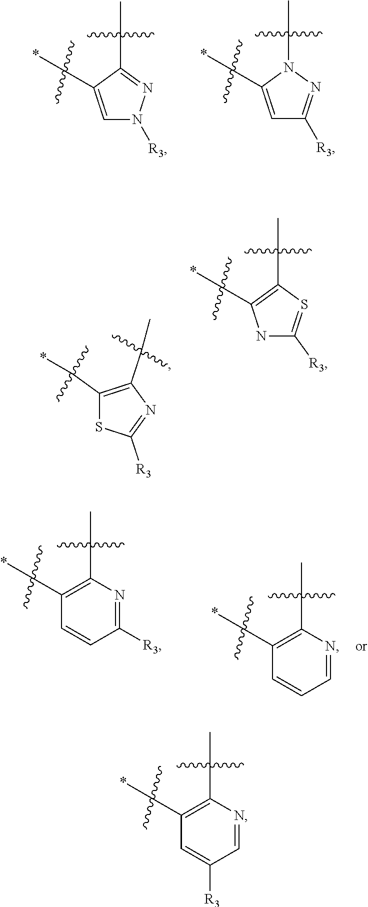
wherein * indicates the point of attachment of Y to the L group bonded to X and Y.

18. The compound of any one of claims 1 to 17, wherein Q is CH.

19. The compound of any one of claims 1 to 17, wherein Q is N.

20. The compound of any one of claims 1 to 19, wherein Z is CR₅.

21. The compound of any one of claims 1 to 20, wherein CR₅ is H.

22. The compound of any one of claims 1 to 21, wherein R₁ is methyl.

23. The compound of any one of claims 1 to 22, wherein R₄ is F.

24. The compound of any one of claims 1 to 23, wherein one R₂ is 5-membered heteroaryl substituted with 1, 2, or 3 occurrences of C₁₋₄ alkyl.

25. The compound of any one of claims 1 to 23, wherein each R₂ is independently selected from the group consisting of CN, —CH₂-cyclopropyl, —CH₂CH₂OCH₃, —CO₂Et, methyl, ethyl, —C(═O)—N(CH₃)₂, —C(═O)—N(CH₃)ⁱPr, —C(═O)—N(CH₃)Et, —C(═O)—NH₂, —C(═O)—N(CH₃)(CH₂CF₃), —C(═O)—N(CH₃)(cyclopropyl), —C(═O)—CH₃, —C(═O)—N(CH₃)—OCH₃, —CH₂—O(CH₂)₂—Si(CH₃)₃, —CH₂-cyclobutyl, CH₂-cyclopropyl, (CH₂)₂—OCH₃, 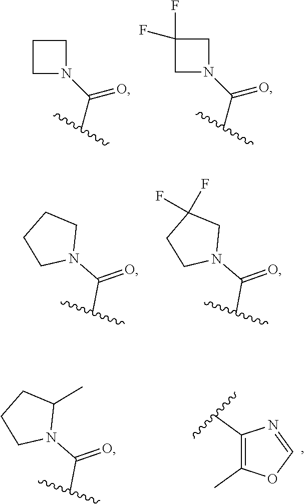
—SO—CH₃, and —SO₂—CH₃.

26. The compound of any one of claims 1 to 23, wherein each R₂ is independently selected from the group consisting of H, fluoro, chloro, CN, methyl, and ethyl.

27. The compound of any one of claims 1 to 26, wherein each R₃ is independently selected from the group consisting of H, fluoro, chloro, CN, methyl, and ethyl.

28. The compound of claim 27, wherein R₃ is methyl.

29. A compound in Table 1, Table 1A, Table 1B, or a pharmaceutically acceptable salt thereof.

30. A pharmaceutical composition, comprising the compound of any one of claims 1 to 29, and a pharmaceutically acceptable carrier or excipient.

31. A method of treating cancer, comprising administering to a subject in need thereof a therapeutically effective amount of a compound of any one of claims 1 to 29 or a pharmaceutical composition of claim 30.

32. The method of claim 31, wherein the subject is a human.

33. The method of claim 31 or 32, wherein the cancer is an ALK positive or ROS1 positive cancer.

34. The method of any one of claims 31 to 33, wherein the compound is an inhibitor of ROS1 and ALK.

35. The method of any one of claims 31 to 33, wherein the compound or salt is an inhibitor of ROS1.

36. The method of any one of claims 31 to 33, wherein the compound is an inhibitor of ALK.

37. The method of any one of claims 31 to 36, wherein the cancer is a solid tumor or a hematologic malignancy.

38. The method of claim 37, wherein the cancer is a solid tumor; and the solid tumor is selected from lung cancer, glioblastoma, inflammatory myofibroblastic tumor (IMT), bile duct cancer, ovarian cancer, gastric cancer, colorectal cancer, angiosarcoma, melanoma, epithelioid hemangioendothelioma, esophageal cancer, kidney cancer, breast cancer, colon cancer, thyroid cancer, spitzoid tumor, and neuroblastoma.

39. The method of claim 37, wherein the cancer is a hematologic malignancy; and the hematologic malignancy is anaplastic large cell lymphoma (ALCL), diffuse large B-cell lymphoma (DLBCL), or large B-cell lymphoma.

40. The method of claim 37, wherein the cancer is non-small cell lung cancer.

41. The method of claim 37, wherein the cancer is inflammatory myofibroblastic tumor.

42. The method of claim 37, wherein the cancer is ovarian cancer.

43. The method of claim 37, wherein the cancer is spitzoid melanoma.

44. The method of claim 37, wherein the cancer is glioblastoma.

45. The method of claim 37, wherein the cancer is cholangiocarcinoma.

46. The method of claim 37, wherein the cancer is gastric cancer.

47. The method of claim 37, wherein the cancer is colorectal cancer.

48. The method of claim 37, wherein the cancer is angiosarcoma.

49. The method of claim 37, wherein the cancer is anaplastic large cell lymphoma.

50. The method of claim 37, wherein the cancer is diffuse large B-cell lymphoma.

51. The method of claim 37, wherein the cancer is esophageal squamous cell carcinoma.

52. The method of claim 37, wherein the cancer is renal medullary carcinoma.

53. The method of claim 37, wherein the cancer is renal cell carcinoma.

54. The method of claim 37, wherein the cancer is breast cancer.

55. The method of claim 37, wherein the cancer is papillary thyroid cancer.

56. The method of claim 37, wherein the cancer is neuroblastoma.

57. The method of any one of claims 31 to 56, wherein the cancer is ROS1 positive.

58. The method of claim 57, wherein the cancer comprises expression of an oncogenic ROS1 gene or oncogenic ROS1 gene-fusion.

59. The method of claim 58, wherein the oncogenic ROS1 gene or oncogenic ROS1 gene-fusion contains one or more mutations of the human ROS1 gene.

60. The method of claim 59, wherein the mutations in the oncogenic ROS1 gene or oncogenic ROS1 gene-fusion results in expression of a ROS1 protein with a G2032R mutation.

61. The method of any one of claims 31 to 56, wherein the cancer is ALK positive.

62. The method of claim 61, wherein the cancer comprises expression of an oncogenic ALK gene or oncogenic ALK gene-fusion.

63. The method of claim 62, wherein the oncogenic ALK gene or the oncogenic ALK gene-fusion contains one or more mutations of the human ALK gene.

64. The method of claim 63, wherein the ALK mutation comprises one or more ALK fusions.

65. The method of claim 64, wherein the ALK fusion is with one of the fusion partners selected from EML4, TMP1, WDCP, GTF2IRD1, TPM3, TPM4, CLTC, LMNA, PRKAR1A, RANBP2, TFG, FN1, KLC1, VCL, STRN, HIP1, NPM1, DCTN1, SQSTM1, TPR, CRIM1, PTPN3, FBXO36, ATIC and KIF5B.

66. The method of claim 65, wherein the ALK fusion is with NPM1, STRN, or EML4.

67. The method of any one of claims 63 to 66, wherein the ALK mutation comprises G1202R, F1174C, F1174L, I1171N, I1171S, I1171T, L1196M, V1180L, C1156Y, G1202del, G1202K, G1269A, F1174S, S1206Y, E1210K, T1151M, T1151_L1152insT, D1203N, S1206C, L1152R, L1196Q, L1198P, L1198F, R1275Q, L1152P, C1156T, or F1245V, or a combination thereof.

68. The method of claim 67, wherein the ALK mutation comprises G1202R.

69. The method of claim 67, wherein the ALK mutation comprises F1174S or F1174L.

70. The method of claim 67, wherein the ALK mutation comprises R1275Q.

71. The method of claim 67, wherein the ALK mutation comprises T1151M.

72. The method of claim 67, wherein the ALK mutation comprises I1171T, I1171S, or I1171N.

73. The method of any one of claims 63 to 72, wherein the ALK mutation comprises one or more compound mutations.

74. The method of claim 73, wherein the compound mutation is selected from G1202R/T1151M, G1202R/L1196M, G1202R/G1269A, G1202R/L1198F, G1202R/F1174S, I1171T/D1203N, I1171T/L1198Y, I1171T/1198F, I1171T/1198I, I1171S/D1203N, I1171S/L1198Y, I1171S/1198F, I1171S/1198I, I1171N/D1203N, I1171N/L1198Y, I1171N/1198F, and I1171N/1198I.

75. The method of claim 61, wherein the cancer is characterized by the presence of a partially deleted ALK protein.

76. The method of any one of claims 31 to 75, wherein the subject has received one prior cancer therapy.

77. The method of any one of claims 31 to 75, wherein the subject has received at least two prior cancer therapies.

78. The method of any one of claims 31 to 77, wherein the compound is an inhibitor of human tropomyosin receptor kinase A, B, or C.

79. The method of claim 78, wherein the IC₅₀ of the compound for inhibition of mutant or non-mutant ROS1 or ALK is no more than one-fifth of the IC₅₀ of the compound for inhibition of wild-type tropomyosin receptor kinase A, B, or C.

80. A method for selectively inhibiting ROS1 over TRK, wherein the inhibition takes place in a subject suffering from cancer, said method comprising administering an effective amount of a compound of any one of claims 1 to 29 or a pharmaceutical composition of claim 30 to said subject.

81. A method for selectively inhibiting ALK over TRK, wherein the inhibition takes place in a subject suffering from cancer, said method comprising administering an effective amount of a compound of any one of claims 1 to 29 or a pharmaceutical composition of claim 30 to said subject.

82. The method of any one of claims 31 to 81, further comprising administering to the subject one or more additional therapeutic agents.

83. The method of claim 82, wherein the additional therapeutic agent is a TKI.

84. The method of claim 83, wherein the TKI is crizotinib, ceritinib, alectinib, brigatinib, lorlatinib, entrectinib, repotrectinib, cabozantinib, foretinib, taletrectinib, merestinib, masitinib, or ensartinib.

85. A method of decreasing a level of ROS1 or ALK in a cell, comprising contacting the cell with a compound of any one of claims 1 to 29 or a pharmaceutical composition of claim 30.

86. The method of claim 85, further comprising contacting the cell with one or more additional therapeutic agents.

87. The method of claim 86, wherein the additional therapeutic agent is a TKI.

88. The method of claim 87, wherein the TKI is crizotinib, ceritinib, alectinib, brigatinib, lorlatinib, entrectinib, repotrectinib, cabozantinib, foretinib, taletrectinib, merestinib, masitinib, or ensartinib.
